(19) United States
(12) Patent Application Publication
(10) Pub. No.: US 2023/0313185 A1
(43) Pub. Date: Oct. 5, 2023

(54) METHODS FOR THE TREATMENT OF NUCLEOTIDE REPEAT EXPANSION DISORDERS ASSOCIATED WITH MSH3 ACTIVITY

(71) Applicant: Triplet Therapeutics, Inc., Cambridge, MA (US)

(72) Inventors: Nessan Anthony BERMINGHAM, Boston, MA (US); Brian R. BETTENCOURT, Groton, MA (US); Peter Edward BIALEK, Littleton, MA (US)

(21) Appl. No.: 17/998,085

(22) PCT Filed: May 7, 2021
(86) PCT No.: PCT/US2021/031428

§ 371 Date: Nov. 7, 2022

Related U.S. Application Data
(60) Provisional application No. 63/022,134, filed on May 8, 2020.

(51) Int. Cl. C12N 15/113; A61P 25/28; A61K 31/713

(52) U.S. Cl. CPC C12N 15/113; A61P 25/28; A61K 31/713; C12N 2310/531; C12N 2310/14

(57) ABSTRACT

The present disclosure features useful compositions and methods to treat repeat expansion disorders (e.g., trinucleotide repeat expansion disorders), in a subject in need thereof. In some aspects, the compositions and methods described herein are useful in the treatment of disorders associated with MSH3 activity.

### CROSS-REFERENCE TO RELATED APPLICATION

This application claims priority to U.S. Provisional Application No. 63/022,134, filed on May 8, 2020, which is incorporated herein by reference in its entirety.

### INCORPORATION BY REFERENCE OF SEQUENCE LISTING

The content of the electronically submitted sequence listing in ASCII text file (Name 4398_026 PC02_Seqlisting_ST25; Size: 611,414 Bytes; and Date of Creation: May 7, 2021) filed with the application is incorporated herein by reference in its entirety.

### BACKGROUND

Nucleotide repeat expansion disorders (e.g., trinucleotide repeat expansion disorders) are genetic disorders caused by nucleotide repeat expansions (e.g., trinucleotide repeats). Nucleotide repeat expansions (e.g., trinucleotide repeat expansions) are a type of genetic mutation where nucleotide repeats in certain genes or introns exceed the normal, stable threshold for that gene. The nucleotide repeats (e.g., trinucleotide repeats) can result in defective or toxic gene products, impair RNA transcription, and/or cause toxic effects by forming toxic mRNA transcripts.

Nucleotide repeat expansion disorders (e.g., trinucleotide repeat expansion disorders) are generally categorized by the type of repeat expansion. For example, Type 1 disorders such as Huntington's disease are caused by CAG repeats which result in a series of glutamine residues known as a polyglutamine tract, Type 2 disorders are caused by heterogeneous expansions that are generally small in magnitude, and Type 3 disorders such as fragile X syndrome are characterized by large repeat expansions that are generally located outside of the protein coding region of the genes. Nucleotide repeat expansion disorders (e.g., trinucleotide repeat expansion disorders) are characterized by a wide variety of symptoms such as progressive degeneration of nerve cells that is common in the Type 1 disorders.

Subjects with a nucleotide repeat expansion disorder (e.g., a trinucleotide repeat expansion disorder) or those who are considered at risk for developing a nucleotide repeat expansion disorder (e.g., a trinucleotide repeat expansion disorder) have a constitutive nucleotide expansion in a gene associated with disease (i.e., the nucleotide repeat expansion is present in the gene during embryogenesis). Constitutive nucleotide repeat expansions (e.g., trinucleotide repeat expansions) can undergo expansion after embryogenesis (i.e., somatic nucleotide repeat expansion). Both constitutive nucleotide repeat expansion and somatic nucleotide repeat expansion can be associated with presence of disease, age at onset of disease, and/or rate of progression of disease.

### BRIEF DESCRIPTION OF DRAWINGS

FIG. 1 shows the dose response curve for dsRNA of SENSE OLIGO NO: 156/ANTISENSE OLIGO NO: 157 tested at 10 nM and 0.5 nM.

FIG. 2 shows the dose response curve for of SENSE OLIGO NO: 906/ANTISENSE OLIGO NO: 907 tested at 10 nM and 0.5 nM.

FIG. 3 shows the dose response curve for dsRNA of SENSE OLIGO NO: 968/ANTISENSE OLIGO NO: 969 tested at 10 nM and 0.5 nM.

FIG. 4 shows the dose response curve for dsRNA of SENSE OLIGO NO: 1392/ANTISENSE OLIGO NO: 1393 tested at 10 nM and 0.5 nM.

FIG. 5 shows the dose response curve for dsRNA of SENSE OLIGO NO: 1874/SEQ ID NO: 1875, tested at 10 nM and 0.5 nM.

FIG. 6 shows the dose response curve for dsRNA of SENSE OLIGO NO: 1366/ANTISENSE OLIGO NO: 1367, tested at 10 nM and 0.5 nM.

FIG. 7 shows the non linear regression curves depicting mean, standard deviation, and RQ values for each the dsRNA shown, at ten concentrations. (See Example 7.)

FIG. 8A shows the dose response curve for dsRNA of SENSE OLIGO NO: 420/ANTISENSE OLIGO NO: 421 at ten concentrations. (See Example 7.)

FIG. 8B shows the dose response curve for dose response curve for dsRNA of SENSE OLIGO NO: 1302/ANTISENSE OLIGO NO: 1303 at ten concentrations. (See Example 7.)

FIG. 8C shows the dose response curve for dsRNA of SENSE OLIGO NO: 550/ANTISENSE OLIGO NO: 551 at ten concentrations. (See Example 7.)

FIG. 8D shows the dose response curve for dsRNA of SENSE OLIGO NO: 672/ANTISENSE OLIGO NO: 673 at ten concentrations. (See Example 7.)

FIGS. 9A-9I show the IC₅₀ analysis for the target knock down measured by qPCR for siRNAs with highest activity in the dual-dose screen. The X-axis represents the concentration of siRNA transfected and the Y-axis represents the relative MSH3 target remaining. (See Example 8.)

FIG. 9A shows the IC₅₀ analysis for the target knock down measured by qPCR for siRNA of SENSE OLIGO NO: 1366/ANTISENSE OLIGO NO: 1367.

FIG. 9B shows the IC₅₀ analysis for the target knock down measured by qPCR for siRNA of SENSE OLIGO NO: 1874/ANTISENSE OLIGO NO: 1875.

FIG. 9C shows the IC₅₀ analysis for the target knock down measured by qPCR for siRNA of SENSE OLIGO NO: 388/ANTISENSE OLIGO NO: 389.

FIG. 9D shows the IC₅₀ analysis for the target knock down measured by qPCR for siRNA of SENSE OLIGO NO: 392/ANTISENSE OLIGO NO: 393.

FIG. 9E shows the IC₅₀ analysis for the target knock down measured by qPCR for siRNA of SENSE OLIGO NO: 402/ANTISENSE OLIGO NO: 403.

FIG. 9F shows the IC₅₀ analysis for the target knock down measured by qPCR for siRNA of SENSE OLIGO NO: 1302/ANTISENSE OLIGO NO: 1303.

FIG. 9G shows the IC₅₀ analysis for the target knock down measured by qPCR for siRNA of SENSE OLIGO NO: 648/ANTISENSE OLIGO NO: 649.

FIG. 9H shows the IC₅₀ analysis for the target knock down measured by qPCR for siRNA of SENSE OLIGO NO: 656/ANTISENSE OLIGO NO: 657.

FIG. 9I shows the IC₅₀ analysis for the target knock down measured by qPCR for siRNA of SENSE OLIGO NO: 832/ANTISENSE OLIGO NO: 833.

FIG. 10 shows the fold change in MSH3 expression relative to a luciferase control from four plates. The X-axis represents the concentration of siRNA transfection on different plates and the Y-axis represents the percentage of target remaining.

### SUMMARY OF THE DISCLOSURE

The present disclosure features useful compositions and methods to treat nucleotide repeat expansion disorders (e.g., trinucleotide repeat expansion disorders) e.g., in a subject in need thereof. In some aspects, the compositions and methods described herein are useful in the treatment of disorders associated with MSH3 activity.

Some aspects of this disclosure are directed to a double-stranded ribonucleic acid (dsRNA), wherein the dsRNA comprises a sense strand and an antisense strand, wherein the antisense strand is complementary to at least 15 contiguous nucleobases of an MSH3 gene, and wherein the dsRNA comprises a duplex structure of between 15 and 30 linked nucleosides in length.

In some aspects, this disclosure is directed to a dsRNA for reducing expression of MSH3 in a cell, wherein the dsRNA comprises a sense strand and an antisense strand, wherein the antisense strand is complementary to at least 15 contiguous nucleobases of an MSH3 gene, and wherein the dsRNA comprises a duplex structure of between 15 and 30 linked nucleosides in length.

In some aspects, the dsRNA comprises a duplex structure of between 19 and 23 linked nucleosides in length.

In some aspects, the dsRNA further comprises a loop region joining the sense strand and antisense strand, wherein the loop region is characterized by a lack of base pairing between nucleobases within the loop region.

In some aspects, the region the sense or antisense strand is complementary to is at least 15 contiguous nucleotides of an MSH3 gene corresponding to a sequence of reference mRNA NM 002439.4 at one or more of positions 566-589, 678-701, 875-989, 1019-1088, 1370-1393, 1466-1569, 1721-1849, 1879-2038, 2086-2171, 2783-2806, 2847-2922, 3043-3119, 3241-3314, 3330-3353, or 3701-3792 of the MSH3 gene.

In some aspects, the region the sense or antisense strand is complementary to is at least 15 contiguous nucleotides of an MSH3 gene corresponding to a sequence of reference mRNA NM 002439.4 at one or more of positions 876-989, 1019-1088, 1370-1393, 1889-1938, or 3241-3314 of the MSH3 gene.

In some aspects, the region the sense or antisense strand is complementary to is at least 15 contiguous nucleotides of an MSH3 gene corresponding to a sequence of reference mRNA NM 002439.4 at one or more of positions 876-989, 1019-1088, 1370-1393, 1466-1569, 1756-1849, 1879-2038, 2086-2171, 2783-2806, 2847-2922, 3043-3119, 3241-3314, 3330-3353, or 3701-3792 of the MSH3 gene.

In some aspects, the region the sense or antisense strand is complementary to is at least 15 contiguous nucleotides of an MSH3 gene corresponding to a sequence of reference mRNA NM 002439.4 at one or more of positions 678-701, 875-989, 1019-1088, 1370-1393, 1466-1569, 1721-1849, 1879-2038, 2086-2171, 2783-2806, 2847-2922, 3043-3119, 3241-3314, 3330-3353, or 3701-3792 of the MSH3 gene.

In some aspects, the region the sense or antisense strand is complementary to is at least 15 contiguous nucleotides of an MSH3 gene corresponding to a sequence of reference mRNA NM 002439.4 at positions 879-921 of the MSH3 gene.

In some aspects, the region the sense or antisense strand is complementary to is at least 15 contiguous nucleotides of an MSH3 gene corresponding to a sequence of reference mRNA NM 002439.4 at one or more of positions 875-989, 1019-1088, 1370-1393, 1466-1569, 1721-1849, 1879-1970, 2783-2806, 2847-2922, 3043-3119, 3241-3314, 3330-3353, or 3703-3792 of the MSH3 gene.

In some aspects, the region the sense or antisense strand is complementary to is at least 15 contiguous nucleotides of an MSH3 gene corresponding to a sequence of reference mRNA NM 002439.4 at one or more of positions 566-589, 678-701, 875-989, 1019-1088, 1370-1393, 1466-1569, 1721-1849, 1879-2038, 2086-2171, 2783-2806, 2847-2922, 3043-3119, 3241-3314, 3330-3353, or 3701-3792 of the MSH3 gene.

In some aspects, the antisense strand comprises an antisense nucleobase sequence selected from Table 3, wherein the 5′ nucleotide represented by U can be any nucleotide (e.g., U, A, C, G), and the sense strand comprises a sense nucleobase sequence complementary to the antisense nucleobase sequence. In some aspects, the antisense nucleobase sequence consists of an antisense strand in Table 3, wherein the 5′ nucleotide represented by U can be any nucleotide (e.g., U, A, C, G), and the sense nucleobase sequence consists of a sequence complementary to the antisense nucleobase sequence. In some aspects, the sense strand comprises a sense nucleobase sequence selected from Table 3, and the antisense strand comprises an antisense nucleobase sequence complementary to the sense nucleobase sequence. In some aspects, the sense nucleobase sequence consists of a sense strand in Table 3, and the antisense nucleobase sequence consists of a sequence complementary to the sense nucleobase sequence.

In some aspects, the sense strand comprises a sense nucleobase sequence selected from Tables 4-10, and the antisense strand comprises an antisense nucleobase sequence complementary to the sense nucleobase sequence. In some aspects, the sense nucleobase sequence consists of a sense strand in any one of Tables 4-10, and the antisense nucleobase sequence consists of a sequence complementary to the sense nucleobase sequence.

In some aspects, the antisense strand comprises an antisense nucleobase sequence selected from Table 11, wherein the 5′ nucleotide represented by U can be any nucleotide (e.g., U, A, C, G), and the sense strand comprises a sense nucleobase sequence complementary to the antisense nucleobase sequence. In some aspects, the antisense nucleobase sequence consists of an antisense strand in Table 11, wherein the 5′ nucleotide represented by U can be any nucleotide (e.g., U, A, C, G), and the sense nucleobase sequence consists of a sequence complementary to the antisense nucleobase sequence In some aspects, the sense strand comprises a sense nucleobase sequence selected from Table 11, and the antisense strand comprises an antisense nucleobase sequence complementary to the sense nucleobase sequence. In some aspects, the sense nucleobase sequence consists of a sense strand in Table 11, and the antisense nucleobase sequence consists of a sequence complementary to the sense nucleobase sequence.

In some aspects, the dsRNA comprises at least one alternative nucleobase, at least one alternative internucleoside linkage, and/or at least one alternative sugar moiety. In some aspects, at least one alternative internucleoside linkage is a phosphorothioate internucleoside linkage. In some aspects, at least one alternative internucleoside linkage is a 2′-alkoxy internucleoside linkage. In some aspects, at least one alternative internucleoside linkage is an alkyl phosphate internucleoside linkage. In some aspects, at least one alternative nucleobase is 5′-methylcytosine, pseudouridine, or 5-methoxyuridine. In some aspects, at least one alternative sugar moiety is 2′-OMe or a bicyclic nucleic acid. In some aspects, the dsRNA comprises at least one 2′-OMe sugar moiety and at least one phosphorothioate internucleoside linkage.

In some aspects, the dsRNA further comprises a ligand conjugated to the 3′ end of the sense strand through a monovalent or branched bivalent or trivalent linker.

In some aspects, the sense strand comprises a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

In some aspects, the sense strand comprises a nucleobase sequence of any one of SEQ ID NOs: 380, 382, 386, 388, 396, 406, 418, 464, 478, 520, 540, 564, 568, 750, 1166, 1168, 1170, 1182, 1192, 1212, 1214, 1216, 1222, 2264, 2290, 2308, or 2318.

In some aspects, the sense strand comprises a nucleobase sequence of any one of SEQ ID NOs: 380, 382, 386, 388, 396, 406, 418, 464, 478, 520, 540, 564, 568, 750, 822, 830, 844, 868, 870, 874, 904, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

In some aspects, the sense strand comprises a nucleobase sequence of any one of SEQ ID NOs: 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

In some aspects, the sense strand comprises a nucleobase sequence of any one of SEQ ID NOs: 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

In some aspects, the sense strand comprises a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

In some aspects, the antisense strand comprises a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

In some aspects, the antisense strand comprises a nucleobase sequence of any one of SEQ ID NOs: 381, 383, 387, 389, 397, 407, 419, 465, 479, 521, 541, 565, 569, 751, 1167, 1169, 1171, 1183, 1193, 1213, 1215, 1217, 1223, 2265, 2291, 2309, or 2319, wherein the 5′ nucleotide represented by U can be any nucleotide (e.g., U, A, C, G).

In some aspects, the antisense strand comprises a nucleobase sequence of any one of SEQ ID NOs: 381, 383, 387, 389, 397, 407, 419, 465, 479, 521, 541, 565, 569, 751, 823, 831, 845, 869, 871, 875, 905, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

In some aspects, the antisense strand comprises a nucleobase sequence of any one of SEQ ID NOs: 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

In some aspects, the antisense strand comprises a nucleobase sequence of any one of SEQ ID NOs: 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

In some aspects, the antisense strand comprises a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

In some aspects, the sense strand consists of a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

In some aspects, the sense strand consists of a nucleobase sequence of any one of SEQ ID NOs: 380, 382, 386, 388, 396, 406, 418, 464, 478, 520, 540, 564, 568, 750, 1166, 1168, 1170, 1182, 1192, 1212, 1214, 1216, 1222, 2264, 2290, 2308, or 2318.

In some aspects, the sense strand consists of a nucleobase sequence of any one of SEQ ID NOs: 380, 382, 386, 388, 396, 406, 418, 464, 478, 520, 540, 564, 568, 750, 822, 830, 844, 868, 870, 874, 904, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

In some aspects, the sense strand consists of a nucleobase sequence of any one of SEQ ID NOs: 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

In some aspects, the sense strand consists of a nucleobase sequence of any one of SEQ ID NOs: 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

In some aspects, the sense strand consists of a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

In some aspects, the sense strand consists of a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691.

In some aspects, the antisense strand consists of a nucleobase sequence of any one of SEQ ID NOs: 381, 383, 387, 389, 397, 407, 419, 465, 479, 521, 541, 565, 569, 751, 1167, 1169, 1171, 1183, 1193, 1213, 1215, 1217, 1223, 2265, 2291, 2309, or 2319, wherein the 5′ nucleotide represented by U can be any nucleotide (e.g., U, A, C, G).

In some aspects, the antisense strand consists of a nucleobase sequence of any one of SEQ ID NOs: 381, 383, 387, 389, 397, 407, 419, 465, 479, 521, 541, 565, 569, 751, 823, 831, 845, 869, 871, 875, 905, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

In some aspects, the antisense strand consists of a nucleobase sequence of any one of SEQ ID NOs: 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

In some aspects, the antisense strand consists of a nucleobase sequence of any one of SEQ ID NOs: 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

In some aspects, the antisense strand consists of a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

In some aspects, the dsRNA exhibits at least 50% mRNA inhibition at a 0.5 nM dsRNA concentration when determined using a cell assay when compared with a control cell. In some aspects, the dsRNA exhibits at least 40% mRNA inhibition at a 0.5 nM dsRNA concentration when determined using a cell assay when compared with a control cell. In some aspects, the dsRNA exhibits at least 30% mRNA inhibition at a 0.5 nM dsRNA concentration when determined using a cell assay when compared with a control cell. In some aspects, the dsRNA exhibits at least 70% mRNA inhibition at a 10 nM dsRNA concentration when determined using a cell assay when compared with a control cell. In some aspects, the dsRNA exhibits at least 60% mRNA inhibition at a 10 nM dsRNA concentration when determined using a cell assay when compared with a control cell. In some aspects, the dsRNA exhibits at least 50% mRNA inhibition at a 10 nM dsRNA concentration when determined using a cell assay when compared with a control cell.

In some aspects, the antisense strand is complementary to at least 17 contiguous nucleotides of an MSH3 gene. In some aspects, the antisense strand is complementary to at least 19 contiguous nucleotides of an MSH3 gene. In some aspects, the antisense strand is complementary to 19 contiguous nucleotides of an MSH3 gene. In some aspects, the sense strand is complementary to at least 17 contiguous nucleotides of an MSH3 gene. In some aspects, the sense strand is complementary to at least 19 contiguous nucleotides of an MSH3 gene. In some aspects, the sense strand is complementary to 19 contiguous nucleotides of an MSH3 gene.

In some aspects, the antisense strand and/or the sense strand comprises a 3′ overhang of at least 1 linked nucleoside; or a 3′ overhang of at least 2 linked nucleosides.

In some aspects, this disclosure is directed to a pharmaceutical composition comprising one or more of the dsRNAs described herein and a pharmaceutically acceptable carrier.

In some aspects, this disclosure is directed to a composition comprising one or more of the dsRNAs described herein and a lipid nanoparticle, a polyplex nanoparticle, a lipoplex nanoparticle, or a liposome.

In some aspects, this disclosure is directed to a vector encoding at least one strand of any one of the dsRNAs described herein.

In some aspects, this disclosure is directed to a cell comprising the vector encoding at least one strand of any one of the dsRNAs described herein.

In some aspects, this disclosure is directed to a method of reducing transcription of MSH3 in a cell, the method comprising contacting the cell with one or more of the dsRNAs described herein, a pharmaceutical composition of one or more the dsRNAs described herein, a composition of one or more of the dsRNAs described herein and a lipid nanoparticle, a polyplex nanoparticle, a lipoplex nanoparticle, or a liposome, the vector encoding at least one strand of any one of the dsRNAs described herein, or the cell comprising the vector encoding at least one strand of any one of the dsRNAs described herein, for a time sufficient to obtain degradation of an mRNA transcript of MSH3, thereby reducing expression of MSH3 in the cell.

In some aspects, this disclosure is directed to a method of treating, preventing, or delaying progression of a trinucleotide repeat expansion disorder in a subject in need thereof, the method comprising contacting the cell with one or more of the dsRNAs described herein, a pharmaceutical composition of one or more the dsRNAs described herein, a composition of one or more of the dsRNAs described herein and a lipid nanoparticle, a polyplex nanoparticle, a lipoplex nanoparticle, or a liposome, the vector encoding at least one strand of any one of the dsRNAs described herein, or the cell comprising the vector encoding at least one strand of any one of the dsRNAs described herein.

In some aspects, this disclosure is directed to a method of reducing the level and/or activity of MSH3 in a cell of a subject identified as having a trinucleotide repeat expansion disorder, the method comprising contacting the cell with one or more of the dsRNAs described herein, a pharmaceutical composition of one or more the dsRNAs described herein, a composition of one or more of the dsRNAs described herein and a lipid nanoparticle, a polyplex nanoparticle, a lipoplex nanoparticle, or a liposome, the vector encoding at least one strand of any one of the dsRNAs described herein, or the cell comprising the vector encoding at least one strand of any one of the dsRNAs described herein.

In some aspects, this disclosure is directed to a method for reducing expression of MSH3 in a cell the method comprising contacting the cell with one or more of the dsRNAs described herein, a pharmaceutical composition of one or more the dsRNAs described herein, a composition of one or more of the dsRNAs described herein and a lipid nanoparticle, a polyplex nanoparticle, a lipoplex nanoparticle, or a liposome, the vector encoding at least one strand of any one of the dsRNAs described herein, or the cell comprising the vector encoding at least one strand of any one of the dsRNAs described herein, thereby reducing expression of MSH3 in the cell.

In some aspects, this disclosure is directed to a method of decreasing nucleotide repeat expansion (e.g., trinucleotide repeat expansion) in a cell, the method comprising contacting the cell with one or more of the dsRNAs described herein, a pharmaceutical composition of one or more the dsRNAs described herein, a composition of one or more of the dsRNAs described herein and a lipid nanoparticle, a polyplex nanoparticle, a lipoplex nanoparticle, or a liposome, the vector encoding at least one strand of any one of the dsRNAs described herein, or the cell comprising the vector encoding at least one strand of any one of the dsRNAs described herein.

In some aspects, the cell is in a subject. In some aspects, the subject is a human. In some aspects, the cell is a cell of the central nervous system or a muscle cell.

In some aspects, the subject is identified as having a nucleotide repeat expansion disorder (e.g., a trinucleotide repeat expansion). In some aspects, the nucleotide repeat expansion disorder (e.g., a trinucleotide repeat expansion) is a polyglutamine disease. In some aspects, the polyglutamine disease is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, and Huntington's disease-like 2. In some aspects, the nucleotide repeat expansion disorder (e.g., a trinucleotide repeat expansion disorder) is Huntington's disease.

In some aspects, the inucleotide repeat expansion disorder (e.g., a trinucleotide repeat expansion disorder) is a non-polyglutamine disease. In some aspects, the non-polyglutamine disease is selected from the group consisting of fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy. In some aspects, the nucleotide repeat expansion disorder (e.g., a trinucleotide repeat expansion disorder) is Friedreich's ataxia. In some aspects, the nucleotide repeat expansion disorder (e.g., trinucleotide repeat expansion disorder) is myotonic dystrophy type 1.

In some aspects, this disclosure is directed to one or more of the dsRNAs described herein, a pharmaceutical composition of one or more the dsRNAs described herein, a composition of one or more of the dsRNAs described herein and a lipid nanoparticle, a polyplex nanoparticle, a lipoplex nanoparticle, or a liposome, the vector encoding at least one strand of any one of the dsRNAs described herein, or the cell comprising the vector encoding at least one strand of any one of the dsRNAs described herein for use in prevention or treatment of a nucleotide repeat expansion disorder (e.g., a trinucleotide repeat expansion disorder).

In some aspects, the one or more of the dsRNAs described herein, the pharmaceutical composition of one or more the dsRNAs described herein, the composition of one or more of the dsRNAs described herein and a lipid nanoparticle, a polyplex nanoparticle, a lipoplex nanoparticle, or a liposome, the vector encoding at least one strand of any one of the dsRNAs described herein, or the cell comprising the vector encoding at least one strand of any one of the dsRNAs described herein is administered intrathecally.

In some aspects, the one or more of the dsRNAs described herein, the pharmaceutical composition of one or more the dsRNAs described herein, the composition of one or more of the dsRNAs described herein and a lipid nanoparticle, a polyplex nanoparticle, a lipoplex nanoparticle, or a liposome, the vector encoding at least one strand of any one of the dsRNAs described herein, or the cell comprising the vector encoding at least one strand of any one of the dsRNAs described herein is administered intraventricularly.

In some aspects, the one or more of the dsRNAs described herein, the pharmaceutical composition of one or more the dsRNAs described herein, the composition of one or more of the dsRNAs described herein and a lipid nanoparticle, a polyplex nanoparticle, a lipoplex nanoparticle, or a liposome, the vector encoding at least one strand of any one of the dsRNAs described herein, or the cell comprising the vector encoding at least one strand of any one of the dsRNAs described herein is administered intramuscularly.

In some aspects, this disclosure is directed to a method of treating, preventing, or delaying progression of a disorder in a subject in need thereof wherein the subject is suffering from a nucleotide repeat expansion disorder (e.g., a trinucleotide repeat expansion disorder), comprising administering to said subject one or more of the dsRNAs described herein, the pharmaceutical composition of one or more the dsRNAs described herein, the composition of one or more of the dsRNAs described herein and a lipid nanoparticle, a polyplex nanoparticle, a lipoplex nanoparticle, or a liposome, the vector encoding at least one strand of any one of the dsRNAs described herein, or the cell comprising the vector encoding at least one strand of any one of the dsRNAs described herein.

In some aspects, the method of treating, preventing, or delaying progression of a disorder in a subject further comprises administering at least one additional therapeutic agent. In some aspects, the at least one additional therapeutic agent is another oligonucleotide, or pharmaceutically acceptable salt thereof, that hybridizes to an mRNA encoding the Huntingtin gene.

In some aspects, the method of treating, preventing, or delaying progression of a disorder in a subject delays progression of the nucleotide repeat expansion disorder (e.g., a trinucleotide repeat expansion disorder) by at least 120 days, for example, at least 6 months, at least 12 months, at least 2 years, at least 3 years, at least 4 years, at least 5 years, at least 10 years or more, when compared with a predicted progression.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular aspects, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

In this application, unless otherwise clear from context, (i) the term “a” can be understood to mean “at least one”; (ii) the term “or” can be understood to mean “and/or”; and (iii) the terms “including” and “comprising” can be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, and 5.18% without consideration of the number of significant figures.

As used herein, “no more than” or “less than” is understood as the value adjacent to the phrase and logical lower values or integers, as logical from context, to zero. For example, a duplex with an overhang of “no more than two linked nucleosides” has a 2, 1, or 0 linked nucleoside overhang. When “no more than” is present before a series of numbers or a range, it is understood that “no more than” can modify each of the numbers in the series or range.

As used herein, the term “administration” refers to the administration of a composition (e.g., a compound or a preparation that includes a compound as described herein) to a subject or system. Administration to an animal subject (e.g., to a human) can be by any appropriate route, such as one described herein.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some aspects, the delivery of the two or more agents is simultaneous or concurrent and the agents can be co-formulated. In some aspects, the two or more agents are not co-formulated and are administered in a sequential manner as part of a prescribed regimen. In some aspects, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intraocular routes, subcutaneous routes, intra cisterna magna routes, intravenous routes, intramuscular routes, intracerebroventricular routes, intrathecal routes, and direct absorption through mucous membrane tissues. The therapeutic agents can be administered by the same route or by different routes. For example, one therapeutic agent of the combination can be administered by intravenous injection while an additional therapeutic agent of the combination can be administered orally.

As used herein, the term “MSH3” refers to MutS Homolog 3, a DNA mismatch repair protein, having an amino acid sequence from any vertebrate or mammalian source, including, but not limited to, human, bovine, chicken, rodent, mouse, rat, porcine, ovine, primate, monkey, and guinea pig, unless specified otherwise. The term also refers to fragments and variants of native MSH3 that maintain at least one in vivo or in vitro activity of a native MSH3. The term encompasses full-length unprocessed precursor forms of MSH3 as well as mature forms resulting from post-translational cleavage of the signal peptide. MSH3 is encoded by the MSH3 gene. The nucleic acid sequence of an exemplary Homo sapiens (human) MSH3 gene is set forth in NCBI Reference NM_002439.4 or in SEQ ID NO: 1. The term “MSH3” also refers to natural variants of the wild-type MSH3 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type human MSH3, which is set forth in NCBI Reference No. NP_002430.3 or in SEQ ID NO: 2. The nucleic acid sequence of an exemplary Mus musculus (mouse) MSH3 gene is set forth in NCBI Reference No. NM_010829.2 or in SEQ ID NO: 3. The nucleic acid sequence of an exemplary Rattus norvegicus (rat) MSH3 gene is set forth in NCBI Reference No. NM_001191957.1 or in SEQ ID NO: 4. The nucleic acid sequence of an exemplary Macaca fascicularis (cyno) MSH3 gene is set forth in NCBI Reference No. XM_005557283.2 or in SEQ ID NO: 5.

The term “MSH3” as used herein also refers to a particular polypeptide expressed in a cell by naturally occurring DNA sequence variations of the MSH3 gene, such as a single nucleotide polymorphism in the MSH3 gene. Numerous SNPs within the MSH3 gene have been identified and can be found at, for example, NCBI dbSNP (see, e.g., www.ncbi.nlm.nih.gov/snp). Non-limiting examples of SNPs within the MSH3 gene can be found at, NCBI dbSNP Accession Nos.: rs1650697, rs70991108, rs10168, rs26279, rs26282, rs26779, rs26784, rs32989, rs33003, rs33008, rs33013, rs40139, rs181747, rs184967, rs245346, rs245397, rs249633, rs380691, rs408626, rs442767, rs836802, rs836808, rs863221, rs1105525, rs1428030, rs1478834, rs1650694, rs1650737, rs1677626, rs1677658, rs1805355, rs2897298, rs3045983, rs3797897, rs4703819, rs6151627, rs6151640, rs6151662, rs6151670, rs6151735, rs6151838, rs7709909, rs7712332, rs10079641, rs12513549, and rs12522132.

As used herein, “target sequence” refers to a contiguous portion of the nucleotide sequence of an mRNA molecule formed during the transcription of an MSH3 gene, including mRNA that is a product of RNA processing of a primary transcription product. In one aspect, the target portion of the sequence will be at least long enough to serve as a substrate for dsRNA-directed cleavage at or near that portion of the nucleotide sequence of an mRNA molecule formed during the transcription of a MSH3 gene. The target sequence can be, for example, from about 9-36 nucleotides in length, e.g., about 15-30 nucleotides in length. For example, the target sequence can be 15, 16, 17, 18, 19, 20, 21, 22, 23, 24, 25, 26, 27, 28, 29, or 30 or from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated.

“G,” “C,” “A,” “T,” and “U” each generally stand for a naturally-occurring nucleotide that contains guanine, cytosine, adenine, thymidine, and uracil as a base, respectively. However, it will be understood that the term “nucleotide” can refer to an alternative nucleotide, as further detailed below, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured herein by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured herein.

The terms “nucleobase” and “base” include the purine (e.g. adenine and guanine) and pyrimidine (e.g. uracil, thymine, and cytosine) moiety present in nucleosides and nucleotides which form hydrogen bonds in nucleic acid hybridization. The term nucleobase also encompasses alternative nucleobases which can differ from naturally-occurring nucleobases, but are functional during nucleic acid hybridization. In this context “nucleobase” refers to both naturally occurring nucleobases such as adenine, guanine, cytosine, thymidine, uracil, xanthine, and hypoxanthine, as well as alternative nucleobases. Such variants are for example described in Hirao et al (2012) Accounts of Chemical Research vol 45 page 2055 and Bergstrom (2009) Current Protocols in Nucleic Acid Chemistry Suppl. 37 1.4.1.

The term “nucleoside” refers to a monomeric unit of an oligonucleotide or a polynucleotide having a nucleobase and a sugar moiety. A nucleoside can include those that are naturally-occurring as well as alternative nucleosides, such as those described herein. The nucleobase of a nucleoside can be a naturally-occurring nucleobase or an alternative nucleobase. Similarly, the sugar moiety of a nucleoside can be a naturally-occurring sugar or an alternative sugar.

The term “alternative nucleoside” refers to a nucleoside having an alternative sugar or an alternative nucleobase, such as those described herein.

In some aspects, the nucleobase moiety is modified by changing the purine or pyrimidine into a modified purine or pyrimidine, such as substituted purine or substituted pyrimidine, such as an “alternative nucleobase” selected from isocytosine, pseudoisocytosine, 5-methyl cytosine, 5-thiozolo-cytosine, 5-propynyl-cytosine, 5-propynyl-uridine, 5-bromouridine 5-thiazolo-uridine, 2-thio-uridine, pseudouridine, 1-methylpseudouridine, 5-methoxyuridine, 2′-thio-thymine, inosine, diaminopurine, 6-aminopurine, 2-aminopurine, 2,6-diaminopurine, and 2-chloro-6-aminopurine.

The nucleobase moieties can be indicated by the letter code for each corresponding nucleobase, e.g. A, T, G, C, or U, wherein each letter can include alternative nucleobases of equivalent function.

A “sugar” or “sugar moiety,” includes naturally occurring sugars having a furanose ring. A sugar also includes an “alternative sugar,” defined as a structure that is capable of replacing the furanose ring of a nucleoside. In some aspects, alternative sugars are non-furanose (or 4′-substituted furanose) rings or ring systems or open systems. Such structures include simple changes relative to the natural furanose ring, such as a six-membered ring, or can be more complicated as is the case with the non-ring system used in peptide nucleic acid. Alternative sugars can include sugar surrogates wherein the furanose ring has been replaced with another ring system such as, for example, a morpholino or hexitol ring system. Sugar moieties useful in the preparation of oligonucleotides having motifs include, without limitation, β-D-ribose, β-D-2′-deoxyribose, substituted sugars (such as 2′, 5′ and bis substituted sugars), 4′-S-sugars (such as 4′-S-ribose, 4′-S-2′-deoxyribose and 4′-S-2′-substituted ribose), bicyclic alternative sugars (such as the 2′-O—CH₂-4′ or 2′-O—(CH₂)₂-4′ bridged ribose derived bicyclic sugars) and sugar surrogates (such as when the ribose ring has been replaced with a morpholino or a hexitol ring system). The type of heterocyclic base and internucleoside linkage used at each position is variable and is not a factor in determining the motif. In most nucleosides having an alternative sugar moiety, the heterocyclic nucleobase is generally maintained to permit hybridization.

A “nucleotide,” as used herein, refers to a monomeric unit of an oligonucleotide or polynucleotide that comprises a nucleoside and an internucleosidic linkage. The internucleosidic linkage can include a phosphate linkage. Similarly, “linked nucleosides” can be linked by phosphate linkages. Many “alternative internucleosidic linkages” are known in the art, including, but not limited to, phosphate, phosphorothioate, and boronophosphate linkages. Alternative nucleosides include bicyclic nucleosides (BNAs) (e.g., locked nucleosides (LNAs (e.g., A-LNA, 5mC L-NA, G-LNA, T-LNA)) and constrained ethyl (cEt) nucleosides), peptide nucleosides (PNAs), phosphotriesters, phosphorothionates, phosphoramidates, and other variants of the phosphate backbone of native nucleoside, including those described herein.

An “alternative nucleotide,” as used herein, refers to a nucleotide having an alternative nucleoside or an alternative sugar, and an internucleoside linkage, which can include alternative nucleoside linkages.

The terms “oligonucleotide” and “polynucleotide,” as used herein, are defined as it is generally understood by the skilled person as a molecule comprising two or more covalently linked nucleosides. Such covalently bound nucleosides can be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide can be man-made. For example, the oligonucleotide can be chemically synthesized and be purified or isolated. Oligonucleotide is also intended to include (i) compounds that have one or more furanose moieties that are replaced by furanose derivatives or by any structure, cyclic or acyclic, that can be used as a point of covalent attachment for the base moiety, (ii) compounds that have one or more phosphodiester linkages that are either modified, as in the case of phosphoramidate or phosphorothioate linkages, or completely replaced by a suitable linking moiety as in the case of formacetal or riboacetal linkages, and/or (iii) compounds that have one or more linked furanose-phosphodiester linkage moieties replaced by any structure, cyclic or acyclic, that can be used as a point of covalent attachment for the base moiety. The oligonucleotides can comprise one or more alternative nucleosides or nucleotides (e.g., including those described herein). It is also understood that oligonucleotide includes compositions lacking a sugar moiety or nucleobase but is still capable of forming a pairing with or hybridizing to a target sequence.

“Oligonucleotide” refers to a short polynucleotide (e.g., of 100 or fewer linked nucleosides).

As used herein, the term “strand” refers to an oligonucleotide comprising a chain of linked nucleosides. A “strand comprising a nucleobase sequence” refers to an oligonucleotide comprising a chain of linked nucleosides that is described by the sequence referred to using the standard nucleobase nomenclature.

The term “antisense,” as used herein, refers to a nucleic acid comprising an oligonucleotide or polynucleotide that is sufficiently complementary to all or a portion of a gene, primary transcript, or processed mRNA, so as to interfere with expression of the endogenous gene (e.g., MSH3). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides can hybridize to each other even if they are not completely complementary to each other, provided that each has at least one region that is substantially complementary to the other.

The terms “antisense strand” and “guide strand” refer to the strand of a dsRNA that includes a region that is substantially complementary to a target sequence, e.g., an MSH3 mRNA.

The terms “sense strand” and “passenger strand,” as used herein, refer to the strand of a dsRNA that includes a region that is substantially complementary to a region of the antisense strand as that term is defined herein.

The term “dsRNA” refers to an agent that includes a sense strand and antisense strand that contains linked nucleosides as that term is defined herein. dsRNA includes, for example, siRNAs and shRNAs, which mediate the targeted cleavage of an RNA transcript via an RNA-induced silencing complex (RISC) pathway. dsRNA directs the sequence-specific degradation of mRNA through a process known as RNA interference (RNAi). The dsRNA reduces the expression of MSH3 in a cell, e.g., a cell within a subject, such as a mammalian subject. In general, the majority of linked nucleosides of each strand of a dsRNA are ribonucleosides, but as described in detail herein, each or both strands can include one or more non-ribonucleosides, e.g., deoxyribonucleosides and/or alternative nucleosides.

The terms “siRNA” and “short interfering RNA” (also known as “small interfering RNA”) refer to an RNA agent, such as a double-stranded agent, of about 10-50 nucleotides in length, the strands optionally having overhanging ends comprising, for example 1, 2, or 3 overhanging linked nucleosides, which is capable of directing or mediating RNA interference. Naturally-occurring siRNAs are generated from longer dsRNA molecules (e.g., >25 linked nucleosides in length) by a cell's RNAi machinery (e.g., Dicer or a homolog thereof).

The terms “shRNA” and “short hairpin RNA,” as used herein, refer to an RNA agent having a stem-loop structure, comprising at least two regions of complementary sequence, the degree of complementarity and orientation of the regions being sufficient such that base pairing occurs between the regions, at least two of the regions being joined by a loop region which results from a lack of base pairing between nucleobases within the loop region.

“Chimeric” dsRNA or “chimera” is a dsRNA which contains two or more chemically distinct regions, each made up of at least one monomer unit, i.e., a nucleoside or nucleotide.

The duplex region can be of any length that permits specific degradation of a desired target RNA through a RISC pathway, and can range from about 9 to 36 base pairs in length, e.g., about 10-30 base pairs in length, e.g., about 15-30 base pairs in length or about 18-20 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated.

The two strands forming the duplex structure can be different portions of one longer oligonucleotide molecule, or they can be separate oligonucleotide molecules. Where the two strands are part of one larger molecule, and therefore are connected by an uninterrupted chain of linked nucleosides between the 3′-end of one strand and the 5′-end of the respective other strand forming the duplex structure, the connecting chain is referred to as a “hairpin loop.” A hairpin loop can comprise at least one unpaired nucleobase. In some aspects, the hairpin loop can comprise at least 2, at least 3, at least 4, at least 5, at least 6, at least 7, at least 8, at least 9, at least 10, at least 20, at least 23 or more unpaired nucleobases. In some aspects, the hairpin loop can be 10 or fewer linked nucleosides. In some aspects, the hairpin loop can be 8 or fewer unpaired nucleobases. In some aspects, the hairpin loop can be 4-10 unpaired nucleobases. In some aspects, the hairpin loop can be 4-8 linked nucleosides.

Multiple dsRNAs can be joined together by a linker. The linker can be cleavable or non-cleavable. The dsRNAs can be the same or different.

In one aspect, each strand of the dsRNA includes 19-23 linked nucleosides that interacts with a target RNA sequence, e.g., an MSH3 target mRNA sequence, to direct the cleavage of the target RNA. Without wishing to be bound by theory, long double stranded RNA introduced into cells is broken down by a Type III endonuclease known as Dicer (Sharp et al. (2001) Genes Dev. 15:485). Dicer, a ribonuclease-III-like enzyme, processes the RNA into 19-23 base pair short interfering RNAs with characteristic two-base 3′ overhangs (Bernstein, et al., (2001) Nature 409:363). The dsRNAs are then incorporated into an RNA-induced silencing complex (RISC) where one or more helicases unwind the dsRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Where the two substantially complementary strands of a dsRNA are comprised of separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.”

“Linker” or “linking group” means an organic moiety that connects two parts of a compound, e.g., covalently attaches two parts of a compound. The RNA strands can have the same or a different number of linked nucleosides. The maximum number of base pairs is the number of linked nucleosides in the shortest strand of the dsRNA minus any overhangs that are present in the duplex. In addition to the duplex structure, a dsRNA can comprise one or more nucleoside overhangs. In one aspect of the dsRNA, at least one strand comprises a 3′ overhang of at least 1 nucleoside. In another aspect, at least one strand comprises a 3′ overhang of at least 2 linked nucleosides, e.g., 2, 3, 4, 5, 6, 7, 9, 10, 11, 12, 13, 14, or 15 linked nucleosides. In other aspects, at least one strand of the dsRNA comprises a 5′ overhang of at least 1 nucleoside. In some aspects, at least one strand comprises a 5′ overhang of at least 2 linked nucleosides, e.g., 2, 3, 4, 5, 6, 7, 9, 10, 11, 12, 13, 14, or 15 linked nucleosides. In still other aspects, both the 3′ and the 5′ end of one strand of the dsRNA comprise an overhang of at least 1 nucleoside.

A linker or linking group is a connection between two atoms that links one chemical group or segment of interest to another chemical group or segment of interest via one or more covalent bonds. Conjugate moieties can be attached to the dsRNA directly or through a linking moiety (e.g. linker or tether). Linkers serve to covalently connect a third region, e.g. a conjugate moiety to a dsRNA (e.g. the termini of region A or C). In some aspects, the conjugate or dsRNA conjugate can comprise a linker region which is positioned between the dsRNA and the conjugate moiety. In some aspects, the linker between the conjugate and dsRNA is biocleavable. Phosphodiester containing biocleavable linkers are described in more detail in WO 2014/076195 (herein incorporated by reference).

As used herein, the term “nucleoside overhang” refers to at least one unpaired nucleobase that protrudes from the duplex structure of a dsRNA. For example, when a 3′-end of one strand of a dsRNA extends beyond the 5′-end of the other strand, or vice versa, there is a nucleoside overhang. A dsRNA can comprise an overhang of at least one nucleoside; alternatively, the overhang can comprise at least two nucleosides, at least three nucleosides, at least four nucleosides, at least five nucleosides or more. A nucleoside overhang can comprise or consist of an alternative nucleoside, including a deoxynucleotide/nucleoside. A nucleoside overhang can comprise or consist of one or more phosphorothioates bonds. The overhang(s) can be on the sense strand, the antisense strand, or any combination thereof. Furthermore, the nucleoside(s) of an overhang can be present on the 5′-end, 3′-end or both ends of either an antisense or sense strand of a dsRNA. In some aspects, the overhang includes a self-complementary portion such that the overhang is capable of forming a hairpin structure that is stable under physiological conditions.

The terms “blunt” and “blunt ended” mean that there are no unpaired nucleobases at a given terminal end of a dsRNA, i.e., no nucleoside overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleoside overhang at either end of the molecule. Most often, such a molecule will be double stranded over its entire length. As used herein, the term “cleavage region” refers to a region that is located immediately adjacent to the cleavage site. The cleavage site is the site on the target at which cleavage occurs. In some aspects, the cleavage region comprises three bases on either end of, and immediately adjacent to, the cleavage site. In some aspects, the cleavage region comprises two bases on either end of, and immediately adjacent to, the cleavage site. In some aspects, the cleavage site specifically occurs at the site bound by nucleosides 10 and 11 of the antisense strand, and the cleavage region comprises nucleosides 11, 12, and 13.

The term “contiguous nucleobase region” refers to the region of the dsRNA (e.g., the antisense strand of the dsRNA) which is complementary to the target nucleic acid. The term can be used interchangeably herein with the term “contiguous nucleotide sequence” or “contiguous nucleobase sequence.” In some aspects, all the nucleotides of the dsRNA are present in the contiguous nucleotide or nucleoside region. In some aspects, the dsRNA comprises the contiguous nucleotide region and can comprise further nucleotide(s) or nucleoside(s), for example a nucleotide linker region which can be used to attach a functional group to the contiguous nucleotide sequence. The nucleotide linker region can be complementary to the target nucleic acid. In some aspects, the internucleoside linkages present between the nucleotides of the contiguous nucleotide region are all phosphorothioate internucleoside linkages. In some aspects, the contiguous nucleotide region comprises one or more sugar-modified nucleosides.

As used herein, and unless otherwise indicated, the term “complementary,” when used to describe a first nucleotide or nucleoside sequence in relation to a second nucleotide or nucleoside sequence, refers to the ability of an oligonucleotide or polynucleotide comprising the first nucleotide or nucleoside sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can be used. The skilled person will be able to determine the set of conditions most appropriate for a test of complementarity of two sequences in accordance with the ultimate application of the hybridized nucleotides or nucleosides.

“Complementary” sequences, as used herein, can include, or be formed entirely from, non-Watson-Crick base pairs and/or base pairs formed from non-natural and alternative nucleotides or nucleosides, in so far as the above requirements with respect to their ability to hybridize are fulfilled. Such non-Watson-Crick base pairs include, but are not limited to, G:U Wobble or Hoogstein base pairing. Complementary sequences within a dsRNA, or between an oligonucleotide and a target sequence as described herein, include base-pairing of the oligonucleotide or polynucleotide comprising a first nucleotide or nucleoside sequence to an oligonucleotide or polynucleotide comprising a second nucleotide or nucleoside sequence over the entire length of one or both nucleotide or nucleoside sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. “Substantially complementary” can refer to a polynucleotide that is substantially complementary to a contiguous portion of the mRNA of interest (e.g., an mRNA encoding MSH3). For example, a polynucleotide is complementary to at least a part of an MSH3 mRNA if the sequence is substantially complementary to a non-interrupted portion of an mRNA encoding MSH3. However, where two oligonucleotides are designed to form, upon hybridization, one or more single stranded overhangs, such overhangs shall not be regarded as mismatches with regard to the determination of complementarity. For example, a dsRNA comprising one oligonucleotide of 21 linked nucleosides in length and another oligonucleotide of 23 nucleosides in length, wherein the longer oligonucleotide comprises a sequence of 21 linked nucleosides that is fully complementary to the shorter oligonucleotide, can be referred to as “fully complementary” for the purposes described herein.

As used herein, the term “region of complementarity” refers to the region on the oligonucleotide that is substantially complementary to all or a portion of a gene, primary transcript, a sequence (e.g., a target sequence, e.g., an MSH3 nucleotide sequence), or processed mRNA, so as to interfere with expression of the endogenous gene (e.g., MSH3). Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the oligonucleotide.

As used herein, an “agent that reduces the level and/or activity of MSH3” refers to any polynucleotide agent (e.g., a dsRNA) that reduces the level of or inhibits expression of MSH3 in a cell or subject. By “reducing the level of MSH3,” “reducing expression of MSH3,” and “reducing transcription of MSH3” is meant decreasing the level, decreasing the expression, or decreasing the transcription of MSH3 in a cell or subject, e.g., by administering a dsRNA to the cell or subject. The level of MSH3 can be measured using any method known in the art (e.g., by measuring the levels of MSH3 mRNA or levels of MSH3 protein in a cell or a subject). The reduction can be a decrease in the level, expression, or transcription of MSH3 of about 5% or more (e.g., about 10%, 20%, 30%, 40%, 50%, 60%, 70%, 80%, 90%, 95%, 96%, 97%, 98%, 99%, or 100%) in a cell or subject compared to prior to treatment. The MSH3 can be any MSH3 gene (such as, e.g., a mouse MSH3 gene, a rat MSH3 gene, a monkey MSH3 gene, or a human MSH3 gene) as well as variants or mutants of a MSH3 gene that encode a MSH3 protein. Thus, the MSH3 gene can be a wild-type MSH3 gene, a mutant MSH3 gene, or a transgenic MSH3 gene in the context of a genetically manipulated cell, group of cells, or organism.

By “reducing the activity of MSH3” is meant decreasing the level of an activity related to MSH3 (e.g., by reducing the amount of nucleotide repeats in a gene associated with a nucleotide repeat expansion disorder, e.g., a trinucleotide repeat expansion disorder, that is related to MSH3 activity). The activity level of MSH3 can be measured using any method known in the art (e.g., by directly sequencing a gene associated with a nucleotide repeat expansion disorder to measure the levels of nucleotide repeats).

By “reducing the level of MSH3” is meant decreasing the level of MSH3 in a cell or subject, e.g., by administering an oligonucleotide, or pharmaceutically acceptable salt thereof, to the cell or subject. The level of MSH3 can be measured using any method known in the art (e.g., by measuring the levels of MSH3 mRNA or levels of MSH3 protein in a cell or a subject).

By “modulating the activity of a MutSβ heterodimer comprising MSH3” is meant altering the level of an activity related to a MutSβ heterodimer, or a related downstream effect. The activity level of a MutSβ heterodimer can be measured using any method known in the art.

As used herein, the term “inhibitor” refers to any agent which reduces the level and/or activity of a protein (e.g., MSH3). Non-limiting examples of inhibitors include polynucleotides (e.g., dsRNA, e.g., siRNA or shRNA). The term “inhibiting,” as used herein, is used interchangeably with “reducing,” “silencing,” “downregulating,” “suppressing,” and other similar terms, and includes any level of inhibition.

The phrase “contacting a cell with a dsRNA,” as used herein, includes contacting a cell by any possible means. Contacting a cell with a dsRNA includes contacting a cell in vitro with the dsRNA or contacting a cell in vivo with the dsRNA. The contacting can be done directly or indirectly. Thus, for example, the dsRNA can be put into physical contact with the cell by the individual performing the method, or alternatively, the dsRNA agent can be put into a situation that will permit or cause it to subsequently come into contact with the cell.

Contacting a cell in vitro can be done, for example, by incubating the cell with the dsRNA. Contacting a cell in vivo can be done, for example, by injecting the dsRNA into or near the tissue where the cell is located, or by injecting the dsRNA agent into another area, e.g., the bloodstream or the subcutaneous space, such that the agent will subsequently reach the tissue where the cell to be contacted is located. For example, the dsRNA can contain and/or be coupled to a ligand that directs the dsRNA to a site of interest. Combinations of in vitro and in vivo methods of contacting are also possible. For example, a cell can be contacted in vitro with a dsRNA and subsequently transplanted into a subject.

In one aspect, contacting a cell with a dsRNA includes “introducing” or “delivering the dsRNA into the cell” by facilitating or effecting uptake or absorption into the cell. Absorption or uptake of a dsRNA can occur through unaided diffusive or active cellular processes, or by auxiliary agents or devices. Introducing a dsRNA into a cell can be in vitro and/or in vivo. For example, for in vivo introduction, dsRNA can be injected into a tissue site or administered systemically. In vitro introduction into a cell includes methods known in the art such as electroporation and lipofection. Further approaches are described herein below and/or are known in the art.

As used herein, “lipid nanoparticle” or “LNP” is a vesicle comprising a lipid layer encapsulating a pharmaceutically active molecule, such as a nucleic acid molecule, e.g., a dsRNA or a plasmid from which a dsRNA is transcribed. LNP refers to a stable nucleic acid-lipid particle. LNPs typically contain a cationic lipid, a non-cationic lipid, and a lipid that prevents aggregation of the particle (e.g., a PEG-lipid conjugate). LNPs are described in, for example, U.S. Pat. Nos. 6,858,225; 6,815,432; 8,158,601; and 8,058,069, the entire contents of which are hereby incorporated herein by reference.

As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the dsRNA composition. The lipophilic material isolates the aqueous interior from an aqueous exterior, which typically does not include the dsRNA composition, although in some examples, it can. Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids.

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

As used herein, the terms “effective amount,” “therapeutically effective amount,” and a “sufficient amount” of an agent that reduces the level and/or activity of MSH3 (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating a nucleotide repeat expansion disorder (e.g., a trinucleotide repeat expansion disorder), it is an amount of the agent that reduces the level and/or activity of MSH3 sufficient to achieve a treatment response as compared to the response obtained without administration of the agent that reduces the level and/or activity of MSH3. The amount of a given agent that reduces the level and/or activity of MSH3 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of MSH3 of the present disclosure is an amount which results in a beneficial or desired result in a subject as compared to a control. As defined herein, a therapeutically effective amount of an agent that reduces the level and/or activity of MSH3 of the present disclosure can be readily determined by one of ordinary skill by routine methods known in the art. Dosage regimen can be adjusted to provide the optimum therapeutic response.

“Prophylactically effective amount,” as used herein, is intended to include the amount of a dsRNA that, when administered to a subject having or predisposed to have a nucleotide repeat expansion disorder (e.g., a trinucleotide repeat expansion disorder), is sufficient to prevent or ameliorate the disease or one or more symptoms of the disease. Ameliorating the disease includes slowing the course of the disease or reducing the severity of later-developing disease. The “prophylactically effective amount” can vary depending on the dsRNA, how the agent is administered, the degree of risk of disease, and the history, age, weight, family history, genetic makeup, the types of preceding or concomitant treatments, if any, and other individual characteristics of the patient to be treated. A prophylactically effective amount can refer to, for example, an amount of the agent that reduces the level and/or activity of MSH3 (e.g., in a cell or a subject) described herein or can refer to a quantity sufficient to, when administered to the subject, including a human, delay the onset of one or more of the nucleotide repeat expansion disorders (e.g., trinucleotide repeat expansion disorders) described herein by at least 120 days, for example, at least 6 months, at least 12 months, at least 2 years, at least 3 years, at least 4 years, at least 5 years, at least 10 years or more, when compared with the predicted onset.

A “therapeutically-effective amount” or “prophylactically effective amount” also includes an amount (either administered in a single or in multiple doses) of a dsRNA that produces some desired local or systemic effect at a reasonable benefit/risk ratio applicable to any treatment. dsRNAs employed in the methods herein can be administered in a sufficient amount to produce a reasonable benefit/risk ratio applicable to such treatment.

As used herein, the term “region of complementarity” refers to the region on the antisense strand that is substantially complementary to all or a portion of a gene, primary transcript, a sequence (e.g., a target sequence, e.g., an MSH3 nucleotide sequence), or processed mRNA, so as to interfere with expression of the endogenous gene (e.g., MSH3). Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the dsRNA.

An “amount effective to reduce nucleotide repeat expansion” of a particular gene refers to an amount of the agent that reduces the level and/or activity of MSH3 (e.g., in a cell or a subject) described herein or to a quantity sufficient to, when administered to the subject, including a human, to reduce the nucleotide repeat expansion of a particular gene (e.g., a gene associated with a nucleotide repeat expansion disorder, e.g., a trinucleotide repeat expansion disorder described herein).

As used herein, the term “a subject identified as having a nucleotide repeat expansion disorder” refers to a subject identified as having a molecular or pathological state, disease or condition of or associated with a nucleotide repeat expansion disorder, such as the identification of a nucleotide repeat expansion disorder or symptoms thereof, or to identification of a subject having or suspected of having a nucleotide repeat expansion disorder who can benefit from a particular treatment regimen.

As used herein, “trinucleotide repeat expansion disorder” refers to a class of genetic diseases or disorders characterized by excessive trinucleotide repeats (e.g., trinucleotide repeats such as CAG) in a gene or intron in the subject which exceed the normal, stable threshold, for the gene or intron. Nucleotide repeats are common in the human genome and are not normally associated with disease. In some cases, however, the number of repeats expands beyond a stable threshold and can lead to disease, with the severity of symptoms generally correlated with the number of repeats. Nucleotide repeat expansion disorders include “polyglutamine” and “non-polyglutamine” disorders.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps (DNA core sequences), if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

By “level” is meant a level or activity of a protein, or mRNA encoding the protein (e.g., MSH3), optionally as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than 10%, 15%, 20%, 50%, 75%, 100%, or 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein can be expressed in mass/vol (e.g., g/dL, mg/mL, μg/mL, ng/mL) or percentage relative to total protein or mRNA in a sample.

The term “pharmaceutical composition,” as used herein, represents a composition containing a compound described herein formulated with a pharmaceutically acceptable excipient, and can be manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for intrathecal injection; for intracerebroventricular injections; for intraparenchymal injection; for intraocular administration (e.g., for intravitreal or subretinal administration); or in any other pharmaceutically acceptable formulation.

In some aspects, provided herein are pharmaceutical compositions that are formulated for intracerebroventricular injection.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient. Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein can have ionizable groups so as to be capable of preparation as pharmaceutically acceptable salts. These salts can be acid addition salts involving inorganic or organic acids or the salts can, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts can be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., a nucleotide or trinucleotide repeat expansion disorder); a subject that has been treated with a compound described herein. In some aspects, the reference sample, standard, or level is matched to the sample subject sample by at least one of the following criteria: age, weight, sex, disease stage, and overall health. A standard curve of levels of a purified protein, e.g., any described herein, within the normal reference range can be used as a reference.

As used herein, the term “subject” refers to any organism to which a composition can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein can retain or improve upon the biological activity of the original material.

The details of one or more aspects are set forth in the description below. Other features, objects, and advantages will be apparent from the description and from the claims.

### DETAILED DESCRIPTION

The present inventors have found that inhibition or depletion of MSH3 level and/or activity in a cell is effective in the treatment of a nucleotide repeat expansion disorder (e.g., a trinucleotide repeat expansion disorder). Accordingly, useful compositions and methods to treat nucleotide repeat expansion disorders (e.g., a trinucleotide repeat expansion disorders), e.g., in a subject in need thereof are provided herein.

### I. Nucleotide Repeat Expansion Disorders

Nucleotide repeat expansion disorders (e.g., trinucleotide repeat expansion disorders) are a family of genetic disorders characterized by the pathogenic expansion of a repeat region within a genomic region. In such disorders, the number of repeats exceeds that of a gene's normal, stable threshold, expanding into a diseased range.

Nucleotide repeat expansion disorders (e.g., trinucleotide repeat expansion disorders) generally can be categorized as “polyglutamine” or “non-polyglutamine.” Polyglutamine disorders, including Huntington's disease (HD) and several spinocerebellar ataxias, are caused by a CAG (glutamine) repeats in the protein-coding regions of specific genes. Non-polyglutamine disorders are more heterogeneous and can be caused by CAG nucleotide repeat expansions in non-coding regions, as in Myotonic dystrophy, or by the expansion of nucleotide repeats other than CAG that can be in coding or non-coding regions such as the CGG repeat expansion responsible for Fragile X Syndrome.

Nucleotide repeat expansion disorders (e.g., trinucleotide repeat expansion disorders) are dynamic in the sense that the number of repeats can vary from generation-to-generation, or even from cell-to-cell in the same individual. Repeat expansion is believed to be caused by polymerase “slipping” during DNA replication. Tandem repeats in the DNA sequence can “loop out” while maintaining complementary base pairing between the parent strand and daughter strands. If the loop structure is formed from the daughter strand, the number of repeats will increase.

Conversely, if the loop structure is formed from the parent strand, the number of repeats will decrease. It appears that expansion is more common than reduction. In general, the length of repeat expansion is negatively correlated with prognosis; longer repeats are correlated with an earlier age of onset and worsened disease severity. Thus, nucleotide repeat expansion disorders (e.g., trinucleotide repeat expansion disorders) are subject to “anticipation,” meaning the severity of symptoms and/or age of onset worsen through successive generations of affected families due to the expansion of these repeats from one generation to the next.

Nucleotide repeat expansion disorders (e.g., trinucleotide repeat expansion disorders) are well known in the art. For example, frontotemporal dementia (FTD) is a hexanucleotide repeat string of nucleotides GGGGCC that is repeated many more times in an individual than an individual without FTD. Additionally, an individual having spinocerebellar ataxia type 36 (SCA36) has many more GGCCTG repeats than an individual without SCA36.

Exemplary trinucleotide repeat expansion disorders and the trinucleotide repeats of the genes commonly associated with them are included in Table 1.

TABLE 1
Exemplary Trinucleotide Repeat Expansion Disorders

| | | Nucleotide |
|---|---|---|
| Disorder | Gene | Repeat |
| ARX-nonsyndromic X-linked | ARX | GCG |
| mental retardation (XLMR) | | |
| Baratela-Scott Syndrome | XYLT1 | GGC |
| Blepharophimosis/Ptosis (BPES) | FOXL2 | GCG |
| Epicanthus inversus syndrome | | |
| (BPES) types I and II | | |
| Cleidocranial dysplasia (CCD) | RUNX2 | GCG |
| Congenital central hypoventilation | PHOX-2B | GCG |
| Congenital central hypoventilation | PHOX2B | GCG |
| syndrome (CCHS) | | |
| Creutzfeldt-Jakob disease | PRNP | |
| Dentatorubral-pallidoluysian | ATN1 | CAG |
| atrophy (DRPLA)/Haw River | | |
| syndrome | | |
| Early infantile epileptic | ARX | GCG |
| encephalopathy (Ohtahara | | |
| syndrome) | | |
| FRA2A syndrome | AFF3 | CGC |
| FRA7A syndrome | ZNF713 | CGG |
| Fragile X mental retardation | AFF2/FMR2 | GCC |
| (FRAX-E) | | |
| Fragile X Syndrome (FXS) | FMR1 | CGG |
| Fragile X-associated Primary | FMR1 | CGG |
| Ovarian Insufficiency (FXPOI) | | |
| Fragile X-associated Tremor Ataxia | FMR1 | CGG |
| Syndrome (FXTAS) | | |
| Friedreich ataxia (FRDA) | FXN | GAA |
| Fuchs' Corneal Endothelial | TCF4 | CTG |
| Dystrophy (FECD) | | |
| Hand-foot genital syndrome (HFGS) | HOXA13 | GCG |
| Holoprosencephaly disorder (HPE) | ZIC2 | GCG |
| Huntington disease-like 2 (HDL2) | JPH3 | CTG |
| Huntington's Disease (HD) | HTT | CAG |
| Infantile spasm syndrome/West | ARX | GCG |
| syndrome (ISS) | | |
| KCNN3-associated (e.g., | KCNN3 | CAG |
| schizophrenia) | | |
| Multiple Skeletal dysplasias | COMP | GAC |
| Myotonic Dystrophy type 1 (DM1) | DMPK | CTG |
| Myotonic Dystrophy type 2 (DM2) | CNBP | CCTG |
| NCOA3-associated (e.g., increased | NCOA3 | CAG |
| risk of prostate cancer) | | |
| Neuronal intranuclear inclusion | NOTCH2NLC | GGC |
| disease (NIID) | | |
| Oculopharyngeal Muscular | PABPN1 | GCG |
| Dystrophy (OPMD) | | |
| Spinal Muscular Bulbar Atrophy | AR | CAG |
| (SMBA) | | |
| Spinocerebellar ataxia type 1 | ATXN1 | CAG |
| (SCA1) | | |
| Spinocerebellar ataxia type 10 | ATXN10 | ATTCT |
| (SCA10) | | |
| Spinocerebellar ataxia type 12 | PPP2R2B | CAG |
| (SCA12) | | |
| Spinocerebellar ataxia type 17 | TBP/ATXN17 | CAG |
| (SCA17) | | |
| Spinocerebellar ataxia type 2 | ATXN2 | CAG |
| (SCA2) | | |
| Spinocerebellar ataxia type 3 | ATXN3 | CAG |
| (SCA3)/Machado-Joseph Disease | | |
| Spinocerebellar ataxia type 45 | FAT2 | CAG |
| (SCA45) | | |
| Spinocerebellar ataxia type 6 | CACNA1A | CAG |
| (SCA6) | | |
| Spinocerebellar ataxia type 7 | ATXN7 | CAG |
| (SCA7) | | |
| Spinocerebellar ataxia type 8 | ATXN8 | CTG |
| (SCA8) | | |
| Syndromic neurodevelopmental | MAB21L1 | CAG |
| disorder with cerebellar, ocular, | | |
| craniofacial, and genital features | | |
| (COFG syndrome) | | |
| Synpolydactyly (SPD I) | HOXD13 | GCG |
| Synpolydactyly (SPD II) | HOXD12 | GCG |

The proteins associated with nucleotide repeat expansion disorders (e.g., trinucleotide repeat expansion disorders) are typically selected based on an experimental association of the protein associated with a nucleotide repeat expansion disorders (e.g., trinucleotide repeat expansion disorder) to a nucleotide repeat expansion disorder. For example, the production rate or circulating concentration of a protein associated with a nucleotide repeat expansion disorder (e.g., trinucleotide repeat expansion disorder) can be elevated or depressed in a population having a nucleotide repeat expansion disorder (e.g., a trinucleotide repeat expansion disorder) relative to a population lacking the nucleotide repeat expansion disorder (e.g., a trinucleotide repeat expansion disorder). Differences in protein levels can be assessed using proteomic techniques including but not limited to Western blot, immunohistochemical staining, enzyme linked immunosorbent assay (ELISA), and mass spectrometry. Alternatively, the proteins associated with nucleotide repeat expansion disorder (e.g., trinucleotide repeat expansion disorders) can be identified by obtaining gene expression profiles of the genes encoding the proteins using genomic techniques including, but not limited to, DNA microarray analysis, serial analysis of gene expression (SAGE), and quantitative real-time polymerase chain reaction (qPCR).

### II. Evidence for the Involvement of Mismatch Repair Pathway in Nucleotide Repeat Expansion

There is growing evidence that DNA repair pathways, particularly mismatch repair (MMR), are involved in the expansion of nucleotide repeats (e.g., trinucleotide repeats (Liu & Wilson (2012) Trends Biochem Sci. 37: 162-172). A recent genome-wide association (GWA) analysis led to the identification of loci harboring genetic variations that alter the age at neurological onset of Huntington's disease (HD) (GEM-HD Consortium, Cell. 2015 Jul. 30; 162(3):516-26). The study identified MLH1, the human homolog of the E. coli DNA mismatch repair gene mutL. A subsequent GWA study in polyglutamine disease patients found significant association of age at onset when grouping all polyglutamine diseases (HD and SCAs) with DNA repair genes as a group, as well as significant associations for specific SNPs in FAN1 and PMS2 with the diseases (Bettencourt et al., (2016) Ann. Neurol., 79: 983-990). These results were consistent with those from an earlier study comparing differences in repeat expansion in two different mouse models of Huntington's Disease, which identified Mlh1 and Mlh3 as novel critical modifiers of CAG instability (Pinto et al., (2013) Mismatch Repair Genes Mlh1 and Mlh3 Modify CAG Instability in Huntington's Disease Mice: Genome-Wide and Candidate Approaches. PLoS Genet 9(10): e1003930). Another member of the mismatch repair pathway, 8-oxo-guanine glycosylase (OGG1) has also been implicated in expansion, as somatic expansion was found to be reduced in transgenic mice lacking OGG1 (Kovtun I. V. et al. (2007) Nature 447, 447-452). However, another study found that human subjects containing a Ser326Cys polymorphism in hOGG1, which results in reduced OGG1 activity, results in increased mutant huntingtin (Coppede et al., (2009) Toxicol., 278: 199-203). Likewise, complete inactivation of Fan1, another component of the DNA repair pathway, in a mouse HD model produces somatic CAG expansions (Long et al. (2018) J. Hum Genet., 103: 1-9). MSH3, another component of the mismatch repair pathway, has been reported to be linked to somatic expansion: polymorphisms in Msh3 was associated with somatic instability of the expanded CTG trinucleotide repeat in myotonic dystrophy type 1 (DM1) patients (Morales et al., (2016) DNA Repair 40: 57-66). Furthermore, natural polymorphisms in Msh3 and Mlh1 have been revealed as mediators of mouse strain specific differences in CTG⋅CAG repeat instability (Pinto et al. (2013) ibid; Tome et al., (2013) PLoS Genet. 9 e1003280). Likewise, mice lacking MSH2 or MSH3 have attenuated expansion in the human HD gene (Manley et al., (1999) Nat. Genet. 23, 471-473), the human myotonic dystrophy 1 protein kinase transgene (van den Broek et al. (2002) Hum. Mol. Genet. 11, 191-198), the FAX gene in Friedreich's ataxia (FRDA) (Bourn et al. (2012) PLoS One 7, e47085) and the fragile mental retardation gene in fragile X syndrome (FXS) (Lokanga et al., (2012) Hum. Mutat. 35, 129-136). Further evidence of Msh2 and Msh3's involvement in expansion repeats was reported in a study in which short hairpin RNA (shRNA) knockdown of either MSH2 or MSH3 slowed, and ectopic expression of either MSH2 or MSH3 induced GAA trinucleotide repeat expansion of the Friedreich Ataxia (FRDA) gene in fibroblasts derived from FRDA patients (Halabi et al., (2012) J. Biol. Chem. 287, 29958-29967). In spite of some inconsistent results provided above, there is strong evidence that the MMR pathway plays some role in the expansion of trinucleotide repeats in various disorders. Moreover, they are the first to recognize that the inhibition of the MMR pathway provides for the treatment or prevention of these repeat expansion disorders; however, no therapy is currently available or in development which modulates MMR for purposes of treating or preventing these repeat expansion disorders.

III. dsRNA Agents

Agents described herein that reduce the level and/or activity of MSH3 in a cell can be, for example, a polynucleotide, e.g., a double stranded nucleotide, or pharmaceutically acceptable salt thereof. These agents reduce the level of an activity related to MSH3, or a related downstream effect, or reduce the level of MSH3 in a cell or subject.

In some aspects, the agent that reduces the level and/or activity of MSH3 is a polynucleotide. In some aspects, the polynucleotide is an inhibitory RNA molecule, e.g., that acts by way of the RNA interference (RNAi) pathway. An inhibitory RNA molecule can decrease the expression level (e.g., protein level or mRNA level) of MSH3. Inhibitory RNA molecules can be double stranded (dsRNA) molecules. For example, a dsRNA includes a short interfering RNA (siRNA) that targets full-length MSH3. A siRNA is a double-stranded RNA molecule that typically has a length of about 19-25 base pairs. In other aspects, the dsRNA is a short hairpin RNA (shRNA) that targets full-length MSH3. A shRNA is a dsRNA molecule including a hairpin turn that decreases expression of target genes via the RNAi pathway. In some aspects, the dsRNA molecule recruits an RNAse H enzyme. Degradation is caused by an enzymatic, RNA-induced silencing complex (RISC).

In some aspects, the dsRNA or pharmaceutically acceptable salt thereof decreases the level and/or activity of a positive regulator of function. In other aspects, the dsRNA or pharmaceutically acceptable salt thereof increases the level and/or activity of an inhibitor of a positive regulator of function. In some aspects, the dsRNA increases the level and/or activity of a negative regulator of function.

In some aspects, the dsRNA, or pharmaceutically acceptable salt thereof, decreases the level and/or activity or function of MSH3. In some aspects, the dsRNA, or pharmaceutically acceptable salt thereof, inhibits expression of MSH3. In other aspects, the dsRNA, or pharmaceutically acceptable salt thereof, increases degradation of MSH3 and/or decreases the stability (i.e., half-life) of MSH3. The dsRNA can be chemically synthesized or transcribed in vitro.

The dsRNA, or pharmaceutically acceptable salt thereof, includes an antisense strand having a region of complementarity (e.g., a contiguous nucleobase region) which is complementary to at least a part of an mRNA formed in the expression of a MSH3 gene. The region of complementarity can be about 30 nucleotides or less in length (e.g., about 30, 29, 28, 27, 26, 25, 24, 23, 22, 21, 20, 19, or 18 nucleotides or less in length). Upon contact with a cell expressing the MSH3 gene, the dsRNA, or pharmaceutically acceptable salt thereof, can reduce the expression of MSH3 (e.g., a human, a primate, a non-primate, or a bird MSH3) by at least about 10% as assayed by, for example, a PCR or branched DNA (bDNA)-based method, or by a protein-based method, such as by immunofluorescence analysis, using, for example, Western Blotting or flowcytometric techniques.

A dsRNA, or pharmaceutically acceptable salt thereof, includes two RNA strands that are complementary and hybridize to form a duplex structure under conditions in which the dsRNA can be used. One strand of a dsRNA (the antisense strand) includes a region of complementarity that is substantially complementary, and generally fully complementary, to a target sequence. The target sequence can be derived from the sequence of an mRNA formed during the expression of a MSH3 gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can be contained as self-complementary regions of a single nucleic acid molecule, as opposed to being on separate oligonucleotides. Generally, the duplex structure is between 15 and 30 linked nucleosides in length, e.g., between 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 or 15, 16, 17, 18, 19, 20, 21, 22, 23, 24, 25, 26, 27, 28, 29, or 30 linked nucleosides in length. Ranges and lengths intermediate to the above recited ranges and lengths are also contemplated.

Similarly, the region of complementarity to the target sequence is between 15 and 30 linked nucleosides in length, e.g., between 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 or 15, 16, 17, 18, 19, 20, 21, 22, 23, 24, 25, 26, 27, 28, 29, or 30 linked nucleosides in length. Ranges and lengths intermediate to the above recited ranges and lengths are also contemplated.

In some aspects, the dsRNA is between about 15 and about 23 linked nucleosides in length, or between about 25 and about 30 linked nucleosides in length. In general, the dsRNA is long enough to serve as a substrate for the Dicer enzyme. For example, it is well known in the art that dsRNAs longer than about 21-23 linked nucleosides can serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA. Thus, in one aspect, to the extent that it becomes processed to a functional duplex, of e.g., 15-30 linked nucleosides, that targets a desired RNA for cleavage, an RNA molecule or complex of RNA molecules having a duplex region greater than 30 linked nucleosides is a dsRNA. Thus, an ordinarily skilled artisan will recognize that in one aspect, a dsRNA is not a naturally occurring dsRNA. In another aspect, a dsRNA agent useful to target MSH3 expression is not generated in the target cell by cleavage of a larger dsRNA.

A dsRNA, or pharmaceutically acceptable salt thereof, as described herein can further include one or more single-stranded nucleoside overhangs e.g., 1, 2, 3, or 4 linked nucleosides. dsRNAs having at least one nucleoside overhang can have unexpectedly superior inhibitory properties relative to their blunt-ended counterparts. A nucleoside overhang can comprise or consist of a deoxyribonucleoside. A nucleoside overhang can comprise or consist of one or more phosphorothioates bonds. The overhang(s) can be on the sense strand, the antisense strand, or any combination thereof. Furthermore, the nucleoside(s) of an overhang can be present on the 5′-end, 3′-end, or both ends of either an antisense or sense strand of a dsRNA. Various dsRNA overhangs are known in the art and can include, but are not limited to: dTdT, UU, or other nucleotides. The overhangs can include phosphorothioate linkages. The overhangs can be different between the sense and antisense oligonucleotides. In some aspects, the dsRNA sequences described herein can include any of the above mentioned overhangs.

A dsRNA, or pharmaceutically acceptable salt thereof, can be synthesized by standard methods known in the art as further discussed below, e.g., by use of an automated DNA synthesizer, such as are commercially available from, for example, Biosearch, Applied Biosystems, Inc.

dsRNA compounds can be prepared using a two-step procedure. For example, the individual strands of the dsRNA can be prepared separately. Then, the component strands can be annealed. The individual strands of the dsRNA can be prepared using solution-phase or solid-phase organic synthesis or both. Organic synthesis offers the advantage that the oligonucleotide strands comprising unnatural or alternative nucleotides can be easily prepared. Double-stranded oligonucleotides can be prepared using solution-phase or solid-phase organic synthesis or both.

In one aspect, a dsRNA includes at least two nucleobase sequences, a sense sequence and an antisense sequence. In some aspects, the antisense strand comprises a nucleobase sequence of an antisense strand in Table 3, wherein the 5′ nucleotide represented by U can be any nucleotide (e.g., U, A, C, G), and the sense strand comprises a nucleobase sequence complementary to the nucleobase sequence of the antisense strand. In other aspects, the sense strand comprises a nucleobase sequence of a sense strand in Table 3, and the antisense strand comprises a nucleobase sequence complementary to the nucleobase sequence of the sense strand. In some aspects, the antisense strand consists of a nucleobase sequence of an antisense strand in Table 3, wherein the 5′ nucleotide represented by U can be any nucleotide (e.g., U, A, C, G), and the sense strand consists of a nucleobase sequence complementary to the nucleobase sequence of the antisense strand. In other aspects, the sense strand consists of a nucleobase sequence of a sense strand in Table 3, and the antisense strand consists of a nucleobase sequence complementary to the nucleobase sequence of the sense strand.

In some aspects, the sense strand comprises a nucleobase sequence of a sense strand in any one of Tables 4-10, and the antisense strand comprises a nucleobase sequence complementary to the nucleobase sequence of the sense strand. In some aspects, the sense strand consists of a nucleobase sequence of a sense strand in any one of Tables 4-10, and the antisense strand consists of a nucleobase sequence complementary to the nucleobase sequence of the sense strand. In some aspects, the antisense strand comprises a nucleobase sequence of an antisense strand in Table 11, wherein the 5′ nucleotide represented by U of the antisense oligonucleotide is any nucleotide (e.g., U, A, G, C, T), and the sense strand comprises a nucleobase sequence complementary to the nucleobase sequence of the antisense strand. In other aspects, the antisense strand consists of a nucleobase sequence of an antisense strand in Table 11, wherein the 5′ nucleotide represented by U of the antisense oligonucleotide is any nucleotide (e.g., U, A, G, C, T), and the sense strand consists of a nucleobase sequence complementary to the nucleobase sequence of the antisense strand. In some aspects, the sense strand comprises a nucleobase sequence of a sense strand in Table 11, and the antisense strand comprises a nucleobase sequence complementary to the nucleobase sequence of the sense strand. In other aspects, the sense strand consists of a nucleobase sequence of a sense strand in Table 11, and the antisense strand consists of a nucleobase sequence complementary to the nucleobase sequence of the sense strand.

In these aspects, one of the two sequences is complementary to the other of the two sequences, with one of the sequences being substantially complementary to a sequence of an mRNA generated in the expression of MLH1. As such, in this aspect, a dsRNA will include two oligonucleotides, where one oligonucleotide is described as the sense strand (passenger strand) in Table 3 or 11, and the second oligonucleotide is described as the corresponding antisense strand (guide strand) of the sense strand in Table 3 or 11, wherein the 5′ nucleotide represented by U of the antisense oligonucleotide is any nucleotide (e.g., U, A, G, C, T). In one aspect, the substantially complementary sequences of the dsRNA are contained on separate oligonucleotides. In another aspect, the substantially complementary sequences of the dsRNA are contained on a single oligonucleotide.

In one aspect, the antisense or sense strand of the dsRNA includes a region of at least 15 contiguous nucleobases having at least 80% (e.g., at least 85%, at least 90%, at least 95%, or at least 99%) complementary to at least 15 contiguous nucleotides of an MSH3 gene. In some aspects, the region of at least 15 contiguous that is complementary to an MSH3 gene corresponding to reference mRNA NM_002439.4 is one or more of positions 566-589, 678-701, 875-989, 1019-1088, 1370-1393, 1466-1569, 1721-1849, 1879-2038, 2086-2171, 2783-2806, 2847-2922, 3043-3119, 3241-3314, 3330-3353, or 3701-3792 of the MSH3 gene. In some aspects, the region of at least 15 contiguous that is complementary to an MSH3 gene corresponding to reference mRNA NM_002439.4 at one or more of positions 876-989, 1019-1088, 1370-1393, 1889-1938, or 3241-3314 of the MSH3 gene. In some aspects, the region of at least 15 contiguous that is complementary to an MSH3 gene corresponding to reference mRNA NM_002439.4 at one or more of positions 876-989, 1019-1088, 1370-1393, 1466-1569, 1756-1849, 1879-2038, 2086-2171, 2783-2806, 2847-2922, 3043-3119, 3241-3314, 3330-3353, or 3701-3792 of the MSH3 gene. In some aspects, the region of at least 15 contiguous that is complementary to an MSH3 gene corresponding to reference mRNA NM_002439.4 at one or more of positions 678-701, 875-989, 1019-1088, 1370-1393, 1466-1569, 1721-1849, 1879-2038, 2086-2171, 2783-2806, 2847-2922, 3043-3119, 3241-3314, 3330-3353, or 3701-3792 of the MSH3 gene. In some aspects, the region of at least 15 contiguous that is complementary to an MSH3 gene corresponding to reference mRNA NM_002439.4 at positions 879-921 of the MSH3 gene. In some aspects, the region the sense or antisense strand is complementary to is at least 15 contiguous nucleotides of an MSH3 gene corresponding to a sequence of reference mRNA NM_002439.4 at one or more of positions 875-989, 1019-1088, 1370-1393, 1466-1569, 1721-1849, 1879-1970, 2783-2806, 2847-2922, 3043-3119, 3241-3314, 3330-3353, or 3703-3792 of the MSH3 gene. In some aspects, the region of at least 15 contiguous that is complementary to an MSH3 gene corresponding to reference mRNA NM_002439.4 at one or more of positions 566-589, 678-701, 875-989, 1019-1088, 1370-1393, 1466-1569, 1721-1849, 1879-2038, 2086-2171, 2783-2806, 2847-2922, 3043-3119, 3241-3314, 3330-3353, or 3701-3792 of the MSH3 gene.

In some aspects, a dsRNA having a sense strand or an antisense strand comprises the nucleobase sequence of any one of SEQ ID NOs: 6-2873, wherein the 5′ nucleotide represented by U of the antisense oligonucleotide is any nucleotide (e.g., U, A, G, C, T). In some aspects, the sense strand comprises a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690. In some aspects, the sense strand comprises a nucleobase sequence of any one of SEQ ID NOs: 380, 382, 386, 388, 396, 406, 418, 464, 478, 520, 540, 564, 568, 750, 1166, 1168, 1170, 1182, 1192, 1212, 1214, 1216, 1222, 2264, 2290, 2308, or 2318. In some aspects, the sense strand comprises a nucleobase sequence of any one of SEQ ID NOs: 380, 382, 386, 388, 396, 406, 418, 464, 478, 520, 540, 564, 568, 750, 822, 830, 844, 868, 870, 874, 904, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690. In some aspects, the sense strand comprises a nucleobase sequence of any one of SEQ ID NOs: 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690. In some aspects, the sense strand comprises a nucleobase sequence of any one of SEQ ID NOs: 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690. In some aspects, the sense strand comprises a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

In some aspects, the antisense strand comprises a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G). In some aspects, the antisense strand comprises a nucleobase sequence of any one of SEQ ID NOs: 381, 383, 387, 389, 397, 407, 419, 465, 479, 521, 541, 565, 569, 751, 1167, 1169, 1171, 1183, 1193, 1213, 1215, 1217, 1223, 2265, 2291, 2309, or 2319, wherein the 5′ nucleotide represented by U can be any nucleotide (e.g., U, A, C, G). In some aspects, the antisense strand comprises a nucleobase sequence of any one of SEQ ID NOs: 381, 383, 387, 389, 397, 407, 419, 465, 479, 521, 541, 565, 569, 751, 823, 831, 845, 869, 871, 875, 905, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G). In some aspects, the antisense strand comprises a nucleobase sequence of any one of SEQ ID NOs: 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G). In some aspects, the antisense strand comprises a nucleobase sequence of any one of SEQ ID NOs: 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G). In some aspects, the antisense strand comprises a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

In some aspects, a dsRNA having a sense strand or an antisense strand consists of the nucleobase sequence of any one of SEQ ID NOs: 6-2873, wherein the 5′ nucleotide represented by U of the antisense oligonucleotide is any nucleotide (e.g., U, A, G, C, T).

In some aspects, the sense strand consists of a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690. In some aspects, the sense strand consists of a nucleobase sequence of any one of SEQ ID NOs: 380, 382, 386, 388, 396, 406, 418, 464, 478, 520, 540, 564, 568, 750, 1166, 1168, 1170, 1182, 1192, 1212, 1214, 1216, 1222, 2264, 2290, 2308, or 2318. In some aspects, the sense strand consists of a nucleobase sequence of any one of SEQ ID NOs: 380, 382, 386, 388, 396, 406, 418, 464, 478, 520, 540, 564, 568, 750, 822, 830, 844, 868, 870, 874, 904, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690. In some aspects, the sense strand consists of a nucleobase sequence of any one of SEQ ID NOs: 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690. In some aspects, the sense strand consists of a nucleobase sequence of any one of SEQ ID NOs: 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690. In some aspects, the sense strand consists of a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690. In some aspects, the sense strand consists of a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691.

In some aspects, the antisense strand consists of a nucleobase sequence of any one of SEQ ID NOs: 381, 383, 387, 389, 397, 407, 419, 465, 479, 521, 541, 565, 569, 751, 1167, 1169, 1171, 1183, 1193, 1213, 1215, 1217, 1223, 2265, 2291, 2309, or 2319, wherein the 5′ nucleotide represented by U can be any nucleotide (e.g., U, A, C, G). In some aspects, the antisense strand consists of a nucleobase sequence of any one of SEQ ID NOs: 381, 383, 387, 389, 397, 407, 419, 465, 479, 521, 541, 565, 569, 751, 823, 831, 845, 869, 871, 875, 905, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G). In some aspects, the antisense strand consists of a nucleobase sequence of any one of SEQ ID NOs: 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G). In some aspects, the antisense strand consists of a nucleobase sequence of any one of SEQ ID NOs: 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G). In some aspects, the antisense strand consists of a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

In some aspects, the dsRNA exhibits at least 50% mRNA inhibition at a 0.5 nM dsRNA concentration when determined using a cell assay when compared with a control cell. In some aspects, the dsRNA exhibits at least 40% mRNA inhibition at a 0.5 nM dsRNA concentration when determined using a cell assay when compared with a control cell. In some aspects, the dsRNA exhibits at least 30% mRNA inhibition at a 0.5 nM dsRNA concentration when determined using a cell assay when compared with a control cell. In some aspects, the dsRNA exhibits at least 70% mRNA inhibition at a 10 nM dsRNA concentration when determined using a cell assay when compared with a control cell. In some aspects, the dsRNA exhibits at least 60% mRNA inhibition at a 10 nM dsRNA concentration when determined using a cell assay when compared with a control cell. In some aspects, the dsRNA exhibits at least 50% mRNA inhibition at a 10 nM dsRNA concentration when determined using a cell assay when compared with a control cell.

In some aspects, the antisense strand is complementary to at least 17 contiguous nucleotides of an MSH3 gene. In some aspects, the antisense strand is complementary to at least 19 contiguous nucleotides of an MSH3 gene. In some aspects, the antisense strand is complementary to 19 contiguous nucleotides of an MSH3 gene. In some aspects, the sense strand is complementary to at least 17 contiguous nucleotides of an MSH3 gene. In some aspects, the sense strand is complementary to at least 19 contiguous nucleotides of an MSH3 gene. In some aspects, the sense strand is complementary to 19 contiguous nucleotides of an MSH3 gene.

Multiple dsRNAs can be joined together by a linker. The linker can be cleavable or non-cleavable. The dsRNAs can be the same or different.

In some aspects, a dsRNA has a sense strand or an antisense strand having a nucleobase sequence with at least 50% (e.g., at least 50%, at least 60%, at least 70%, at least 80%, at least 85%, at least 90%, at least 95%, at least 96%, at least 97%, at least 98%, at least 99%, or 100%) sequence identity to the nucleobase sequence any one of SEQ ID NOs: 6-2873, wherein the 5′ nucleotide represented by U can be any nucleotide (e.g., U, A, C, G). In some aspects, a dsRNA has a sense strand or an antisense strand having a nucleobase sequence with at least 85% sequence identity to the nucleobase sequence of any one of SEQ ID NOs: 6-2873, wherein the 5′ nucleotide represented by U can be any nucleotide (e.g., U, A, C, G).

It will be understood that, although the sequences in SEQ ID NOs: 6-2873 are described as unmodified and/or un-conjugated sequences, the RNA of the dsRNA can comprise any one of the sequences set forth in any one of SEQ ID NOs: 6-2873 that is an alternative nucleoside and/or conjugated as described in detail below.

The skilled person is well aware that dsRNAs having a duplex structure of between about 20 and 23 linked nucleosides, e.g., 21 linked nucleosides, have been hailed as particularly effective in inducing RNA interference (Elbashir et al., (2001) EMBO J., 20:6877-6888). However, others have found that shorter or longer RNA duplex structures can be effective (Chu and Rana (2007) RNA 14:1714-1719; Kim et al. (2005) Nat Biotech 23:222-226). In the aspects described above, by virtue of the nature of the oligonucleotide sequences provided herein, dsRNAs described herein can include at least one strand of a length of minimally 21 linked nucleosides. It can be reasonably expected that shorter duplexes minus only a few linked nucleosides on one or both ends can be similarly effective as compared to the dsRNAs described above. Hence, dsRNAs having a sequence of at least 8, 9, 10, 11, 12, 13, 14, 15, 16, 17, 18, 19, 20, or more contiguous linked nucleosides derived from one of the sequences provided herein, and differing in their ability to reduce the expression of MSH3 by not more than about 5, 10, 15, 20, 25, or 30% reduction from a dsRNA comprising the full sequence, are contemplated.

In addition, the RNAs described herein identify a site(s) in a MSH3 transcript that is susceptible to RISC-mediated cleavage. As used herein, a dsRNA is said to target within a particular site of an RNA transcript if the dsRNA promotes cleavage of the transcript anywhere within that particular site. Such a dsRNA will generally include at least about 15 contiguous linked nucleosides from one of the sequences provided herein coupled to additional linked nucleoside sequences taken from the region contiguous to the selected sequence in a MSH3 gene.

Inhibitory dsRNAs can be designed by methods well known in the art. While a target sequence is generally about 15-30 linked nucleosides in length, there is wide variation in the suitability of particular sequences in this range for directing cleavage of any given target RNA.

dsRNAs (e.g., siRNA and shRNA molecules) with homology sufficient to provide sequence specificity required to uniquely degrade any RNA can be designed using programs known in the art.

Systematic testing of several designed species for optimization of the inhibitory dsRNA sequence can be undertaken in accordance with the teachings provided herein. Considerations when designing interfering oligonucleotides include, but are not limited to, biophysical, thermodynamic, and structural considerations, base preferences at specific positions in the sense strand, and homology. The making and use of inhibitory therapeutic agents based on non-coding RNA such as siRNAs and shRNAs are also known in the art, for example, as described in Sioud, RNA Therapeutics: Function, Design, and Delivery (Methods in Molecular Biology). Humana Press 2010.

Various software packages and the guidelines set out herein provide guidance for the identification of optimal target sequences for any given gene target, but an empirical approach can be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with a dsRNA agent, mediate the best reduction of target gene expression. Thus, while the sequences identified herein represent effective target sequences, it is contemplated that further optimization of reduction efficiency can be achieved by progressively “walking the window” one nucleotide upstream or downstream of the given sequences to identify sequences with equal or better reduction characteristics.

Further, it is contemplated that for any sequence identified herein, further optimization could be achieved by systematically either adding or removing linked nucleosides to generate longer or shorter sequences and testing those sequences generated by walking a window of the longer or shorter size up or down the target RNA from that point. Again, coupling this approach to generating new candidate targets with testing for effectiveness of dsRNAs based on those target sequences in an inhibition assay as known in the art and/or as described herein can lead to further improvements in the efficiency of inhibition.

Further still, such optimized sequences can be adjusted by, e.g., addition or changes in overhang, the introduction of alternative nucleosides, alternative sugar moieties, and/or alternative internucleosidic linkages as described herein or as known in the art, including alternative nucleosides, alternative sugar moieties, and/or alternative internucleosidic linkages as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes) as an expression inhibitor. A dsRNA agent as described herein can contain one or more mismatches to the target sequence. In one aspect, a dsRNA as described herein contains no more than 3 mismatches. In one aspect, if the antisense strand of the dsRNA contains mismatches to a target sequence, the area of mismatch is not located in the center of the region of complementarity. If the antisense strand of the dsRNA contains mismatches to the target sequence, the mismatch can be restricted to be within the last 5 nucleotides from either the 5′- or 3′-end of the region of complementarity. For example, for a 23-nucleotide dsRNA, the strand which is complementary to a region of a MSH3 gene, generally does not contain any mismatch within the central 13 nucleotides. The methods described herein or methods known in the art can be used to determine whether a dsRNA containing a mismatch to a target sequence is effective in reducing the expression of a MSH3 gene. Consideration of the efficacy of dsRNAs with mismatches in reducing expression of MSH3 is important, especially if the particular region of complementarity in MSH3 is known to have polymorphic sequence variation within the population.

Construction of vectors for expression of polynucleotides for use in the methods described herein can be accomplished using conventional techniques which do not require detailed explanation to one of ordinary skill in the art. For generation of efficient expression vectors, it is necessary to have regulatory sequences that control the expression of the polynucleotide. These regulatory sequences include promoter and enhancer sequences and are influenced by specific cellular factors that interact with these sequences, and are well known in the art.

A. Alternative dsRNAs

In one aspect, one or more of the linked nucleosides or internucleosidic linkages of the dsRNA is naturally occurring, and does not comprise, e.g., chemical modifications and/or conjugations known in the art and described herein. In another aspect, one or more of the linked nucleosides or internucleosidic linkages of a dsRNA is chemically modified to enhance stability or other beneficial characteristics. Without being bound by theory, it is believed that certain modifications can increase nuclease resistance and/or serum stability, or decrease immunogenicity. For example, dsRNAs can contain nucleotides found to occur naturally in DNA or RNA (e.g., adenine, thymidine, guanosine, cytidine, uridine, or inosine) or can contain alternative nucleosides or internucleosidic linkages which have one or more chemical modifications to one or more components of the nucleotide (e.g., the nucleobase, sugar, or phospho-linker moiety). dsRNAs can be linked to one another through naturally occurring phosphodiester bonds, or can contain alternative linkages (e.g., covalently linked through phosphorothioate (e.g., Sp phosphorothioate or Rp phosphorothioate), 3′-methylenephosphonate, 5′-methylenephosphonate, 3′-phosphoamidate, 2′-5′ phosphodiester, guanidinium, S-methylthiourea, 2′-alkoxy, alkyl phosphate, and/or peptide bonds).

In some aspects, substantially all of the nucleosides or internucleosidic linkages of a dsRNA are alternative nucleosides. In other aspects, all of the nucleosides or internucleosidic linkages of dsRNA are alternative nucleosides. dsRNA in which “substantially all of the nucleosides are alternative nucleosides” are largely but not wholly modified and can include not more than five, four, three, two, or one naturally-occurring nucleosides. In still other aspects, dsRNAs can include not more than five, four, three, two, or one alternative nucleosides.

The nucleic acids can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Alternative nucleotides and nucleosides include those with modifications including, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. The nucleobase can be an isonucleoside in which the nucleobase is moved from the C1 position of the sugar moiety to a different position (e.g. C2, C3, C4, or C5). Specific examples of dsRNA compounds useful in the aspects described herein include, but are not limited to alternative nucleosides containing modified backbones or no natural internucleoside linkages. Nucleotides and nucleosides having modified backbones include, among others, those that do not have a phosphorus atom in the backbone. For the purposes of this specification, and as sometimes referenced in the art, alternative RNAs that do not have a phosphorus atom in their internucleoside backbone can be considered to be oligonucleosides. In some aspects, a dsRNA will have a phosphorus atom in its internucleoside backbone.

Alternative internucleoside linkages include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boronophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts, and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Alternative internucleoside linkages that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 564,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other aspects, suitable dsRNAs include those in which both the sugar and the internucleoside linkage, i.e., the backbone, of the nucleotide units are replaced. The base units are maintained for hybridization with an appropriate nucleic acid target compound. One such oligomeric compound, a mimetic that has been shown to have excellent hybridization properties, is referred to as a peptide nucleic acid (PNA). In PNA compounds, the sugar of a nucleoside is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the dsRNAs are described in, for example, in Nielsen et al., Science, 1991, 254, 1497-1500.

Some aspects include dsRNAs with phosphorothioate backbones and oligonucleotides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂—CH₂-[wherein the native phosphodiester backbone is represented as —O—P—O—CH₂—] of the above-referenced U.S. Pat. No. 5,489,677, and the amide backbones of the above-referenced U.S. Pat. No. 5,602,240. In some aspects, the dsRNAs featured herein have morpholino backbone structures of the above-referenced U.S. Pat. No. 5,034,506. In other aspects, the dsRNAs described herein include phosphorodiamidate morpholino oligomers (PMO), in which the deoxyribose moiety is replaced by a morpholine ring, and the charged phosphodiester inter-subunit linkage is replaced by an uncharged phophorodiamidate linkage, as described in Summerton, et al., Antisense Nucleic Acid Drug Dev. 1997, 7:63-70.

Alternative nucleosides and nucleotides can contain one or more substituted sugar moieties. The dsRNAs, e.g., siRNAs and shRNAs, featured herein can include one of the following at the 2′-position: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include —O[(CH₂)ₙO]ₘCH₃, —O(CH₂)ₙOCH₃, —O(CH₂)ₙ—NH₂, —O(CH₂)ₙCH₃, —O(CH₂)ₙ—ONH₂, and —O(CH₂)ₙ—ON[(CH₂)ₙCH₃]₂, where n and m are from 1 to about 10. In other aspects, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a dsRNA, or a group for improving the pharmacodynamic properties of a dsRNA, and other substituents having similar properties. In some aspects, the modification includes a 2′-methoxyethoxy (2′-O—CH₂CH₂OCH₃, also known as 2′-O-(2-methoxyethyl) or 2′-MOE) (Martin et al., Helv. Chin. Acta, 1995, 78:486-504) i.e., an alkoxy-alkoxy group. MOE nucleosides confer several beneficial properties to dsRNAs including, but not limited to, increased nuclease resistance, improved pharmacokinetics properties, reduced non-specific protein binding, reduced toxicity, reduced immunostimulatory properties, and enhanced target affinity as compared to unmodified dsRNAs.

Another exemplary alternative contains 2′-dimethylaminooxyethoxy, i.e., a —O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—(CH₂)₂—O—(CH₂)₂—N(CH₃)₂. Further exemplary alternatives include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other alternatives include 2′-methoxy (2′-OCH₃), 2′-aminopropoxy (2′-OCH₂CH₂CH₂NH₂) and 2′-fluoro (2′-F). Similar modifications can be made at other positions on the nucleosides and nucleotides of a dsRNA, particularly the 3′ position of the sugar on the 3′ terminal nucleotide or in 2′-5′ linked dsRNAs and the 5′ position of 5′ terminal nucleotide. dsRNAs can have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

A dsRNA can include nucleobase (often referred to in the art simply as “base”) alternatives (e.g., modifications or substitutions). Unmodified or natural nucleobases include the purine bases adenine (A) and guanine (G), and the pyrimidine bases thymine (T), cytosine (C) and uracil (U). Alternative nucleobases include other synthetic and natural nucleobases such as 5-methylcytidine, 5-hydroxymethylcytidine, 5-formylcytidine, 5-carboxycytidine, pyrrolocytidine, dideoxycytidine, uridine, 5-methoxyuridine, 5-hydroxydeoxyuridine, dihydrouridine, 4-thiourdine, pseudouridine, 1-methyl-pseudouridine, deoxyuridine, 5-hydroxybutynl-2′-deoxyuridine, xanthine, hypoxanthine, 7-deaza-xanthine, thienoguanine, 8-aza-7-deazaguanosine, 7-methylguanosine, 7-deazaguanosine, 6-aminomethyl-7-deazaguanosine, 8-aminoguanine, 2,2,7-trimethylguanosine, 8-methyladenine, 8-azidoadenine, 7-methyladenine, 7-deazaadenine, 3-deazaadenine, 2,6-diaminopurine, 2-aminopurine, 7-deaza-8-aza-adenine, 8-amino-adenine, thymine, dideoxythymine, 5-nitroindole, 2-aminoadenine, 6-methyl and other alkyl derivatives of adenine and guanine, 2-propyl and other alkyl derivatives of adenine and guanine, 2-thiouridine, 2-thiothymine and 2-thiocytosine, 5-halouracil and cytosine, 5-propynyl uridine and cytidine, 6-azo uridine, cytidine and thymine, 4-thiouridine, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uridines and cytidines, 8-azaguanine and 8-azaadenine, and 3-deazaguanine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, Antisense Research and Applications, pages 289-302, Crooke, S. T. and Lebleu, B., Ed., CRC Press, 1993. Certain of these nucleobases are particularly useful for increasing the binding affinity of the oligomeric compounds described herein. These include 5-substituted pyrimidines, 6-azapyrimidines, and N-2, N-6 and 0-6 substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., Antisense Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted alternative nucleobases as well as other alternative nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In other aspects, the sugar moiety in the nucleotide can be a ribose molecule, optionally having a 2′-O-methyl, 2′-O-MOE, 2′-F, 2′-amino, 2′-O-propyl, 2′-aminopropyl, or 2′-OH modification.

A dsRNA can include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In some aspects, the bridge connects the 4′-carbon and the 2′-carbon of the sugar ring. Thus, in some aspects, a dsRNA can include one or more locked nucleosides. A locked nucleoside is a nucleoside having a modified ribose moiety in which the ribose moiety comprises an extra bridge connecting the 2′ and 4′ carbons. In other words, a locked nucleoside is a nucleoside comprising a bicyclic sugar moiety comprising a 4′-CH₂—O-2′ bridge. This structure effectively “locks” the ribose in the 3′-endo structural conformation. The addition of locked nucleosides to dsRNAs has been shown to increase dsRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Examples of bicyclic nucleosides for use in the polynucleotides include without limitation nucleosides comprising a bridge between the 4′ and the 2′ ribosyl ring atoms. In some aspects, the antisense polynucleotide agents include one or more bicyclic nucleosides comprising a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)₂-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. Patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

A dsRNA can be modified to include one or more constrained ethyl nucleosides. As used herein, a “constrained ethyl nucleoside” or “cEt” is a locked nucleoside comprising a bicyclic sugar moiety comprising a 4′-CH(CH₃)—O-2′ bridge. In one aspect, a constrained ethyl nucleoside is in the S conformation referred to herein as “S-cEt.”

A dsRNA described herein can include one or more “conformationally restricted nucleosides” (“CRN”). CRN are nucleoside analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some aspects, a dsRNA comprises one or more monomers that are UNA (unlocked nucleoside) nucleosides. UNA is unlocked acyclic nucleoside, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose molecule can be modified with a cyclopropane ring to produce a tricyclodeoxynucleic acid (tricyclo DNA). The ribose moiety can be substituted for another sugar such as 1,5,-anhydrohexitol, threose to produce a threose nucleoside (TNA), or arabinose to produce an arabino nucleoside. The ribose molecule can be replaced with non-sugars such as cyclohexene to produce cyclohexene nucleoside or glycol to produce glycol nucleosides.

Potentially stabilizing modifications to the ends of nucleoside molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other alternatives chemistries of a dsRNA include a 5′ phosphate or 5′ phosphate mimic, e.g., a 5′-terminal phosphate or phosphate mimic on the antisense strand of a dsRNA. Suitable phosphate mimics are disclosed in, for example US Patent Publication No. 2012/0157511, the entire contents of which are incorporated herein by reference.

Exemplary dsRNAs comprise nucleosides with alternative sugar moieties and can comprise DNA or RNA nucleosides. In some aspects, the dsRNA comprises nucleosides comprising alternative sugar moieties and DNA nucleosides. Incorporation of alternative nucleosides into the dsRNA can enhance the affinity of the dsRNA for the target nucleic acid. In that case, the alternative nucleosides can be referred to as affinity enhancing alternative nucleotides.

In some aspects, the dsRNA comprises at least 1 alternative nucleoside, such as at least 2, at least 3, at least 4, at least 5, at least 6, at least 7, at least 8, at least 9, at least 10, at least 11, at least 12, at least 13, at least 14, at least 15, or at least 16 alternative nucleosides. In other aspects, the dsRNAs comprise from 1 to 10 alternative nucleosides, such as from 2 to 9 alternative nucleosides, such as from 3 to 8 alternative nucleosides, such as from 4 to 7 alternative nucleosides, such as 6 or 7 alternative nucleosides. In an aspect, the dsRNA can comprise alternatives, which are independently selected from these three types of alternative (alternative sugar moiety, alternative nucleobase, and alternative internucleoside linkage), or a combination thereof. In one aspect, the dsRNA comprises one or more nucleosides comprising alternative sugar moieties, e.g., 2′ sugar alternative nucleosides. In some aspects, the dsRNA comprise the one or more 2′ sugar alternative nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA, and BNA (e.g., LNA) nucleosides. In some aspects, the one or more alternative nucleoside is a BNA.

In some aspects, at least 1 of the alternative nucleosides is a BNA (e.g., an LNA), such as at least 2, such as at least 3, at least 4, at least 5, at least 6, at least 7, or at least 8 of the alternative nucleosides are BNAs. In a still further aspect, all the alternative nucleosides are BNAs.

In a further aspect the dsRNA comprises at least one alternative internucleoside linkage. In some aspects, the internucleoside linkages within the contiguous nucleotide sequence are phosphorothioate or boronophosphate internucleoside linkages. In some aspects, all the internucleotide linkages in the contiguous sequence of the oligonucleotide are phosphorothioate linkages. In some aspects, the phosphorothioate linkages are stereochemically pure phosphorothioate linkages. In some aspects, the phosphorothioate linkages are Sp phosphorothioate linkages. In other aspects, the phosphorothioate linkages are Rp phosphorothioate linkages.

In some aspects, the dsRNA comprises at least one alternative nucleoside which is a 2′-MOE-RNA, such as 2, 3, 4, 5, 6, 7, 8, 9, or 10 2′-MOE-RNA nucleoside units. In some aspects, the 2′-MOE-RNA nucleoside units are connected by phosphorothioate linkages. In some aspects, at least one of said alternative nucleoside is 2′-fluoro DNA, such as 2, 3, 4, 5, 6, 7, 8, 9, or 10 2′-fluoro-DNA nucleoside units. In some aspects, the dsRNA comprises at least one BNA unit and at least one 2′ substituted modified nucleoside. In some aspects, the dsRNA comprises both 2′ sugar modified nucleosides and DNA units.

B. dsRNAs Conjugated to Ligands

dsRNAs can be chemically linked to one or more ligands, moieties, or conjugates that enhance the activity, cellular distribution, or cellular uptake of the dsRNA. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

In one aspect, a ligand alters the distribution, targeting, or lifetime of a dsRNA agent into which it is incorporated. In some aspects, a ligand provides an enhanced affinity for a selected target, e.g., molecule, cell or cell type, compartment, e.g., a cellular or organ compartment, tissue, organ, or region of the body, as, e.g., compared to a species absent such a ligand. Some ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can include hormones and hormone receptors. They can include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose.

The ligand can be a substance, e.g., a drug, which can increase the uptake of the dsRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some aspects, a ligand attached to a dsRNA as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. dsRNA that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable as ligands (e.g. as PK modulating ligands). In addition, aptamers that bind serum components (e.g. serum proteins) are also suitable for use as PK modulating ligands in the aspects described herein.

Ligand-conjugated dsRNAs can be synthesized by the use of a dsRNA that bears a pendant reactive functionality, such as that derived from the attachment of a linking molecule onto the dsRNA (described below). This reactive dsRNA can be reacted directly with commercially-available ligands, ligands that are synthesized bearing any of a variety of protecting groups, or ligands that have a linking moiety attached thereto.

The dsRNAs used in the conjugates can be conveniently and routinely made through the well-known technique of solid-phase synthesis. Equipment for such synthesis is sold by several vendors including, for example, Applied Biosystems (Foster City, Calif.). Any other means for such synthesis known in the art can additionally or alternatively be employed. It is also known to use similar techniques to prepare other dsRNA, such as the phosphorothioates and alkylated derivatives.

In the ligand-conjugated dsRNA, such as the ligand-molecule bearing sequence-specific linked nucleosides, the oligonucleotides and oligonucleosides can be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using conjugate precursors that already bear a linking moiety, the synthesis of the sequence-specific linked nucleosides is typically completed, and the ligand molecule is then reacted with the linking moiety to form the ligand-conjugated dsRNAs. In some aspects, the oligonucleotides or linked nucleosides described herein are synthesized by an automated synthesizer using phosphoramidites derived from ligand-nucleoside conjugates in addition to the standard phosphoramidites and non-standard phosphoramidites that are commercially available and routinely used in oligonucleotide synthesis.

i. Lipid Conjugates

In one aspect, the ligand or conjugate is a lipid or lipid-based molecule. Such a lipid or lipid-based molecule can bind a serum protein, e.g., human serum albumin (HSA). An HSA binding ligand allows for distribution of the conjugate to a target tissue, e.g., a non-kidney target tissue of the body. A lipid or lipid-based ligand can (a) increase resistance to degradation of the conjugate, (b) increase targeting or transport into a target cell or cell membrane, and/or (c) can be used to adjust binding to a serum protein, e.g., HSA.

In another aspect, the ligand is a moiety, e.g., a vitamin, which is taken up by a target cell, e.g., a proliferating cell. Exemplary vitamins include vitamin A, E, and K.

ii. Cell Permeation Agents

In another aspect, the ligand is a cell-permeation agent, such as a helical cell-permeation agent. In one aspect, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. In one aspect, the helical agent is an alpha-helical agent, which can have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to dsRNA agents can affect pharmacokinetic distribution of the dsRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP. An RFGF analogue (e.g., amino acid sequence AALLPVLLAAP containing a hydrophobic MTS can be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ and the Drosophila Antennapedia protein (RQIKIWFQNRRMKWKK have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to a dsRNA agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods can be linear or cyclic, and can be modified, e.g., glycosylated or methylated, to facilitate targeting to a specific tissue(s). RGD-containing peptides and peptidiomimemtics can include D-amino acids, as well as synthetic RGD mimics. In addition to RGD, one can use other moieties that target the integrin ligand. Some conjugates of this ligand target PECAM-1 or VEGF.

A cell permeation peptide is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin,β-defensin, or bactenecin), or a peptide containing only one or two dominating amino acids (e.g., PR-39 or indolicidin). A cell permeation peptide can include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

iii. Carbohydrate Conjugates

In some aspects of the compositions and methods, a dsRNA further comprises a carbohydrate. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In one aspect, a carbohydrate conjugate for use in the compositions and methods is a monosaccharide.

In some aspects, the carbohydrate conjugate further comprises one or more additional ligands as described above, such as, but not limited to, a PK modulator and/or a cell permeation peptide.

Additional carbohydrate conjugates (and linkers) suitable for use include those described in PCT Publication Nos. WO 2014/179620 and WO 2014/179627, the entire contents of each of which are incorporated herein by reference.

iv. Linkers

In some aspects, the conjugate or ligand described herein can be attached to a dsRNA with various linkers that can be cleavable or non-cleavable.

Linkers typically comprise a direct bond or an atom such as oxygen or sulfur, a unit such as NR⁸, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R⁸), C(O), substituted or unsubstituted aryl, substituted or unsubstituted heteroaryl, substituted or unsubstituted heterocyclic; where R⁸ is hydrogen, acyl, aliphatic or substituted aliphatic. In one aspect, the linker is between about 1-24, 2-24, 3-24, 4-24, 5-24, 6-24, 6-18, 7-18, 8-18, 7-17, 8-17, 6-16, 7-17, or 8-16 or 1, 2, 3, 4, 5, 6, 7, 8, 9, 10, 11, 12, 13, 14, 15, 16, 17, 18, 19, 20, 21, 22, 23, or 24 atoms.

A cleavable linking group is one which is sufficiently stable outside the cell, but which upon entry into a target cell is cleaved to release the two parts the linker is holding together. In some aspects, the cleavable linking group is cleaved at least about 10 times, 20, times, 30 times, 40 times, 50 times, 60 times, 70 times, 80 times, 90 times, or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selective for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a certain pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It can also be desirable to test the candidate cleavable linking group for the ability to resist cleavage in the blood or when in contact with other non-target tissue. Thus, one can determine the relative susceptibility to cleavage between at least two conditions, where at least one is selected to be indicative of cleavage in a target cell and another is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In some aspects, useful candidate compounds are cleaved at least about 2, 4, 10, 20, 30, 40, 50, 60, 70, 80, 90, or 100 times faster in the cell (or under in vitro conditions selected to mimic intracellular conditions) as compared to blood or serum (or under in vitro conditions selected to mimic extracellular conditions).

a. Redox Cleavable Linking Groups

In one aspect, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular dsRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can be evaluated under conditions which are selected to mimic blood or serum conditions. In one aspect, candidate compounds are cleaved by at most about 10% in the blood. In other aspects, useful candidate compounds are degraded at least about 2, 4, 10, 20, 30, 40, 50, 60, 70, 80, 90, or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

b. Phosphate-Based Cleavable Linking Groups

In another aspect, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(OR^{k})—O—, —O—P(S)(OR^{k})—O—, —O—P(S)(SR^{k})—O, —S—P(O)(OR^{k})—O—, —O—P(O)(OR^{k})—S—, —S—P(O)(OR^{k})—S—, —O—P(S)(OR^{k})—S—, —S—P(S)(OR^{k})—O—, —O—P(O)(R^{k})—O—, —O—P(S)(R^{k})—O—, —S—P(O)(R^{k})—O—, —S—P(S)(R^{k})—O—, —S—P(O)(R^{k})—S—, —O—P(S)(R^{k})—S—. These candidates can be evaluated using methods analogous to those described above.

c. Acid Cleavable Linking Groups

In another aspect, a cleavable linker comprises an acid cleavable linking group. An acid cleavable linking group is a linking group that is cleaved under acidic conditions. In some aspects, acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). In one aspect, the carbon is attached to the oxygen of the ester (the alkoxy group) is an aryl group, substituted alkyl group, or tertiary alkyl group such as dimethyl pentyl or t-butyl. These candidates can be evaluated using methods analogous to those described above.

d. Ester-Based Linking Groups

In another aspect, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

e. Peptide-Based Cleaving Groups

In yet another aspect, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCHR^{B}C(O)—, where RA and R^{B} are the R groups of the two adjacent amino acids. These candidates can be evaluated using methods analogous to those described above.

In one aspect, a dsRNA is conjugated to a carbohydrate through a linker. Linkers include bivalent and trivalent branched linker groups. Linkers for dsRNA carbohydrate conjugates include, but are not limited to, those described in formulas 24-35 of PCT Publication No. WO 2018/195165.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within a dsRNA. dsRNA compounds that are chimeric compounds are also contemplated. Chimeric dsRNAs typically contain at least one region wherein the RNA is modified so as to confer upon the dsRNA increased resistance to nuclease degradation, increased cellular uptake, and/or increased binding affinity for the target nucleic acid. An additional region of the dsRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of dsRNA reduction of expression. Consequently, comparable results can often be obtained with shorter dsRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the nucleosides of a dsRNA can be modified by a non-ligand group. A number of non-ligand molecules have been conjugated to dsRNAs in order to enhance the activity, cellular distribution, or cellular uptake of the dsRNA, and procedures for performing such conjugations are available in the scientific literature. Such non-ligand moieties have included lipid moieties, such as cholesterol (Kubo, T. et al., Biochem. Biophys. Res. Comm, 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such dsRNA conjugates have been listed above. Typical conjugation protocols involve the synthesis of a dsRNA bearing an aminolinker at one or more positions of the sequence. The amino group is then reacted with the molecule being conjugated using appropriate coupling or activating reagents. The conjugation reaction can be performed either with the dsRNA still bound to the solid support or following cleavage of the dsRNA, in solution phase. Purification of the dsRNA conjugate by HPLC typically affords the pure conjugate.

### IV. Pharmaceutical Uses

The dsRNA compositions described herein are useful in the methods and, while not bound by theory, are believed to exert their desirable effects through their ability to modulate the level, status, and/or activity of a MutSβ heterodimer comprising MSH3, e.g., by reducing the activity or level of the MSH3 protein in a cell in a mammal.

Methods of treating disorders related to DNA mismatch repair such as nucleotide repeat expansion disorders (e.g., trinucleotide repeat expansion disorders) in a subject in need thereof are also contemplated. Another aspect includes reducing the level of MSH3 in a cell of a subject identified as having a nucleotide repeat expansion disorder (e.g., trinucleotide repeat expansion disorders). Still another aspect includes a method of reducing expression of MSH3 in a cell in a subject. Further aspects include methods of decreasing nucleotide repeat expansion in a cell (e.g., trinucleotide repeat expansion). The methods include contacting a cell with a dsRNA, in an amount effective to reduce expression of MSH3 in the cell, thereby reducing expression of MSH3 in the cell.

Based on the above methods, a dsRNA, or a composition comprising such a dsRNA, for use in therapy, or for use as a medicament, or for use in treating disorders related to DNA mismatch repair such as trinucleotide repeat expansion disorders in a subject in need thereof, or for use in reducing the level of MSH3 in a cell of a subject identified as having a trinucleotide repeat expansion disorder, or for use in reducing expression of MSH3 in a cell in a subject, or for use in decreasing trinucleotide repeat expansion in a cell is contemplated. The uses include the contacting of a cell with the dsRNA, in an amount effective to reduce expression of MSH3 in the cell, thereby reducing expression of MSH3 in the cell. Aspects described below in relation to the methods are also applicable to these further aspects.

Contacting of a cell with a dsRNA, e.g., a double stranded dsRNA, can be done in vitro or in vivo. Contacting a cell in vivo with the dsRNA includes contacting a cell or group of cells within a subject, e.g., a human subject, with the dsRNA. Combinations of in vitro and in vivo methods of contacting a cell are also possible. Contacting a cell can be direct or indirect, as discussed above. Furthermore, contacting a cell can be accomplished via a targeting ligand, including any ligand described herein or known in the art. In some aspects, the targeting ligand is a carbohydrate moiety, e.g., a GalNAc₃ ligand, or any other ligand that directs the dsRNA to a site of interest. Cells can include those of the central nervous system, or muscle cells.

Reducing expression of MSH3 includes any level of reduction of MSH3, e.g., at least partial suppression of the expression of a MSH3, such as a reduction by at least about 20%. In some aspects, the reduction is by at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

The expression of MSH3 can be assessed based on the level of any variable associated with MSH3 expression, e.g., MSH3 mRNA level or MSH3 protein level.

Reduction can be assessed by a decrease in an absolute or relative level of one or more of these variables compared with a control level. The control level can be any type of control level that is utilized in the art, e.g., a pre-dose baseline level, or a level determined from a similar subject, cell, or sample that is untreated or treated with a control (such as, e.g., buffer only control or inactive agent control).

In some aspects, surrogate markers can be used to detect reduction of MSH3. For example, effective treatment of a trinucleotide repeat expansion disorder, as demonstrated by acceptable diagnostic and monitoring criteria with an agent to reduce MSH3 expression can be understood to demonstrate a clinically relevant reduction in MSH3.

In some aspects of the methods, expression of a MSH3 is reduced by at least 20%, 25%, 30%, 35%, 40%, 45%, 50%, 55%, 60%, 65%, 70%, 75%, 80%, 85%, 90%, or 95%, or to below the level of detection of the assay. In some aspects, the methods include a clinically relevant reduction of expression of MSH3, e.g., as demonstrated by a clinically relevant outcome after treatment of a subject with an agent to reduce the expression of MSH3.

Reduction of the expression of MSH3 can be manifested by a reduction of the amount of mRNA expressed by a first cell or group of cells (such cells can be present, for example, in a sample derived from a subject) in which MSH3 is transcribed and which has or have been treated (e.g., by contacting the cell or cells with a dsRNA, or by administering a dsRNA to a subject in which the cells are or were present) such that the expression of MSH3 is reduced, as compared to a second cell or group of cells substantially identical to the first cell or group of cells but which has not or have not been so treated (control cell(s) not treated with a dsRNA or not treated with a dsRNA targeted to the gene of interest). The degree of reduction can be expressed in terms of:

$$\frac{( mRNA in control cells ) - ( mRNA in treated cells )}{( mRNA in control cells )} \times 100 \%$$

In other aspects, reduction of the expression of MSH3 can be assessed in terms of a reduction of a parameter that is functionally linked to MSH3 expression, e.g., MSH3 protein expression or MSH3 signaling pathways. MSH3 silencing can be determined in any cell expressing MSH3, either endogenous or heterologous from an expression construct, and by any assay known in the art.

Reduction of the expression of a MSH3 protein can be manifested by a reduction in the level of the MSH3 protein that is expressed by a cell or group of cells (e.g., the level of protein expressed in a sample derived from a subject). As explained above, for the assessment of mRNA suppression, the reduction of protein expression levels in a treated cell or group of cells can similarly be expressed as a percentage of the level of protein in a control cell or group of cells.

A control cell or group of cells that can be used to assess the reduction of the expression of MSH3 includes a cell or group of cells that has not yet been contacted with a dsRNA. For example, the control cell or group of cells can be derived from an individual subject (e.g., a human or animal subject) prior to treatment of the subject with a dsRNA.

The level of MSH3 mRNA that is expressed by a cell or group of cells can be determined using any method known in the art for assessing mRNA expression. In one aspect, the level of expression of MSH3 in a sample is determined by detecting a transcribed polynucleotide, or portion thereof, e.g., mRNA of the MSH3 gene. RNA can be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating MSH3 mRNA can be detected using methods the described in PCT Publication WO2012/177906, the entire contents of which are hereby incorporated herein by reference. In some aspects, the level of expression of MSH3 is determined using a nucleic acid probe. The term “probe,” as used herein, refers to any molecule that is capable of selectively binding to a specific MSH3 sequence, e.g. to an mRNA or polypeptide. Probes can be synthesized by one of skill in the art, or derived from appropriate biological preparations. Probes can be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to MSH3 mRNA. In one aspect, the mRNA is immobilized on a solid surface and contacted with a probe, for example by running the isolated mRNA on an agarose gel and transferring the mRNA from the gel to a membrane, such as nitrocellulose. In an alternative aspect, the probe(s) are immobilized on a solid surface and the mRNA is contacted with the probe(s), for example, in an AFFYMETRIX gene chip array. A skilled artisan can readily adapt known mRNA detection methods for use in determining the level of MSH3 mRNA.

An alternative method for determining the level of expression of MSH3 in a sample involves the process of nucleic acid amplification and/or reverse transcriptase (to prepare cDNA) of for example mRNA in the sample, e.g., by RT-PCR (the experimental aspect set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects, the level of expression of MSH3 is determined by quantitative fluorogenic RT-PCR (i.e., the TAQMAN™ System) or the DUAL-GLO® Luciferase assay.

The expression levels of MSH3 mRNA can be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722; 5,874,219; 5,744,305; 5,677,195; and 5,445,934, which are incorporated herein by reference. The determination of MSH3 expression level can comprise using nucleic acid probes in solution.

In some aspects, the level of mRNA expression is assessed using branched DNA (bDNA) assays or real time PCR (qPCR). The use of this PCR method is described and exemplified in the Examples presented herein. Such methods can be used for the detection of MSH3 nucleic acids.

The level of MSH3 protein expression can be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can be used for the detection of proteins indicative of the presence or replication of MSH3 proteins.

In some aspects of the methods, the dsRNA is administered to a subject such that the dsRNA is delivered to a specific site within the subject. The reduction of expression of MSH3 can be assessed using measurements of the level or change in the level of MSH3 mRNA or MSH3 protein in a sample derived from a specific site within the subject. In some aspects, the methods include a clinically relevant reduction of expression of MSH3, e.g., as demonstrated by a clinically relevant outcome after treatment of a subject with an agent to reduce the expression of MSH3.

In other aspects, the dsRNA is administered in an amount and for a time effective to result in one of (or more, e.g., two or more, three or more, four or more of): (a) decrease the number of trinucleotide repeats, (b) decrease the level of polyglutamine, (c) decreased cell death (e.g., CNS cell death and/or muscle cell death), (d) delayed onset of the disorder, (e) increased survival of subject, and (f) increased progression free survival of a subject.

Treating nucleotide repeat expansion disorders (e.g., trinucleotide repeat expansion disorders) can result in an increase in average survival time of an individual or a population of subjects treated with the methods disclosed herein in comparison to a population of untreated subjects. For example, the survival time is of an individual or average survival time a of population is increased by more than 30 days (more than 60 days, 90 days, or 120 days). An increase in survival time of an individual or in average survival time of a population can be measured by any reproducible means. An increase in survival time of an individual can be measured, for example, by calculating for an individual the length of survival time following the initiation of treatment with the compound described herein. An increase in average survival time of a population can be measured, for example, by calculating for the average length of survival time following initiation of treatment with the compound described herein. An increase in survival time of an individual can be measured, for example, by calculating for an individual length of survival time following completion of a first round of treatment with a compound or pharmaceutically acceptable salt of a compound described herein. An increase in average survival time of a population can be measured, for example, by calculating for a population the average length of survival time following completion of a first round of treatment with a compound or pharmaceutically acceptable salt of a compound described herein.

Treating nucleotide repeat expansion disorders (e.g., trinucleotide repeat expansion disorders) can result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects can be measured by any reproducible means, for example, by calculating for a population the average number of disease-related deaths per unit time following initiation of treatment with a compound or pharmaceutically acceptable salt of a compound described herein. A decrease in the mortality rate of a population can be measured, for example, by calculating for a population the average number of disease-related deaths per unit time following completion of a first round of treatment with a compound or pharmaceutically acceptable salt of a compound described herein.

### A. Delivery of Anti-MSH3 Agents

The delivery of a dsRNA to a cell e.g., a cell within a subject, such as a human subject e.g., a subject in need thereof, such as a subject having a nucleotide repeat expansion disorder (e.g., trinucleotide repeat expansion disorders) can be achieved in a number of different ways. For example, delivery can be performed by contacting a cell with a dsRNA either in vitro or in vivo. In vivo delivery can be performed directly by administering a composition comprising a dsRNA, e.g., a siRNA or a shRNA, to a subject. These alternatives are discussed further below.

In general, any method of delivering a nucleic acid molecule (in vitro or in vivo) can be adapted for use with a dsRNA (see e.g., Akhtar S. and Julian R L., (1992) Trends Cell. Biol. 2(5):139-144 and WO94/02595, which are incorporated herein by reference in their entireties). For in vivo delivery, factors to consider in order to deliver a dsRNA molecule include, for example, biological stability of the delivered molecule, prevention of non-specific effects, and accumulation of the delivered molecule in the target tissue. The non-specific effects of a dsRNA can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the dsRNA molecule to be administered.

For administering a dsRNA systemically for the treatment of a disease, the dsRNA can include alternative nucleobases, alternative sugar moieties, and/or alternative internucleoside linkages, or alternatively delivered using a drug delivery system; both methods act to prevent the rapid degradation of the dsRNA by endo- and exo-nucleases in vivo. Modification of the dsRNA or the pharmaceutical carrier can permit targeting of the dsRNA composition to the target tissue and avoid undesirable off-target effects. dsRNA molecules can be modified by chemical conjugation to lipophilic groups such as cholesterol to enhance cellular uptake and prevent degradation. For example, a dsRNA directed against ApoB conjugated to a lipophilic cholesterol moiety was injected systemically into mice and resulted in knockdown of apoB mRNA in both the liver and jejunum (Soutschek, J. et al., (2004) Nature 432:173-178). Conjugation of a dsRNA to an aptamer has been shown to reduce tumor growth and mediate tumor regression in a mouse model of prostate cancer (McNamara, J O. et al., (2006) Nat. Biotechnol. 24:1005-1015). In an alternative aspect, the dsRNA can be delivered using drug delivery systems such as a nanoparticle, a lipid nanoparticle, a polyplex nanoparticle, a lipoplex nanoparticle, a dendrimer, a polymer, liposomes, or a cationic delivery system. Positively charged cationic delivery systems facilitate binding of a dsRNA molecule (negatively charged) and also enhance interactions at the negatively charged cell membrane to permit efficient uptake of a dsRNA by the cell. Cationic lipids, dendrimers, or polymers can either be bound to a dsRNA, or induced to form a vesicle or micelle (see e.g., Kim S H. et al., (2008) Journal of Controlled Release 129(2):107-116) that encases a dsRNA. The formation of vesicles or micelles further prevents degradation of the dsRNA when administered systemically. In general, any methods of delivery of nucleic acids known in the art can be adaptable to the delivery of the dsRNAs. Methods for making and administering cationic-dsRNA complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of dsRNAs include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some aspects, a dsRNA forms a complex with cyclodextrin for systemic administration. Methods for administration and pharmaceutical compositions of dsRNAs and cyclodextrins can be found in U.S. Pat. No. 7,427,605, which is herein incorporated by reference in its entirety. In some aspects, the dsRNAs are delivered by polyplex or lipoplex nanoparticles. Methods for administration and pharmaceutical compositions of dsRNAs and polyplex nanoparticles and lipoplex nanoparticles can be found in U.S. Patent Application Nos. 2017/0121454; 2016/0369269; 2016/0279256; 2016/0251478; 2016/0230189; 2015/0335764; 2015/0307554; 2015/0174549; 2014/0342003; 2014/0135376; and 2013/0317086, which are herein incorporated by reference in their entirety.

i. Vector Delivery Methods

dsRNA targeting MSH3 can be expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can be constructed to permit it to be inherited as an extrachromosomal plasmid (Gassmann, et al., (1995) Proc. Natl. Acad. Sci. USA 92:1292).

The individual strand or strands of a dsRNA can be transcribed from a promoter on an expression vector. Where two separate strands are to be expressed to generate, for example, a dsRNA, two separate expression vectors can be co-introduced (e.g., by transfection or infection) into a target cell. Alternatively, each individual strand of a dsRNA can be transcribed by promoters both of which are located on the same expression plasmid. In one aspect, a dsRNA is expressed as inverted repeat polynucleotides joined by a linker polynucleotide sequence such that the dsRNA has a stem and loop structure.

dsRNA expression vectors are generally DNA plasmids or viral vectors. Expression vectors compatible with eukaryotic cells, such as those compatible with vertebrate cells, can be used to produce recombinant constructs for the expression of a dsRNA as described herein. Eukaryotic cell expression vectors are well known in the art and are available from a number of commercial sources. Typically, such vectors are provided containing convenient restriction sites for insertion of the desired nucleic acid segment. Delivery of dsRNA expressing vectors can be systemic, such as by intravenous or intramuscular administration, by administration to target cells ex-planted from the patient followed by reintroduction into the patient, or by any other means that allows for introduction into a desired target cell.

In some aspects, the dsRNA agent that reduces the level and/or activity of MSH3 is delivered by a viral vector (e.g., a viral vector expressing an anti-MSH3 agent). Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double-stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the vectors of which are incorporated herein by reference.

Exemplary viral vectors include lentiviral vectors, AAVs, and retroviral vectors. Lentiviral vectors and AAVs can integrate into the genome without cell divisions, and both types have been tested in pre-clinical animal studies. Methods for preparation of AAVs are described in the art e.g., in U.S. Pat. Nos. 5,677,158, 6,309,634, and 6,683,058, the methods of which is incorporated herein by reference. Methods for preparation and in vivo administration of lentiviruses are described in US 20020037281, the methods of which are incorporated herein by reference. In one aspect, a lentiviral vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding the fusion protein, an origin of second strand DNA synthesis and a 3′ lentiviral LTR.

Retroviruses are most commonly used in human clinical trials, as they carry 7-8 kb, and have the ability to infect cells and have their genetic material stably integrated into the host cell with high efficiency (see, e.g., WO 95/30761; WO 95/24929, the retroviruses of which is incorporated herein by reference). In one aspect, a retroviral vector is replication defective. This prevents further generation of infectious retroviral particles in the target tissue. Thus, the replication defective virus becomes a “captive” transgene stable incorporated into the target cell genome. This is typically accomplished by deleting the gag, env, and pol genes (along with most of the rest of the viral genome). Heterologous nucleic acids are inserted in place of the deleted viral genes. The heterologous genes can be under the control of the endogenous heterologous promoter, another heterologous promoter active in the target cell, or the retroviral 5′ LTR (the viral LTR is active in diverse tissues).

These delivery vectors described herein can be made target-specific by attaching, for example, a sugar, a glycolipid, or a protein (e.g., an antibody to a target cell receptor).

Reversible delivery expression systems can be used. The Cre-loxP or FLP/FRT system and other similar systems can be used for reversible delivery-expression of one or more of the above-described nucleic acids. See WO2005/112620, WO2005/039643, US20050130919, US20030022375, US20020022018, US20030027335, and US20040216178, the systems of which are herein incorporated by reference. In particular, the reversible delivery-expression system described in US20100284990, the systems of which are herein incorporated by reference, can be used to provide a selective or emergency shut-off.

ii. Membranous Molecular Assembly Delivery Methods

dsRNAs can be delivered using a variety of membranous molecular assembly delivery methods including polymeric, biodegradable microparticle, or microcapsule delivery devices known in the art. For example, a colloidal dispersion system can be used for targeted delivery a dsRNA agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the dsRNA are delivered into the cell where the dsRNA can specifically bind to a target RNA and can mediate RNAi. In some cases, the liposomes are also specifically targeted, e.g., to direct the dsRNA to particular cell types. The composition of the liposome is usually a combination of phospholipids, usually in combination with steroids, especially cholesterol. Other phospholipids or other lipids can be used. The physical characteristics of liposomes depend on pH, ionic strength, and the presence of divalent cations.

A liposome containing a dsRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and can be nonionic. Exemplary detergents include cholate, CHAPS, octylglucoside, deoxycholate, and lauroyl sarcosine. The dsRNA preparation is then added to the micelles that include the lipid component. The cationic groups on the lipid interact with the dsRNA and condense around the dsRNA to form a liposome. After condensation, the detergent is removed, e.g., by dialysis, to yield a liposomal preparation of dsRNA.

If necessary, a carrier compound that assists in condensation can be added during the condensation reaction, e.g., by controlled addition. For example, the carrier compound can be a polymer other than a nucleic acid (e.g., spermine or spermidine). The pH can be adjusted to favor condensation.

Methods for producing stable polynucleotide delivery vehicles, which incorporate a polynucleotide/cationic lipid complex as a structural component of the delivery vehicle, are further described in, e.g., WO 96/37194, the entire contents of which are incorporated herein by reference. Liposome formation can include one or more aspects of exemplary methods described in Feigner, P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169). These methods are readily adapted to packaging dsRNA preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems comprising non-ionic surfactant and cholesterol. Non-ionic liposomal formulations comprising NOVASOME™ I (glyceryl dilaurate/cholesterol/polyoxyethylene-10-stearyl ether) and NOVASOME™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P. Pharma. Sci., 4(6):466).

Liposomes can be sterically stabilized liposomes, comprising one or more specialized lipids that result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside Gm′, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes comprising one or more glycolipids are known in the art. Papahadjopoulos et al. (Ann. N.Y. Acad. Sci., (1987), 507:64) reported the ability of monosialoganglio side G^{M1}, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one aspect, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver dsRNAs to macrophages.

Further advantages of liposomes include: liposomes obtained from natural phospholipids are biocompatible and biodegradable; liposomes can incorporate a wide range of water and lipid soluble drugs; liposomes can protect encapsulated dsRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of dsRNA (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM′, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer dsRNA into the skin. In some implementations, liposomes are used for delivering dsRNA to epidermal cells and also to enhance the penetration of dsRNA into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2, 405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems comprising non-ionic surfactant and cholesterol. Non-ionic liposomal formulations comprising NOVASOME I (glyceryl dilaurate/cholesterol/polyoxyethylene-10-stearyl ether) and NOVASOME II (glyceryl distearate/cholesterol/polyoxyethylene-10-stearyl ether) were used to deliver a drug into the dermis of mouse skin. Such formulations with dsRNA are useful for treating a dermatological disorder.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include dsRNA can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include dsRNA can be delivered, for example, subcutaneously by infection in order to deliver dsRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application No. PCT/US2007/080331, filed Oct. 3, 2007 also describes formulations that are amenable for use in the methods described herein.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The dsRNA for use in the methods can be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

iii. Lipid Nanoparticle-Based Delivery Methods

dsRNAs described herein can be fully encapsulated in a lipid formulation, e.g., a lipid nanoparticle (LNP), or other nucleic acid-lipid particle. LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one aspect, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated.

Non-limiting examples of cationic lipids include N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can comprise, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 60 mol % if cholesterol is included, of the total lipid present in the particle.

The conjugated lipid that reduces aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (C₁₈). The conjugated lipid that prevents aggregation of particles can be, for example, from 0 mol % to about 20 mol % or about 2 mol % of the total lipid present in the particle.

In some aspects, the nucleic acid-lipid particle further includes cholesterol at, e.g., about 10 mol % to about 60 mol % or about 50 mol % of the total lipid present in the particle.

Additional exemplary lipid-dsRNA formulations are described in Table 1 of WO 2018/195165, herein incorporated by reference.

### B. Combination Therapies

A dsRNA can be used alone or in combination with at least one additional therapeutic agent, e.g., other agents that treat nucleotide repeat expansion disorders (e.g., trinucleotide repeat expansion disorders) or symptoms associated therewith, or in combination with other types of therapies to treat trinucleotide repeat expansion disorders. In combination treatments, the dosages of one or more of the therapeutic compounds can be reduced from standard dosages when administered alone. For example, doses can be determined empirically from drug combinations and permutations or can be deduced by isobolographic analysis (e.g., Black et al., Neurology 65:S3-S6 (2005)). In this case, dosages of the compounds when combined should provide a therapeutic effect.

In some aspects, the dsRNA agents described herein can be used in combination with at least one additional therapeutic agent to treat a nucleotide repeat expansion disorder (e.g., trinucleotide repeat expansion disorders) associated with gene having a trinucleotide repeat (e.g., any of the trinucleotide repeat expansion disorders and associated genes having a trinucleotide repeat listed in Table 1). In some aspects, at least one additional therapeutic agent can be an oligonucleotide (e.g., an ASO) that hybridizes with the mRNA of gene associated with a trinucleotide repeat expansion disorder (e.g., any of the genes listed in Table 1). In some aspects, the inucleotide repeat expansion disorder (e.g., trinucleotide repeat expansion disorders) is Huntington's disease (HD). In some aspects, the gene associated with a nucleotide repeat expansion disorder (e.g., trinucleotide repeat expansion disorders) is Huntingtin (HTT). Several allelic variants of the Huntingtin gene have been implicated in the etiology of Huntington's disease. In some cases, these variants are identified on the basis of having unique HD-associated single nucleotide polymorphisms (SNPs). In some aspects, the other oligonucleotide (e.g., an ASO) hybridizes to an mRNA of the Huntingtin gene containing any of the HD-associated SNPs known in the art (e.g., any of the HD-associated SNPs described in Skotte et al., PLoS One 2014, 9(9): e107434, Carroll et al., Mol. Ther. 2011, 19(12): 2178-85, Warby et al., Am. Hum. Gen. 2009, 84(3): 351-66 (herein incorporated by reference)). In some aspects, the other oligonucleotide (e.g., an ASO) hybridizes to an mRNA of the Huntingtin gene lacking any of the HD-associated SNPs. In some of the aspects, the other oligonucleotide (e.g., an ASO) hybridizes to an mRNA of the Huntingtin gene having any of the SNPs selected from the group of rs362307 and rs365331. In some aspects, the other oligonucleotide (e.g., an ASO) can be a modified oligonucleotide (e.g., an oligonucleotide including any of the modifications described herein). In some aspects, the modified oligonucleotides comprise one or more phosphorothioate internucleoside linkages. In some aspects, the modified oligonucleotide comprises one or more 2′-MOE moieties. In some aspects, the other oligonucleotide (e.g., an ASO) that hybridizes to the mRNA of the Huntingtin gene has a sequence selected from the SEQ ID NOs. 6-285 of U.S. Pat. No. 9,006,198; SEQ ID NOs. 6-8 of US Patent Application Publication No. 2017/0044539; SEQ ID NOs. 1-1565 of US Patent Application Publication 2018/0216108; and SEQ ID NOs. 1-2432 of PCT Publication WO 2017/192679, the sequences of which are hereby incorporated by reference.

In some aspects, at least one additional therapeutic agent is a chemotherapeutic agent (e.g., a cytotoxic agent or other chemical compound useful in the treatment of a trinucleotide repeat expansion disorder). In some aspects, at least one additional therapeutic agent can be a therapeutic agent which is a non-drug treatment. For example, at least one therapeutic agent can be physical therapy.

In any of the combination aspects described herein, the two or more therapeutic agents are administered simultaneously or sequentially, in either order. For example, a first therapeutic agent can be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after one of more of the additional therapeutic agents.

### V. Pharmaceutical Compositions

The dsRNAs described herein can be formulated into pharmaceutical compositions for administration to human subjects in a biologically compatible form suitable for administration in vivo.

The compounds described herein can be used in the form of the free base, in the form of salts, solvates, and as prodrugs. All forms are within the methods described herein. In accordance with the methods, the dsRNAs or salts, solvates, or prodrugs thereof can be administered to a patient in a variety of forms depending on the selected route of administration, as will be understood by those skilled in the art. The compounds described herein can be administered, for example, by oral, parenteral, intrathecal, intracerebroventricular, intraparenchymal, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, intracerebroventricular, intraparenchymal, rectal, and topical modes of administration. Parenteral administration can be by continuous infusion over a selected period of time.

A compound described herein can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, a compound described herein can be incorporated with an excipient and used in the form of ingestible tablets, buccal tablets, troches, capsules, elixirs, suspensions, syrups, and wafers. A compound described herein can be administered parenterally. Solutions of a compound described herein can be prepared in water suitably mixed with a surfactant, such as hydroxypropylcellulose. Dispersions can be prepared in glycerol, liquid polyethylene glycols, DMSO, and mixtures thereof with or without alcohol, and in oils. Under ordinary conditions of storage and use, these preparations can contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that can be easily administered via syringe. Compositions for nasal administration can conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container can be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter

The compounds described herein can be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### VI. Dosages

The dosage of the compositions (e.g., a composition including a dsRNA) described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. The compositions described herein can be administered initially in a suitable dosage that can be adjusted as required, depending on the clinical response. In some aspects, the dosage of a composition (e.g., a composition including a dsRNA) is a prophylactically or a therapeutically effective amount.

### VII. Kits

Kits including (a) a pharmaceutical composition including a dsRNA agent that reduces the level and/or activity of MSH3 in a cell or subject described herein, and (b) a package insert with instructions to perform any of the methods described herein are also contemplated. In some aspects, the kit includes (a) a pharmaceutical composition including a dsRNA agent that reduces the level and/or activity of MSH3 in a cell or subject described herein, (b) an additional therapeutic agent, and (c) a package insert with instructions to perform any of the methods described herein.

### EXAMPLES

### Example 1. Design and Selection of dsRNA Agents

### Identification and Selection of Target Transcript

Target transcript selection and off-target scoring (below) utilized NCBI RefSeq sequences, downloaded from NCBI 21 Nov. 2018. Experimentally validated “NM” transcript models were used except for cynomolgus monkey, which only has “XM” predicted models for the large majority of genes. The longest human, mouse, rat, and cynomolgus monkey MSH3 transcript that contained all mapped internal exons was selected (SEQ IDs 1, 3, 4, and 5 for human, mouse, rat, and cynomolgus monkey, respectively; SEQ ID NO:2 is the protein sequence).

TABLE 2
Exemplary Human, Cyno, Mouse, and Rat MSH3 Transcripts

| Human (SEQ | Cyno (SEQ | Mouse (SEQ | Rat (SEQ |
|---|---|---|---|
| ID NO: 1) | ID NO: 3) | ID NO: 4) | ID NO: 5) |
| NM_002439.4 | XM_005557283.2 | NM_010829.2 | NM_001191957.1 |

### Selection of 19Mer Oligonucleotide Sequences

All sense 18mer sub-sequences and complementary antisense sequences per transcript were generated. An A nucleotide was added to the 3′ end of the sense strand, with a complementary U at the 5′ end of the antisense strand, to yield a 19mer duplex. This nucleotide pair was chosen because the antisense (“guide”) strand's first (5′) nucleotide is not exposed and does not bind to target mRNAs when loaded in the RISC complex, and the core AGO protein subunit shows preference for 5′ U nucleotides (Noland and Doudna (2013), RNA, 19: 639-648, Nakanishi (2016), WIREs RNA, 7: 637-660). Candidate 19mer duplexes were selected that met the following thermodynamic and physical characteristics: predicted melting temperature of <60° C., no homopolymers of 5 or longer, and at least 4 U or A nucleotides in the seed region (antisense strand positions 2-9). These selected duplexes were further evaluated for specificity (off-target scoring, below).

The specificity of the selected duplexes was evaluated via alignment of both strands to all unspliced RefSeq transcripts (“NM” models for human, mouse, and rat; “NM” and “XM” models for cynomolgus monkey), using the FASTA algorithm with an E value cutoff of 1000. Duplexes were selected with at least one 8mer seed (positions 2-9) mismatch on each strand to any transcript other than those encoded by the MSH3 gene, since seed mismatches govern specificity of dsRNA activity (Boudreau et al., (2011), Mol. Therapy 19: 2169-2177).

The sequences, positions in human transcript, and conservation in other species of each duplex are given in Table 3. In Table 3 below, the 5′ U of the antisense oligonucleotide can be any nucleotide (e.g., U, A, G, C, T). In some aspects, the 5′ U of the antisense oligonucleotide in Table 3 is U. Each sense and antisense oligonucleotides in Table 3 include a dTdT overhang on the 3′ end.

Additionally, every A and G in each sense oligonucleotide in Table 3 is a ribonucleotide. Every C and U in each sense oligonucleotide is a 2′-O-Methyl ribonucleotide.

Also, every A and G in each antisense oligonucleotide in Table 3 is a ribonucleotide. Every C and U preceding an A in the antisense oligonucleotide is a 2′-O-Methyl ribonucleotide, with one exception: U is the first nucleotide of the antisense strand, and it is a ribonucleotide.

Each nucleotide in the sense and antisense oligonucleotide in Table 3 is linked by a phosphate.

Furthermore, duplexes with sequence conservation in cynologous monkey, mouse, and rat are provided in Tables 4-10.

TABLE 3
Exemplary dsRNAs

| SEQ | | | | | | | |
|---|---|---|---|---|---|---|---|
| ID | | | | | | | |
| NO/ | | | SEQ ID NO/ | | | | |
| Sense | | | Antisense | | | | |
| Oligo | | | Oligo | | | | |
| NO | Sense | Antisense | NO | Pos | Cyno | Mouse | Rat |
| 6 | CCGCCGCACAUAGCUACAA | UUGUAGCUAUGUGCGGCGG | 7 | 306 | No | No | No |
| 8 | CGCCGCACAUAGCUACAGA | UCUGUAGCUAUGUGCGGCG | 9 | 307 | No | No | No |
| 10 | GCCGCACAUAGCUACAGAA | UUCUGUAGCUAUGUGCGGC | 11 | 308 | No | No | No |
| 12 | CGCACAUAGCUACAGAAAA | UUUUCUGUAGCUAUGUGCG | 13 | 310 | No | No | No |
| 14 | GCACAUAGCUACAGAAAUA | UAUUUCUGUAGCUAUGUGC | 15 | 311 | No | No | No |
| 16 | CACAUAGCUACAGAAAUUA | UAAUUUCUGUAGCUAUGUG | 17 | 312 | No | No | No |
| 18 | AUAGCUACAGAAAUUGACA | UGUCAAUUUCUGUAGCUAU | 19 | 315 | No | No | No |
| 20 | GCUACAGAAAUUGACAGAA | UUCUGUCAAUUUCUGUAGC | 21 | 318 | No | No | No |
| 22 | UUGACAGAAGAAAGAAGAA | UUCUUCUUUCUUCUGUCAA | 23 | 328 | No | No | No |
| 24 | CAGAAGAAAGAAGAGACCA | UGGUCUCUUCUUUCUUCUG | 25 | 332 | No | No | No |
| 26 | AGAAGAAAGAAGAGACCAA | UUGGUCUCUUCUUUCUUCU | 27 | 333 | No | No | No |
| 28 | AGAAAGAAGAGACCAUUGA | UCAAUGGUCUCUUCUUUCU | 29 | 336 | No | No | No |
| 30 | GAGACCAUUGGAAAAUGAA | UUCAUUUUCCAAUGGUCUC | 31 | 344 | Yes | No | No |
| 32 | CCAUUGGAAAAUGAUGGGA | UCCCAUCAUUUUCCAAUGG | 33 | 348 | Yes | No | No |
| 34 | AAAAUGAUGGGCCUGUUAA | UUAACAGGCCCAUCAUUUU | 35 | 355 | Yes | No | No |
| 36 | AGAAAGUAAAGAAAGUCCA | UGGACUUUCUUUACUUUCU | 37 | 376 | No | No | No |
| 38 | GAAAGUAAAGAAAGUCCAA | UUGGACUUUCUUUACUUUC | 39 | 377 | No | No | No |
| 40 | AAGUAAAGAAAGUCCAACA | UGUUGGACUUUCUUUACUU | 41 | 379 | No | No | No |
| 42 | AGUAAAGAAAGUCCAACAA | UUGUUGGACUUUCUUUACU | 43 | 380 | No | No | No |
| 44 | GUAAAGAAAGUCCAACAAA | UUUGUUGGACUUUCUUUAC | 45 | 381 | No | No | No |
| 46 | UAAAGAAAGUCCAACAAAA | UUUUGUUGGACUUUCUUUA | 47 | 382 | No | No | No |
| 48 | AGAAAGUCCAACAAAAGGA | UCCUUUUGUUGGACUUUCU | 49 | 385 | No | No | No |
| 50 | GAAAGUCCAACAAAAGGAA | UUCCUUUUGUUGGACUUUC | 51 | 386 | No | No | No |
| 52 | AAAGUCCAACAAAAGGAAA | UUUCCUUUUGUUGGACUUU | 53 | 387 | No | No | No |
| 54 | AGUCCAACAAAAGGAAGGA | UCCUUCCUUUUGUUGGACU | 55 | 389 | No | No | No |
| 56 | AAGGAAGGAGGAAGUGAUA | UAUCACUUCCUCCUUCCUU | 57 | 399 | Yes | No | No |
| 58 | AGGAAGUGAUCUGGGAAUA | UAUUCCCAGAUCACUUCCU | 59 | 407 | Yes | No | No |
| 60 | AGUGAUCUGGGAAUGUCUA | UAGACAUUCCCAGAUCACU | 61 | 411 | Yes | No | No |
| 62 | UCUGGGAAUGUCUGGCAAA | UUUGCCAGACAUUCCCAGA | 63 | 416 | Yes | No | No |
| 64 | UGGGAAUGUCUGGCAACUA | UAGUUGCCAGACAUUCCCA | 65 | 418 | Yes | No | No |
| 66 | GAAUGUCUGGCAACUCUGA | UCAGAGUUGCCAGACAUUC | 67 | 421 | Yes | No | No |
| 68 | AAUGUCUGGCAACUCUGAA | UUCAGAGUUGCCAGACAUU | 69 | 422 | Yes | No | No |
| 70 | AUGUCUGGCAACUCUGAGA | UCUCAGAGUUGCCAGACAU | 71 | 423 | Yes | No | No |
| 72 | UGGCAACUCUGAGCCAAAA | UUUUGGCUCAGAGUUGCCA | 73 | 428 | Yes | No | No |
| 74 | GGCAACUCUGAGCCAAAGA | UCUUUGGCUCAGAGUUGCC | 75 | 429 | Yes | No | No |
| 76 | CAACUCUGAGCCAAAGAAA | UUUCUUUGGCUCAGAGUUG | 77 | 431 | Yes | No | No |
| 78 | ACUCUGAGCCAAAGAAAUA | UAUUUCUUUGGCUCAGAGU | 79 | 433 | Yes | No | No |
| 80 | UCUGAGCCAAAGAAAUGUA | UACAUUUCUUUGGCUCAGA | 81 | 435 | Yes | No | No |
| 82 | UGAGCCAAAGAAAUGUCUA | UAGACAUUUCUUUGGCUCA | 83 | 437 | Yes | No | Yes |
| 84 | GUCUGAGGACCAGGAAUGA | UCAUUCCUGGUCCUCAGAC | 85 | 451 | Yes | No | No |
| 86 | UCUGAGGACCAGGAAUGUA | UACAUUCCUGGUCCUCAGA | 87 | 452 | Yes | No | No |
| 88 | GAGGACCAGGAAUGUUUCA | UGAAACAUUCCUGGUCCUC | 89 | 455 | No | No | No |
| 90 | AGGACCAGGAAUGUUUCAA | UUGAAACAUUCCUGGUCCU | 91 | 456 | No | No | No |
| 92 | GGACCAGGAAUGUUUCAAA | UUUGAAACAUUCCUGGUCC | 93 | 457 | No | No | No |
| 94 | ACCAGGAAUGUUUCAAAGA | UCUUUGAAACAUUCCUGGU | 95 | 459 | No | No | No |
| 96 | GGAAUGUUUCAAAGUCUCA | UGAGACUUUGAAACAUUCC | 97 | 463 | No | No | No |
| 98 | UGUUUCAAAGUCUCUGGAA | UUCCAGAGACUUUGAAACA | 99 | 467 | No | No | No |
| 100 | AAUUCUGCUGCGAUUCUGA | UCAGAAUCGCAGCAGAAUU | 101 | 496 | No | No | No |
| 102 | GAUUCUGCCCUUCCUCAAA | UUUGAGGAAGGGCAGAAUC | 103 | 507 | Yes | No | No |
| 104 | CUGCCCUUCCUCAAAGUAA | UUACUUUGAGGAAGGGCAG | 105 | 511 | Yes | No | No |
| 106 | UGCCCUUCCUCAAAGUAGA | UCUACUUUGAGGAAGGGCA | 107 | 512 | Yes | No | No |
| 108 | CCCUUCCUCAAAGUAGAGA | UCUCUACUUUGAGGAAGGG | 109 | 514 | Yes | No | No |
| 110 | CCUUCCUCAAAGUAGAGUA | UACUCUACUUUGAGGAAGG | 111 | 515 | Yes | No | No |
| 112 | CUUCCUCAAAGUAGAGUCA | UGACUCUACUUUGAGGAAG | 113 | 516 | Yes | No | No |
| 114 | UUCCUCAAAGUAGAGUCCA | UGGACUCUACUUUGAGGAA | 115 | 517 | Yes | No | No |
| 116 | CUCAAAGUAGAGUCCAGAA | UUCUGGACUCUACUUUGAG | 117 | 520 | Yes | No | No |
| 118 | AAGUAGAGUCCAGACAGAA | UUCUGUCUGGACUCUACUU | 119 | 524 | Yes | No | No |
| 120 | AGUAGAGUCCAGACAGAAA | UUUCUGUCUGGACUCUACU | 121 | 525 | Yes | No | No |
| 122 | AGAGUCCAGACAGAAUCUA | UAGAUUCUGUCUGGACUCU | 123 | 528 | Yes | No | No |
| 124 | GAGUCCAGACAGAAUCUCA | UGAGAUUCUGUCUGGACUC | 125 | 529 | Yes | No | No |
| 126 | GUCCAGACAGAAUCUCUGA | UCAGAGAUUCUGUCUGGAC | 127 | 531 | Yes | No | No |
| 128 | UCCAGACAGAAUCUCUGCA | UGCAGAGAUUCUGUCUGGA | 129 | 532 | Yes | No | No |
| 130 | UCUCUGCAGGAGAGAUUUA | UAAAUCUCUCCUGCAGAGA | 131 | 543 | Yes | No | No |
| 132 | GCAGGAGAGAUUUGCAGUA | UACUGCAAAUCUCUCCUGC | 133 | 548 | Yes | No | No |
| 134 | CAGGAGAGAUUUGCAGUUA | UAACUGCAAAUCUCUCCUG | 135 | 549 | Yes | No | No |
| 136 | GAGAGAUUUGCAGUUCUGA | UCAGAACUGCAAAUCUCUC | 137 | 552 | Yes | No | No |
| 138 | AGAGAUUUGCAGUUCUGCA | UGCAGAACUGCAAAUCUCU | 139 | 553 | Yes | No | No |
| 140 | AGAUUUGCAGUUCUGCCAA | UUGGCAGAACUGCAAAUCU | 141 | 555 | Yes | No | No |
| 142 | GAUUUGCAGUUCUGCCAAA | UUUGGCAGAACUGCAAAUC | 143 | 556 | Yes | No | No |
| 144 | AUUUGCAGUUCUGCCAAAA | UUUUGGCAGAACUGCAAAU | 145 | 557 | Yes | No | No |
| 146 | UUGCAGUUCUGCCAAAAUA | UAUUUUGGCAGAACUGCAA | 147 | 559 | Yes | No | No |
| 148 | AGUUCUGCCAAAAUGUACA | UGUACAUUUUGGCAGAACU | 149 | 563 | Yes | No | No |
| 150 | GUUCUGCCAAAAUGUACUA | UAGUACAUUUUGGCAGAAC | 151 | 564 | Yes | No | No |
| 152 | UUCUGCCAAAAUGUACUGA | UCAGUACAUUUUGGCAGAA | 153 | 565 | Yes | No | No |
| 154 | CUGCCAAAAUGUACUGAUA | UAUCAGUACAUUUUGGCAG | 155 | 567 | Yes | No | No |
| 156 | GCCAAAAUGUACUGAUUUA | UAAAUCAGUACAUUUUGGC | 157 | 569 | Yes | No | No |
| 158 | GUACUGAUUUUGAUGAUAA | UUAUCAUCAAAAUCAGUAC | 159 | 577 | Yes | No | No |
| 160 | ACUGAUUUUGAUGAUAUCA | UGAUAUCAUCAAAAUCAGU | 161 | 579 | Yes | No | No |
| 162 | UGAUGAUAUCAGUCUUCUA | UAGAAGACUGAUAUCAUCA | 163 | 587 | Yes | No | No |
| 164 | AUGAUAUCAGUCUUCUACA | UGUAGAAGACUGAUAUCAU | 165 | 589 | Yes | No | No |
| 166 | UGAUAUCAGUCUUCUACAA | UUGUAGAAGACUGAUAUCA | 167 | 590 | No | No | No |
| 168 | AUAUCAGUCUUCUACACGA | UCGUGUAGAAGACUGAUAU | 169 | 592 | No | No | No |
| 170 | UCAGUCUUCUACACGCAAA | UUUGCGUGUAGAAGACUGA | 171 | 595 | No | No | No |
| 172 | CAGUCUUCUACACGCAAAA | UUUUGCGUGUAGAAGACUG | 173 | 596 | No | No | No |
| 174 | AGUCUUCUACACGCAAAGA | UCUUUGCGUGUAGAAGACU | 175 | 597 | No | No | No |
| 176 | UCUUCUACACGCAAAGAAA | UUUCUUUGCGUGUAGAAGA | 177 | 599 | No | No | No |
| 178 | CUUCUACACGCAAAGAAUA | UAUUCUUUGCGUGUAGAAG | 179 | 600 | No | No | No |
| 180 | UUCUACACGCAAAGAAUGA | UCAUUCUUUGCGUGUAGAA | 181 | 601 | No | No | No |
| 182 | UCUACACGCAAAGAAUGCA | UGCAUUCUUUGCGUGUAGA | 183 | 602 | No | No | No |
| 184 | CUACACGCAAAGAAUGCAA | UUGCAUUCUUUGCGUGUAG | 185 | 603 | No | No | No |
| 186 | ACACGCAAAGAAUGCAGUA | UACUGCAUUCUUUGCGUGU | 187 | 605 | No | No | No |
| 188 | CACGCAAAGAAUGCAGUUA | UAACUGCAUUCUUUGCGUG | 189 | 606 | No | No | No |
| 190 | AAGAAUGCAGUUUCUUCUA | UAGAAGAAACUGCAUUCUU | 191 | 612 | Yes | No | No |
| 192 | AGUUUCUUCUGAAGAUUCA | UGAAUCUUCAGAAGAAACU | 193 | 620 | Yes | No | No |
| 194 | UCUGAAGAUUCGAAACGUA | UACGUUUCGAAUCUUCAGA | 195 | 627 | No | No | No |
| 196 | UGAAGAUUCGAAACGUCAA | UUGACGUUUCGAAUCUUCA | 197 | 629 | No | No | No |
| 198 | GAAGAUUCGAAACGUCAAA | UUUGACGUUUCGAAUCUUC | 199 | 630 | No | No | No |
| 200 | AAGAUUCGAAACGUCAAAA | UUUUGACGUUUCGAAUCUU | 201 | 631 | No | No | No |
| 202 | AGAUUCGAAACGUCAAAUA | UAUUUGACGUUUCGAAUCU | 203 | 632 | No | No | No |
| 204 | GAUUCGAAACGUCAAAUUA | UAAUUUGACGUUUCGAAUC | 205 | 633 | No | No | No |
| 206 | UUCGAAACGUCAAAUUAAA | UUUAAUUUGACGUUUCGAA | 207 | 635 | No | No | No |
| 208 | GAAACGUCAAAUUAAUCAA | UUGAUUAAUUUGACGUUUC | 209 | 638 | No | No | No |
| 210 | AAACGUCAAAUUAAUCAAA | UUUGAUUAAUUUGACGUUU | 211 | 639 | No | No | No |
| 212 | AACGUCAAAUUAAUCAAAA | UUUUGAUUAAUUUGACGUU | 213 | 640 | No | No | No |
| 214 | CGUCAAAUUAAUCAAAAGA | UCUUUUGAUUAAUUUGACG | 215 | 642 | No | No | No |
| 216 | GUCAAAUUAAUCAAAAGGA | UCCUUUUGAUUAAUUUGAC | 217 | 643 | No | No | No |
| 218 | UUAAUCAAAAGGACACAAA | UUUGUGUCCUUUUGAUUAA | 219 | 649 | No | No | No |
| 220 | UAAUCAAAAGGACACAACA | UGUUGUGUCCUUUUGAUUA | 221 | 650 | No | No | No |
| 222 | CAAAAGGACACAACACUUA | UAAGUGUUGUGUCCUUUUG | 223 | 654 | No | No | No |
| 224 | AAAGGACACAACACUUUUA | UAAAAGUGUUGUGUCCUUU | 225 | 656 | No | No | No |
| 226 | UUUUGAUCUCAGUCAGUUA | UAACUGACUGAGAUCAAAA | 227 | 671 | No | No | No |
| 228 | UGAUCUCAGUCAGUUUGGA | UCCAAACUGACUGAGAUCA | 229 | 674 | No | No | No |
| 230 | AUCUCAGUCAGUUUGGAUA | UAUCCAAACUGACUGAGAU | 231 | 676 | No | No | No |
| 232 | CUCAGUCAGUUUGGAUCAA | UUGAUCCAAACUGACUGAG | 233 | 678 | No | No | No |
| 234 | UCAGUCAGUUUGGAUCAUA | UAUGAUCCAAACUGACUGA | 235 | 679 | Yes | No | No |
| 236 | CAGUCAGUUUGGAUCAUCA | UGAUGAUCCAAACUGACUG | 237 | 680 | Yes | No | No |
| 238 | AGUCAGUUUGGAUCAUCAA | UUGAUGAUCCAAACUGACU | 239 | 681 | Yes | No | No |
| 240 | CAGUUUGGAUCAUCAAAUA | UAUUUGAUGAUCCAAACUG | 241 | 684 | Yes | No | No |
| 242 | AGUUUGGAUCAUCAAAUAA | UUAUUUGAUGAUCCAAACU | 243 | 685 | Yes | No | No |
| 244 | GUUUGGAUCAUCAAAUACA | UGUAUUUGAUGAUCCAAAC | 245 | 686 | Yes | No | No |
| 246 | UUUGGAUCAUCAAAUACAA | UUGUAUUUGAUGAUCCAAA | 247 | 687 | Yes | No | No |
| 248 | UUGGAUCAUCAAAUACAAA | UUUGUAUUUGAUGAUCCAA | 249 | 688 | Yes | No | No |
| 250 | UGGAUCAUCAAAUACAAGA | UCUUGUAUUUGAUGAUCCA | 251 | 689 | Yes | No | No |
| 252 | GGAUCAUCAAAUACAAGUA | UACUUGUAUUUGAUGAUCC | 253 | 690 | Yes | No | No |
| 254 | CAUCAAAUACAAGUCAUGA | UCAUGACUUGUAUUUGAUG | 255 | 694 | Yes | No | No |
| 256 | UCAAAUACAAGUCAUGAAA | UUUCAUGACUUGUAUUUGA | 257 | 696 | Yes | No | No |
| 258 | AUACAAGUCAUGAAAAUUA | UAAUUUUCAUGACUUGUAU | 259 | 700 | Yes | No | No |
| 260 | UACAAGUCAUGAAAAUUUA | UAAAUUUUCAUGACUUGUA | 261 | 701 | Yes | No | No |
| 262 | UACAGAAAACUGCUUCCAA | UUGGAAGCAGUUUUCUGUA | 263 | 718 | No | No | No |
| 264 | AAAACUGCUUCCAAAUCAA | UUGAUUUGGAAGCAGUUUU | 265 | 723 | No | No | No |
| 266 | AACUGCUUCCAAAUCAGCA | UGCUGAUUUGGAAGCAGUU | 267 | 725 | No | No | No |
| 268 | ACUGCUUCCAAAUCAGCUA | UAGCUGAUUUGGAAGCAGU | 269 | 726 | No | No | No |
| 270 | CUGCUUCCAAAUCAGCUAA | UUAGCUGAUUUGGAAGCAG | 271 | 727 | No | No | No |
| 272 | GCUUCCAAAUCAGCUAACA | UGUUAGCUGAUUUGGAAGC | 273 | 729 | No | No | No |
| 274 | CUUCCAAAUCAGCUAACAA | UUGUUAGCUGAUUUGGAAG | 275 | 730 | No | No | No |
| 276 | CCAAAUCAGCUAACAAACA | UGUUUGUUAGCUGAUUUGG | 277 | 733 | No | No | No |
| 278 | AAAUCAGCUAACAAACGGA | UCCGUUUGUUAGCUGAUUU | 279 | 735 | No | No | No |
| 280 | AAUCAGCUAACAAACGGUA | UACCGUUUGUUAGCUGAUU | 281 | 736 | No | No | No |
| 282 | GCUAACAAACGGUCCAAAA | UUUUGGACCGUUUGUUAGC | 283 | 741 | No | No | No |
| 284 | UAACAAACGGUCCAAAAGA | UCUUUUGGACCGUUUGUUA | 285 | 743 | No | No | No |
| 286 | AACAAACGGUCCAAAAGCA | UGCUUUUGGACCGUUUGUU | 287 | 744 | No | No | No |
| 288 | AAACGGUCCAAAAGCAUCA | UGAUGCUUUUGGACCGUUU | 289 | 747 | No | No | No |
| 290 | AACGGUCCAAAAGCAUCUA | UAGAUGCUUUUGGACCGUU | 291 | 748 | No | No | No |
| 292 | CGGUCCAAAAGCAUCUAUA | UAUAGAUGCUUUUGGACCG | 293 | 750 | No | No | No |
| 294 | UCCAAAAGCAUCUAUACGA | UCGUAUAGAUGCUUUUGGA | 295 | 753 | No | No | No |
| 296 | CCAAAAGCAUCUAUACGCA | UGCGUAUAGAUGCUUUUGG | 297 | 754 | No | No | No |
| 298 | AUCUAUACGCCGCUAGAAA | UUUCUAGCGGCGUAUAGAU | 299 | 762 | No | No | No |
| 300 | CUAUACGCCGCUAGAAUUA | UAAUUCUAGCGGCGUAUAG | 301 | 764 | No | No | No |
| 302 | UAUACGCCGCUAGAAUUAA | UUAAUUCUAGCGGCGUAUA | 303 | 765 | Yes | No | No |
| 304 | AUACGCCGCUAGAAUUACA | UGUAAUUCUAGCGGCGUAU | 305 | 766 | Yes | No | No |
| 306 | ACGCCGCUAGAAUUACAAA | UUUGUAAUUCUAGCGGCGU | 307 | 768 | Yes | No | No |
| 308 | CCGCUAGAAUUACAAUACA | UGUAUUGUAAUUCUAGCGG | 309 | 771 | Yes | No | No |
| 310 | CGCUAGAAUUACAAUACAA | UUGUAUUGUAAUUCUAGCG | 311 | 772 | Yes | No | No |
| 312 | UAGAAUUACAAUACAUAGA | UCUAUGUAUUGUAAUUCUA | 313 | 775 | Yes | No | No |
| 314 | AGAAUUACAAUACAUAGAA | UUCUAUGUAUUGUAAUUCU | 315 | 776 | Yes | No | No |
| 316 | GAAUUACAAUACAUAGAAA | UUUCUAUGUAUUGUAAUUC | 317 | 777 | Yes | No | No |
| 318 | AAUUACAAUACAUAGAAAA | UUUUCUAUGUAUUGUAAUU | 319 | 778 | Yes | No | No |
| 320 | AUACAUAGAAAUGAAGCAA | UUGCUUCAUUUCUAUGUAU | 321 | 785 | Yes | No | No |
| 322 | UACAUAGAAAUGAAGCAGA | UCUGCUUCAUUUCUAUGUA | 323 | 786 | Yes | No | No |
| 324 | GCACAAAGAUGCAGUUUUA | UAAAACUGCAUCUUUGUGC | 325 | 806 | Yes | No | No |
| 326 | CACAAAGAUGCAGUUUUGA | UCAAAACUGCAUCUUUGUG | 327 | 807 | Yes | No | No |
| 328 | CAAAGAUGCAGUUUUGUGA | UCACAAAACUGCAUCUUUG | 329 | 809 | Yes | No | No |
| 330 | AAGAUGCAGUUUUGUGUGA | UCACACAAAACUGCAUCUU | 331 | 811 | Yes | No | No |
| 332 | AGAUGCAGUUUUGUGUGUA | UACACACAAAACUGCAUCU | 333 | 812 | Yes | No | No |
| 334 | GAUGCAGUUUUGUGUGUGA | UCACACACAAAACUGCAUC | 335 | 813 | Yes | No | No |
| 336 | UGCAGUUUUGUGUGUGGAA | UUCCACACACAAAACUGCA | 337 | 815 | Yes | No | No |
| 338 | GCAGUUUUGUGUGUGGAAA | UUUCCACACACAAAACUGC | 339 | 816 | Yes | No | No |
| 340 | AGUUUUGUGUGUGGAAUGA | UCAUUCCACACACAAAACU | 341 | 818 | Yes | No | No |
| 342 | GUUUUGUGUGUGGAAUGUA | UACAUUCCACACACAAAAC | 343 | 819 | Yes | No | Yes |
| 344 | UUUUGUGUGUGGAAUGUGA | UCACAUUCCACACACAAAA | 345 | 820 | Yes | No | Yes |
| 346 | UUUGUGUGUGGAAUGUGGA | UCCACAUUCCACACACAAA | 347 | 821 | Yes | No | Yes |
| 348 | UUGUGUGUGGAAUGUGGAA | UUCCACAUUCCACACACAA | 349 | 822 | Yes | No | No |
| 350 | UGUGUGUGGAAUGUGGAUA | UAUCCACAUUCCACACACA | 351 | 823 | Yes | No | No |
| 352 | GUGUGUGGAAUGUGGAUAA | UUAUCCACAUUCCACACAC | 353 | 824 | Yes | No | No |
| 354 | GUGUGGAAUGUGGAUAUAA | UUAUAUCCACAUUCCACAC | 355 | 826 | Yes | NO | No |
| 356 | UGUGGAAUGUGGAUAUAAA | UUUAUAUCCACAUUCCACA | 357 | 827 | Yes | No | No |
| 358 | GUGGAAUGUGGAUAUAAGA | UCUUAUAUCCACAUUCCAC | 359 | 828 | Yes | No | No |
| 360 | UGGAAUGUGGAUAUAAGUA | UACUUAUAUCCACAUUCCA | 361 | 829 | Yes | No | No |
| 362 | GGAAUGUGGAUAUAAGUAA | UUACUUAUAUCCACAUUCC | 363 | 830 | Yes | No | No |
| 364 | GAAUGUGGAUAUAAGUAUA | UAUACUUAUAUCCACAUUC | 365 | 831 | Yes | No | No |
| 366 | AAUGUGGAUAUAAGUAUAA | UUAUACUUAUAUCCACAUU | 367 | 832 | Yes | No | No |
| 368 | AUGUGGAUAUAAGUAUAGA | UCUAUACUUAUAUCCACAU | 369 | 833 | Yes | No | No |
| 370 | UGUGGAUAUAAGUAUAGAA | UUCUAUACUUAUAUCCACA | 371 | 834 | Yes | No | No |
| 372 | UGGAUAUAAGUAUAGAUUA | UAAUCUAUACUUAUAUCCA | 373 | 836 | Yes | No | No |
| 374 | GAUAUAAGUAUAGAUUCUA | UAGAAUCUAUACUUAUAUC | 375 | 838 | Yes | No | No |
| 376 | UAUAAGUAUAGAUUCUUUA | UAAAGAAUCUAUACUUAUA | 377 | 840 | Yes | No | No |
| 378 | UAAGUAUAGAUUCUUUGGA | UCCAAAGAAUCUAUACUUA | 379 | 842 | Yes | No | No |
| 380 | AAGUAUAGAUUCUUUGGGA | UCCCAAAGAAUCUAUACUU | 381 | 843 | Yes | No | No |
| 382 | UAUAGAUUCUUUGGGGAAA | UUUCCCCAAAGAAUCUAUA | 383 | 846 | Yes | No | No |
| 384 | AGAUUCUUUGGGGAAGAUA | UAUCUUCCCCAAAGAAUCU | 385 | 849 | Yes | Yes | No |
| 386 | UGCAGCCCGAGAGCUCAAA | UUUGAGCUCUCGGGCUGCA | 387 | 875 | Yes | No | No |
| 388 | AGCCCGAGAGCUCAAUAUA | UAUAUUGAGCUCUCGGGCU | 389 | 878 | Yes | No | No |
| 390 | GCCCGAGAGCUCAAUAUUA | UAAUAUUGAGCUCUCGGGC | 391 | 879 | Yes | No | No |
| 392 | CGAGAGCUCAAUAUUUAUA | UAUAAAUAUUGAGCUCUCG | 393 | 882 | Yes | No | No |
| 394 | GAGAGCUCAAUAUUUAUUA | UAAUAAAUAUUGAGCUCUC | 395 | 883 | Yes | No | No |
| 396 | GAGCUCAAUAUUUAUUGCA | UGCAAUAAAUAUUGAGCUC | 397 | 885 | Yes | No | No |
| 398 | CUCAAUAUUUAUUGCCAUA | UAUGGCAAUAAAUAUUGAG | 399 | 888 | Yes | No | No |
| 400 | UAUUUAUUGCCAUUUAGAA | UUCUAAAUGGCAAUAAAUA | 401 | 893 | Yes | No | No |
| 402 | UUUAUUGCCAUUUAGAUCA | UGAUCUAAAUGGCAAUAAA | 403 | 895 | Yes | No | No |
| 404 | UUAUUGCCAUUUAGAUCAA | UUGAUCUAAAUGGCAAUAA | 405 | 896 | Yes | No | No |
| 406 | UAUUGCCAUUUAGAUCACA | UGUGAUCUAAAUGGCAAUA | 407 | 897 | Yes | No | No |
| 408 | AUUGCCAUUUAGAUCACAA | UUGUGAUCUAAAUGGCAAU | 409 | 898 | Yes | No | No |
| 410 | UUGCCAUUUAGAUCACAAA | UUUGUGAUCUAAAUGGCAA | 411 | 899 | Yes | No | No |
| 412 | GCCAUUUAGAUCACAACUA | UAGUUGUGAUCUAAAUGGC | 413 | 901 | Yes | No | No |
| 414 | UUUAGAUCACAACUUUAUA | UAUAAAGUUGUGAUCUAAA | 415 | 905 | Yes | No | No |
| 416 | UUAGAUCACAACUUUAUGA | UCAUAAAGUUGUGAUCUAA | 417 | 906 | Yes | No | No |
| 418 | UAGAUCACAACUUUAUGAA | UUCAUAAAGUUGUGAUCUA | 419 | 907 | Yes | No | No |
| 420 | AGAUCACAACUUUAUGACA | UGUCAUAAAGUUGUGAUCU | 421 | 908 | Yes | No | No |
| 422 | AUCACAACUUUAUGACAGA | UCUGUCAUAAAGUUGUGAU | 423 | 910 | Yes | No | No |
| 424 | UCACAACUUUAUGACAGCA | UGCUGUCAUAAAGUUGUGA | 425 | 911 | Yes | No | No |
| 426 | CACAACUUUAUGACAGCAA | UUGCUGUCAUAAAGUUGUG | 427 | 912 | Yes | No | No |
| 428 | ACAACUUUAUGACAGCAAA | UUUGCUGUCAUAAAGUUGU | 429 | 913 | Yes | No | No |
| 430 | CAACUUUAUGACAGCAAGA | UCUUGCUGUCAUAAAGUUG | 431 | 914 | Yes | No | No |
| 432 | AACUUUAUGACAGCAAGUA | UACUUGCUGUCAUAAAGUU | 433 | 915 | Yes | No | No |
| 434 | CUUUAUGACAGCAAGUAUA | UAUACUUGCUGUCAUAAAG | 435 | 917 | Yes | No | No |
| 436 | AUGACAGCAAGUAUACCUA | UAGGUAUACUUGCUGUCAU | 437 | 921 | Yes | No | No |
| 438 | UGACAGCAAGUAUACCUAA | UUAGGUAUACUUGCUGUCA | 439 | 922 | Yes | No | No |
| 440 | GACAGCAAGUAUACCUACA | UGUAGGUAUACUUGCUGUC | 441 | 923 | Yes | No | No |
| 442 | CAGCAAGUAUACCUACUCA | UGAGUAGGUAUACUUGCUG | 443 | 925 | Yes | No | No |
| 444 | GCAAGUAUACCUACUCACA | UGUGAGUAGGUAUACUUGC | 445 | 927 | Yes | No | No |
| 446 | AGUAUACCUACUCACAGAA | UUCUGUGAGUAGGUAUACU | 447 | 930 | Yes | No | No |
| 448 | GUAUACCUACUCACAGACA | UGUCUGUGAGUAGGUAUAC | 449 | 931 | Yes | No | No |
| 450 | AUACCUACUCACAGACUGA | UCAGUCUGUGAGUAGGUAU | 451 | 933 | Yes | No | No |
| 452 | UACCUACUCACAGACUGUA | UACAGUCUGUGAGUAGGUA | 453 | 934 | Yes | No | No |
| 454 | ACCUACUCACAGACUGUUA | UAACAGUCUGUGAGUAGGU | 455 | 935 | Yes | No | No |
| 456 | ACUCACAGACUGUUUGUUA | UAACAAACAGUCUGUGAGU | 457 | 939 | Yes | No | No |
| 458 | CUCACAGACUGUUUGUUCA | UGAACAAACAGUCUGUGAG | 459 | 940 | Yes | No | No |
| 460 | CACAGACUGUUUGUUCAUA | UAUGAACAAACAGUCUGUG | 461 | 942 | Yes | No | No |
| 462 | ACAGACUGUUUGUUCAUGA | UCAUGAACAAACAGUCUGU | 463 | 943 | Yes | No | No |
| 464 | ACUGUUUGUUCAUGUACGA | UCGUACAUGAACAAACAGU | 465 | 947 | Yes | No | No |
| 466 | CUGUUUGUUCAUGUACGCA | UGCGUACAUGAACAAACAG | 467 | 948 | Yes | No | No |
| 468 | CGCCUGGUGGCAAAAGGAA | UUCCUUUUGCCACCAGGCG | 469 | 966 | Yes | No | No |
| 470 | CUGGUGGCAAAAGGAUAUA | UAUAUCCUUUUGCCACCAG | 471 | 969 | Yes | No | No |
| 472 | UGGUGGCAAAAGGAUAUAA | UUAUAUCCUUUUGCCACCA | 473 | 970 | Yes | No | No |
| 474 | GGUGGCAAAAGGAUAUAAA | UUUAUAUCCUUUUGCCACC | 475 | 971 | Yes | No | No |
| 476 | GUGGCAAAAGGAUAUAAGA | UCUUAUAUCCUUUUGCCAC | 477 | 972 | Yes | No | No |
| 478 | UGGCAAAAGGAUAUAAGGA | UCCUUAUAUCCUUUUGCCA | 479 | 973 | Yes | No | No |
| 480 | GGCAAAAGGAUAUAAGGUA | UACCUUAUAUCCUUUUGCC | 481 | 974 | Yes | No | No |
| 482 | GCAAAAGGAUAUAAGGUGA | UCACCUUAUAUCCUUUUGC | 483 | 975 | Yes | No | No |
| 484 | CAAAAGGAUAUAAGGUGGA | UCCACCUUAUAUCCUUUUG | 485 | 976 | Yes | No | No |
| 486 | GGAUAUAAGGUGGGAGUUA | UAACUCCCACCUUAUAUCC | 487 | 981 | Yes | No | No |
| 488 | UAUAAGGUGGGAGUUGUGA | UCACAACUCCCACCUUAUA | 489 | 984 | Yes | No | No |
| 490 | UAAGGUGGGAGUUGUGAAA | UUUCACAACUCCCACCUUA | 491 | 986 | Yes | No | No |
| 492 | AAGGUGGGAGUUGUGAAGA | UCUUCACAACUCCCACCUU | 493 | 987 | Yes | Yes | Yes |
| 494 | GUGGGAGUUGUGAAGCAAA | UUUGCUUCACAACUCCCAC | 495 | 990 | Yes | Yes | Yes |
| 496 | GGGAGUUGUGAAGCAAACA | UGUUUGCUUCACAACUCCC | 497 | 992 | Yes | Yes | Yes |
| 498 | GAGUUGUGAAGCAAACUGA | UCAGUUUGCUUCACAACUC | 499 | 994 | Yes | Yes | Yes |
| 500 | AGUUGUGAAGCAAACUGAA | UUCAGUUUGCUUCACAACU | 501 | 995 | Yes | Yes | Yes |
| 502 | GUUGUGAAGCAAACUGAAA | UUUCAGUUUGCUUCACAAC | 503 | 996 | Yes | Yes | Yes |
| 504 | UGUGAAGCAAACUGAAACA | UGUUUCAGUUUGCUUCACA | 505 | 998 | Yes | Yes | Yes |
| 506 | GAAGCAAACUGAAACUGCA | UGCAGUUUCAGUUUGCUUC | 507 | 1001 | Yes | Yes | Yes |
| 508 | AACUGAAACUGCAGCAUUA | UAAUGCUGCAGUUUCAGUU | 509 | 1007 | Yes | No | No |
| 510 | ACUGAAACUGCAGCAUUAA | UUAAUGCUGCAGUUUCAGU | 511 | 1008 | Yes | No | No |
| 512 | CUGAAACUGCAGCAUUAAA | UUUAAUGCUGCAGUUUCAG | 513 | 1009 | Yes | No | No |
| 514 | UGAAACUGCAGCAUUAAAA | UUUUAAUGCUGCAGUUUCA | 515 | 1010 | Yes | No | No |
| 516 | AACUGCAGCAUUAAAGGCA | UGCCUUUAAUGCUGCAGUU | 517 | 1013 | Yes | No | No |
| 518 | CUGCAGCAUUAAAGGCCAA | UUGGCCUUUAAUGCUGCAG | 519 | 1015 | Yes | No | No |
| 520 | GCAUUAAAGGCCAUUGGAA | UUCCAAUGGCCUUUAAUGC | 521 | 1020 | Yes | No | Yes |
| 522 | AUUAAAGGCCAUUGGAGAA | UUCUCCAAUGGCCUUUAAU | 523 | 1022 | Yes | No | Yes |
| 524 | UUAAAGGCCAUUGGAGACA | UGUCUCCAAUGGCCUUUAA | 525 | 1023 | Yes | No | Yes |
| 526 | UAAAGGCCAUUGGAGACAA | UUGUCUCCAAUGGCCUUUA | 527 | 1024 | Yes | No | Yes |
| 528 | AAAGGCCAUUGGAGACAAA | UUUGUCUCCAAUGGCCUUU | 529 | 1025 | Yes | No | Yes |
| 530 | AAGGCCAUUGGAGACAACA | UGUUGUCUCCAAUGGCCUU | 531 | 1026 | Yes | No | No |
| 532 | GGCCAUUGGAGACAACAGA | UCUGUUGUCUCCAAUGGCC | 533 | 1028 | Yes | No | No |
| 534 | GCCAUUGGAGACAACAGAA | UUCUGUUGUCUCCAAUGGC | 535 | 1029 | Yes | No | No |
| 536 | CCAUUGGAGACAACAGAAA | UUUCUGUUGUCUCCAAUGG | 537 | 1030 | Yes | No | No |
| 538 | CAUUGGAGACAACAGAAGA | UCUUCUGUUGUCUCCAAUG | 539 | 1031 | Yes | No | No |
| 540 | AUUGGAGACAACAGAAGUA | UACUUCUGUUGUCUCCAAU | 541 | 1032 | Yes | No | No |
| 542 | UUGGAGACAACAGAAGUUA | UAACUUCUGUUGUCUCCAA | 543 | 1033 | Yes | No | No |
| 544 | UGGAGACAACAGAAGUUCA | UGAACUUCUGUUGUCUCCA | 545 | 1034 | Yes | No | No |
| 546 | GAGACAACAGAAGUUCACA | UGUGAACUUCUGUUGUCUC | 547 | 1036 | Yes | No | No |
| 548 | ACAACAGAAGUUCACUCUA | UAGAGUGAACUUCUGUUGU | 549 | 1039 | Yes | No | No |
| 550 | CAACAGAAGUUCACUCUUA | UAAGAGUGAACUUCUGUUG | 551 | 1040 | Yes | No | No |
| 552 | ACAGAAGUUCACUCUUUUA | UAAAAGAGUGAACUUCUGU | 553 | 1042 | Yes | No | No |
| 554 | CAGAAGUUCACUCUUUUCA | UGAAAAGAGUGAACUUCUG | 555 | 1043 | Yes | No | No |
| 556 | GAAGUUCACUCUUUUCCCA | UGGGAAAAGAGUGAACUUC | 557 | 1045 | Yes | No | No |
| 558 | UCUUUUCCCGGAAAUUGAA | UUCAAUUUCCGGGAAAAGA | 559 | 1054 | Yes | No | Yes |
| 560 | CUUUUCCCGGAAAUUGACA | UGUCAAUUUCCGGGAAAAG | 561 | 1055 | Yes | No | Yes |
| 562 | UUUUCCCGGAAAUUGACUA | UAGUCAAUUUCCGGGAAAA | 563 | 1056 | Yes | No | Yes |
| 564 | UUCCCGGAAAUUGACUGCA | UGCAGUCAAUUUCCGGGAA | 565 | 1058 | Yes | No | Yes |
| 566 | GGAAAUUGACUGCCCUUUA | UAAAGGGCAGUCAAUUUCC | 567 | 1063 | Yes | No | No |
| 568 | GAAAUUGACUGCCCUUUAA | UUAAAGGGCAGUCAAUUUC | 569 | 1064 | Yes | No | No |
| 570 | AAAUUGACUGCCCUUUAUA | UAUAAAGGGCAGUCAAUUU | 571 | 1065 | Yes | No | No |
| 572 | UUGACUGCCCUUUAUACAA | UUGUAUAAAGGGCAGUCAA | 573 | 1068 | Yes | No | No |
| 574 | CUGCCCUUUAUACAAAAUA | UAUUUUGUAUAAAGGGCAG | 575 | 1072 | Yes | No | No |
| 576 | UGCCCUUUAUACAAAAUCA | UGAUUUUGUAUAAAGGGCA | 577 | 1073 | Yes | No | No |
| 578 | GCCCUUUAUACAAAAUCUA | UAGAUUUUGUAUAAAGGGC | 579 | 1074 | Yes | No | No |
| 580 | CCCUUUAUACAAAAUCUAA | UUAGAUUUUGUAUAAAGGG | 581 | 1075 | Yes | No | No |
| 582 | CCUUUAUACAAAAUCUACA | UGUAGAUUUUGUAUAAAGG | 583 | 1076 | Yes | No | No |
| 584 | UUAUACAAAAUCUACACUA | UAGUGUAGAUUUUGUAUAA | 585 | 1079 | Yes | No | No |
| 586 | UAUACAAAAUCUACACUUA | UAAGUGUAGAUUUUGUAUA | 587 | 1080 | No | No | No |
| 588 | AUACAAAAUCUACACUUAA | UUAAGUGUAGAUUUUGUAU | 589 | 1081 | No | No | No |
| 590 | CAAAAUCUACACUUAUUGA | UCAAUAAGUGUAGAUUUUG | 591 | 1084 | No | No | No |
| 592 | AAAAUCUACACUUAUUGGA | UCCAAUAAGUGUAGAUUUU | 593 | 1085 | No | No | No |
| 594 | AAAUCUACACUUAUUGGAA | UUCCAAUAAGUGUAGAUUU | 595 | 1086 | No | No | No |
| 596 | AUCUACACUUAUUGGAGAA | UUCUCCAAUAAGUGUAGAU | 597 | 1088 | No | No | No |
| 598 | UCUACACUUAUUGGAGAAA | UUUCUCCAAUAAGUGUAGA | 599 | 1089 | No | No | No |
| 600 | CACUUAUUGGAGAAGAUGA | UCAUCUUCUCCAAUAAGUG | 601 | 1093 | No | No | No |
| 602 | ACUUAUUGGAGAAGAUGUA | UACAUCUUCUCCAAUAAGU | 603 | 1094 | No | No | No |
| 604 | CUUAUUGGAGAAGAUGUGA | UCACAUCUUCUCCAAUAAG | 605 | 1095 | No | No | No |
| 606 | UUAUUGGAGAAGAUGUGAA | UUCACAUCUUCUCCAAUAA | 607 | 1096 | No | No | No |
| 608 | AGAAGAUGUGAAUCCCCUA | UAGGGGAUUCACAUCUUCU | 609 | 1103 | Yes | No | No |
| 610 | GAAGAUGUGAAUCCCCUAA | UUAGGGGAUUCACAUCUUC | 611 | 1104 | Yes | No | No |
| 612 | AGAUGUGAAUCCCCUAAUA | UAUUAGGGGAUUCACAUCU | 613 | 1106 | Yes | No | No |
| 614 | GAUGUGAAUCCCCUAAUCA | UGAUUAGGGGAUUCACAUC | 615 | 1107 | Yes | No | No |
| 616 | UGUGAAUCCCCUAAUCAAA | UUUGAUUAGGGGAUUCACA | 617 | 1109 | Yes | No | No |
| 618 | GUGAAUCCCCUAAUCAAGA | UCUUGAUUAGGGGAUUCAC | 619 | 1110 | Yes | No | No |
| 620 | UGAAUCCCCUAAUCAAGCA | UGCUUGAUUAGGGGAUUCA | 621 | 1111 | Yes | No | No |
| 622 | GAAUCCCCUAAUCAAGCUA | UAGCUUGAUUAGGGGAUUC | 623 | 1112 | Yes | No | No |
| 624 | CUAAUCAAGCUGGAUGAUA | UAUCAUCCAGCUUGAUUAG | 625 | 1119 | Yes | No | No |
| 626 | UAAUCAAGCUGGAUGAUGA | UCAUCAUCCAGCUUGAUUA | 627 | 1120 | Yes | No | No |
| 628 | AAUCAAGCUGGAUGAUGCA | UGCAUCAUCCAGCUUGAUU | 629 | 1121 | Yes | No | No |
| 630 | CAAGCUGGAUGAUGCUGUA | UACAGCAUCAUCCAGCUUG | 631 | 1124 | Yes | No | No |
| 632 | AGCUGGAUGAUGCUGUAAA | UUUACAGCAUCAUCCAGCU | 633 | 1126 | Yes | No | No |
| 634 | UGGAUGAUGCUGUAAAUGA | UCAUUUACAGCAUCAUCCA | 635 | 1129 | Yes | No | No |
| 636 | GAUGAUGCUGUAAAUGUUA | UAACAUUUACAGCAUCAUC | 637 | 1131 | Yes | No | No |
| 638 | UGUAAAUGUUGAUGAGAUA | UAUCUCAUCAACAUUUACA | 639 | 1139 | Yes | No | No |
| 640 | GUAAAUGUUGAUGAGAUAA | UUAUCUCAUCAACAUUUAC | 641 | 1140 | Yes | No | No |
| 642 | UAAAUGUUGAUGAGAUAAA | UUUAUCUCAUCAACAUUUA | 643 | 1141 | Yes | No | No |
| 644 | AUGUUGAUGAGAUAAUGAA | UUCAUUAUCUCAUCAACAU | 645 | 1144 | Yes | No | No |
| 646 | UGUUGAUGAGAUAAUGACA | UGUCAUUAUCUCAUCAACA | 647 | 1145 | Yes | No | No |
| 648 | GUUGAUGAGAUAAUGACUA | UAGUCAUUAUCUCAUCAAC | 649 | 1146 | Yes | No | No |
| 650 | UUGAUGAGAUAAUGACUGA | UCAGUCAUUAUCUCAUCAA | 651 | 1147 | Yes | No | No |
| 652 | UGAUGAGAUAAUGACUGAA | UUCAGUCAUUAUCUCAUCA | 653 | 1148 | Yes | No | No |
| 654 | GAUGAGAUAAUGACUGAUA | UAUCAGUCAUUAUCUCAUC | 655 | 1149 | Yes | No | No |
| 656 | AGAUAAUGACUGAUACUUA | UAAGUAUCAGUCAUUAUCU | 657 | 1153 | Yes | No | No |
| 658 | GAUAAUGACUGAUACUUCA | UGAAGUAUCAGUCAUUAUC | 659 | 1154 | Yes | No | No |
| 660 | AUAAUGACUGAUACUUCUA | UAGAAGUAUCAGUCAUUAU | 661 | 1155 | Yes | No | No |
| 662 | AAUGACUGAUACUUCUACA | UGUAGAAGUAUCAGUCAUU | 663 | 1157 | Yes | No | No |
| 664 | UGACUGAUACUUCUACCAA | UUGGUAGAAGUAUCAGUCA | 665 | 1159 | Yes | No | No |
| 666 | GACUGAUACUUCUACCAGA | UCUGGUAGAAGUAUCAGUC | 667 | 1160 | Yes | No | No |
| 668 | UGAUACUUCUACCAGCUAA | UUAGCUGGUAGAAGUAUCA | 669 | 1163 | Yes | No | No |
| 670 | GAUACUUCUACCAGCUAUA | UAUAGCUGGUAGAAGUAUC | 671 | 1164 | Yes | No | No |
| 672 | ACUUCUACCAGCUAUCUUA | UAAGAUAGCUGGUAGAAGU | 673 | 1167 | Yes | No | No |
| 674 | UUCUACCAGCUAUCUUCUA | UAGAAGAUAGCUGGUAGAA | 675 | 1169 | Yes | No | No |
| 676 | UCUACCAGCUAUCUUCUGA | UCAGAAGAUAGCUGGUAGA | 677 | 1170 | Yes | No | No |
| 678 | CUACCAGCUAUCUUCUGUA | UACAGAAGAUAGCUGGUAG | 679 | 1171 | Yes | No | No |
| 680 | UACCAGCUAUCUUCUGUGA | UCACAGAAGAUAGCUGGUA | 681 | 1172 | Yes | No | No |
| 682 | ACCAGCUAUCUUCUGUGCA | UGCACAGAAGAUAGCUGGU | 683 | 1173 | Yes | No | No |
| 684 | CCAGCUAUCUUCUGUGCAA | UUGCACAGAAGAUAGCUGG | 685 | 1174 | Yes | No | No |
| 686 | GCUAUCUUCUGUGCAUCUA | UAGAUGCACAGAAGAUAGC | 687 | 1177 | Yes | No | No |
| 688 | CUAUCUUCUGUGCAUCUCA | UGAGAUGCACAGAAGAUAG | 689 | 1178 | Yes | No | No |
| 690 | AUCUCUGAAAAUAAGGAAA | UUUCCUUAUUUUCAGAGAU | 691 | 1191 | Yes | No | No |
| 692 | GAAAAUAAGGAAAAUGUUA | UAACAUUUUCCUUAUUUUC | 693 | 1197 | Yes | No | No |
| 694 | AAAAUAAGGAAAAUGUUAA | UUAACAUUUUCCUUAUUUU | 695 | 1198 | Yes | No | No |
| 696 | AGGAAAAUGUUAGGGACAA | UUGUCCCUAACAUUUUCCU | 697 | 1204 | Yes | No | No |
| 698 | GGAAAAUGUUAGGGACAAA | UUUGUCCCUAACAUUUUCC | 699 | 1205 | Yes | No | No |
| 700 | UUUUAUUGGCAUUGUGGGA | UCCCACAAUGCCAAUAAAA | 701 | 1238 | Yes | No | No |
| 702 | UGCCACAGGCGAGGUUGUA | UACAACCUCGCCUGUGGCA | 703 | 1265 | Yes | No | No |
| 704 | CCACAGGCGAGGUUGUGUA | UACACAACCUCGCCUGUGG | 705 | 1267 | Yes | No | No |
| 706 | CACAGGCGAGGUUGUGUUA | UAACACAACCUCGCCUGUG | 707 | 1268 | Yes | No | No |
| 708 | CAGGCGAGGUUGUGUUUGA | UCAAACACAACCUCGCCUG | 709 | 1270 | Yes | No | No |
| 710 | AGGCGAGGUUGUGUUUGAA | UUCAAACACAACCUCGCCU | 711 | 1271 | Yes | No | No |
| 712 | GGCGAGGUUGUGUUUGAUA | UAUCAAACACAACCUCGCC | 713 | 1272 | Yes | No | No |
| 714 | GCGAGGUUGUGUUUGAUAA | UUAUCAAACACAACCUCGC | 715 | 1273 | Yes | No | No |
| 716 | CGAGGUUGUGUUUGAUAGA | UCUAUCAAACACAACCUCG | 717 | 1274 | Yes | No | No |
| 718 | GAGGUUGUGUUUGAUAGUA | UACUAUCAAACACAACCUC | 719 | 1275 | Yes | No | No |
| 720 | AGGUUGUGUUUGAUAGUUA | UAACUAUCAAACACAACCU | 721 | 1276 | Yes | No | No |
| 722 | GGUUGUGUUUGAUAGUUUA | UAAACUAUCAAACACAACC | 723 | 1277 | Yes | No | No |
| 724 | GUUGUGUUUGAUAGUUUCA | UGAAACUAUCAAACACAAC | 725 | 1278 | Yes | No | No |
| 726 | GUUUGAUAGUUUCCAGGAA | UUCCUGGAAACUAUCAAAC | 727 | 1283 | Yes | No | No |
| 728 | CAGGACUCUGCUUCUCGUA | UACGAGAAGCAGAGUCCUG | 729 | 1296 | Yes | No | No |
| 730 | AGGACUCUGCUUCUCGUUA | UAACGAGAAGCAGAGUCCU | 731 | 1297 | Yes | No | No |
| 732 | GGACUCUGCUUCUCGUUCA | UGAACGAGAAGCAGAGUCC | 733 | 1298 | Yes | No | No |
| 734 | GACUCUGCUUCUCGUUCAA | UUGAACGAGAAGCAGAGUC | 735 | 1299 | Yes | No | No |
| 736 | ACUCUGCUUCUCGUUCAGA | UCUGAACGAGAAGCAGAGU | 737 | 1300 | Yes | No | No |
| 738 | GCUUCUCGUUCAGAGCUAA | UUAGCUCUGAACGAGAAGC | 739 | 1305 | Yes | No | No |
| 740 | CUUCUCGUUCAGAGCUAGA | UCUAGCUCUGAACGAGAAG | 741 | 1306 | Yes | No | No |
| 742 | UUCUCGUUCAGAGCUAGAA | UUCUAGCUCUGAACGAGAA | 743 | 1307 | Yes | No | No |
| 744 | UCUCGUUCAGAGCUAGAAA | UUUCUAGCUCUGAACGAGA | 745 | 1308 | Yes | No | No |
| 746 | UCGUUCAGAGCUAGAAACA | UGUUUCUAGCUCUGAACGA | 747 | 1310 | Yes | No | No |
| 748 | UAGAAACCCGGAUGUCAAA | UUUGACAUCCGGGUUUCUA | 749 | 1321 | Yes | No | No |
| 750 | GAAACCCGGAUGUCAAGCA | UGCUUGACAUCCGGGUUUC | 751 | 1323 | No | No | No |
| 752 | AAGCCUGCAGCCAGUAGAA | UUCUACUGGCUGCAGGCUU | 753 | 1337 | No | No | No |
| 754 | CUGCAGCCAGUAGAGCUGA | UCAGCUCUACUGGCUGCAG | 755 | 1341 | Yes | No | No |
| 756 | UCCUUCGGCCUUGUCCGAA | UUCGGACAAGGCCGAAGGA | 757 | 1364 | Yes | No | No |
| 758 | CGGCCUUGUCCGAGCAAAA | UUUUGCUCGGACAAGGCCG | 759 | 1369 | Yes | No | No |
| 760 | CCUUGUCCGAGCAAACAGA | UCUGUUUGCUCGGACAAGG | 761 | 1372 | Yes | No | No |
| 762 | CUUGUCCGAGCAAACAGAA | UUCUGUUUGCUCGGACAAG | 763 | 1373 | Yes | No | No |
| 764 | UGUCCGAGCAAACAGAGGA | UCCUCUGUUUGCUCGGACA | 765 | 1375 | No | No | No |
| 766 | AACAGAGGCGCUCAUCCAA | UUGGAUGAGCGCCUCUGUU | 767 | 1385 | Vo | No | No |
| 768 | ACAGAGGCGCUCAUCCACA | UGUGGAUGAGCGCCUCUGU | 769 | 1386 | No | No | No |
| 770 | CAGAGGCGCUCAUCCACAA | UUGUGGAUGAGCGCCUCUG | 771 | 1387 | No | No | No |
| 772 | AGAGGCGCUCAUCCACAGA | UCUGUGGAUGAGCGCCUCU | 773 | 1388 | No | No | No |
| 774 | GAGGCGCUCAUCCACAGAA | UUCUGUGGAUGAGCGCCUC | 775 | 1389 | No | No | No |
| 776 | CACAGAGCCACAUCUGUUA | UAACAGAUGUGGCUCUGUG | 777 | 1401 | Yes | No | No |
| 778 | AGAGCCACAUCUGUUAGUA | UACUAACAGAUGUGGCUCU | 779 | 1404 | Yes | No | No |
| 780 | AGCCACAUCUGUUAGUGUA | UACACUAACAGAUGUGGCU | 781 | 1406 | Yes | No | No |
| 782 | GCCACAUCUGUUAGUGUGA | UCACACUAACAGAUGUGGC | 783 | 1407 | Yes | No | No |
| 784 | CCACAUCUGUUAGUGUGCA | UGCACACUAACAGAUGUGG | 785 | 1408 | Yes | No | No |
| 786 | UCUGUUAGUGUGCAGGAUA | UAUCCUGCACACUAACAGA | 787 | 1413 | Yes | No | No |
| 788 | GUUAGUGUGCAGGAUGACA | UGUCAUCCUGCACACUAAC | 789 | 1416 | Yes | No | No |
| 790 | UUAGUGUGCAGGAUGACAA | UUGUCAUCCUGCACACUAA | 791 | 1417 | Yes | No | No |
| 792 | GUGUGCAGGAUGACAGAAA | UUUCUGUCAUCCUGCACAC | 793 | 1420 | Yes | No | No |
| 794 | UGUGCAGGAUGACAGAAUA | UAUUCUGUCAUCCUGCACA | 795 | 1421 | Yes | No | No |
| 796 | GUGCAGGAUGACAGAAUUA | UAAUUCUGUCAUCCUGCAC | 797 | 1422 | Yes | No | No |
| 798 | AGGAUGACAGAAUUCGAGA | UCUCGAAUUCUGUCAUCCU | 799 | 1426 | Yes | No | No |
| 800 | ACAGAAUUCGAGUCGAAAA | UUUUCGACUCGAAUUCUGU | 801 | 1432 | No | No | No |
| 802 | CAGAAUUCGAGUCGAAAGA | UCUUUCGACUCGAAUUCUG | 803 | 1433 | No | No | No |
| 804 | AGAAUUCGAGUCGAAAGGA | UCCUUUCGACUCGAAUUCU | 805 | 1434 | No | No | No |
| 806 | UUCGAGUCGAAAGGAUGGA | UCCAUCCUUUCGACUCGAA | 807 | 1438 | No | No | No |
| 808 | CGAGUCGAAAGGAUGGAUA | UAUCCAUCCUUUCGACUCG | 809 | 1440 | No | No | No |
| 810 | GAGUCGAAAGGAUGGAUAA | UUAUCCAUCCUUUCGACUC | 811 | 1441 | No | No | No |
| 812 | AGUCGAAAGGAUGGAUAAA | UUUAUCCAUCCUUUCGACU | 813 | 1442 | No | No | No |
| 814 | GUCGAAAGGAUGGAUAACA | UGUUAUCCAUCCUUUCGAC | 815 | 1443 | No | No | No |
| 816 | UCGAAAGGAUGGAUAACAA | UUGUUAUCCAUCCUUUCGA | 817 | 1444 | No | No | No |
| 818 | CGAAAGGAUGGAUAACAUA | UAUGUUAUCCAUCCUUUCG | 819 | 1445 | No | No | No |
| 820 | GAAAGGAUGGAUAACAUUA | UAAUGUUAUCCAUCCUUUC | 821 | 1446 | Yes | No | No |
| 822 | AAAGGAUGGAUAACAUUUA | UAAAUGUUAUCCAUCCUUU | 823 | 1447 | Yes | No | No |
| 824 | AGGAUGGAUAACAUUUAUA | UAUAAAUGUUAUCCAUCCU | 825 | 1449 | Yes | No | No |
| 826 | GGAUAACAUUUAUUUUGAA | UUCAAAAUAAAUGUUAUCC | 827 | 1454 | Yes | No | No |
| 828 | GAUAACAUUUAUUUUGAAA | UUUCAAAAUAAAUGUUAUC | 829 | 1455 | Yes | No | No |
| 830 | AUAACAUUUAUUUUGAAUA | UAUUCAAAAUAAAUGUUAU | 831 | 1456 | Yes | No | No |
| 832 | UGAAUACAGCCAUGCUUUA | UAAAGCAUGGCUGUAUUCA | 833 | 1469 | Yes | Yes | Yes |
| 834 | CAUGCUUUCCAGGCAGUUA | UAACUGCCUGGAAAGCAUG | 835 | 1479 | Yes | No | No |
| 836 | AUGCUUUCCAGGCAGUUAA | UUAACUGCCUGGAAAGCAU | 837 | 1480 | Yes | No | No |
| 838 | UGCUUUCCAGGCAGUUACA | UGUAACUGCCUGGAAAGCA | 839 | 1481 | Yes | No | No |
| 840 | GCUUUCCAGGCAGUUACAA | UUGUAACUGCCUGGAAAGC | 841 | 1482 | Yes | No | No |
| 842 | UCCAGGCAGUUACAGAGUA | UACUCUGUAACUGCCUGGA | 843 | 1486 | Yes | No | No |
| 844 | CAGGCAGUUACAGAGUUUA | UAAACUCUGUAACUGCCUG | 845 | 1488 | Yes | No | No |
| 846 | GCAGUUACAGAGUUUUAUA | UAUAAAACUCUGUAACUGC | 847 | 1491 | Yes | No | No |
| 848 | CAGUUACAGAGUUUUAUGA | UCAUAAAACUCUGUAACUG | 849 | 1492 | Yes | No | No |
| 850 | GUUACAGAGUUUUAUGCAA | UUGCAUAAAACUCUGUAAC | 851 | 1494 | Yes | No | No |
| 852 | UUACAGAGUUUUAUGCAAA | UUUGCAUAAAACUCUGUAA | 853 | 1495 | Yes | No | No |
| 854 | UACAGAGUUUUAUGCAAAA | UUUUGCAUAAAACUCUGUA | 855 | 1496 | Yes | No | No |
| 856 | UUUUAUGCAAAAGAUACAA | UUGUAUCUUUUGCAUAAAA | 857 | 1503 | No | No | No |
| 858 | UAUGCAAAAGAUACAGUUA | UAACUGUAUCUUUUGCAUA | 859 | 1506 | No | No | No |
| 860 | UGCAAAAGAUACAGUUGAA | UUCAACUGUAUCUUUUGCA | 861 | 1508 | No | No | No |
| 862 | GCAAAAGAUACAGUUGACA | UGUCAACUGUAUCUUUUGC | 863 | 1509 | No | No | No |
| 864 | CAAAAGAUACAGUUGACAA | UUGUCAACUGUAUCUUUUG | 865 | 1510 | No | No | No |
| 866 | AAAAGAUACAGUUGACAUA | UAUGUCAACUGUAUCUUUU | 867 | 1511 | No | No | No |
| 868 | GAUACAGUUGACAUCAAAA | UUUUGAUGUCAACUGUAUC | 869 | 1515 | No | No | No |
| 870 | AUACAGUUGACAUCAAAGA | UCUUUGAUGUCAACUGUAU | 871 | 1516 | No | No | No |
| 872 | UACAGUUGACAUCAAAGGA | UCCUUUGAUGUCAACUGUA | 873 | 1517 | No | No | No |
| 874 | ACAGUUGACAUCAAAGGUA | UACCUUUGAUGUCAACUGU | 875 | 1518 | Yes | No | No |
| 876 | CAGUUGACAUCAAAGGUUA | UAACCUUUGAUGUCAACUG | 877 | 1519 | Yes | No | No |
| 878 | AGUUGACAUCAAAGGUUCA | UGAACCUUUGAUGUCAACU | 879 | 1520 | Yes | No | No |
| 880 | GUUGACAUCAAAGGUUCUA | UAGAACCUUUGAUGUCAAC | 881 | 1521 | Yes | No | No |
| 882 | UUGACAUCAAAGGUUCUCA | UGAGAACCUUUGAUGUCAA | 883 | 1522 | Yes | No | No |
| 884 | UGACAUCAAAGGUUCUCAA | UUGAGAACCUUUGAUGUCA | 885 | 1523 | Yes | No | No |
| 886 | GACAUCAAAGGUUCUCAAA | UUUGAGAACCUUUGAUGUC | 887 | 1524 | Yes | No | No |
| 888 | UCAAAGGUUCUCAAAUUAA | UUAAUUUGAGAACCUUUGA | 889 | 1528 | Yes | No | No |
| 890 | AAGGUUCUCAAAUUAUUUA | UAAAUAAUUUGAGAACCUU | 891 | 1531 | Yes | No | No |
| 892 | UCAAAUUAUUUCUGGCAUA | UAUGCCAGAAAUAAUUUGA | 893 | 1538 | Yes | No | No |
| 894 | CAAAUUAUUUCUGGCAUUA | UAAUGCCAGAAAUAAUUUG | 895 | 1539 | Yes | No | No |
| 896 | AAUUAUUUCUGGCAUUGUA | UACAAUGCCAGAAAUAAUU | 897 | 1541 | Yes | No | No |
| 898 | AUUAUUUCUGGCAUUGUUA | UAACAAUGCCAGAAAUAAU | 899 | 1542 | Yes | No | No |
| 900 | UUAUUUCUGGCAUUGUUAA | UUAACAAUGCCAGAAAUAA | 901 | 1543 | Yes | No | No |
| 902 | UUUCUGGCAUUGUUAACUA | UAGUUAACAAUGCCAGAAA | 903 | 1546 | Yes | No | No |
| 904 | UUCUGGCAUUGUUAACUUA | UAAGUUAACAAUGCCAGAA | 905 | 1547 | Yes | No | No |
| 906 | CUGGCAUUGUUAACUUAGA | UCUAAGUUAACAAUGCCAG | 907 | 1549 | Yes | No | No |
| 908 | GCAUUGUUAACUUAGAGAA | UUCUCUAAGUUAACAAUGC | 909 | 1552 | Yes | No | No |
| 910 | GUUAACUUAGAGAAGCCUA | UAGGCUUCUCUAAGUUAAC | 911 | 1557 | Yes | No | No |
| 912 | UAACUUAGAGAAGCCUGUA | UACAGGCUUCUCUAAGUUA | 913 | 1559 | Yes | No | No |
| 914 | UAGAGAAGCCUGUGAUUUA | UAAAUCACAGGCUUCUCUA | 915 | 1564 | Yes | No | No |
| 916 | AAGCCUGUGAUUUGCUCUA | UAGAGCAAAUCACAGGCUU | 917 | 1569 | Yes | No | No |
| 918 | GCCUGUGAUUUGCUCUUUA | UAAAGAGCAAAUCACAGGC | 919 | 1571 | Yes | No | No |
| 920 | UGUGAUUUGCUCUUUGGCA | UGCCAAAGAGCAAAUCACA | 921 | 1574 | Yes | No | No |
| 922 | GUGAUUUGCUCUUUGGCUA | UAGCCAAAGAGCAAAUCAC | 923 | 1575 | Yes | No | No |
| 924 | GCUCUUUGGCUGCCAUCAA | UUGAUGGCAGCCAAAGAGC | 925 | 1582 | Yes | No | No |
| 926 | UCUUUGGCUGCCAUCAUAA | UUAUGAUGGCAGCCAAAGA | 927 | 1584 | Yes | No | No |
| 928 | CUUUGGCUGCCAUCAUAAA | UUUAUGAUGGCAGCCAAAG | 929 | 1585 | Yes | No | No |
| 930 | UUUGGCUGCCAUCAUAAAA | UUUUAUGAUGGCAGCCAAA | 931 | 1586 | Yes | No | No |
| 932 | UGGCUGCCAUCAUAAAAUA | UAUUUUAUGAUGGCAGCCA | 933 | 1588 | Yes | No | No |
| 934 | GGCUGCCAUCAUAAAAUAA | UUAUUUUAUGAUGGCAGCC | 935 | 1589 | Yes | No | No |
| 936 | GCUGCCAUCAUAAAAUACA | UGUAUUUUAUGAUGGCAGC | 937 | 1590 | Yes | No | No |
| 938 | UGCCAUCAUAAAAUACCUA | UAGGUAUUUUAUGAUGGCA | 939 | 1592 | Yes | No | No |
| 940 | GCCAUCAUAAAAUACCUCA | UGAGGUAUUUUAUGAUGGC | 941 | 1593 | Yes | No | No |
| 942 | CAUCAUAAAAUACCUCAAA | UUUGAGGUAUUUUAUGAUG | 943 | 1595 | Yes | No | No |
| 944 | CAUAAAAUACCUCAAAGAA | UUCUUUGAGGUAUUUUAUG | 945 | 1598 | Yes | No | No |
| 946 | UACCUCAAAGAAUUCAACA | UGUUGAAUUCUUUGAGGUA | 947 | 1605 | Yes | No | No |
| 948 | CCUCAAAGAAUUCAACUUA | UAAGUUGAAUUCUUUGAGG | 949 | 1607 | Yes | No | No |
| 950 | UCAACUUGGAAAAGAUGCA | UGCAUCUUUUCCAAGUUGA | 951 | 1618 | No | No | No |
| 952 | CAACUUGGAAAAGAUGCUA | UAGCAUCUUUUCCAAGUUG | 953 | 1619 | No | No | No |
| 954 | GGAAAAGAUGCUCUCCAAA | UUUGGAGAGCAUCUUUUCC | 955 | 1625 | No | No | No |
| 956 | AAAGAUGCUCUCCAAACCA | UGGUUUGGAGAGCAUCUUU | 957 | 1628 | No | No | No |
| 958 | GAUGCUCUCCAAACCUGAA | UUCAGGUUUGGAGAGCAUC | 959 | 1631 | No | No | No |
| 960 | AUGCUCUCCAAACCUGAGA | UCUCAGGUUUGGAGAGCAU | 961 | 1632 | Yes | No | No |
| 962 | GCUCUCCAAACCUGAGAAA | UUUCUCAGGUUUGGAGAGC | 963 | 1634 | Yes | No | No |
| 964 | CUCUCCAAACCUGAGAAUA | UAUUCUCAGGUUUGGAGAG | 965 | 1635 | Yes | No | No |
| 966 | UCUCCAAACCUGAGAAUUA | UAAUUCUCAGGUUUGGAGA | 967 | 1636 | Yes | No | No |
| 968 | CUCCAAACCUGAGAAUUUA | UAAAUUCUCAGGUUUGGAG | 969 | 1637 | Yes | No | No |
| 970 | AACCUGAGAAUUUUAAACA | UGUUUAAAAUUCUCAGGUU | 971 | 1642 | Yes | No | No |
| 972 | CCUGAGAAUUUUAAACAGA | UCUGUUUAAAAUUCUCAGG | 973 | 1644 | Yes | No | No |
| 974 | CUGAGAAUUUUAAACAGCA | UGCUGUUUAAAAUUCUCAG | 975 | 1645 | Yes | No | No |
| 976 | GAGAAUUUUAAACAGCUAA | UUAGCUGUUUAAAAUUCUC | 977 | 1647 | Yes | No | No |
| 978 | AGAAUUUUAAACAGCUAUA | UAUAGCUGUUUAAAAUUCU | 979 | 1648 | Yes | No | No |
| 980 | GAAUUUUAAACAGCUAUCA | UGAUAGCUGUUUAAAAUUC | 981 | 1649 | Yes | No | No |
| 982 | UAAACAGCUAUCAAGUAAA | UUUACUUGAUAGCUGUUUA | 983 | 1655 | Yes | No | No |
| 984 | AAACAGCUAUCAAGUAAAA | UUUUACUUGAUAGCUGUUU | 985 | 1656 | Yes | No | No |
| 986 | CAGCUAUCAAGUAAAAUGA | UCAUUUUACUUGAUAGCUG | 987 | 1659 | Yes | No | No |
| 988 | GCUAUCAAGUAAAAUGGAA | UUCCAUUUUACUUGAUAGC | 989 | 1661 | Yes | No | No |
| 990 | CUAUCAAGUAAAAUGGAAA | UUUCCAUUUUACUUGAUAG | 991 | 1662 | Yes | No | No |
| 992 | UAUCAAGUAAAAUGGAAUA | UAUUCCAUUUUACUUGAUA | 993 | 1663 | Yes | No | No |
| 994 | UCAAGUAAAAUGGAAUUUA | UAAAUUCCAUUUUACUUGA | 995 | 1665 | Yes | No | No |
| 996 | CAAGUAAAAUGGAAUUUAA | UUAAAUUCCAUUUUACUUG | 997 | 1666 | Yes | No | No |
| 998 | AAAAUGGAAUUUAUGACAA | UUGUCAUAAAUUCCAUUUU | 999 | 1671 | Yes | No | No |
| 1000 | AAUGGAAUUUAUGACAAUA | UAUUGUCAUAAAUUCCAUU | 1001 | 1673 | Yes | No | No |
| 1002 | AUGGAAUUUAUGACAAUUA | UAAUUGUCAUAAAUUCCAU | 1003 | 1674 | Yes | No | No |
| 1004 | AAUUUAUGACAAUUAAUGA | UCAUUAAUUGUCAUAAAUU | 1005 | 1678 | Yes | No | No |
| 1006 | UUAUGACAAUUAAUGGAAA | UUUCCAUUAAUUGUCAUAA | 1007 | 1681 | Yes | No | No |
| 1008 | UAUGACAAUUAAUGGAACA | UGUUCCAUUAAUUGUCAUA | 1009 | 1682 | Yes | No | No |
| 1010 | AUGACAAUUAAUGGAACAA | UUGUUCCAUUAAUUGUCAU | 1011 | 1683 | Yes | No | No |
| 1012 | UGACAAUUAAUGGAACAAA | UUUGUUCCAUUAAUUGUCA | 1013 | 1684 | Yes | No | No |
| 1014 | GACAAUUAAUGGAACAACA | UGUUGUUCCAUUAAUUGUC | 1015 | 1685 | Yes | No | No |
| 1016 | ACAAUUAAUGGAACAACAA | UUGUUGUUCCAUUAAUUGU | 1017 | 1686 | Yes | No | No |
| 1018 | AAUGGAACAACAUUAAGGA | UCCUUAAUGUUGUUCCAUU | 1019 | 1692 | Yes | No | No |
| 1020 | GGAACAACAUUAAGGAAUA | UAUUCCUUAAUGUUGUUCC | 1021 | 1695 | Yes | No | No |
| 1022 | CAACAUUAAGGAAUCUGGA | UCCAGAUUCCUUAAUGUUG | 1023 | 1699 | Yes | No | No |
| 1024 | UAAGGAAUCUGGAAAUCCA | UGGAUUUCCAGAUUCCUUA | 1025 | 1705 | Yes | No | Yes |
| 1026 | GGAAUCUGGAAAUCCUACA | UGUAGGAUUUCCAGAUUCC | 1027 | 1708 | Yes | No | Yes |
| 1028 | GAAUCUGGAAAUCCUACAA | UUGUAGGAUUUCCAGAUUC | 1029 | 1709 | Yes | No | Yes |
| 1030 | UCUGGAAAUCCUACAGAAA | UUUCUGUAGGAUUUCCAGA | 1031 | 1712 | Yes | No | Yes |
| 1032 | CUGGAAAUCCUACAGAAUA | UAUUCUGUAGGAUUUCCAG | 1033 | 1713 | Yes | No | Yes |
| 1034 | UGGAAAUCCUACAGAAUCA | UGAUUCUGUAGGAUUUCCA | 1035 | 1714 | Yes | No | Yes |
| 1036 | GAAAUCCUACAGAAUCAGA | UCUGAUUCUGUAGGAUUUC | 1037 | 1716 | Yes | No | Yes |
| 1038 | AAUCCUACAGAAUCAGACA | UGUCUGAUUCUGUAGGAUU | 1039 | 1718 | Yes | No | Yes |
| 1040 | AUCCUACAGAAUCAGACUA | UAGUCUGAUUCUGUAGGAU | 1041 | 1719 | Yes | No | Yes |
| 1042 | UCCUACAGAAUCAGACUGA | UCAGUCUGAUUCUGUAGGA | 1043 | 1720 | Yes | No | Yes |
| 1044 | CCUACAGAAUCAGACUGAA | UUCAGUCUGAUUCUGUAGG | 1045 | 1721 | Yes | No | Yes |
| 1046 | CUACAGAAUCAGACUGAUA | UAUCAGUCUGAUUCUGUAG | 1047 | 1722 | Yes | No | Yes |
| 1048 | UACAGAAUCAGACUGAUAA | UUAUCAGUCUGAUUCUGUA | 1049 | 1723 | Yes | No | Yes |
| 1050 | ACAGAAUCAGACUGAUAUA | UAUAUCAGUCUGAUUCUGU | 1051 | 1724 | Yes | No | Yes |
| 1052 | CAGAAUCAGACUGAUAUGA | UCAUAUCAGUCUGAUUCUG | 1053 | 1725 | Yes | Yes | Yes |
| 1054 | UCAGACUGAUAUGAAAACA | UGUUUUCAUAUCAGUCUGA | 1055 | 1730 | Yes | No | No |
| 1056 | GACUGAUAUGAAAACCAAA | UUUGGUUUUCAUAUCAGUC | 1057 | 1733 | Yes | No | No |
| 1058 | ACUGAUAUGAAAACCAAAA | UUUUGGUUUUCAUAUCAGU | 1059 | 1734 | Yes | No | No |
| 1060 | CUGAUAUGAAAACCAAAGA | UCUUUGGUUUUCAUAUCAG | 1061 | 1735 | Yes | No | No |
| 1062 | GAUAUGAAAACCAAAGGAA | UUCCUUUGGUUUUCAUAUC | 1063 | 1737 | Yes | No | No |
| 1064 | GAAAACCAAAGGAAGUUUA | UAAACUUCCUUUGGUUUUC | 1065 | 1742 | Yes | No | No |
| 1066 | AAAACCAAAGGAAGUUUGA | UCAAACUUCCUUUGGUUUU | 1067 | 1743 | Yes | No | No |
| 1068 | AAACCAAAGGAAGUUUGCA | UGCAAACUUCCUUUGGUUU | 1069 | 1744 | Yes | No | No |
| 1070 | ACCAAAGGAAGUUUGCUGA | UCAGCAAACUUCCUUUGGU | 1071 | 1746 | Yes | No | No |
| 1072 | GGAAGUUUGCUGUGGGUUA | UAACCCACAGCAAACUUCC | 1073 | 1752 | Yes | No | No |
| 1074 | AGUUUGCUGUGGGUUUUAA | UUAAAACCCACAGCAAACU | 1075 | 1755 | Yes | No | No |
| 1076 | UUGCUGUGGGUUUUAGACA | UGUCUAAAACCCACAGCAA | 1077 | 1758 | Yes | No | No |
| 1078 | UGCUGUGGGUUUUAGACCA | UGGUCUAAAACCCACAGCA | 1079 | 1759 | Yes | No | No |
| 1080 | GCUGUGGGUUUUAGACCAA | UUGGUCUAAAACCCACAGC | 1081 | 1760 | Yes | No | No |
| 1082 | CUGUGGGUUUUAGACCACA | UGUGGUCUAAAACCCACAG | 1083 | 1761 | Yes | No | No |
| 1084 | UGGGUUUUAGACCACACUA | UAGUGUGGUCUAAAACCCA | 1085 | 1764 | Yes | Yes | Yes |
| 1086 | GGUUUUAGACCACACUAAA | UUUAGUGUGGUCUAAAACC | 1087 | 1766 | Yes | Yes | Yes |
| 1088 | GUUUUAGACCACACUAAAA | UUUUAGUGUGGUCUAAAAC | 1089 | 1767 | Yes | Yes | Yes |
| 1090 | UUUAGACCACACUAAAACA | UGUUUUAGUGUGGUCUAAA | 1091 | 1769 | Yes | Yes | Yes |
| 1092 | UUAGACCACACUAAAACUA | UAGUUUUAGUGUGGUCUAA | 1093 | 1770 | Yes | Yes | No |
| 1094 | UAGACCACACUAAAACUUA | UAAGUUUUAGUGUGGUCUA | 1095 | 1771 | Yes | Yes | No |
| 1096 | AGACCACACUAAAACUUCA | UGAAGUUUUAGUGUGGUCU | 1097 | 1772 | Yes | Yes | No |
| 1098 | CCACACUAAAACUUCAUUA | UAAUGAAGUUUUAGUGUGG | 1099 | 1775 | Yes | Yes | No |
| 1100 | CACUAAAACUUCAUUUGGA | UCCAAAUGAAGUUUUAGUG | 1101 | 1778 | Yes | Yes | No |
| 1102 | ACUAAAACUUCAUUUGGGA | UCCCAAAUGAAGUUUUAGU | 1103 | 1779 | Yes | Yes | No |
| 1104 | UAAAACUUCAUUUGGGAGA | UCUCCCAAAUGAAGUUUUA | 1105 | 1781 | Yes | Yes | No |
| 1106 | AUUUGGGAGACGGAAGUUA | UAACUUCCGUCUCCCAAAU | 1107 | 1790 | Yes | No | No |
| 1108 | UUGGGAGACGGAAGUUAAA | UUUAACUUCCGUCUCCCAA | 1109 | 1792 | Yes | No | No |
| 1110 | GGGAGACGGAAGUUAAAGA | UCUUUAACUUCCGUCUCCC | 1111 | 1794 | Yes | No | No |
| 1112 | GGAGACGGAAGUUAAAGAA | UUCUUUAACUUCCGUCUCC | 1113 | 1795 | Yes | No | No |
| 1114 | GACGGAAGUUAAAGAAGUA | UACUUCUUUAACUUCCGUC | 1115 | 1798 | Yes | No | No |
| 1116 | ACGGAAGUUAAAGAAGUGA | UCACUUCUUUAACUUCCGU | 1117 | 1799 | Yes | No | No |
| 1118 | CGGAAGUUAAAGAAGUGGA | UCCACUUCUUUAACUUCCG | 1119 | 1800 | Yes | No | No |
| 1120 | GUGACCCAGCCACUCCUUA | UAAGGAGUGGCUGGGUCAC | 1121 | 1818 | Yes | No | No |
| 1122 | UGACCCAGCCACUCCUUAA | UUAAGGAGUGGCUGGGUCA | 1123 | 1819 | Yes | No | No |
| 1124 | ACCCAGCCACUCCUUAAAA | UUUUAAGGAGUGGCUGGGU | 1125 | 1821 | Yes | No | No |
| 1126 | CCCAGCCACUCCUUAAAUA | UAUUUAAGGAGUGGCUGGG | 1127 | 1822 | Yes | No | No |
| 1128 | GCCACUCCUUAAAUUAAGA | UCUUAAUUUAAGGAGUGGC | 1129 | 1826 | Yes | No | No |
| 1130 | CACUCCUUAAAUUAAGGGA | UCCCUUAAUUUAAGGAGUG | 1131 | 1828 | Yes | No | No |
| 1132 | ACUCCUUAAAUUAAGGGAA | UUCCCUUAAUUUAAGGAGU | 1133 | 1829 | Yes | No | No |
| 1134 | CUCCUUAAAUUAAGGGAAA | UUUCCCUUAAUUUAAGGAG | 1135 | 1830 | Yes | No | No |
| 1136 | CCUUAAAUUAAGGGAAAUA | UAUUUCCCUUAAUUUAAGG | 1137 | 1832 | Yes | No | No |
| 1138 | UAAAUUAAGGGAAAUAAAA | UUUUAUUUCCCUUAAUUUA | 1139 | 1835 | Yes | Yes | No |
| 1140 | UUAAGGGAAAUAAAUGCCA | UGGCAUUUAUUUCCCUUAA | 1141 | 1839 | Yes | Yes | No |
| 1142 | AUAAAUGCCCGGCUUGAUA | UAUCAAGCCGGGCAUUUAU | 1143 | 1848 | Yes | No | No |
| 1144 | AAAUGCCCGGCUUGAUGCA | UGCAUCAAGCCGGGCAUUU | 1145 | 1850 | Yes | No | No |
| 1146 | AAUGCCCGGCUUGAUGCUA | UAGCAUCAAGCCGGGCAUU | 1147 | 1851 | Yes | No | No |
| 1148 | AUGCCCGGCUUGAUGCUGA | UCAGCAUCAAGCCGGGCAU | 1149 | 1852 | Yes | No | No |
| 1150 | UGCCCGGCUUGAUGCUGUA | UACAGCAUCAAGCCGGGCA | 1151 | 1853 | Yes | No | No |
| 1152 | GCCCGGCUUGAUGCUGUAA | UUACAGCAUCAAGCCGGGC | 1153 | 1854 | Yes | No | No |
| 1154 | CGGCUUGAUGCUGUAUCGA | UCGAUACAGCAUCAAGCCG | 1155 | 1857 | No | No | No |
| 1156 | GGCUUGAUGCUGUAUCGGA | UCCGAUACAGCAUCAAGCC | 1157 | 1858 | No | No | No |
| 1158 | GCUUGAUGCUGUAUCGGAA | UUCCGAUACAGCAUCAAGO | 1159 | 1859 | No | No | No |
| 1160 | UUGAUGCUGUAUCGGAAGA | UCUUCCGAUACAGCAUCAA | 1161 | 1861 | No | No | No |
| 1162 | UGAUGCUGUAUCGGAAGUA | UACUUCCGAUACAGCAUCA | 1163 | 1862 | No | No | No |
| 1164 | GAUGCUGUAUCGGAAGUUA | UAACUUCCGAUACAGCAUC | 1165 | 1863 | No | No | No |
| 1166 | AUGCUGUAUCGGAAGUUCA | UGAACUUCCGAUACAGCAU | 1167 | 1864 | No | No | No |
| 1168 | GCUGUAUCGGAAGUUCUCA | UGAGAACUUCCGAUACAGC | 1169 | 1866 | No | No | No |
| 1170 | CUGUAUCGGAAGUUCUCCA | UGGAGAACUUCCGAUACAG | 1171 | 1867 | No | No | No |
| 1172 | UGUAUCGGAAGUUCUCCAA | UUGGAGAACUUCCGAUACA | 1173 | 1868 | No | No | No |
| 1174 | GUAUCGGAAGUUCUCCAUA | UAUGGAGAACUUCCGAUAC | 1175 | 1869 | No | No | No |
| 1176 | AUCGGAAGUUCUCCAUUCA | UGAAUGGAGAACUUCCGAU | 1177 | 1871 | No | No | No |
| 1178 | UCGGAAGUUCUCCAUUCAA | UUGAAUGGAGAACUUCCGA | 1179 | 1872 | No | No | No |
| 1180 | CGGAAGUUCUCCAUUCAGA | UCUGAAUGGAGAACUUCCG | 1181 | 1873 | No | No | No |
| 1182 | AGUUCUCCAUUCAGAAUCA | UGAUUCUGAAUGGAGAACU | 1183 | 1877 | Yes | No | No |
| 1184 | GUUCUCCAUUCAGAAUCUA | UAGAUUCUGAAUGGAGAAC | 1185 | 1878 | Yes | No | No |
| 1186 | CUCCAUUCAGAAUCUAGUA | UACUAGAUUCUGAAUGGAG | 1187 | 1881 | Yes | No | No |
| 1188 | UCCAUUCAGAAUCUAGUGA | UCACUAGAUUCUGAAUGGA | 1189 | 1882 | Yes | No | No |
| 1190 | CCAUUCAGAAUCUAGUGUA | UACACUAGAUUCUGAAUGG | 1191 | 1883 | Yes | No | No |
| 1192 | CAUUCAGAAUCUAGUGUGA | UCACACUAGAUUCUGAAUG | 1193 | 1884 | Yes | No | NO |
| 1194 | AUUCAGAAUCUAGUGUGUA | UACACACUAGAUUCUGAAU | 1195 | 1885 | Yes | No | No |
| 1196 | UUCAGAAUCUAGUGUGUUA | UAACACACUAGAUUCUGAA | 1197 | 1886 | Yes | No | No |
| 1198 | AGAAUCUAGUGUGUUUGGA | UCCAAACACACUAGAUUCU | 1199 | 1889 | Yes | No | No |
| 1200 | GAAUCUAGUGUGUUUGGUA | UACCAAACACACUAGAUUC | 1201 | 1890 | Yes | No | No |
| 1202 | AAUCUAGUGUGUUUGGUCA | UGACCAAACACACUAGAUU | 1203 | 1891 | Yes | No | No |
| 1204 | AUCUAGUGUGUUUGGUCAA | UUGACCAAACACACUAGAU | 1205 | 1892 | Yes | No | No |
| 1206 | CUAGUGUGUUUGGUCAGAA | UUCUGACCAAACACACUAG | 1207 | 1894 | Yes | No | No |
| 1208 | UGUGUUUGGUCAGAUAGAA | UUCUAUCUGACCAAACACA | 1209 | 1898 | Yes | No | No |
| 1210 | GUGUUUGGUCAGAUAGAAA | UUUCUAUCUGACCAAACAC | 1211 | 1899 | Yes | No | No |
| 1212 | UGUUUGGUCAGAUAGAAAA | UUUUCUAUCUGACCAAACA | 1213 | 1900 | Yes | No | No |
| 1214 | UUUGGUCAGAUAGAAAAUA | UAUUUUCUAUCUGACCAAA | 1215 | 1902 | Yes | No | No |
| 1216 | UUGGUCAGAUAGAAAAUCA | UGAUUUUCUAUCUGACCAA | 1217 | 1903 | Yes | No | No |
| 1218 | GUCAGAUAGAAAAUCAUCA | UGAUGAUUUUCUAUCUGAC | 1219 | 1906 | Yes | No | No |
| 1220 | CAGAUAGAAAAUCAUCUAA | UUAGAUGAUUUUCUAUCUG | 1221 | 1908 | Yes | No | No |
| 1222 | AUAGAAAAUCAUCUACGUA | UACGUAGAUGAUUUUCUAU | 1223 | 1911 | Yes | No | No |
| 1224 | UAGAAAAUCAUCUACGUAA | UUACGUAGAUGAUUUUCUA | 1225 | 1912 | Yes | No | No |
| 1226 | AGAAAAUCAUCUACGUAAA | UUUACGUAGAUGAUUUUCU | 1227 | 1913 | Yes | No | No |
| 1228 | AAAAUCAUCUACGUAAAUA | UAUUUACGUAGAUGAUUUU | 1229 | 1915 | Yes | No | No |
| 1230 | AAUCAUCUACGUAAAUUGA | UCAAUUUACGUAGAUGAUU | 1231 | 1917 | Yes | No | No |
| 1232 | AUCAUCUACGUAAAUUGCA | UGCAAUUUACGUAGAUGAU | 1233 | 1918 | Yes | No | No |
| 1234 | CAUCUACGUAAAUUGCCCA | UGGGCAAUUUACGUAGAUG | 1235 | 1920 | No | No | No |
| 1236 | GUAAAUUGCCCGACAUAGA | UCUAUGUCGGGCAAUUUAC | 1237 | 1927 | No | No | No |
| 1238 | UAAAUUGCCCGACAUAGAA | UUCUAUGUCGGGCAAUUUA | 1239 | 1928 | No | No | No |
| 1240 | AAAUUGCCCGACAUAGAGA | UCUCUAUGUCGGGCAAUUU | 1241 | 1929 | No | No | No |
| 1242 | AUUGCCCGACAUAGAGAGA | UCUCUCUAUGUCGGGCAAU | 1243 | 1931 | No | No | No |
| 1244 | UUGCCCGACAUAGAGAGGA | UCCUCUCUAUGUCGGGCAA | 1245 | 1932 | No | No | No |
| 1246 | AUAGAGAGGGGACUCUGUA | UACAGAGUCCCCUCUCUAU | 1247 | 1941 | No | No | No |
| 1248 | AGAGGGGACUCUGUAGCAA | UUGCUACAGAGUCCCCUCU | 1249 | 1945 | No | No | No |
| 1250 | GGACUCUGUAGCAUUUAUA | UAUAAAUGCUACAGAGUCC | 1251 | 1950 | Yes | No | No |
| 1252 | GACUCUGUAGCAUUUAUCA | UGAUAAAUGCUACAGAGUC | 1253 | 1951 | Yes | No | No |
| 1254 | CUCUGUAGCAUUUAUCACA | UGUGAUAAAUGCUACAGAG | 1255 | 1953 | Yes | No | No |
| 1256 | AAAAUGUUCUACCCAAGAA | UUCUUGGGUAGAACAUUUU | 1257 | 1973 | Yes | No | No |
| 1258 | AAUGUUCUACCCAAGAGUA | UACUCUUGGGUAGAACAUU | 1259 | 1975 | Yes | No | No |
| 1260 | AUGUUCUACCCAAGAGUUA | UAACUCUUGGGUAGAACAU | 1261 | 1976 | Yes | No | No |
| 1262 | UGUUCUACCCAAGAGUUCA | UGAACUCUUGGGUAGAACA | 1263 | 1977 | Yes | No | No |
| 1264 | UUCUACCCAAGAGUUCUUA | UAAGAACUCUUGGGUAGAA | 1265 | 1979 | Yes | No | No |
| 1266 | UCUACCCAAGAGUUCUUCA | UGAAGAACUCUUGGGUAGA | 1267 | 1980 | Yes | Yes | No |
| 1268 | UACCCAAGAGUUCUUCUUA | UAAGAAGAACUCUUGGGUA | 1269 | 1982 | Yes | Yes | No |
| 1270 | ACCCAAGAGUUCUUCUUGA | UCAAGAAGAACUCUUGGGU | 1271 | 1983 | Yes | Yes | No |
| 1272 | GAGUUCUUCUUGAUUGUCA | UGACAAUCAAGAAGAACUC | 1273 | 1989 | Yes | No | No |
| 1274 | UCUUGAUUGUCAAAACUUA | UAAGUUUUGACAAUCAAGA | 1275 | 1996 | No | No | No |
| 1276 | UUGAUUGUCAAAACUUUAA | UUAAAGUUUUGACAAUCAA | 1277 | 1998 | No | No | No |
| 1278 | UGAUUGUCAAAACUUUAUA | UAUAAAGUUUUGACAAUCA | 1279 | 1999 | No | No | No |
| 1280 | UUGUCAAAACUUUAUAUCA | UGAUAUAAAGUUUUGACAA | 1281 | 2002 | No | No | No |
| 1282 | UGUCAAAACUUUAUAUCAA | UUGAUAUAAAGUUUUGACA | 1283 | 2003 | No | No | No |
| 1284 | CAAAACUUUAUAUCACCUA | UAGGUGAUAUAAAGUUUUG | 1285 | 2006 | No | No | No |
| 1286 | AAAACUUUAUAUCACCUAA | UUAGGUGAUAUAAAGUUUU | 1287 | 2007 | No | No | No |
| 1288 | AAACUUUAUAUCACCUAAA | UUUAGGUGAUAUAAAGUUU | 1289 | 2008 | No | No | No |
| 1290 | ACUUUAUAUCACCUAAAGA | UCUUUAGGUGAUAUAAAGU | 1291 | 2010 | No | No | No |
| 1292 | CUUUAUAUCACCUAAAGUA | UACUUUAGGUGAUAUAAAG | 1293 | 2011 | No | No | No |
| 1294 | UUAUAUCACCUAAAGUCAA | UUGACUUUAGGUGAUAUAA | 1295 | 2013 | Yes | No | No |
| 1296 | UAUAUCACCUAAAGUCAGA | UCUGACUUUAGGUGAUAUA | 1297 | 2014 | Yes | No | No |
| 1298 | UAUCACCUAAAGUCAGAAA | UUUCUGACUUUAGGUGAUA | 1299 | 2016 | Yes | No | No |
| 1300 | AUCACCUAAAGUCAGAAUA | UAUUCUGACUUUAGGUGAU | 1301 | 2017 | Yes | No | No |
| 1302 | UCACCUAAAGUCAGAAUUA | UAAUUCUGACUUUAGGUGA | 1303 | 2018 | Yes | No | No |
| 1304 | GUCAGAAUUUCAAGCAAUA | UAUUGCUUGAAAUUCUGAC | 1305 | 2027 | Yes | No | No |
| 1306 | AGAAUUUCAAGCAAUAAUA | UAUUAUUGCUUGAAAUUCU | 1307 | 2030 | Yes | No | No |
| 1308 | AUUUCAAGCAAUAAUACCA | UGGUAUUAUUGCUUGAAAU | 1309 | 2033 | Yes | No | No |
| 1310 | UUUCAAGCAAUAAUACCUA | UAGGUAUUAUUGCUUGAAA | 1311 | 2034 | Yes | No | No |
| 1312 | UCAAGCAAUAAUACCUGCA | UGCAGGUAUUAUUGCUUGA | 1313 | 2036 | Yes | No | No |
| 1314 | GCAAUAAUACCUGCUGUUA | UAACAGCAGGUAUUAUUGC | 1315 | 2040 | Yes | No | No |
| 1316 | UAAUACCUGCUGUUAAUUA | UAAUUAACAGCAGGUAUUA | 1317 | 2044 | Yes | No | No |
| 1318 | AUACCUGCUGUUAAUUCCA | UGGAAUUAACAGCAGGUAU | 1319 | 2046 | Yes | No | No |
| 1320 | UGCUGUUAAUUCCCACAUA | UAUGUGGGAAUUAACAGCA | 1321 | 2051 | No | No | No |
| 1322 | GCUGUUAAUUCCCACAUUA | UAAUGUGGGAAUUAACAGC | 1323 | 2052 | No | No | No |
| 1324 | CUGUUAAUUCCCACAUUCA | UGAAUGUGGGAAUUAACAG | 1325 | 2053 | No | No | No |
| 1326 | UGUUAAUUCCCACAUUCAA | UUGAAUGUGGGAAUUAACA | 1327 | 2054 | No | No | No |
| 1328 | UUAAUUCCCACAUUCAGUA | UACUGAAUGUGGGAAUUAA | 1329 | 2056 | No | No | No |
| 1330 | UAAUUCCCACAUUCAGUCA | UGACUGAAUGUGGGAAUUA | 1331 | 2057 | No | No | No |
| 1332 | UUCCCACAUUCAGUCAGAA | UUCUGACUGAAUGUGGGAA | 1333 | 2060 | No | No | No |
| 1334 | UCCCACAUUCAGUCAGACA | UGUCUGACUGAAUGUGGGA | 1335 | 2061 | No | No | No |
| 1336 | CCCACAUUCAGUCAGACUA | UAGUCUGACUGAAUGUGGG | 1337 | 2062 | No | No | No |
| 1338 | CACAUUCAGUCAGACUUGA | UCAAGUCUGACUGAAUGUG | 1339 | 2064 | No | No | No |
| 1340 | ACAUUCAGUCAGACUUGCA | UGCAAGUCUGACUGAAUGU | 1341 | 2065 | No | No | No |
| 1342 | CAUUCAGUCAGACUUGCUA | UAGCAAGUCUGACUGAAUG | 1343 | 2066 | No | No | No |
| 1344 | AUUCAGUCAGACUUGCUCA | UGAGCAAGUCUGACUGAAU | 1345 | 2067 | No | No | No |
| 1346 | ACUUGCUCCGGACCGUUAA | UUAACGGUCCGGAGCAAGU | 1347 | 2077 | No | No | No |
| 1348 | CUUGCUCCGGACCGUUAUA | UAUAACGGUCCGGAGCAAG | 1349 | 2078 | No | No | No |
| 1350 | UUGCUCCGGACCGUUAUUA | UAAUAACGGUCCGGAGCAA | 1351 | 2079 | No | No | No |
| 1352 | GCUCCGGACCGUUAUUUUA | UAAAAUAACGGUCCGGAGC | 1353 | 2081 | No | No | No |
| 1354 | CUCCGGACCGUUAUUUUAA | UUAAAAUAACGGUCCGGAG | 1355 | 2082 | No | No | No |
| 1356 | UCCGGACCGUUAUUUUAGA | UCUAAAAUAACGGUCCGGA | 1357 | 2083 | No | No | No |
| 1358 | GGACCGUUAUUUUAGAAAA | UUUUCUAAAAUAACGGUCC | 1359 | 2086 | No | No | No |
| 1360 | GACCGUUAUUUUAGAAAUA | UAUUUCUAAAAUAACGGUC | 1361 | 2087 | No | No | No |
| 1362 | ACCGUUAUUUUAGAAAUUA | UAAUUUCUAAAAUAACGGU | 1363 | 2088 | Yes | No | No |
| 1364 | CCGUUAUUUUAGAAAUUCA | UGAAUUUCUAAAAUAACGG | 1365 | 2089 | Yes | No | No |
| 1366 | CGUUAUUUUAGAAAUUCCA | UGGAAUUUCUAAAAUAACG | 1367 | 2090 | Yes | No | No |
| 1368 | UUUAGAAAUUCCUGAACUA | UAGUUCAGGAAUUUCUAAA | 1369 | 2096 | Yes | No | No |
| 1370 | UUAGAAAUUCCUGAACUCA | UGAGUUCAGGAAUUUCUAA | 1371 | 2097 | Yes | No | No |
| 1372 | GAAAUUCCUGAACUCCUCA | UGAGGAGUUCAGGAAUUUC | 1373 | 2100 | Yes | No | No |
| 1374 | AAAUUCCUGAACUCCUCAA | UUGAGGAGUUCAGGAAUUU | 1375 | 2101 | Yes | No | No |
| 1376 | CCUGAACUCCUCAGUCCAA | UUGGACUGAGGAGUUCAGG | 1377 | 2106 | Yes | No | No |
| 1378 | UGAACUCCUCAGUCCAGUA | UACUGGACUGAGGAGUUCA | 1379 | 2108 | Yes | No | No |
| 1380 | CUCAGUCCAGUGGAGCAUA | UAUGCUCCACUGGACUGAG | 1381 | 2115 | Yes | No | No |
| 1382 | UCAGUCCAGUGGAGCAUUA | UAAUGCUCCACUGGACUGA | 1383 | 2116 | Yes | No | No |
| 1384 | GUCCAGUGGAGCAUUACUA | UAGUAAUGCUCCACUGGAC | 1385 | 2119 | Yes | No | No |
| 1386 | UCCAGUGGAGCAUUACUUA | UAAGUAAUGCUCCACUGGA | 1387 | 2120 | Yes | No | No |
| 1388 | AGUGGAGCAUUACUUAAAA | UUUUAAGUAAUGCUCCACU | 1389 | 2123 | Yes | No | No |
| 1390 | GUGGAGCAUUACUUAAAGA | UCUUUAAGUAAUGCUCCAC | 1391 | 2124 | Yes | No | No |
| 1392 | GGAGCAUUACUUAAAGAUA | UAUCUUUAAGUAAUGCUCC | 1393 | 2126 | Yes | No | No |
| 1394 | GAGCAUUACUUAAAGAUAA | UUAUCUUUAAGUAAUGCUC | 1395 | 2127 | Yes | No | No |
| 1396 | AGCAUUACUUAAAGAUACA | UGUAUCUUUAAGUAAUGCU | 1397 | 2128 | Yes | No | No |
| 1398 | CAUUACUUAAAGAUACUCA | UGAGUAUCUUUAAGUAAUG | 1399 | 2130 | Yes | No | No |
| 1400 | UACUUAAAGAUACUCAAUA | UAUUGAGUAUCUUUAAGUA | 1401 | 2133 | Yes | No | No |
| 1402 | CUUAAAGAUACUCAAUGAA | UUCAUUGAGUAUCUUUAAG | 1403 | 2135 | Yes | No | No |
| 1404 | UUAAAGAUACUCAAUGAAA | UUUCAUUGAGUAUCUUUAA | 1405 | 2136 | Yes | No | No |
| 1406 | UAAAGAUACUCAAUGAACA | UGUUCAUUGAGUAUCUUUA | 1407 | 2137 | Yes | No | No |
| 1408 | GAUACUCAAUGAACAAGCA | UGCUUGUUCAUUGAGUAUC | 1409 | 2141 | Yes | No | No |
| 1410 | AUGAACAAGCUGCCAAAGA | UCUUUGGCAGCUUGUUCAU | 1411 | 2149 | Yes | No | Yes |
| 1412 | UGAACAAGCUGCCAAAGUA | UACUUUGGCAGCUUGUUCA | 1413 | 2150 | Yes | No | No |
| 1414 | GAACAAGCUGCCAAAGUUA | UAACUUUGGCAGCUUGUUC | 1415 | 2151 | Yes | No | No |
| 1416 | ACAAGCUGCCAAAGUUGGA | UCCAACUUUGGCAGCUUGU | 1417 | 2153 | Yes | No | No |
| 1418 | CUGCCAAAGUUGGGGAUAA | UUAUCCCCAACUUUGGCAG | 1419 | 2158 | Yes | V | No |
| 1420 | UGCCAAAGUUGGGGAUAAA | UUUAUCCCCAACUUUGGCA | 1421 | 2159 | Yes | No | No |
| 1422 | AAGUUGGGGAUAAAACUGA | UCAGUUUUAUCCCCAACUU | 1423 | 2164 | Yes | No | No |
| 1424 | GUUGGGGAUAAAACUGAAA | UUUCAGUUUUAUCCCCAAC | 1425 | 2166 | Yes | No | No |
| 1426 | UUGGGGAUAAAACUGAAUA | UAUUCAGUUUUAUCCCCAA | 1427 | 2167 | Yes | No | No |
| 1428 | GGGGAUAAAACUGAAUUAA | UUAAUUCAGUUUUAUCCCC | 1429 | 2169 | Yes | No | No |
| 1430 | GGGAUAAAACUGAAUUAUA | UAUAAUUCAGUUUUAUCCC | 1431 | 2170 | Yes | No | No |
| 1432 | GGAUAAAACUGAAUUAUUA | UAAUAAUUCAGUUUUAUCC | 1433 | 2171 | Yes | No | No |
| 1434 | CUGAAUUAUUUAAAGACCA | UGGUCUUUAAAUAAUUCAG | 1435 | 2179 | Yes | No | No |
| 1436 | UAUUUAAAGACCUUUCUGA | UCAGAAAGGUCUUUAAAUA | 1437 | 2185 | Yes | No | No |
| 1438 | AUUUAAAGACCUUUCUGAA | UUCAGAAAGGUCUUUAAAU | 1439 | 2186 | Yes | No | No |
| 1440 | UAAAGACCUUUCUGACUUA | UAAGUCAGAAAGGUCUUUA | 1441 | 2189 | Yes | No | No |
| 1442 | AAGACCUUUCUGACUUCCA | UGGAAGUCAGAAAGGUCUU | 1443 | 2191 | Yes | No | No |
| 1444 | GACCUUUCUGACUUCCCUA | UAGGGAAGUCAGAAAGGUC | 1445 | 2193 | Yes | No | No |
| 1446 | ACCUUUCUGACUUCCCUUA | UAAGGGAAGUCAGAAAGGU | 1447 | 2194 | Yes | No | No |
| 1448 | UUCUGACUUCCCUUUAAUA | UAUUAAAGGGAAGUCAGAA | 1449 | 2198 | Yes | No | No |
| 1450 | UCUGACUUCCCUUUAAUAA | UUAUUAAAGGGAAGUCAGA | 1451 | 2199 | Yes | Yes | Yes |
| 1452 | UGACUUCCCUUUAAUAAAA | UUUUAUUAAAGGGAAGUCA | 1453 | 2201 | Yes | Yes | Yes |
| 1454 | AGAGGAAGGAUGAAAUUCA | UGAAUUUCAUCCUUCCUCU | 1455 | 2221 | Yes | No | No |
| 1456 | GGAUGAAAUUCAAGGUGUA | UACACCUUGAAUUUCAUCC | 1457 | 2228 | Yes | No | No |
| 1458 | UGAAAUUCAAGGUGUUAUA | UAUAACACCUUGAAUUUCA | 1459 | 2231 | No | No | No |
| 1460 | GAAAUUCAAGGUGUUAUUA | UAAUAACACCUUGAAUUUC | 1461 | 2232 | No | No | No |
| 1462 | AAUUCAAGGUGUUAUUGAA | UUCAAUAACACCUUGAAUU | 1463 | 2234 | No | No | No |
| 1464 | AUUCAAGGUGUUAUUGACA | UGUCAAUAACACCUUGAAU | 1465 | 2235 | No | No | No |
| 1466 | UUCAAGGUGUUAUUGACGA | UCGUCAAUAACACCUUGAA | 1467 | 2236 | No | No | No |
| 1468 | CAAGGUGUUAUUGACGAGA | UCUCGUCAAUAACACCUUG | 1469 | 2238 | No | No | No |
| 1470 | AAGGUGUUAUUGACGAGAA | UUCUCGUCAAUAACACCUU | 1471 | 2239 | No | No | No |
| 1472 | GGUGUUAUUGACGAGAUCA | UGAUCUCGUCAAUAACACC | 1473 | 2241 | No | No | No |
| 1474 | GUUAUUGACGAGAUCCGAA | UUCGGAUCUCGUCAAUAAC | 1475 | 2244 | No | No | No |
| 1476 | UUAUUGACGAGAUCCGAAA | UUUCGGAUCUCGUCAAUAA | 1477 | 2245 | No | No | No |
| 1478 | UAUUGACGAGAUCCGAAUA | UAUUCGGAUCUCGUCAAUA | 1479 | 2246 | No | No | No |
| 1480 | AUUGACGAGAUCCGAAUGA | UCAUUCGGAUCUCGUCAAU | 1481 | 2247 | No | No | No |
| 1482 | UGACGAGAUCCGAAUGCAA | UUGCAUUCGGAUCUCGUCA | 1483 | 2249 | No | No | No |
| 1484 | GACGAGAUCCGAAUGCAUA | UAUGCAUUCGGAUCUCGUC | 1485 | 2250 | No | No | No |
| 1486 | ACGAGAUCCGAAUGCAUUA | UAAUGCAUUCGGAUCUCGU | 1487 | 2251 | No | No | No |
| 1488 | CGAGAUCCGAAUGCAUUUA | UAAAUGCAUUCGGAUCUCG | 1489 | 2252 | No | No | No |
| 1490 | GAGAUCCGAAUGCAUUUGA | UCAAAUGCAUUCGGAUCUC | 1491 | 2253 | No | No | No |
| 1492 | AGAUCCGAAUGCAUUUGCA | UGCAAAUGCAUUCGGAUCU | 1493 | 2254 | Yes | No | No |
| 1494 | GAUCCGAAUGCAUUUGCAA | UUGCAAAUGCAUUCGGAUC | 1495 | 2255 | Yes | No | No |
| 1496 | UCCGAAUGCAUUUGCAAGA | UCUUGCAAAUGCAUUCGGA | 1497 | 2257 | Yes | No | No |
| 1498 | CCGAAUGCAUUUGCAAGAA | UUCUUGCAAAUGCAUUCGG | 1499 | 2258 | Yes | No | No |
| 1500 | GCAUUUGCAAGAAAUACGA | UCGUAUUUCUUGCAAAUGC | 1501 | 2264 | Yes | No | No |
| 1502 | AAAAUCCUUCUGCACAAUA | UAUUGUGCAGAAGGAUUUU | 1503 | 2293 | Yes | No | No |
| 1504 | AAAUCCUUCUGCACAAUAA | UUAUUGUGCAGAAGGAUUU | 1505 | 2294 | Yes | No | No |
| 1506 | AUCCUUCUGCACAAUAUGA | UCAUAUUGUGCAGAAGGAU | 1507 | 2296 | Yes | No | No |
| 1508 | CUUCUGCACAAUAUGUGAA | UUCACAUAUUGUGCAGAAG | 1509 | 2299 | Yes | No | No |
| 1510 | UCUGCACAAUAUGUGACAA | UUGUCACAUAUUGUGCAGA | 1511 | 2301 | Yes | No | No |
| 1512 | UGCACAAUAUGUGACAGUA | UACUGUCACAUAUUGUGCA | 1513 | 2303 | Yes | No | No |
| 1514 | GCACAAUAUGUGACAGUAA | UUACUGUCACAUAUUGUGC | 1515 | 2304 | Yes | No | No |
| 1516 | UAUGUGACAGUAUCAGGAA | UUCCUGAUACUGUCACAUA | 1517 | 2310 | Yes | No | No |
| 1518 | AUGUGACAGUAUCAGGACA | UGUCCUGAUACUGUCACAU | 1519 | 2311 | Yes | No | No |
| 1520 | GUAUCAGGACAGGAGUUUA | UAAACUCCUGUCCUGAUAC | 1521 | 2319 | Yes | No | No |
| 1522 | UAUCAGGACAGGAGUUUAA | UUAAACUCCUGUCCUGAUA | 1523 | 2320 | Yes | No | No |
| 1524 | CAGGACAGGAGUUUAUGAA | UUCAUAAACUCCUGUCCUG | 1525 | 2323 | Yes | No | No |
| 1526 | AGGACAGGAGUUUAUGAUA | UAUCAUAAACUCCUGUCCU | 1527 | 2324 | Yes | No | No |
| 1528 | GGACAGGAGUUUAUGAUAA | UUAUCAUAAACUCCUGUCC | 1529 | 2325 | No | No | No |
| 1530 | UUAUGAUAGAAAUAAAGAA | UUCUUUAUUUCUAUCAUAA | 1531 | 2335 | No | No | No |
| 1532 | GAAAUAAAGAACUCUGCUA | UAGCAGAGUUCUUUAUUUC | 1533 | 2343 | Yes | No | No |
| 1534 | AAUAAAGAACUCUGCUGUA | UACAGCAGAGUUCUUUAUU | 1535 | 2345 | Yes | No | No |
| 1536 | AUAAAGAACUCUGCUGUAA | UUACAGCAGAGUUCUUUAU | 1537 | 2346 | Yes | No | No |
| 1538 | UAAAGAACUCUGCUGUAUA | UAUACAGCAGAGUUCUUUA | 1539 | 2347 | Yes | No | No |
| 1540 | AGAACUCUGCUGUAUCUUA | UAAGAUACAGCAGAGUUCU | 1541 | 2350 | Yes | No | No |
| 1542 | GAACUCUGCUGUAUCUUGA | UCAAGAUACAGCAGAGUUC | 1543 | 2351 | Yes | No | No |
| 1544 | ACUCUGCUGUAUCUUGUAA | UUACAAGAUACAGCAGAGU | 1545 | 2353 | Yes | No | No |
| 1546 | CUCUGCUGUAUCUUGUAUA | UAUACAAGAUACAGCAGAG | 1547 | 2354 | Yes | No | No |
| 1548 | UGCUGUAUCUUGUAUACCA | UGGUAUACAAGAUACAGCA | 1549 | 2357 | Yes | No | No |
| 1550 | GCUGUAUCUUGUAUACCAA | UUGGUAUACAAGAUACAGC | 1551 | 2358 | Yes | No | No |
| 1552 | GUAUCUUGUAUACCAACUA | UAGUUGGUAUACAAGAUAC | 1553 | 2361 | Yes | No | No |
| 1554 | UAUCUUGUAUACCAACUGA | UCAGUUGGUAUACAAGAUA | 1555 | 2362 | Yes | No | No |
| 1556 | UCUUGUAUACCAACUGAUA | UAUCAGUUGGUAUACAAGA | 1557 | 2364 | Yes | No | No |
| 1558 | UGUAUACCAACUGAUUGGA | UCCAAUCAGUUGGUAUACA | 1559 | 2367 | Yes | No | No |
| 1560 | UAUACCAACUGAUUGGGUA | UACCCAAUCAGUUGGUAUA | 1561 | 2369 | Yes | No | No |
| 1562 | AUACCAACUGAUUGGGUAA | UUACCCAAUCAGUUGGUAU | 1563 | 2370 | Yes | No | No |
| 1564 | ACCAACUGAUUGGGUAAAA | UUUUACCCAAUCAGUUGGU | 1565 | 2372 | Yes | No | No |
| 1566 | CCAACUGAUUGGGUAAAGA | UCUUUACCCAAUCAGUUGG | 1567 | 2373 | Yes | No | No |
| 1568 | CAACUGAUUGGGUAAAGGA | UCCUUUACCCAAUCAGUUG | 1569 | 2374 | Yes | No | No |
| 1570 | AACUGAUUGGGUAAAGGUA | UACCUUUACCCAAUCAGUU | 1571 | 2375 | Yes | No | No |
| 1572 | CUGAUUGGGUAAAGGUUGA | UCAACCUUUACCCAAUCAG | 1573 | 2377 | Yes | No | No |
| 1574 | UGAUUGGGUAAAGGUUGGA | UCCAACCUUUACCCAAUCA | 1575 | 2378 | Yes | No | No |
| 1576 | GAUUGGGUAAAGGUUGGAA | UUCCAACCUUUACCCAAUC | 1577 | 2379 | Yes | No | No |
| 1578 | AUUGGGUAAAGGUUGGAAA | UUUCCAACCUUUACCCAAU | 1579 | 2380 | Yes | No | No |
| 1580 | UGGGUAAAGGUUGGAAGCA | UGCUUCCAACCUUUACCCA | 1581 | 2382 | Yes | No | No |
| 1582 | GUAAAGGUUGGAAGCACAA | UUGUGCUUCCAACCUUUAC | 1583 | 2385 | Yes | No | No |
| 1584 | GUUGGAAGCACAAAAGCUA | UAGCUUUUGUGCUUCCAAC | 1585 | 2391 | Yes | Yes | Yes |
| 1586 | UUGGAAGCACAAAAGCUGA | UCAGCUUUUGUGCUUCCAA | 1587 | 2392 | Yes | Yes | Yes |
| 1588 | UGGAAGCACAAAAGCUGUA | UACAGCUUUUGUGCUUCCA | 1589 | 2393 | Yes | Yes | Yes |
| 1590 | GGAAGCACAAAAGCUGUGA | UCACAGCUUUUGUGCUUCC | 1591 | 2394 | Yes | Yes | Yes |
| 1592 | GAAGCACAAAAGCUGUGAA | UUCACAGCUUUUGUGCUUC | 1593 | 2395 | Yes | Yes | Yes |
| 1594 | AGCUGUGAGCCGCUUUCAA | UUGAAAGCGGCUCACAGCU | 1595 | 2405 | No | Yes | Yes |
| 1596 | UGUGAGCCGCUUUCACUCA | UGAGUGAAAGCGGCUCACA | 1597 | 2408 | No | No | Yes |
| 1598 | GUGAGCCGCUUUCACUCUA | UAGAGUGAAAGCGGCUCAC | 1599 | 2409 | No | No | Yes |
| 1600 | AGCCGCUUUCACUCUCCUA | UAGGAGAGUGAAAGCGGCU | 1601 | 2412 | No | No | Yes |
| 1602 | GCCGCUUUCACUCUCCUUA | UAAGGAGAGUGAAAGCGGC | 1603 | 2413 | No | No | Yes |
| 1604 | GCUUUCACUCUCCUUUUAA | UUAAAAGGAGAGUGAAAGC | 1605 | 2416 | No | No | No |
| 1606 | CUUUCACUCUCCUUUUAUA | UAUAAAAGGAGAGUGAAAG | 1607 | 2417 | No | No | No |
| 1608 | UUUCACUCUCCUUUUAUUA | UAAUAAAAGGAGAGUGAAA | 1609 | 2418 | No | No | No |
| 1610 | CACUCUCCUUUUAUUGUAA | UUACAAUAAAAGGAGAGUG | 1611 | 2421 | No | No | No |
| 1612 | ACUCUCCUUUUAUUGUAGA | UCUACAAUAAAAGGAGAGU | 1613 | 2422 | No | No | No |
| 1614 | CUUUUAUUGUAGAAAAUUA | UAAUUUUCUACAAUAAAAG | 1615 | 2428 | No | No | No |
| 1616 | UUUAUUGUAGAAAAUUACA | UGUAAUUUUCUACAAUAAA | 1617 | 2430 | No | No | No |
| 1618 | AUUGUAGAAAAUUACAGAA | UUCUGUAAUUUUCUACAAU | 1619 | 2433 | No | No | No |
| 1620 | GUAGAAAAUUACAGACAUA | UAUGUCUGUAAUUUUCUAC | 1621 | 2436 | Yes | No | No |
| 1622 | AGAAAAUUACAGACAUCUA | UAGAUGUCUGUAAUUUUCU | 1623 | 2438 | Yes | No | No |
| 1624 | GAAAAUUACAGACAUCUGA | UCAGAUGUCUGUAAUUUUC | 1625 | 2439 | Yes | No | No |
| 1626 | UUACAGACAUCUGAAUCAA | UUGAUUCAGAUGUCUGUAA | 1627 | 2444 | Yes | No | No |
| 1628 | AGACAUCUGAAUCAGCUCA | UGAGCUGAUUCAGAUGUCU | 1629 | 2448 | No | No | No |
| 1630 | CGGGAGCAGCUAGUCCUUA | UAAGGACUAGCUGCUCCCG | 1631 | 2466 | Yes | Yes | Yes |
| 1632 | GGGAGCAGCUAGUCCUUGA | UCAAGGACUAGCUGCUCCC | 1633 | 2467 | Yes | Yes | Yes |
| 1634 | GGAGCAGCUAGUCCUUGAA | UUCAAGGACUAGCUGCUCC | 1635 | 2468 | Yes | Yes | Yes |
| 1636 | GAGCAGCUAGUCCUUGACA | UGUCAAGGACUAGCUGCUC | 1637 | 2469 | Yes | Yes | Yes |
| 1638 | AGCAGCUAGUCCUUGACUA | UAGUCAAGGACUAGCUGCU | 1639 | 2470 | Yes | Yes | Yes |
| 1640 | CAGCUAGUCCUUGACUGCA | UGCAGUCAAGGACUAGCUG | 1641 | 2472 | Yes | Yes | Yes |
| 1642 | AGCUAGUCCUUGACUGCAA | UUGCAGUCAAGGACUAGCU | 1643 | 2473 | Yes | Yes | Yes |
| 1644 | CCUUGACUGCAGUGCUGAA | UUCAGCACUGCAGUCAAGG | 1645 | 2480 | Yes | No | No |
| 1646 | UGACUGCAGUGCUGAAUGA | UCAUUCAGCACUGCAGUCA | 1647 | 2483 | Yes | No | No |
| 1648 | ACUGCAGUGCUGAAUGGCA | UGCCAUUCAGCACUGCAGU | 1649 | 2485 | Yes | No | No |
| 1650 | CUGCAGUGCUGAAUGGCUA | UAGCCAUUCAGCACUGCAG | 1651 | 2486 | Yes | No | No |
| 1652 | UGCAGUGCUGAAUGGCUUA | UAAGCCAUUCAGCACUGCA | 1653 | 2487 | Yes | No | No |
| 1654 | GCAGUGCUGAAUGGCUUGA | UCAAGCCAUUCAGCACUGC | 1655 | 2488 | Yes | No | No |
| 1656 | GAAUGGCUUGAUUUUCUAA | UUAGAAAAUCAAGCCAUUC | 1657 | 2496 | Yes | No | No |
| 1658 | AAUGGCUUGAUUUUCUAGA | UCUAGAAAAUCAAGCCAUU | 1659 | 2497 | Yes | No | No |
| 1660 | AUGGCUUGAUUUUCUAGAA | UUCUAGAAAAUCAAGCCAU | 1661 | 2498 | Yes | No | No |
| 1662 | UGGCUUGAUUUUCUAGAGA | UCUCUAGAAAAUCAAGCCA | 1663 | 2499 | Yes | No | No |
| 1664 | GAUUUUCUAGAGAAAUUCA | UGAAUUUCUCUAGAAAAUC | 1665 | 2505 | Yes | No | No |
| 1666 | UUUCUAGAGAAAUUCAGUA | UACUGAAUUUCUCUAGAAA | 1667 | 2508 | Yes | No | No |
| 1668 | UUCUAGAGAAAUUCAGUGA | UCACUGAAUUUCUCUAGAA | 1669 | 2509 | Yes | No | No |
| 1670 | UCUAGAGAAAUUCAGUGAA | UUCACUGAAUUUCUCUAGA | 1671 | 2510 | Yes | No | No |
| 1672 | UAGAGAAAUUCAGUGAACA | UGUUCACUGAAUUUCUCUA | 1673 | 2512 | Yes | No | No |
| 1674 | AAAUUCAGUGAACAUUAUA | UAUAAUGUUCACUGAAUUU | 1675 | 2517 | Yes | No | No |
| 1676 | AAUUCAGUGAACAUUAUCA | UGAUAAUGUUCACUGAAUU | 1677 | 2518 | Yes | No | No |
| 1678 | AUUCAGUGAACAUUAUCAA | UUGAUAAUGUUCACUGAAU | 1679 | 2519 | Yes | No | No |
| 1680 | GUGAACAUUAUCACUCCUA | UAGGAGUGAUAAUGUUCAC | 1681 | 2524 | No | No | No |
| 1682 | UGAACAUUAUCACUCCUUA | UAAGGAGUGAUAAUGUUCA | 1683 | 2525 | No | No | No |
| 1684 | GAACAUUAUCACUCCUUGA | UCAAGGAGUGAUAAUGUUC | 1685 | 2526 | No | No | No |
| 1686 | ACAUUAUCACUCCUUGUGA | UCACAAGGAGUGAUAAUGU | 1687 | 2528 | No | No | No |
| 1688 | AUCACUCCUUGUGUAAAGA | UCUUUACACAAGGAGUGAU | 1689 | 2533 | No | No | No |
| 1690 | ACUCCUUGUGUAAAGCAGA | UCUGCUUUACACAAGGAGU | 1691 | 2536 | No | No | No |
| 1692 | UUGUGUAAAGCAGUGCAUA | UAUGCACUGCUUUACACAA | 1693 | 2541 | Yes | No | No |
| 1694 | UAAAGCAGUGCAUCACCUA | UAGGUGAUGCACUGCUUUA | 1695 | 2546 | Yes | No | No |
| 1696 | AAAGCAGUGCAUCACCUAA | UUAGGUGAUGCACUGCUUU | 1697 | 2547 | Yes | No | No |
| 1698 | GUGCAUCACCUAGCAACUA | UAGUUGCUAGGUGAUGCAC | 1699 | 2553 | Yes | No | No |
| 1700 | GCAUCACCUAGCAACUGUA | UACAGUUGCUAGGUGAUGC | 1701 | 2555 | Yes | No | No |
| 1702 | UCACCUAGCAACUGUUGAA | UUCAACAGUUGCUAGGUGA | 1703 | 2558 | Yes | Yes | No |
| 1704 | ACCUAGCAACUGUUGACUA | UAGUCAACAGUUGCUAGGU | 1705 | 2560 | Yes | Yes | No |
| 1706 | CCUAGCAACUGUUGACUGA | UCAGUCAACAGUUGCUAGG | 1707 | 2561 | Yes | Yes | No |
| 1708 | CUAGCAACUGUUGACUGCA | UGCAGUCAACAGUUGCUAG | 1709 | 2562 | Yes | No | No |
| 1710 | UAGCAACUGUUGACUGCAA | UUGCAGUCAACAGUUGCUA | 1711 | 2563 | Yes | No | No |
| 1712 | AGCAACUGUUGACUGCAUA | UAUGCAGUCAACAGUUGCU | 1713 | 2564 | Yes | No | No |
| 1714 | GCAACUGUUGACUGCAUUA | UAAUGCAGUCAACAGUUGC | 1715 | 2565 | Yes | No | No |
| 1716 | AACUGUUGACUGCAUUUUA | UAAAAUGCAGUCAACAGUU | 1717 | 2567 | Yes | No | No |
| 1718 | ACUGUUGACUGCAUUUUCA | UGAAAAUGCAGUCAACAGU | 1719 | 2568 | Yes | No | No |
| 1720 | CUGUUGACUGCAUUUUCUA | UAGAAAAUGCAGUCAACAG | 1721 | 2569 | Yes | No | No |
| 1722 | UGUUGACUGCAUUUUCUCA | UGAGAAAAUGCAGUCAACA | 1723 | 2570 | Yes | No | No |
| 1724 | CCUGGCCAAGGUCGCUAAA | UUUAGCGACCUUGGCCAGG | 1725 | 2588 | Yes | Yes | No |
| 1726 | CCAAGGUCGCUAAGCAAGA | UCUUGCUUAGCGACCUUGG | 1727 | 2593 | Yes | No | No |
| 1728 | CAAGGUCGCUAAGCAAGGA | UCCUUGCUUAGCGACCUUG | 1729 | 2594 | Yes | No | No |
| 1730 | AAGGUCGCUAAGCAAGGAA | UUCCUUGCUUAGCGACCUU | 1731 | 2595 | Yes | No | No |
| 1732 | GCUAAGCAAGGAGAUUACA | UGUAAUCUCCUUGCUUAGC | 1733 | 2601 | No | No | No |
| 1734 | CUAAGCAAGGAGAUUACUA | UAGUAAUCUCCUUGCUUAG | 1735 | 2602 | No | No | No |
| 1736 | UAAGCAAGGAGAUUACUGA | UCAGUAAUCUCCUUGCUUA | 1737 | 2603 | No | No | No |
| 1738 | AAGCAAGGAGAUUACUGCA | UGCAGUAAUCUCCUUGCUU | 1739 | 2604 | No | No | No |
| 1740 | GCAAGGAGAUUACUGCAGA | UCUGCAGUAAUCUCCUUGC | 1741 | 2606 | No | No | No |
| 1742 | CAAGGAGAUUACUGCAGAA | UUCUGCAGUAAUCUCCUUG | 1743 | 2607 | No | No | No |
| 1744 | GAGAUUACUGCAGACCAAA | UUUGGUCUGCAGUAAUCUC | 1745 | 2611 | No | No | No |
| 1746 | AGAUUACUGCAGACCAACA | UGUUGGUCUGCAGUAAUCU | 1747 | 2612 | No | No | No |
| 1748 | GAUUACUGCAGACCAACUA | UAGUUGGUCUGCAGUAAUC | 1749 | 2613 | No | No | No |
| 1750 | AUUACUGCAGACCAACUGA | UCAGUUGGUCUGCAGUAAU | 1751 | 2614 | Yes | No | No |
| 1752 | UACUGCAGACCAACUGUAA | UUACAGUUGGUCUGCAGUA | 1753 | 2616 | Yes | No | No |
| 1754 | CUGCAGACCAACUGUACAA | UUGUACAGUUGGUCUGCAG | 1755 | 2618 | Yes | No | No |
| 1756 | UGCAGACCAACUGUACAAA | UUUGUACAGUUGGUCUGCA | 1757 | 2619 | Yes | No | No |
| 1758 | GCAGACCAACUGUACAAGA | UCUUGUACAGUUGGUCUGC | 1759 | 2620 | Yes | No | No |
| 1760 | CAGACCAACUGUACAAGAA | UUCUUGUACAGUUGGUCUG | 1761 | 2621 | Yes | No | No |
| 1762 | AGACCAACUGUACAAGAAA | UUUCUUGUACAGUUGGUCU | 1763 | 2622 | Yes | No | No |
| 1764 | CAACUGUACAAGAAGAAAA | UUUUCUUCUUGUACAGUUG | 1765 | 2626 | Yes | No | No |
| 1766 | ACUGUACAAGAAGAAAGAA | UUCUUUCUUCUUGUACAGU | 1767 | 2628 | Yes | No | No |
| 1768 | CUGUACAAGAAGAAAGAAA | UUUCUUUCUUCUUGUACAG | 1769 | 2629 | Yes | No | No |
| 1770 | UGUACAAGAAGAAAGAAAA | UUUUCUUUCUUCUUGUACA | 1771 | 2630 | Yes | No | No |
| 1772 | UGGAAGGCACCCUGUGAUA | UAUCACAGGGUGCCUUCCA | 1773 | 2663 | No | No | No |
| 1774 | GGAAGGCACCCUGUGAUUA | UAAUCACAGGGUGCCUUCC | 1775 | 2664 | No | No | No |
| 1776 | AAGGCACCCUGUGAUUGAA | UUCAAUCACAGGGUGCCUU | 1777 | 2666 | No | No | No |
| 1778 | AGGCACCCUGUGAUUGAUA | UAUCAAUCACAGGGUGCCU | 1779 | 2667 | No | No | No |
| 1780 | GGCACCCUGUGAUUGAUGA | UCAUCAAUCACAGGGUGCC | 1781 | 2668 | No | No | No |
| 1782 | GCACCCUGUGAUUGAUGUA | UACAUCAAUCACAGGGUGC | 1783 | 2669 | No | No | No |
| 1784 | CACCCUGUGAUUGAUGUGA | UCACAUCAAUCACAGGGUG | 1785 | 2670 | No | No | No |
| 1786 | CUGUGAUUGAUGUGUUGCA | UGCAACACAUCAAUCACAG | 1787 | 2674 | Yes | No | No |
| 1788 | UGUGAUUGAUGUGUUGCUA | UAGCAACACAUCAAUCACA | 1789 | 2675 | Yes | No | No |
| 1790 | GUGAUUGAUGUGUUGCUGA | UCAGCAACACAUCAAUCAC | 1791 | 2676 | Yes | No | No |
| 1792 | UGUGUUGCUGGGAGAACAA | UUGUUCUCCCAGCAACACA | 1793 | 2684 | Yes | No | No |
| 1794 | CUGGGAGAACAGGAUCAAA | UUUGAUCCUGUUCUCCCAG | 1795 | 2691 | Yes | No | No |
| 1796 | UGGGAGAACAGGAUCAAUA | UAUUGAUCCUGUUCUCCCA | 1797 | 2692 | Yes | No | No |
| 1798 | GGGAGAACAGGAUCAAUAA | UUAUUGAUCCUGUUCUCCC | 1799 | 2693 | Yes | No | No |
| 1800 | GAGAACAGGAUCAAUAUGA | UCAUAUUGAUCCUGUUCUC | 1801 | 2695 | Yes | No | No |
| 1802 | AGAACAGGAUCAAUAUGUA | UACAUAUUGAUCCUGUUCU | 1803 | 2696 | Yes | No | No |
| 1804 | AGGAUCAAUAUGUCCCAAA | UUUGGGACAUAUUGAUCCU | 1805 | 2701 | No | No | No |
| 1806 | GGAUCAAUAUGUCCCAAAA | UUUUGGGACAUAUUGAUCC | 1807 | 2702 | No | No | No |
| 1808 | GAUCAAUAUGUCCCAAAUA | UAUUUGGGACAUAUUGAUC | 1809 | 2703 | No | No | No |
| 1810 | AUCAAUAUGUCCCAAAUAA | UUAUUUGGGACAUAUUGAU | 1811 | 2704 | No | No | No |
| 1812 | CAAUAUGUCCCAAAUAAUA | UAUUAUUUGGGACAUAUUG | 1813 | 2706 | No | No | No |
| 1814 | AUAUGUCCCAAAUAAUACA | UGUAUUAUUUGGGACAUAU | 1815 | 2708 | No | No | No |
| 1816 | GUCCCAAAUAAUACAGAUA | UAUCUGUAUUAUUUGGGAC | 1817 | 2712 | No | No | No |
| 1818 | CCCAAAUAAUACAGAUUUA | UAAAUCUGUAUUAUUUGGG | 1819 | 2714 | No | No | No |
| 1820 | AUAAUACAGAUUUAUCAGA | UCUGAUAAAUCUGUAUUAU | 1821 | 2719 | No | No | No |
| 1822 | UAAUACAGAUUUAUCAGAA | UUCUGAUAAAUCUGUAUUA | 1823 | 2720 | No | No | No |
| 1824 | AAUACAGAUUUAUCAGAGA | UCUCUGAUAAAUCUGUAUU | 1825 | 2721 | No | No | No |
| 1826 | UACAGAUUUAUCAGAGGAA | UUCCUCUGAUAAAUCUGUA | 1827 | 2723 | Yes | No | No |
| 1828 | GAUUUAUCAGAGGACUCAA | UUGAGUCCUCUGAUAAAUC | 1829 | 2727 | Yes | No | No |
| 1830 | UUAUCAGAGGACUCAGAGA | UCUCUGAGUCCUCUGAUAA | 1831 | 2730 | Yes | No | No |
| 1832 | GGACUCAGAGAGAGUAAUA | UAUUACUCUCUCUGAGUCC | 1833 | 2738 | Yes | No | No |
| 1834 | GACUCAGAGAGAGUAAUGA | UCAUUACUCUCUCUGAGUC | 1835 | 2739 | Yes | No | No |
| 1836 | ACUCAGAGAGAGUAAUGAA | UUCAUUACUCUCUCUGAGU | 1837 | 2740 | Yes | No | No |
| 1838 | UCAGAGAGAGUAAUGAUAA | UUAUCAUUACUCUCUCUGA | 1839 | 2742 | Yes | No | No |
| 1840 | CAGAGAGAGUAAUGAUAAA | UUUAUCAUUACUCUCUCUG | 1841 | 2743 | Yes | No | No |
| 1842 | AGAGAGAGUAAUGAUAAUA | UAUUAUCAUUACUCUCUCU | 1843 | 2744 | Yes | No | No |
| 1844 | AGAGUAAUGAUAAUUACCA | UGGUAAUUAUCAUUACUCU | 1845 | 2748 | No | No | No |
| 1846 | AGUAAUGAUAAUUACCGGA | UCCGGUAAUUAUCAUUACU | 1847 | 2750 | No | No | No |
| 1848 | AUAAUUACCGGACCAAACA | UGUUUGGUCCGGUAAUUAU | 1849 | 2757 | No | No | No |
| 1850 | UAAUUACCGGACCAAACAA | UUGUUUGGUCCGGUAAUUA | 1851 | 2758 | No | No | No |
| 1852 | AAUUACCGGACCAAACAUA | UAUGUUUGGUCCGGUAAUU | 1853 | 2759 | No | No | No |
| 1854 | AUUACCGGACCAAACAUGA | UCAUGUUUGGUCCGGUAAU | 1855 | 2760 | No | No | No |
| 1856 | UUACCGGACCAAACAUGGA | UCCAUGUUUGGUCCGGUAA | 1857 | 2761 | No | No | No |
| 1858 | ACCAAACAUGGGUGGAAAA | UUUUCCACCCAUGUUUGGU | 1859 | 2768 | Yes | No | No |
| 1860 | CAAACAUGGGUGGAAAGAA | UUCUUUCCACCCAUGUUUG | 1861 | 2770 | Yes | No | No |
| 1862 | AAACAUGGGUGGAAAGAGA | UCUCUUUCCACCCAUGUUU | 1863 | 2771 | Yes | No | No |
| 1864 | AACAUGGGUGGAAAGAGCA | UGCUCUUUCCACCCAUGUU | 1865 | 2772 | Yes | No | No |
| 1866 | UGGAAAGAGCUCCUACAUA | UAUGUAGGAGCUCUUUCCA | 1867 | 2780 | Yes | No | No |
| 1868 | GAAAGAGCUCCUACAUAAA | UUUAUGUAGGAGCUCUUUC | 1869 | 2782 | Yes | No | No |
| 1870 | GAGCUCCUACAUAAAACAA | UUGUUUUAUGUAGGAGCUC | 1871 | 2786 | Yes | No | Yes |
| 1872 | AGCUCCUACAUAAAACAAA | UUUGUUUUAUGUAGGAGCU | 1873 | 2787 | Yes | No | No |
| 1874 | GCUCCUACAUAAAACAAGA | UCUUGUUUUAUGUAGGAGC | 1875 | 2788 | Yes | No | No |
| 1876 | UCCUACAUAAAACAAGUUA | UAACUUGUUUUAUGUAGGA | 1877 | 2790 | Yes | No | No |
| 1878 | CUACAUAAAACAAGUUGCA | UGCAACUUGUUUUAUGUAG | 1879 | 2792 | Yes | No | No |
| 1880 | UACAUAAAACAAGUUGCAA | UUGCAACUUGUUUUAUGUA | 1881 | 2793 | Yes | No | No |
| 1882 | UAAAACAAGUUGCAUUGAA | UUCAAUGCAACUUGUUUUA | 1883 | 2797 | Yes | No | No |
| 1884 | AAAACAAGUUGCAUUGAUA | UAUCAAUGCAACUUGUUUU | 1885 | 2798 | Yes | No | No |
| 1886 | AAACAAGUUGCAUUGAUUA | UAAUCAAUGCAACUUGUUU | 1887 | 2799 | Yes | No | No |
| 1888 | AACAAGUUGCAUUGAUUAA | UUAAUCAAUGCAACUUGUU | 1889 | 2800 | Yes | No | No |
| 1890 | ACAAGUUGCAUUGAUUACA | UGUAAUCAAUGCAACUUGU | 1891 | 2801 | Yes | No | No |
| 1892 | CAAGUUGCAUUGAUUACCA | UGGUAAUCAAUGCAACUUG | 1893 | 2802 | Yes | No | No |
| 1894 | AGUUGCAUUGAUUACCAUA | UAUGGUAAUCAAUGCAACU | 1895 | 2804 | Yes | No | No |
| 1896 | GUUGCAUUGAUUACCAUCA | UGAUGGUAAUCAAUGCAAC | 1897 | 2805 | Yes | No | No |
| 1898 | UUGCAUUGAUUACCAUCAA | UUGAUGGUAAUCAAUGCAA | 1899 | 2806 | Yes | No | No |
| 1900 | UGCAUUGAUUACCAUCAUA | UAUGAUGGUAAUCAAUGCA | 1901 | 2807 | Yes | No | No |
| 1902 | CAUUGAUUACCAUCAUGGA | UCCAUGAUGGUAAUCAAUG | 1903 | 2809 | Yes | No | No |
| 1904 | AUUGAUUACCAUCAUGGCA | UGCCAUGAUGGUAAUCAAU | 1905 | 2810 | Yes | No | No |
| 1906 | UACCAUCAUGGCUCAGAUA | UAUCUGAGCCAUGAUGGUA | 1907 | 2816 | Yes | Yes | No |
| 1908 | ACCAUCAUGGCUCAGAUUA | UAAUCUGAGCCAUGAUGGU | 1909 | 2817 | Yes | Yes | No |
| 1910 | CCAUCAUGGCUCAGAUUGA | UCAAUCUGAGCCAUGAUGG | 1911 | 2818 | Yes | Yes | No |
| 1912 | CAUCAUGGCUCAGAUUGGA | UCCAAUCUGAGCCAUGAUG | 1913 | 2819 | Yes | Yes | No |
| 1914 | AUCAUGGCUCAGAUUGGCA | UGCCAAUCUGAGCCAUGAU | 1915 | 2820 | Yes | Yes | No |
| 1916 | CAUGGCUCAGAUUGGCUCA | UGAGCCAAUCUGAGCCAUG | 1917 | 2822 | Yes | Yes | No |
| 1918 | CUCAGAUUGGCUCCUAUGA | UCAUAGGAGCCAAUCUGAG | 1919 | 2827 | Yes | No | No |
| 1920 | CAGAUUGGCUCCUAUGUUA | UAACAUAGGAGCCAAUCUG | 1921 | 2829 | Yes | No | No |
| 1922 | AUUGGCUCCUAUGUUCCUA | UAGGAACAUAGGAGCCAAU | 1923 | 2832 | Yes | No | No |
| 1924 | UUGGCUCCUAUGUUCCUGA | UCAGGAACAUAGGAGCCAA | 1925 | 2833 | Yes | No | No |
| 1926 | GGCUCCUAUGUUCCUGCAA | UUGCAGGAACAUAGGAGCC | 1927 | 2835 | Yes | No | No |
| 1928 | UCCUAUGUUCCUGCAGAAA | UUUCUGCAGGAACAUAGGA | 1929 | 2838 | Yes | No | No |
| 1930 | CCUAUGUUCCUGCAGAAGA | UCUUCUGCAGGAACAUAGG | 1931 | 2839 | Yes | No | No |
| 1932 | CUAUGUUCCUGCAGAAGAA | UUCUUCUGCAGGAACAUAG | 1933 | 2840 | Yes | No | No |
| 1934 | CUGCAGAAGAAGCGACAAA | UUUGUCGCUUCUUCUGCAG | 1935 | 2848 | Yes | No | No |
| 1936 | UGCAGAAGAAGCGACAAUA | UAUUGUCGCUUCUUCUGCA | 1937 | 2849 | Yes | No | No |
| 1938 | GCAGAAGAAGCGACAAUUA | UAAUUGUCGCUUCUUCUGC | 1939 | 2850 | Yes | No | No |
| 1940 | AGAAGAAGCGACAAUUGGA | UCCAAUUGUCGCUUCUUCU | 1941 | 2852 | Yes | No | No |
| 1942 | AAGAAGCGACAAUUGGGAA | UUCCCAAUUGUCGCUUCUU | 1943 | 2854 | Yes | No | No |
| 1944 | AGAAGCGACAAUUGGGAUA | UAUCCCAAUUGUCGCUUCU | 1945 | 2855 | Yes | No | No |
| 1946 | GAAGCGACAAUUGGGAUUA | UAAUCCCAAUUGUCGCUUC | 1947 | 2856 | Yes | No | No |
| 1948 | AGCGACAAUUGGGAUUGUA | UACAAUCCCAAUUGUCGCU | 1949 | 2858 | Yes | No | No |
| 1950 | GCGACAAUUGGGAUUGUGA | UCACAAUCCCAAUUGUCGC | 1951 | 2859 | Yes | No | No |
| 1952 | CAAUUGGGAUUGUGGAUGA | UCAUCCACAAUCCCAAUUG | 1953 | 2863 | Yes | Yes | No |
| 1954 | UGGGAUUGUGGAUGGCAUA | UAUGCCAUCCACAAUCCCA | 1955 | 2867 | Yes | Yes | No |
| 1956 | GGGAUUGUGGAUGGCAUUA | UAAUGCCAUCCACAAUCCC | 1957 | 2868 | Yes | Yes | No |
| 1958 | GGAUUGUGGAUGGCAUUUA | UAAAUGCCAUCCACAAUCC | 1959 | 2869 | Yes | Yes | No |
| 1960 | GAUUGUGGAUGGCAUUUUA | UAAAAUGCCAUCCACAAUC | 1961 | 2870 | Yes | Yes | No |
| 1962 | UGUGGAUGGCAUUUUCACA | UGUGAAAAUGCCAUCCACA | 1963 | 2873 | Yes | Yes | No |
| 1964 | GUGGAUGGCAUUUUCACAA | UUGUGAAAAUGCCAUCCAC | 1965 | 2874 | Yes | Yes | No |
| 1966 | GGAUGGCAUUUUCACAAGA | UCUUGUGAAAAUGCCAUCC | 1967 | 2876 | Yes | Yes | No |
| 1968 | GAUGGCAUUUUCACAAGGA | UCCUUGUGAAAAUGCCAUC | 1969 | 2877 | Yes | Yes | No |
| 1970 | UGGCAUUUUCACAAGGAUA | UAUCCUUGUGAAAAUGCCA | 1971 | 2879 | Yes | Yes | No |
| 1972 | GGCAUUUUCACAAGGAUGA | UCAUCCUUGUGAAAAUGCC | 1973 | 2880 | Yes | Yes | No |
| 1974 | GGAUGGGUGCUGCAGACAA | UUGUCUGCAGCACCCAUCC | 1975 | 2893 | Yes | Yes | No |
| 1976 | GAUGGGUGCUGCAGACAAA | UUUGUCUGCAGCACCCAUC | 1977 | 2894 | Yes | Yes | No |
| 1978 | AUGGGUGCUGCAGACAAUA | UAUUGUCUGCAGCACCCAU | 1979 | 2895 | Yes | Yes | No |
| 1980 | UGGGUGCUGCAGACAAUAA | UUAUUGUCUGCAGCACCCA | 1981 | 2896 | Yes | Yes | No |
| 1982 | GGGUGCUGCAGACAAUAUA | UAUAUUGUCUGCAGCACCC | 1983 | 2897 | Yes | Yes | Yes |
| 1984 | GUGCUGCAGACAAUAUAUA | UAUAUAUUGUCUGCAGCAC | 1985 | 2899 | Yes | Yes | Yes |
| 1986 | GCUGCAGACAAUAUAUAUA | UAUAUAUAUUGUCUGCAGC | 1987 | 2901 | Yes | No | No |
| 1988 | CUGCAGACAAUAUAUAUAA | UUAUAUAUAUUGUCUGCAG | 1989 | 2902 | Yes | No | No |
| 1990 | UGCAGACAAUAUAUAUAAA | UUUAUAUAUAUUGUCUGCA | 1991 | 2903 | Yes | No | No |
| 1992 | GACAAUAUAUAUAAAGGAA | UUCCUUUAUAUAUAUUGUC | 1993 | 2907 | Yes | No | No |
| 1994 | ACAAUAUAUAUAAAGGACA | UGUCCUUUAUAUAUAUUGU | 1995 | 2908 | Yes | No | No |
| 1996 | CAAUAUAUAUAAAGGACAA | UUGUCCUUUAUAUAUAUUG | 1997 | 2909 | No | No | No |
| 1998 | AUAUAUAAAGGACAGAGUA | UACUCUGUCCUUUAUAUAU | 1999 | 2913 | No | No | No |
| 2000 | UAUAUAAAGGACAGAGUAA | UUACUCUGUCCUUUAUAUA | 2001 | 2914 | No | No | No |
| 2002 | UAUAAAGGACAGAGUACAA | UUGUACUCUGUCCUUUAUA | 2003 | 2916 | No | No | No |
| 2004 | AUAAAGGACAGAGUACAUA | UAUGUACUCUGUCCUUUAU | 2005 | 2917 | No | No | No |
| 2006 | UAAAGGACAGAGUACAUUA | UAAUGUACUCUGUCCUUUA | 2007 | 2918 | No | No | No |
| 2008 | AAAGGACAGAGUACAUUUA | UAAAUGUACUCUGUCCUUU | 2009 | 2919 | No | No | No |
| 2010 | CAGAGUACAUUUAUGGAAA | UUUCCAUAAAUGUACUCUG | 2011 | 2925 | No | No | No |
| 2012 | AGAGUACAUUUAUGGAAGA | UCUUCCAUAAAUGUACUCU | 2013 | 2926 | No | No | No |
| 2014 | GUACAUUUAUGGAAGAACA | UGUUCUUCCAUAAAUGUAC | 2015 | 2929 | Yes | No | No |
| 2016 | UACAUUUAUGGAAGAACUA | UAGUUCUUCCAUAAAUGUA | 2017 | 2930 | Yes | No | No |
| 2018 | AUUUAUGGAAGAACUGACA | UGUCAGUUCUUCCAUAAAU | 2019 | 2933 | Yes | No | No |
| 2020 | UUUAUGGAAGAACUGACUA | UAGUCAGUUCUUCCAUAAA | 2021 | 2934 | Yes | No | No |
| 2022 | UUAUGGAAGAACUGACUGA | UCAGUCAGUUCUUCCAUAA | 2023 | 2935 | Yes | No | No |
| 2024 | GGAAGAACUGACUGACACA | UGUGUCAGUCAGUUCUUCC | 2025 | 2939 | Yes | No | No |
| 2026 | GAACUGACUGACACAGCAA | UUGCUGUGUCAGUCAGUUC | 2027 | 2943 | Yes | No | No |
| 2028 | UGACACAGCAGAAAUAAUA | UAUUAUUUCUGCUGUGUCA | 2029 | 2951 | Yes | No | No |
| 2030 | GACACAGCAGAAAUAAUCA | UGAUUAUUUCUGCUGUGUC | 2031 | 2952 | Yes | No | No |
| 2032 | CAGAAAAGCAACAUCACAA | UUGUGAUGUUGCUUUUCUG | 2033 | 2969 | Yes | No | No |
| 2034 | AGAAAAGCAACAUCACAGA | UCUGUGAUGUUGCUUUUCU | 2035 | 2970 | Yes | No | No |
| 2036 | GCAACAUCACAGUCCUUGA | UCAAGGACUGUGAUGUUGC | 2037 | 2976 | Yes | No | No |
| 2038 | CAUCACAGUCCUUGGUUAA | UUAACCAAGGACUGUGAUG | 2039 | 2980 | Yes | No | No |
| 2040 | UCACAGUCCUUGGUUAUCA | UGAUAACCAAGGACUGUGA | 2041 | 2982 | Yes | No | No |
| 2042 | CACAGUCCUUGGUUAUCUA | UAGAUAACCAAGGACUGUG | 2043 | 2983 | Yes | No | No |
| 2044 | CAGUCCUUGGUUAUCUUGA | UCAAGAUAACCAAGGACUG | 2045 | 2985 | Yes | No | No |
| 2046 | UCCUUGGUUAUCUUGGAUA | UAUCCAAGAUAACCAAGGA | 2047 | 2988 | Yes | No | No |
| 2048 | CCUUGGUUAUCUUGGAUGA | UCAUCCAAGAUAACCAAGG | 2049 | 2989 | Yes | No | No |
| 2050 | GGUUAUCUUGGAUGAACUA | UAGUUCAUCCAAGAUAACC | 2051 | 2993 | No | No | No |
| 2052 | GUUAUCUUGGAUGAACUAA | UUAGUUCAUCCAAGAUAAC | 2053 | 2994 | No | No | No |
| 2054 | AUCUUGGAUGAACUAGGAA | UUCCUAGUUCAUCCAAGAU | 2055 | 2997 | No | No | No |
| 2056 | UCUUGGAUGAACUAGGAAA | UUUCCUAGUUCAUCCAAGA | 2057 | 2998 | No | No | No |
| 2058 | CUUGGAUGAACUAGGAAGA | UCUUCCUAGUUCAUCCAAG | 2059 | 2999 | No | No | No |
| 2060 | UUGGAUGAACUAGGAAGAA | UUCUUCCUAGUUCAUCCAA | 2061 | 3000 | No | No | No |
| 2062 | AAGAGGGACGAGCACUCAA | UUGAGUGCUCGUCCCUCUU | 2063 | 3014 | No | No | No |
| 2064 | AGAGGGACGAGCACUCAUA | UAUGAGUGCUCGUCCCUCU | 2065 | 3015 | No | No | No |
| 2066 | AGGGACGAGCACUCAUGAA | UUCAUGAGUGCUCGUCCCU | 2067 | 3017 | No | No | No |
| 2068 | GGGACGAGCACUCAUGAUA | UAUCAUGAGUGCUCGUCCC | 2069 | 3018 | No | No | No |
| 2070 | GGACGAGCACUCAUGAUGA | UCAUCAUGAGUGCUCGUCC | 2071 | 3019 | No | No | No |
| 2072 | ACGAGCACUCAUGAUGGAA | UUCCAUCAUGAGUGCUCGU | 2073 | 3021 | No | No | No |
| 2074 | CGAGCACUCAUGAUGGAAA | UUUCCAUCAUGAGUGCUCG | 2075 | 3022 | No | No | No |
| 2076 | GAGCACUCAUGAUGGAAUA | UAUUCCAUCAUGAGUGCUC | 2077 | 3023 | No | No | No |
| 2078 | CACUCAUGAUGGAAUUGCA | UGCAAUUCCAUCAUGAGUG | 2079 | 3026 | No | No | No |
| 2080 | ACUCAUGAUGGAAUUGCCA | UGGCAAUUCCAUCAUGAGU | 2081 | 3027 | No | No | No |
| 2082 | CUCAUGAUGGAAUUGCCAA | UUGGCAAUUCCAUCAUGAG | 2083 | 3028 | No | No | No |
| 2084 | UCAUGAUGGAAUUGCCAUA | UAUGGCAAUUCCAUCAUGA | 2085 | 3029 | No | No | No |
| 2086 | GAAUUGCCAUUGCCUAUGA | UCAUAGGCAAUGGCAAUUC | 2087 | 3037 | Yes | No | No |
| 2088 | AUUGCCAUUGCCUAUGCUA | UAGCAUAGGCAAUGGCAAU | 2089 | 3039 | Yes | No | No |
| 2090 | UUGCCAUUGCCUAUGCUAA | UUAGCAUAGGCAAUGGCAA | 2091 | 3040 | Yes | No | No |
| 2092 | UGCCAUUGCCUAUGCUACA | UGUAGCAUAGGCAAUGGCA | 2093 | 3041 | Yes | No | No |
| 2094 | CAUUGCCUAUGCUACACUA | UAGUGUAGCAUAGGCAAUG | 2095 | 3044 | Yes | No | No |
| 2096 | AUUGCCUAUGCUACACUUA | UAAGUGUAGCAUAGGCAAU | 2097 | 3045 | Yes | No | No |
| 2098 | UUGCCUAUGCUACACUUGA | UCAAGUGUAGCAUAGGCAA | 2099 | 3046 | Yes | No | No |
| 2100 | GCCUAUGCUACACUUGAGA | UCUCAAGUGUAGCAUAGGC | 2101 | 3048 | Yes | No | No |
| 2102 | CCUAUGCUACACUUGAGUA | UACUCAAGUGUAGCAUAGG | 2103 | 3049 | Yes | No | No |
| 2104 | CUAUGCUACACUUGAGUAA | UUACUCAAGUGUAGCAUAG | 2105 | 3050 | Yes | No | No |
| 2106 | UAUGCUACACUUGAGUAUA | UAUACUCAAGUGUAGCAUA | 2107 | 3051 | Yes | No | No |
| 2108 | AUGCUACACUUGAGUAUUA | UAAUACUCAAGUGUAGCAU | 2109 | 3052 | Yes | No | No |
| 2110 | GCUACACUUGAGUAUUUCA | UGAAAUACUCAAGUGUAGC | 2111 | 3054 | Yes | No | No |
| 2112 | CUACACUUGAGUAUUUCAA | UUGAAAUACUCAAGUGUAG | 2113 | 3055 | Yes | No | No |
| 2114 | CUUGAGUAUUUCAUCAGAA | UUCUGAUGAAAUACUCAAG | 2115 | 3060 | Yes | No | No |
| 2116 | UUGAGUAUUUCAUCAGAGA | UCUCUGAUGAAAUACUCAA | 2117 | 3061 | Yes | No | No |
| 2118 | UGAGUAUUUCAUCAGAGAA | UUCUCUGAUGAAAUACUCA | 2119 | 3062 | Yes | No | No |
| 2120 | GAGUAUUUCAUCAGAGAUA | UAUCUCUGAUGAAAUACUC | 2121 | 3063 | Yes | No | No |
| 2122 | UUCAUCAGAGAUGUGAAAA | UUUUCACAUCUCUGAUGAA | 2123 | 3069 | Yes | No | No |
| 2124 | CAUCAGAGAUGUGAAAUCA | UGAUUUCACAUCUCUGAUG | 2125 | 3071 | Yes | No | No |
| 2126 | AGAGAUGUGAAAUCCUUAA | UUAAGGAUUUCACAUCUCU | 2127 | 3075 | Yes | No | No |
| 2128 | GAGAUGUGAAAUCCUUAAA | UUUAAGGAUUUCACAUCUC | 2129 | 3076 | Yes | No | No |
| 2130 | AGAUGUGAAAUCCUUAACA | UGUUAAGGAUUUCACAUCU | 2131 | 3077 | Yes | No | No |
| 2132 | GAUGUGAAAUCCUUAACCA | UGGUUAAGGAUUUCACAUC | 2133 | 3078 | Yes | No | No |
| 2134 | UGUGAAAUCCUUAACCCUA | UAGGGUUAAGGAUUUCACA | 2135 | 3080 | Yes | No | No |
| 2136 | AUCCUUAACCCUGUUUGUA | UACAAACAGGGUUAAGGAU | 2137 | 3086 | Yes | No | No |
| 2138 | CCUUAACCCUGUUUGUCAA | UUGACAAACAGGGUUAAGG | 2139 | 3088 | Yes | No | No |
| 2140 | CUUAACCCUGUUUGUCACA | UGUGACAAACAGGGUUAAG | 2141 | 3089 | Yes | No | No |
| 2142 | ACCCUGUUUGUCACCCAUA | UAUGGGUGACAAACAGGGU | 2143 | 3093 | Yes | No | No |
| 2144 | CCCUGUUUGUCACCCAUUA | UAAUGGGUGACAAACAGGG | 2145 | 3094 | Yes | No | No |
| 2146 | CUGUUUGUCACCCAUUAUA | UAUAAUGGGUGACAAACAG | 2147 | 3096 | Yes | No | No |
| 2148 | GUUUGUCACCCAUUAUCCA | UGGAUAAUGGGUGACAAAC | 2149 | 3098 | Yes | No | No |
| 2150 | UUUGUCACCCAUUAUCCGA | UCGGAUAAUGGGUGACAAA | 2151 | 3099 | Yes | No | No |
| 2152 | CCCAUUAUCCGCCAGUUUA | UAAACUGGCGGAUAAUGGG | 2153 | 3106 | Yes | No | No |
| 2154 | CCAUUAUCCGCCAGUUUGA | UCAAACUGGCGGAUAAUGG | 2155 | 3107 | Yes | No | No |
| 2156 | UUAUCCGCCAGUUUGUGAA | UUCACAAACUGGCGGAUAA | 2157 | 3110 | Yes | No | No |
| 2158 | UAUCCGCCAGUUUGUGAAA | UUUCACAAACUGGCGGAUA | 2159 | 3111 | No | No | No |
| 2160 | AUCCGCCAGUUUGUGAACA | UGUUCACAAACUGGCGGAU | 2161 | 3112 | No | No | No |
| 2162 | CCGCCAGUUUGUGAACUAA | UUAGUUCACAAACUGGCGG | 2163 | 3114 | No | No | No |
| 2164 | CGCCAGUUUGUGAACUAGA | UCUAGUUCACAAACUGGCG | 2165 | 3115 | No | No | No |
| 2166 | GCCAGUUUGUGAACUAGAA | UUCUAGUUCACAAACUGGC | 2167 | 3116 | No | No | No |
| 2168 | CCAGUUUGUGAACUAGAAA | UUUCUAGUUCACAAACUGG | 2169 | 3117 | No | No | No |
| 2170 | CAGUUUGUGAACUAGAAAA | UUUUCUAGUUCACAAACUG | 2171 | 3118 | No | No | No |
| 2172 | UCACACCAGGUGGGGAAUA | UAUUCCCCACCUGGUGUGA | 2173 | 3144 | Yes | No | No |
| 2174 | CACACCAGGUGGGGAAUUA | UAAUUCCCCACCUGGUGUG | 2175 | 3145 | Yes | No | No |
| 2176 | ACACCAGGUGGGGAAUUAA | UUAAUUCCCCACCUGGUGU | 2177 | 3146 | Yes | No | No |
| 2178 | CACCAGGUGGGGAAUUACA | UGUAAUUCCCCACCUGGUG | 2179 | 3147 | Yes | No | No |
| 2180 | ACCAGGUGGGGAAUUACCA | UGGUAAUUCCCCACCUGGU | 2181 | 3148 | Yes | No | No |
| 2182 | CCAGGUGGGGAAUUACCAA | UUGGUAAUUCCCCACCUGG | 2183 | 3149 | Yes | No | No |
| 2184 | AGGUGGGGAAUUACCACAA | UUGUGGUAAUUCCCCACCU | 2185 | 3151 | Yes | No | Yes |
| 2186 | GGUGGGGAAUUACCACAUA | UAUGUGGUAAUUCCCCACC | 2187 | 3152 | Yes | No | Yes |
| 2188 | GUGGGGAAUUACCACAUGA | UCAUGUGGUAAUUCCCCAC | 2189 | 3153 | Yes | Yes | Yes |
| 2190 | GGGAAUUACCACAUGGGAA | UUCCCAUGUGGUAAUUCCC | 2191 | 3156 | Yes | Yes | No |
| 2192 | GGAAUUACCACAUGGGAUA | UAUCCCAUGUGGUAAUUCC | 2193 | 3157 | Yes | Yes | No |
| 2194 | GAAUUACCACAUGGGAUUA | UAAUCCCAUGUGGUAAUUC | 2195 | 3158 | Yes | Yes | No |
| 2196 | AAUUACCACAUGGGAUUCA | UGAAUCCCAUGUGGUAAUU | 2197 | 3159 | Yes | Yes | No |
| 2198 | AUUACCACAUGGGAUUCUA | UAGAAUCCCAUGUGGUAAU | 2199 | 3160 | Yes | Yes | No |
| 2200 | UACCACAUGGGAUUCUUGA | UCAAGAAUCCCAUGUGGUA | 2201 | 3162 | Yes | Yes | No |
| 2202 | CCACAUGGGAUUCUUGGUA | UACCAAGAAUCCCAUGUGG | 2203 | 3164 | Yes | Yes | No |
| 2204 | CAUGGGAUUCUUGGUCAGA | UCUGACCAAGAAUCCCAUG | 2205 | 3167 | Yes | No | No |
| 2206 | GGGAUUCUUGGUCAGUGAA | UUCACUGACCAAGAAUCCC | 2207 | 3170 | No | No | No |
| 2208 | GGAUUCUUGGUCAGUGAGA | UCUCACUGACCAAGAAUCC | 2209 | 3171 | No | No | No |
| 2210 | GUCAGUGAGGAUGAAAGCA | UGCUUUCAUCCUCACUGAC | 2211 | 3180 | No | No | No |
| 2212 | CAGUGAGGAUGAAAGCAAA | UUUGCUUUCAUCCUCACUG | 2213 | 3182 | No | No | No |
| 2214 | GAUGAAAGCAAACUGGAUA | UAUCCAGUUUGCUUUCAUC | 2215 | 3189 | No | No | No |
| 2216 | GAAAGCAAACUGGAUCCAA | UUGGAUCCAGUUUGCUUUC | 2217 | 3192 | No | No | No |
| 2218 | CCAGGCGCAGCAGAACAAA | UUUGUUCUGCUGCGCCUGG | 2219 | 3207 | No | No | No |
| 2220 | CAGGCGCAGCAGAACAAGA | UCUUGUUCUGCUGCGCCUG | 2221 | 3208 | No | No | No |
| 2222 | GGCGCAGCAGAACAAGUCA | UGACUUGUUCUGCUGCGCC | 2223 | 3210 | No | No | No |
| 2224 | GCGCAGCAGAACAAGUCCA | UGGACUUGUUCUGCUGCGC | 2225 | 3211 | No | No | No |
| 2226 | GCAGCAGAACAAGUCCCUA | UAGGGACUUGUUCUGCUGC | 2227 | 3213 | No | No | No |
| 2228 | AGAACAAGUCCCUGAUUUA | UAAAUCAGGGACUUGUUCU | 2229 | 3218 | Yes | No | No |
| 2230 | AACAAGUCCCUGAUUUUGA | UCAAAAUCAGGGACUUGUU | 2231 | 3220 | Yes | No | No |
| 2232 | GUCCCUGAUUUUGUCACCA | UGGUGACAAAAUCAGGGAC | 2233 | 3225 | Yes | No | No |
| 2234 | CCCUGAUUUUGUCACCUUA | UAAGGUGACAAAAUCAGGG | 2235 | 3227 | Yes | No | No |
| 2236 | CCUGAUUUUGUCACCUUCA | UGAAGGUGACAAAAUCAGG | 2237 | 3228 | Yes | No | No |
| 2238 | UGAUUUUGUCACCUUCCUA | UAGGAAGGUGACAAAAUCA | 2239 | 3230 | Yes | No | No |
| 2240 | AUUUUGUCACCUUCCUUUA | UAAAGGAAGGUGACAAAAU | 2241 | 3232 | Yes | No | No |
| 2242 | UUUUGUCACCUUCCUUUAA | UUAAAGGAAGGUGACAAAA | 2243 | 3233 | Yes | No | No |
| 2244 | UGUCACCUUCCUUUACCAA | UUGGUAAAGGAAGGUGACA | 2245 | 3236 | Yes | No | No |
| 2246 | UCACCUUCCUUUACCAAAA | UUUUGGUAAAGGAAGGUGA | 2247 | 3238 | Yes | No | No |
| 2248 | UUCCUUUACCAAAUAACUA | UAGUUAUUUGGUAAAGGAA | 2249 | 3243 | Yes | No | No |
| 2250 | UCCUUUACCAAAUAACUAA | UUAGUUAUUUGGUAAAGGA | 2251 | 3244 | Yes | No | No |
| 2252 | CCUUUACCAAAUAACUAGA | UCUAGUUAUUUGGUAAAGG | 2253 | 3245 | Yes | No | No |
| 2254 | CUUUACCAAAUAACUAGAA | UUCUAGUUAUUUGGUAAAG | 2255 | 3246 | Yes | No | No |
| 2256 | UACCAAAUAACUAGAGGAA | UUCCUCUAGUUAUUUGGUA | 2257 | 3249 | Yes | No | No |
| 2258 | CAAAUAACUAGAGGAAUUA | UAAUUCCUCUAGUUAUUUG | 2259 | 3252 | Yes | No | No |
| 2260 | UAACUAGAGGAAUUGCAGA | UCUGCAAUUCCUCUAGUUA | 2261 | 3256 | Yes | No | No |
| 2262 | CUAGAGGAAUUGCAGCAAA | UUUGCUGCAAUUCCUCUAG | 2263 | 3259 | Yes | No | No |
| 2264 | GGAAUUGCAGCAAGGAGUA | UACUCCUUGCUGCAAUUCC | 2265 | 3264 | Yes | No | No |
| 2266 | GAAUUGCAGCAAGGAGUUA | UAACUCCUUGCUGCAAUUC | 2267 | 3265 | Yes | No | No |
| 2268 | AUUGCAGCAAGGAGUUAUA | UAUAACUCCUUGCUGCAAU | 2269 | 3267 | Yes | No | No |
| 2270 | UUGCAGCAAGGAGUUAUGA | UCAUAACUCCUUGCUGCAA | 2271 | 3268 | Yes | No | No |
| 2272 | GCAAGGAGUUAUGGAUUAA | UUAAUCCAUAACUCCUUGC | 2273 | 3273 | Yes | No | No |
| 2274 | AAGGAGUUAUGGAUUAAAA | UUUUAAUCCAUAACUCCUU | 2275 | 3275 | Yes | No | No |
| 2276 | AGGAGUUAUGGAUUAAAUA | UAUUUAAUCCAUAACUCCU | 2277 | 3276 | Yes | No | No |
| 2278 | GUUAUGGAUUAAAUGUGGA | UCCACAUUUAAUCCAUAAC | 2279 | 3280 | Yes | No | No |
| 2280 | UUAUGGAUUAAAUGUGGCA | UGCCACAUUUAAUCCAUAA | 2281 | 3281 | Yes | No | No |
| 2282 | UAUGGAUUAAAUGUGGCUA | UAGCCACAUUUAAUCCAUA | 2283 | 3282 | Yes | No | No |
| 2284 | AUGGAUUAAAUGUGGCUAA | UUAGCCACAUUUAAUCCAU | 2285 | 3283 | Yes | No | No |
| 2286 | UGGAUUAAAUGUGGCUAAA | UUUAGCCACAUUUAAUCCA | 2287 | 3284 | Yes | No | No |
| 2288 | GAUUAAAUGUGGCUAAACA | UGUUUAGCCACAUUUAAUC | 2289 | 3286 | Yes | No | No |
| 2290 | AUUAAAUGUGGCUAAACUA | UAGUUUAGCCACAUUUAAU | 2291 | 3287 | Yes | No | No |
| 2292 | UUAAAUGUGGCUAAACUAA | UUAGUUUAGCCACAUUUAA | 2293 | 3288 | Yes | No | No |
| 2294 | UAAAUGUGGCUAAACUAGA | UCUAGUUUAGCCACAUUUA | 2295 | 3289 | Yes | No | No |
| 2296 | AAUGUGGCUAAACUAGCAA | UUGCUAGUUUAGCCACAUU | 2297 | 3291 | Yes | No | No |
| 2298 | AUGUGGCUAAACUAGCAGA | UCUGCUAGUUUAGCCACAU | 2299 | 3292 | Yes | No | No |
| 2300 | UGUGGCUAAACUAGCAGAA | UUCUGCUAGUUUAGCCACA | 2301 | 3293 | Yes | No | No |
| 2302 | GUGGCUAAACUAGCAGAUA | UAUCUGCUAGUUUAGCCAC | 2303 | 3294 | Yes | No | No |
| 2304 | UGGCUAAACUAGCAGAUGA | UCAUCUGCUAGUUUAGCCA | 2305 | 3295 | No | No | No |
| 2306 | GCUAAACUAGCAGAUGUUA | UAACAUCUGCUAGUUUAGC | 2307 | 3297 | No | No | No |
| 2308 | CUAAACUAGCAGAUGUUCA | UGAACAUCUGCUAGUUUAG | 2309 | 3298 | No | No | No |
| 2310 | UAAACUAGCAGAUGUUCCA | UGGAACAUCUGCUAGUUUA | 2311 | 3299 | No | No | No |
| 2312 | AAACUAGCAGAUGUUCCUA | UAGGAACAUCUGCUAGUUU | 2313 | 3300 | No | No | No |
| 2314 | CAGAUGUUCCUGGAGAAAA | UUUUCUCCAGGAACAUCUG | 2315 | 3307 | No | No | No |
| 2316 | AUGUUCCUGGAGAAAUUUA | UAAAUUUCUCCAGGAACAU | 2317 | 3310 | No | No | No |
| 2318 | AGAAAGCAGCUCACAAGUA | UACUUGUGAGCUGCUUUCU | 2319 | 3331 | Yes | No | No |
| 2320 | GAAAGCAGCUCACAAGUCA | UGACUUGUGAGCUGCUUUC | 2321 | 3332 | Yes | No | No |
| 2322 | AAAGCAGCUCACAAGUCAA | UUGACUUGUGAGCUGCUUU | 2323 | 3333 | Yes | No | No |
| 2324 | AGCUCACAAGUCAAAAGAA | UUCUUUUGACUUGUGAGCU | 2325 | 3338 | Yes | No | No |
| 2326 | CUCACAAGUCAAAAGAGCA | UGCUCUUUUGACUUGUGAG | 2327 | 3340 | Yes | No | No |
| 2328 | UCACAAGUCAAAAGAGCUA | UAGCUCUUUUGACUUGUGA | 2329 | 3341 | Yes | No | No |
| 2330 | GAGCUGGAAGGAUUAAUAA | UUAUUAAUCCUUCCAGCUC | 2331 | 3354 | Yes | No | No |
| 2332 | GAAGGAUUAAUAAAUACGA | UCGUAUUUAUUAAUCCUUC | 2333 | 3360 | Yes | No | No |
| 2334 | GGAUUAAUAAAUACGAAAA | UUUUCGUAUUUAUUAAUCC | 2335 | 3363 | Yes | No | No |
| 2336 | AUUAAUAAAUACGAAAAGA | UCUUUUCGUAUUUAUUAAU | 2337 | 3365 | Yes | No | No |
| 2338 | AUACGAAAAGAAAGAGACA | UGUCUCUUUCUUUUCGUAU | 2339 | 3373 | No | No | No |
| 2340 | UACGAAAAGAAAGAGACUA | UAGUCUCUUUCUUUUCGUA | 2341 | 3374 | No | No | No |
| 2342 | AAAGAAAGAGACUCAAGUA | UACUUGAGUCUCUUUCUUU | 2343 | 3379 | No | No | No |
| 2344 | AAGAAAGAGACUCAAGUAA | UUACUUGAGUCUCUUUCUU | 2345 | 3380 | No | No | No |
| 2346 | AGAAAGAGACUCAAGUAUA | UAUACUUGAGUCUCUUUCU | 2347 | 3381 | No | No | No |
| 2348 | GAAAGAGACUCAAGUAUUA | UAAUACUUGAGUCUCUUUC | 2349 | 3382 | No | No | No |
| 2350 | AAAGAGACUCAAGUAUUUA | UAAAUACUUGAGUCUCUUU | 2351 | 3383 | No | No | No |
| 2352 | AGAGACUCAAGUAUUUUGA | UCAAAAUACUUGAGUCUCU | 2353 | 3385 | Yes | No | No |
| 2354 | AGACUCAAGUAUUUUGCAA | UUGCAAAAUACUUGAGUCU | 2355 | 3387 | Yes | No | No |
| 2356 | ACUCAAGUAUUUUGCAAAA | UUUUGCAAAAUACUUGAGU | 2357 | 3389 | Yes | No | No |
| 2358 | CUCAAGUAUUUUGCAAAGA | UCUUUGCAAAAUACUUGAG | 2359 | 3390 | Yes | No | No |
| 2360 | AAGUAUUUUGCAAAGUUAA | UUAACUUUGCAAAAUACUU | 2361 | 3393 | Yes | No | No |
| 2362 | GUAUUUUGCAAAGUUAUGA | UCAUAACUUUGCAAAAUAC | 2363 | 3395 | Yes | No | No |
| 2364 | AUUUUGCAAAGUUAUGGAA | UUCCAUAACUUUGCAAAAU | 2365 | 3397 | Yes | No | No |
| 2366 | UUUUGCAAAGUUAUGGACA | UGUCCAUAACUUUGCAAAA | 2367 | 3398 | Yes | No | No |
| 2368 | UUUGCAAAGUUAUGGACGA | UCGUCCAUAACUUUGCAAA | 2369 | 3399 | Yes | No | No |
| 2370 | UUGCAAAGUUAUGGACGAA | UUCGUCCAUAACUUUGCAA | 2371 | 3400 | Yes | No | No |
| 2372 | UGCAAAGUUAUGGACGAUA | UAUCGUCCAUAACUUUGCA | 2373 | 3401 | Yes | No | No |
| 2374 | AAGUUAUGGACGAUGCAUA | UAUGCAUCGUCCAUAACUU | 2375 | 3405 | Yes | No | No |
| 2376 | AGUUAUGGACGAUGCAUAA | UUAUGCAUCGUCCAUAACU | 2377 | 3406 | Yes | No | No |
| 2378 | GUUAUGGACGAUGCAUAAA | UUUAUGCAUCGUCCAUAAC | 2379 | 3407 | Yes | No | No |
| 2380 | UAUGGACGAUGCAUAAUGA | UCAUUAUGCAUCGUCCAUA | 2381 | 3409 | Yes | No | No |
| 2382 | AUGGACGAUGCAUAAUGCA | UGCAUUAUGCAUCGUCCAU | 2383 | 3410 | Yes | No | No |
| 2384 | UGGACGAUGCAUAAUGCAA | UUGCAUUAUGCAUCGUCCA | 2385 | 3411 | Yes | No | No |
| 2386 | GGACGAUGCAUAAUGCACA | UGUGCAUUAUGCAUCGUCC | 2387 | 3412 | Yes | No | No |
| 2388 | GACGAUGCAUAAUGCACAA | UUGUGCAUUAUGCAUCGUC | 2389 | 3413 | Yes | No | No |
| 2390 | CGAUGCAUAAUGCACAAGA | UCUUGUGCAUUAUGCAUCG | 2391 | 3415 | Yes | No | No |
| 2392 | GAUGCAUAAUGCACAAGAA | UUCUUGUGCAUUAUGCAUC | 2393 | 3416 | Yes | No | No |
| 2394 | AUGCAUAAUGCACAAGACA | UGUCUUGUGCAUUAUGCAU | 2395 | 3417 | Yes | No | No |
| 2396 | UGCAUAAUGCACAAGACCA | UGGUCUUGUGCAUUAUGCA | 2397 | 3418 | Yes | No | No |
| 2398 | UGCACAAGACCUGCAGAAA | UUUCUGCAGGUCUUGUGCA | 2399 | 3425 | Yes | No | No |
| 2400 | GCACAAGACCUGCAGAAGA | UCUUCUGCAGGUCUUGUGC | 2401 | 3426 | Yes | No | No |
| 2402 | ACAAGACCUGCAGAAGUGA | UCACUUCUGCAGGUCUUGU | 2403 | 3428 | Yes | No | No |
| 2404 | AGACCUGCAGAAGUGGACA | UGUCCACUUCUGCAGGUCU | 2405 | 3431 | Yes | No | No |
| 2406 | GACCUGCAGAAGUGGACAA | UUGUCCACUUCUGCAGGUC | 2407 | 3432 | Yes | No | No |
| 2408 | AAGUGGACAGAGGAGUUCA | UGAACUCCUCUGUCCACUU | 2409 | 3441 | Yes | No | No |
| 2410 | AGUGGACAGAGGAGUUCAA | UUGAACUCCUCUGUCCACU | 2411 | 3442 | No | No | No |
| 2412 | GGACAGAGGAGUUCAACAA | UUGUUGAACUCCUCUGUCC | 2413 | 3445 | No | No | No |
| 2414 | GACAGAGGAGUUCAACAUA | UAUGUUGAACUCCUCUGUC | 2415 | 3446 | No | No | No |
| 2416 | ACAGAGGAGUUCAACAUGA | UCAUGUUGAACUCCUCUGU | 2417 | 3447 | No | No | No |
| 2418 | AGAGGAGUUCAACAUGGAA | UUCCAUGUUGAACUCCUCU | 2419 | 3449 | No | No | No |
| 2420 | GAGGAGUUCAACAUGGAAA | UUUCCAUGUUGAACUCCUC | 2421 | 3450 | No | No | No |
| 2422 | GGAGUUCAACAUGGAAGAA | UUCUUCCAUGUUGAACUCC | 2423 | 3452 | No | No | No |
| 2424 | AUGGAAGAAACACAGACUA | UAGUCUGUGUUUCUUCCAU | 2425 | 3462 | Yes | No | No |
| 2426 | GAAGAAACACAGACUUCUA | UAGAAGUCUGUGUUUCUUC | 2427 | 3465 | Yes | No | No |
| 2428 | AAGAAACACAGACUUCUCA | UGAGAAGUCUGUGUUUCUU | 2429 | 3466 | Yes | No | No |
| 2430 | AGAAACACAGACUUCUCUA | UAGAGAAGUCUGUGUUUCU | 2431 | 3467 | Yes | No | No |
| 2432 | ACACAGACUUCUCUUCUUA | UAAGAAGAGAAGUCUGUGU | 2433 | 3471 | No | No | No |
| 2434 | UCUCUUCUUCAUUAAAAUA | UAUUUUAAUGAAGAAGAGA | 2435 | 3480 | No | No | No |
| 2436 | UUAAAAUGAAGACUACAUA | UAUGUAGUCUUCAUUUUAA | 2437 | 3491 | No | No | No |
| 2438 | UGAAGACUACAUUUGUGAA | UUCACAAAUGUAGUCUUCA | 2439 | 3497 | No | No | No |
| 2440 | GAAGACUACAUUUGUGAAA | UUUCACAAAUGUAGUCUUC | 2441 | 3498 | No | No | No |
| 2442 | AAGACUACAUUUGUGAACA | UGUUCACAAAUGUAGUCUU | 2443 | 3499 | No | No | No |
| 2444 | AGACUACAUUUGUGAACAA | UUGUUCACAAAUGUAGUCU | 2445 | 3500 | No | No | No |
| 2446 | AAAAUACCAACUGUACAAA | UUUGUACAGUUGGUAUUUU | 2447 | 3533 | Yes | No | No |
| 2448 | CAACUGUACAAAAUAACUA | UAGUUAUUUUGUACAGUUG | 2449 | 3540 | Yes | No | No |
| 2450 | ACUGUACAAAAUAACUCUA | UAGAGUUAUUUUGUACAGU | 2451 | 3542 | Yes | No | No |
| 2452 | UGUACAAAAUAACUCUCCA | UGGAGAGUUAUUUUGUACA | 2453 | 3544 | Yes | No | No |
| 2454 | CAAAAUAACUCUCCAGUAA | UUACUGGAGAGUUAUUUUG | 2455 | 3548 | Yes | No | No |
| 2456 | AAAAUAACUCUCCAGUAAA | UUUACUGGAGAGUUAUUUU | 2457 | 3549 | Yes | No | No |
| 2458 | AAAUAACUCUCCAGUAACA | UGUUACUGGAGAGUUAUUU | 2459 | 3550 | Yes | No | No |
| 2460 | AAUAACUCUCCAGUAACAA | UUGUUACUGGAGAGUUAUU | 2461 | 3551 | Yes | No | No |
| 2462 | AUAACUCUCCAGUAACAGA | UCUGUUACUGGAGAGUUAU | 2463 | 3552 | Yes | No | No |
| 2464 | UAACUCUCCAGUAACAGCA | UGCUGUUACUGGAGAGUUA | 2465 | 3553 | Yes | No | No |
| 2466 | AACUCUCCAGUAACAGCCA | UGGCUGUUACUGGAGAGUU | 2467 | 3554 | Yes | No | No |
| 2468 | CUCUCCAGUAACAGCCUAA | UUAGGCUGUUACUGGAGAG | 2469 | 3556 | Yes | No | No |
| 2470 | CUCCAGUAACAGCCUAUCA | UGAUAGGCUGUUACUGGAG | 2471 | 3558 | No | No | No |
| 2472 | CCAGUAACAGCCUAUCUUA | UAAGAUAGGCUGUUACUGG | 2473 | 3560 | No | No | No |
| 2474 | CAGUAACAGCCUAUCUUUA | UAAAGAUAGGCUGUUACUG | 2475 | 3561 | No | No | No |
| 2476 | AGUAACAGCCUAUCUUUGA | UCAAAGAUAGGCUGUUACU | 2477 | 3562 | No | No | No |
| 2478 | GUAACAGCCUAUCUUUGUA | UACAAAGAUAGGCUGUUAC | 2479 | 3563 | No | No | No |
| 2480 | UAACAGCCUAUCUUUGUGA | UCACAAAGAUAGGCUGUUA | 2481 | 3564 | No | No | No |
| 2482 | ACAGCCUAUCUUUGUGUGA | UCACACAAAGAUAGGCUGU | 2483 | 3566 | No | No | No |
| 2484 | CAGCCUAUCUUUGUGUGAA | UUCACACAAAGAUAGGCUG | 2485 | 3567 | No | No | No |
| 2486 | AGCCUAUCUUUGUGUGACA | UGUCACACAAAGAUAGGCU | 2487 | 3568 | No | No | No |
| 2488 | GCCUAUCUUUGUGUGACAA | UUGUCACACAAAGAUAGGC | 2489 | 3569 | No | No | No |
| 2490 | UAUCUUUGUGUGACAUGUA | UACAUGUCACACAAAGAUA | 2491 | 3572 | No | No | No |
| 2492 | AUCUUUGUGUGACAUGUGA | UCACAUGUCACACAAAGAU | 2493 | 3573 | No | No | No |
| 2494 | UCUUUGUGUGACAUGUGAA | UUCACAUGUCACACAAAGA | 2495 | 3574 | No | No | No |
| 2496 | CUUUGUGUGACAUGUGAGA | UCUCACAUGUCACACAAAG | 2497 | 3575 | No | No | No |
| 2498 | UGUGACAUGUGAGCAUAAA | UUUAUGCUCACAUGUCACA | 2499 | 3580 | Yes | No | No |
| 2500 | GACAUGUGAGCAUAAAAUA | UAUUUUAUGCUCACAUGUC | 2501 | 3583 | Yes | No | No |
| 2502 | ACAUGUGAGCAUAAAAUUA | UAAUUUUAUGCUCACAUGU | 2503 | 3584 | Yes | No | No |
| 2504 | CAUGUGAGCAUAAAAUUAA | UUAAUUUUAUGCUCACAUG | 2505 | 3585 | Yes | No | No |
| 2506 | UGUGAGCAUAAAAUUAUGA | UCAUAAUUUUAUGCUCACA | 2507 | 3587 | Yes | No | No |
| 2508 | GUGAGCAUAAAAUUAUGAA | UUCAUAAUUUUAUGCUCAC | 2509 | 3588 | Yes | No | No |
| 2510 | UGAGCAUAAAAUUAUGACA | UGUCAUAAUUUUAUGCUCA | 2511 | 3589 | Yes | No | No |
| 2512 | UAAAAUUAUGACCAUGGUA | UACCAUGGUCAUAAUUUUA | 2513 | 3595 | Yes | No | No |
| 2514 | AAAAUUAUGACCAUGGUAA | UUACCAUGGUCAUAAUUUU | 2515 | 3596 | Yes | No | No |
| 2516 | AAUUAUGACCAUGGUAUAA | UUAUACCAUGGUCAUAAUU | 2517 | 3598 | Yes | No | No |
| 2518 | AUUAUGACCAUGGUAUAUA | UAUAUACCAUGGUCAUAAU | 2519 | 3599 | Yes | No | No |
| 2520 | UGACCAUGGUAUAUUCCUA | UAGGAAUAUACCAUGGUCA | 2521 | 3603 | Yes | No | No |
| 2522 | GACCAUGGUAUAUUCCUAA | UUAGGAAUAUACCAUGGUC | 2523 | 3604 | Yes | No | No |
| 2524 | ACCAUGGUAUAUUCCUAUA | UAUAGGAAUAUACCAUGGU | 2525 | 3605 | No | No | No |
| 2526 | AUGGUAUAUUCCUAUUGGA | UCCAAUAGGAAUAUACCAU | 2527 | 3608 | No | No | No |
| 2528 | UGGUAUAUUCCUAUUGGAA | UUCCAAUAGGAAUAUACCA | 2529 | 3609 | No | No | No |
| 2530 | GGUAUAUUCCUAUUGGAAA | UUUCCAAUAGGAAUAUACC | 2531 | 3610 | No | No | No |
| 2532 | AUUCCUAUUGGAAACAGAA | UUCUGUUUCCAAUAGGAAU | 2533 | 3615 | No | No | No |
| 2534 | UUCCUAUUGGAAACAGAGA | UCUCUGUUUCCAAUAGGAA | 2535 | 3616 | No | No | No |
| 2536 | UCCUAUUGGAAACAGAGAA | UUCUCUGUUUCCAAUAGGA | 2537 | 3617 | No | No | No |
| 2538 | CCUAUUGGAAACAGAGAGA | UCUCUCUGUUUCCAAUAGG | 2539 | 3618 | No | No | No |
| 2540 | AUUGGAAACAGAGAGGUUA | UAACCUCUCUGUUUCCAAU | 2541 | 3621 | No | No | No |
| 2542 | UUGGAAACAGAGAGGUUUA | UAAACCUCUCUGUUUCCAA | 2543 | 3622 | No | No | No |
| 2544 | UGGAAACAGAGAGGUUUUA | UAAAACCUCUCUGUUUCCA | 2545 | 3623 | Yes | No | No |
| 2546 | GUUUCUGUCUUCCUAACUA | UAGUUAGGAAGACAGAAAC | 2547 | 3662 | No | No | No |
| 2548 | UCUGUCUUCCUAACUUUUA | UAAAAGUUAGGAAGACAGA | 2549 | 3665 | No | No | No |
| 2550 | CUGUCUUCCUAACUUUUCA | UGAAAAGUUAGGAAGACAG | 2551 | 3666 | Yes | No | No |
| 2552 | GUCUUCCUAACUUUUCUAA | UUAGAAAAGUUAGGAAGAC | 2553 | 3668 | Yes | No | No |
| 2554 | UCUUCCUAACUUUUCUACA | UGUAGAAAAGUUAGGAAGA | 2555 | 3669 | No | No | No |
| 2556 | CCUAACUUUUCUACGUAUA | UAUACGUAGAAAAGUUAGG | 2557 | 3673 | No | No | No |
| 2558 | CUAACUUUUCUACGUAUAA | UUAUACGUAGAAAAGUUAG | 2559 | 3674 | No | No | No |
| 2560 | UAACUUUUCUACGUAUAAA | UUUAUACGUAGAAAAGUUA | 2561 | 3675 | No | No | No |
| 2562 | AACUUUUCUACGUAUAAAA | UUUUAUACGUAGAAAAGUU | 2563 | 3676 | No | No | No |
| 2564 | ACUUUUCUACGUAUAAACA | UGUUUAUACGUAGAAAAGU | 2565 | 3677 | No | No | No |
| 2566 | UUUUCUACGUAUAAACACA | UGUGUUUAUACGUAGAAAA | 2567 | 3679 | No | No | No |
| 2568 | UUUCUACGUAUAAACACUA | UAGUGUUUAUACGUAGAAA | 2569 | 3680 | No | No | No |
| 2570 | UCUACGUAUAAACACUCUA | UAGAGUGUUUAUACGUAGA | 2571 | 3682 | No | No | No |
| 2572 | UACGUAUAAACACUCUUGA | UCAAGAGUGUUUAUACGUA | 2573 | 3684 | No | No | No |
| 2574 | ACGUAUAAACACUCUUGAA | UUCAAGAGUGUUUAUACGU | 2575 | 3685 | No | No | No |
| 2576 | CGUAUAAACACUCUUGAAA | UUUCAAGAGUGUUUAUACG | 2577 | 3686 | No | No | No |
| 2578 | AUAAACACUCUUGAAUAGA | UCUAUUCAAGAGUGUUUAU | 2579 | 3689 | Yes | No | No |
| 2580 | UAAACACUCUUGAAUAGAA | UUCUAUUCAAGAGUGUUUA | 2581 | 3690 | Yes | No | No |
| 2582 | ACACUCUUGAAUAGACUUA | UAAGUCUAUUCAAGAGUGU | 2583 | 3693 | Yes | No | No |
| 2584 | ACUCUUGAAUAGACUUCCA | UGGAAGUCUAUUCAAGAGU | 2585 | 3695 | Yes | No | No |
| 2586 | CUCUUGAAUAGACUUCCAA | UUGGAAGUCUAUUCAAGAG | 2587 | 3696 | Yes | No | No |
| 2588 | GAAUAGACUUCCACUUUGA | UCAAAGUGGAAGUCUAUUC | 2589 | 3701 | Yes | No | No |
| 2590 | AAUAGACUUCCACUUUGUA | UACAAAGUGGAAGUCUAUU | 2591 | 3702 | Yes | No | No |
| 2592 | AUAGACUUCCACUUUGUAA | UUACAAAGUGGAAGUCUAU | 2593 | 3703 | Yes | No | No |
| 2594 | UAGACUUCCACUUUGUAAA | UUUACAAAGUGGAAGUCUA | 2595 | 3704 | Yes | No | No |
| 2596 | AGACUUCCACUUUGUAAUA | UAUUACAAAGUGGAAGUCU | 2597 | 3705 | Yes | No | No |
| 2598 | GACUUCCACUUUGUAAUUA | UAAUUACAAAGUGGAAGUC | 2599 | 3706 | Yes | No | No |
| 2600 | ACUUCCACUUUGUAAUUAA | UUAAUUACAAAGUGGAAGU | 2601 | 3707 | Yes | No | No |
| 2602 | CUUCCACUUUGUAAUUAGA | UCUAAUUACAAAGUGGAAG | 2603 | 3708 | Yes | No | No |
| 2604 | UCCACUUUGUAAUUAGAAA | UUUCUAAUUACAAAGUGGA | 2605 | 3710 | Yes | No | No |
| 2606 | ACUUUGUAAUUAGAAAAUA | UAUUUUCUAAUUACAAAGU | 2607 | 3713 | Yes | No | No |
| 2608 | AGAAAAUUUUAUGGACAGA | UCUGUCCAUAAAAUUUUCU | 2609 | 3724 | No | No | No |
| 2610 | GAAAAUUUUAUGGACAGUA | UACUGUCCAUAAAAUUUUC | 2611 | 3725 | No | No | No |
| 2612 | AAUUUUAUGGACAGUAAGA | UCUUACUGUCCAUAAAAUU | 2613 | 3728 | No | No | No |
| 2614 | AUUUUAUGGACAGUAAGUA | UACUUACUGUCCAUAAAAU | 2615 | 3729 | No | No | No |
| 2616 | UUUAUGGACAGUAAGUCCA | UGGACUUACUGUCCAUAAA | 2617 | 3731 | No | No | No |
| 2618 | UUAUGGACAGUAAGUCCAA | UUGGACUUACUGUCCAUAA | 2619 | 3732 | No | No | No |
| 2620 | UGGACAGUAAGUCCAGUAA | UUACUGGACUUACUGUCCA | 2621 | 3735 | Yes | No | No |
| 2622 | GACAGUAAGUCCAGUAAAA | UUUUACUGGACUUACUGUC | 2623 | 3737 | Yes | No | No |
| 2624 | ACAGUAAGUCCAGUAAAGA | UCUUUACUGGACUUACUGU | 2625 | 3738 | Yes | No | No |
| 2626 | CAGUAAGUCCAGUAAAGCA | UGCUUUACUGGACUUACUG | 2627 | 3739 | Yes | No | No |
| 2628 | AGUAAGUCCAGUAAAGCCA | UGGCUUUACUGGACUUACU | 2629 | 3740 | Yes | No | No |
| 2630 | GUAAGUCCAGUAAAGCCUA | UAGGCUUUACUGGACUUAC | 2631 | 3741 | Yes | No | No |
| 2632 | UAAGUCCAGUAAAGCCUUA | UAAGGCUUUACUGGACUUA | 2633 | 3742 | Yes | No | No |
| 2634 | AAGUCCAGUAAAGCCUUAA | UUAAGGCUUUACUGGACUU | 2635 | 3743 | Yes | No | No |
| 2636 | AGUCCAGUAAAGCCUUAAA | UUUAAGGCUUUACUGGACU | 2637 | 3744 | Yes | No | No |
| 2638 | GUCCAGUAAAGCCUUAAGA | UCUUAAGGCUUUACUGGAC | 2639 | 3745 | Yes | No | No |
| 2640 | UCCAGUAAAGCCUUAAGUA | UACUUAAGGCUUUACUGGA | 2641 | 3746 | Yes | No | No |
| 2642 | CCAGUAAAGCCUUAAGUGA | UCACUUAAGGCUUUACUGG | 2643 | 3747 | Yes | No | No |
| 2644 | CAGUAAAGCCUUAAGUGGA | UCCACUUAAGGCUUUACUG | 2645 | 3748 | Yes | No | No |
| 2646 | GUAAAGCCUUAAGUGGCAA | UUGCCACUUAAGGCUUUAC | 2647 | 3750 | Yes | No | No |
| 2648 | AAGCCUUAAGUGGCAGAAA | UUUCUGCCACUUAAGGCUU | 2649 | 3753 | Yes | No | No |
| 2650 | AGCCUUAAGUGGCAGAAUA | UAUUCUGCCACUUAAGGCU | 2651 | 3754 | Yes | No | No |
| 2652 | CCUUAAGUGGCAGAAUAUA | UAUAUUCUGCCACUUAAGG | 2653 | 3756 | Yes | No | No |
| 2654 | CUUAAGUGGCAGAAUAUAA | UUAUAUUCUGCCACUUAAG | 2655 | 3757 | Yes | No | No |
| 2656 | UUAAGUGGCAGAAUAUAAA | UUUAUAUUCUGCCACUUAA | 2657 | 3758 | Yes | No | No |
| 2658 | UAAGUGGCAGAAUAUAAUA | UAUUAUAUUCUGCCACUUA | 2659 | 3759 | Yes | No | No |
| 2660 | GUGGCAGAAUAUAAUUCCA | UGGAAUUAUAUUCUGCCAC | 2661 | 3762 | Yes | No | No |
| 2662 | UGGCAGAAUAUAAUUCCCA | UGGGAAUUAUAUUCUGCCA | 2663 | 3763 | Yes | No | No |
| 2664 | GCAGAAUAUAAUUCCCAAA | UUUGGGAAUUAUAUUCUGC | 2665 | 3765 | Yes | No | No |
| 2666 | AAUAUAAUUCCCAAGCUUA | UAAGCUUGGGAAUUAUAUU | 2667 | 3769 | Yes | No | No |
| 2668 | UAUAAUUCCCAAGCUUUUA | UAAAAGCUUGGGAAUUAUA | 2669 | 3771 | Yes | No | No |
| 2670 | AUAAUUCCCAAGCUUUUGA | UCAAAAGCUUGGGAAUUAU | 2671 | 3772 | Yes | No | No |
| 2672 | UAAUUCCCAAGCUUUUGGA | UCCAAAAGCUUGGGAAUUA | 2673 | 3773 | Yes | No | No |
| 2674 | AAUUCCCAAGCUUUUGGAA | UUCCAAAAGCUUGGGAAUU | 2675 | 3774 | Yes | No | No |
| 2676 | AUUCCCAAGCUUUUGGAGA | UCUCCAAAAGCUUGGGAAU | 2677 | 3775 | Yes | No | No |
| 2678 | AAGCUUUUGGAGGGUGAUA | UAUCACCCUCCAAAAGCUU | 2679 | 3781 | Yes | No | No |
| 2680 | AGCUUUUGGAGGGUGAUAA | UUAUCACCCUCCAAAAGCU | 2681 | 3782 | Yes | No | No |
| 2682 | GCUUUUGGAGGGUGAUAUA | UAUAUCACCCUCCAAAAGC | 2683 | 3783 | Yes | No | No |
| 2684 | CUUUUGGAGGGUGAUAUAA | UUAUAUCACCCUCCAAAAG | 2685 | 3784 | Yes | No | No |
| 2686 | UUUUGGAGGGUGAUAUAAA | UUUAUAUCACCCUCCAAAA | 2687 | 3785 | Yes | No | No |
| 2688 | UUUGUUUCAGUUCAGAUAA | UUAUCUGAACUGAAACAAA | 2689 | 3823 | Yes | No | No |
| 2690 | UCAGUUCAGAUAAUUGGCA | UGCCAAUUAUCUGAACUGA | 2691 | 3829 | Yes | No | No |
| 2692 | UUGGCAACUGGGUGAAUCA | UGAUUCACCCAGUUGCCAA | 2693 | 3842 | No | No | No |
| 2694 | GGCAACUGGGUGAAUCUGA | UCAGAUUCACCCAGUUGCC | 2695 | 3844 | No | No | No |
| 2696 | GCAACUGGGUGAAUCUGGA | UCCAGAUUCACCCAGUUGC | 2697 | 3845 | No | No | No |
| 2698 | CAACUGGGUGAAUCUGGCA | UGCCAGAUUCACCCAGUUG | 2699 | 3846 | No | No | No |
| 2700 | GUGAAUCUGGCAGGAAUCA | UGAUUCCUGCCAGAUUCAC | 2701 | 3853 | Yes | No | No |
| 2702 | UGAAUCUGGCAGGAAUCUA | UAGAUUCCUGCCAGAUUCA | 2703 | 3854 | Yes | No | No |
| 2704 | GAAUCUGGCAGGAAUCUAA | UUAGAUUCCUGCCAGAUUC | 2705 | 3855 | No | No | No |
| 2706 | AAUCUGGCAGGAAUCUAUA | UAUAGAUUCCUGCCAGAUU | 2707 | 3856 | No | No | No |
| 2708 | AUCUGGCAGGAAUCUAUCA | UGAUAGAUUCCUGCCAGAU | 2709 | 3857 | No | No | No |
| 2710 | CUGGCAGGAAUCUAUCCAA | UUGGAUAGAUUCCUGCCAG | 2711 | 3859 | No | No | No |
| 2712 | UGGCAGGAAUCUAUCCAUA | UAUGGAUAGAUUCCUGCCA | 2713 | 3860 | No | No | No |
| 2714 | GGCAGGAAUCUAUCCAUUA | UAAUGGAUAGAUUCCUGCC | 2715 | 3861 | No | No | No |
| 2716 | GCAGGAAUCUAUCCAUUGA | UCAAUGGAUAGAUUCCUGC | 2717 | 3862 | No | No | No |
| 2718 | CAGGAAUCUAUCCAUUGAA | UUCAAUGGAUAGAUUCCUG | 2719 | 3863 | No | No | No |
| 2720 | AGGAAUCUAUCCAUUGAAA | UUUCAAUGGAUAGAUUCCU | 2721 | 3864 | No | No | No |
| 2722 | GGAAUCUAUCCAUUGAACA | UGUUCAAUGGAUAGAUUCC | 2723 | 3865 | No | No | No |
| 2724 | GAAUCUAUCCAUUGAACUA | UAGUUCAAUGGAUAGAUUC | 2725 | 3866 | No | No | No |
| 2726 | AAUCUAUCCAUUGAACUAA | UUAGUUCAAUGGAUAGAUU | 2727 | 3867 | No | No | No |
| 2728 | AUCUAUCCAUUGAACUAAA | UUUAGUUCAAUGGAUAGAU | 2729 | 3868 | No | No | No |
| 2730 | UCUAUCCAUUGAACUAAAA | UUUUAGUUCAAUGGAUAGA | 2731 | 3869 | No | No | No |
| 2732 | UAUCCAUUGAACUAAAAUA | UAUUUUAGUUCAAUGGAUA | 2733 | 3871 | No | No | No |
| 2734 | CCAUUGAACUAAAAUAAUA | UAUUAUUUUAGUUCAAUGG | 2735 | 3874 | No | No | No |
| 2736 | UGAACUAAAAUAAUUUUAA | UUAAAAUUAUUUUAGUUCA | 2737 | 3878 | Yes | No | No |
| 2738 | ACUAAAAUAAUUUUAUUAA | UUAAUAAAAUUAUUUUAGU | 2739 | 3881 | Yes | No | No |
| 2740 | UUUAUUAUGCAACCAGUUA | UAACUGGUUGCAUAAUAAA | 2741 | 3892 | No | No | No |
| 2742 | UUAUUAUGCAACCAGUUUA | UAAACUGGUUGCAUAAUAA | 2743 | 3893 | No | No | No |
| 2744 | UAUUAUGCAACCAGUUUAA | UUAAACUGGUUGCAUAAUA | 2745 | 3894 | No | No | No |
| 2746 | AUUAUGCAACCAGUUUAUA | UAUAAACUGGUUGCAUAAU | 2747 | 3895 | No | No | No |
| 2748 | UAUGCAACCAGUUUAUCCA | UGGAUAAACUGGUUGCAUA | 2749 | 3897 | No | No | No |
| 2750 | AUGCAACCAGUUUAUCCAA | UUGGAUAAACUGGUUGCAU | 2751 | 3898 | No | No | No |
| 2752 | CCAGUUUAUCCACCAAGAA | UUCUUGGUGGAUAAACUGG | 2753 | 3904 | Yes | No | No |
| 2754 | AGUUUAUCCACCAAGAACA | UGUUCUUGGUGGAUAAACU | 2755 | 3906 | Yes | No | No |
| 2756 | UUAUCCACCAAGAACAUAA | UUAUGUUCUUGGUGGAUAA | 2757 | 3909 | Yes | No | No |
| 2758 | UAUCCACCAAGAACAUAAA | UUUAUGUUCUUGGUGGAUA | 2759 | 3910 | Yes | No | No |
| 2760 | AUCCACCAAGAACAUAAGA | UCUUAUGUUCUUGGUGGAU | 2761 | 3911 | Yes | No | No |
| 2762 | UCCACCAAGAACAUAAGAA | UUCUUAUGUUCUUGGUGGA | 2763 | 3912 | Yes | No | No |
| 2764 | CCACCAAGAACAUAAGAAA | UUUCUUAUGUUCUUGGUGG | 2765 | 3913 | Yes | No | No |
| 2766 | CACCAAGAACAUAAGAAUA | UAUUCUUAUGUUCUUGGUG | 2767 | 3914 | Yes | No | No |
| 2768 | ACCAAGAACAUAAGAAUUA | UAAUUCUUAUGUUCUUGGU | 2769 | 3915 | Yes | No | No |
| 2770 | UAAGUAGAAAGAAUUGGCA | UGCCAAUUCUUUCUACUUA | 2771 | 3938 | No | No | No |
| 2772 | AGUAGAAAGAAUUGGCCAA | UUGGCCAAUUCUUUCUACU | 2773 | 3940 | No | No | No |
| 2774 | AUAAAGUACAUCUCUACUA | UAGUAGAGAUGUACUUUAU | 2775 | 4079 | Yes | No | No |
| 2776 | AAUGAGCCGAGAUCACGUA | UACGUGAUCUCGGCUCAUU | 2777 | 4202 | No | No | No |
| 2778 | GAAAUAGAAUUAUCAAGCA | UGCUUGAUAAUUCUAUUUC | 2779 | 4286 | Yes | No | No |
| 2780 | AAUAGAAUUAUCAAGCUUA | UAAGCUUGAUAAUUCUAUU | 2781 | 4288 | Yes | No | No |
| 2782 | UAGAAUUAUCAAGCUUUUA | UAAAAGCUUGAUAAUUCUA | 2783 | 4290 | Yes | No | No |
| 2784 | AGAAUUAUCAAGCUUUUAA | UUAAAAGCUUGAUAAUUCU | 2785 | 4291 | Yes | No | No |
| 2786 | GAAUUAUCAAGCUUUUAAA | UUUAAAAGCUUGAUAAUUC | 2787 | 4292 | Yes | No | No |
| 2788 | AAUUAUCAAGCUUUUAAAA | UUUUAAAAGCUUGAUAAUU | 2789 | 4293 | Yes | No | No |
| 2790 | UAGAGCACAGAAGGAAUAA | UUAUUCCUUCUGUGCUCUA | 2791 | 4314 | No | No | No |
| 2792 | GCACAGAAGGAAUAAGGUA | UACCUUAUUCCUUCUGUGC | 2793 | 4318 | No | No | No |
| 2794 | CACAGAAGGAAUAAGGUCA | UGACCUUAUUCCUUCUGUG | 2795 | 4319 | No | No | No |
| 2796 | ACAGAAGGAAUAAGGUCAA | UUGACCUUAUUCCUUCUGU | 2797 | 4320 | No | No | No |
| 2798 | CAGAAGGAAUAAGGUCAUA | UAUGACCUUAUUCCUUCUG | 2799 | 4321 | No | No | No |
| 2800 | AGAAGGAAUAAGGUCAUGA | UCAUGACCUUAUUCCUUCU | 2801 | 4322 | No | No | No |
| 2802 | GAAGGAAUAAGGUCAUGAA | UUCAUGACCUUAUUCCUUC | 2803 | 4323 | No | No | No |
| 2804 | AAGGAAUAAGGUCAUGAAA | UUUCAUGACCUUAUUCCUU | 2805 | 4324 | No | No | No |
| 2806 | AGGAAUAAGGUCAUGAAAA | UUUUCAUGACCUUAUUCCU | 2807 | 4325 | No | No | No |
| 2808 | GGAAUAAGGUCAUGAAAUA | UAUUUCAUGACCUUAUUCC | 2809 | 4326 | No | No | No |
| 2810 | AAUAAGGUCAUGAAAUUUA | UAAAUUUCAUGACCUUAUU | 2811 | 4328 | Yes | No | No |
| 2812 | AUAAGGUCAUGAAAUUUAA | UUAAAUUUCAUGACCUUAU | 2813 | 4329 | Yes | No | No |
| 2814 | GAAAUUUAAAAGGUUAAAA | UUUUAACCUUUUAAAUUUC | 2815 | 4339 | No | No | No |
| 2816 | GGUUAAAUAUUGUCAUAGA | UCUAUGACAAUAUUUAACC | 2817 | 4350 | No | No | No |
| 2818 | UUAAAUAUUGUCAUAGGAA | UUCCUAUGACAAUAUUUAA | 2819 | 4352 | No | No | No |
| 2820 | AAUAUUGUCAUAGGAUUAA | UUAAUCCUAUGACAAUAUU | 2821 | 4355 | No | No | No |
| 2822 | UAUUGUCAUAGGAUUAAGA | UCUUAAUCCUAUGACAAUA | 2823 | 4357 | No | No | No |
| 2824 | UUGUCAUAGGAUUAAGCAA | UUGCUUAAUCCUAUGACAA | 2825 | 4359 | No | No | No |
| 2826 | GUCAUAGGAUUAAGCAGUA | UACUGCUUAAUCCUAUGAC | 2827 | 4361 | No | No | No |
| 2828 | UAGGAUUAAGCAGUUUAAA | UUUAAACUGCUUAAUCCUA | 2829 | 4365 | No | No | No |
| 2830 | UUAAGCAGUUUAAAGAUUA | UAAUCUUUAAACUGCUUAA | 2831 | 4370 | No | No | No |
| 2832 | AGCAGUUUAAAGAUUGUUA | UAACAAUCUUUAAACUGCU | 2833 | 4373 | No | No | No |
| 2834 | GCAGUUUAAAGAUUGUUGA | UCAACAAUCUUUAAACUGC | 2835 | 4374 | No | No | No |
| 2836 | CAGUUUAAAGAUUGUUGGA | UCCAACAAUCUUUAAACUG | 2837 | 4375 | No | No | No |
| 2838 | GUUUAAAGAUUGUUGGAUA | UAUCCAACAAUCUUUAAAC | 2839 | 4377 | No | No | No |
| 2840 | UUAAAGAUUGUUGGAUGAA | UUCAUCCAACAAUCUUUAA | 2841 | 4379 | No | No | No |
| 2842 | GAUUGUUGGAUGAAAUUAA | UUAAUUUCAUCCAACAAUC | 2843 | 4384 | No | No | No |
| 2844 | UUGGAUGAAAUUAUUUGUA | UACAAAUAAUUUCAUCCAA | 2845 | 4389 | Yes | No | No |
| 2846 | GAUGAAAUUAUUUGUCAUA | UAUGACAAAUAAUUUCAUC | 2847 | 4392 | Yes | No | No |
| 2848 | UAUUUGUCAUUCAUUCAAA | UUUGAAUGAAUGACAAAUA | 2849 | 4400 | No | No | No |
| 2850 | UGUCAUUCAUUCAAGUAAA | UUUACUUGAAUGAAUGACA | 2851 | 4404 | No | No | No |
| 2852 | GUCAUUCAUUCAAGUAAUA | UAUUACUUGAAUGAAUGAC | 2853 | 4405 | No | No | No |
| 2854 | AUUCAUUCAAGUAAUAAAA | UUUUAUUACUUGAAUGAAU | 2855 | 4408 | No | No | No |
| 2856 | UUCAUUCAAGUAAUAAAUA | UAUUUAUUACUUGAAUGAA | 2857 | 4409 | No | No | No |
| 2858 | UCAAGUAAUAAAUAUUUAA | UUAAAUAUUUAUUACUUGA | 2859 | 4414 | No | No | No |
| 2860 | CAAGUAAUAAAUAUUUAAA | UUUAAAUAUUUAUUACUUG | 2861 | 4415 | No | No | No |
| 2862 | AGUAAUAAAUAUUUAAUGA | UCAUUAAAUAUUUAUUACU | 2863 | 4417 | Yes | No | No |
| 2864 | GUAAUAAAUAUUUAAUGAA | UUCAUUAAAUAUUUAUUAC | 2865 | 4418 | Yes | No | No |
| 2866 | AAAUAUUUAAUGAAUACUA | UAGUAUUCAUUAAAUAUUU | 2867 | 4423 | Yes | No | No |
| 2868 | UAAUGAAUACUUGCUAUAA | UUAUAGCAAGUAUUCAUUA | 2869 | 4430 | Yes | No | No |
| 2870 | AAUGAAUACUUGCUAUAAA | UUUAUAGCAAGUAUUCAUU | 2871 | 4431 | No | No | No |
| 2872 | AUGAAUACUUGCUAUAAAA | UUUUAUAGCAAGUAUUCAU | 2873 | 4432 | No | No | No |

TABLE 4
Sense strands with cross-species compatibility with Human and Cyno MSH3
SENSE STRAND SEQ ID NOS/SENSE OLIGO NOS
30, 32, 34, 56, 58, 60, 62, 64, 66, 68, 70, 72, 74, 76, 78, 80, 82, 84, 86, 102, 104, 106, 108,
110, 112, 114, 116, 118, 120, 122, 124, 126, 128, 130, 132, 134, 136, 138, 140, 142, 144, 146,
148, 150, 152, 154, 156, 158, 160, 162, 164, 190, 192, 234, 236, 238, 240, 242, 244, 246, 248,
250, 252, 254, 256, 258, 260, 302, 304, 306, 308, 310, 312, 314, 316, 318, 320, 322, 324, 326,
328, 330, 332, 334, 336, 338, 340, 342, 344, 346, 348, 350, 352, 354, 356, 358, 360, 362, 364,
366, 368, 370, 372, 374, 376, 378, 380, 382, 384, 386, 388, 390, 392, 394, 396, 398, 400, 402,
404, 406, 408, 410, 412, 414, 416, 418, 420, 422, 424, 426, 428, 430, 432, 434, 436, 438, 440,
442, 444, 446, 448, 450, 452, 454, 456, 458, 460, 462, 464, 466, 468, 470, 472, 474, 476, 478,
480, 482, 484, 486, 488, 490, 492, 494, 496, 498, 500, 502, 504, 506, 508, 510, 512, 514, 516,
518, 520, 522, 524, 526, 528, 530, 532, 534, 536, 538, 540, 542, 544, 546, 548, 550, 552, 554,
556, 558, 560, 562, 564, 566, 568, 570, 572, 574, 576, 578, 580, 582, 584, 608, 610, 612, 614,
616, 618, 620, 622, 624, 626, 628, 630, 632, 634, 636, 638, 640, 642, 644, 646, 648, 650, 652,
654, 656, 658, 660, 662, 664, 666, 668, 670, 672, 674, 676, 678, 680, 682, 684, 686, 688, 690,
692, 694, 696, 698, 700, 702, 704, 706, 708, 710, 712, 714, 716, 718, 720, 722, 724, 726, 728,
730, 732, 734, 736, 738, 740, 742, 744, 746, 748, 754, 756, 758, 760, 762, 776, 778, 780, 782,
784, 786, 788, 790, 792, 794, 796, 798, 820, 822, 824, 826, 828, 830, 832, 834, 836, 838, 840,
842, 844, 846, 848, 850, 852, 854, 874, 876, 878, 880, 882, 884, 886, 888, 890, 892, 894, 896,
898, 900, 902, 904, 906, 908, 910, 912, 914, 916, 918, 920, 922, 924, 926, 928, 930, 932, 934,
936, 938, 940, 942, 944, 946, 948, 960, 962, 964, 966, 968, 970, 972, 974, 976, 978, 980, 982,
984, 986, 988, 990, 992, 994, 996, 998, 1000, 1002, 1004, 1006, 1008, 1010, 1012, 1014,
1016, 1018, 1020, 1022, 1024, 1026, 1028, 1030, 1032, 1034, 1036, 1038, 1040, 1042, 1044,
1046, 1048, 1050, 1052, 1054, 1056, 1058, 1060, 1062, 1064, 1066, 1068, 1070, 1072, 1074,
1076, 1078, 1080, 1082, 1084, 1086, 1088, 1090, 1092, 1094, 1096, 1098, 1100, 1102, 1104,
1106, 1108, 1110, 1112, 1114, 1116, 1118, 1120, 1122, 1124, 1126, 1128, 1130, 1132, 1134,
1136, 1138, 1140, 1142, 1144, 1146, 1148, 1150, 1152, 1182, 1184, 1186, 1188, 1190, 1192,
1194, 1196, 1198, 1200, 1202, 1204, 1206, 1208, 1210, 1212, 1214, 1216, 1218, 1220, 1222,
1224, 1226, 1228, 1230, 1232, 1250, 1252, 1254, 1256, 1258, 1260, 1262, 1264, 1266, 1268,
1270, 1272, 1294, 1296, 1298, 1300, 1302, 1304, 1306, 1308, 1310, 1312, 1314, 1316, 1318,
1362, 1364, 1366, 1368, 1370, 1372, 1374, 1376, 1378, 1380, 1382, 1384, 1386, 1388, 1390,
1392, 1394, 1396, 1398, 1400, 1402, 1404, 1406, 1408, 1410, 1412, 1414, 1416, 1418, 1420,
1422, 1424, 1426, 1428, 1430, 1432, 1434, 1436, 1438, 1440, 1442, 1444, 1446, 1448, 1450,
1452, 1454, 1456, 1492, 1494, 1496, 1498, 1500, 1502, 1504, 1506, 1508, 1510, 1512, 1514,
1516, 1518, 1520, 1522, 1524, 1526, 1532, 1534, 1536, 1538, 1540, 1542, 1544, 1546, 1548,
1550, 1552, 1554, 1556, 1558, 1560, 1562, 1564, 1566, 1568, 1570, 1572, 1574, 1576, 1578,
1580, 1582, 1584, 1586, 1588, 1590, 1592, 1620, 1622, 1624, 1626, 1630, 1632, 1634, 1636,
1638, 1640, 1642, 1644, 1646, 1648, 1650, 1652, 1654, 1656, 1658, 1660, 1662, 1664, 1666,
1668, 1670, 1672, 1674, 1676, 1678, 1692, 1694, 1696, 1698, 1700, 1702, 1704, 1706, 1708,
1710, 1712, 1714, 1716, 1718, 1720, 1722, 1724, 1726, 1728, 1730, 1750, 1752, 1754, 1756,
1758, 1760, 1762, 1764, 1766, 1768, 1770, 1786, 1788, 1790, 1792, 1794, 1796, 1798, 1800,
1802, 1826, 1828, 1830, 1832, 1834, 1836, 1838, 1840, 1842, 1858, 1860, 1862, 1864, 1866,
1868, 1870, 1872, 1874, 1876, 1878, 1880, 1882, 1884, 1886, 1888, 1890, 1892, 1894, 1896,
1898, 1900, 1902, 1904, 1906, 1908, 1910, 1912, 1914, 1916, 1918, 1920, 1922, 1924, 1926,
1928, 1930, 1932, 1934, 1936, 1938, 1940, 1942, 1944, 1946, 1948, 1950, 1952, 1954, 1956,
1958, 1960, 1962, 1964, 1966, 1968, 1970, 1972, 1974, 1976, 1978, 1980, 1982, 1984, 1986,
1988, 1990, 1992, 1994, 2014, 2016, 2018, 2020, 2022, 2024, 2026, 2028, 2030, 2032, 2034,
2036, 2038, 2040, 2042, 2044, 2046, 2048, 2086, 2088, 2090, 2092, 2094, 2096, 2098, 2100,
2102, 2104, 2106, 2108, 2110, 2112, 2114, 2116, 2118, 2120, 2122, 2124, 2126, 2128, 2130,
2132, 2134, 2136, 2138, 2140, 2142, 2144, 2146, 2148, 2150, 2152, 2154, 2156, 2172, 2174,
2176, 2178, 2180, 2182, 2184, 2186, 2188, 2190, 2192, 2194, 2196, 2198, 2200, 2202, 2204,
2214, 2216, 2228, 2230, 2232, 2234, 2236, 2238, 2240, 2242, 2244, 2246, 2248, 2250, 2252,
2254, 2256, 2258, 2260, 2262, 2264, 2266, 2268, 2270, 2272, 2274, 2276, 2278, 2280, 2282,
2284, 2286, 2288, 2290, 2292, 2294, 2296, 2298, 2300, 2302, 2318, 2320, 2322, 2324, 2326,
2328, 2330, 2332, 2334, 2336, 2352, 2354, 2356, 2358, 2360, 2362, 2364, 2366, 2368, 2370,
2372, 2374, 2376, 2378, 2380, 2382, 2384, 2386, 2388, 2390, 2392, 2394, 2396, 2398, 2400,
2402, 2404, 2406, 2408, 2424, 2426, 2428, 2430, 2446, 2448, 2450, 2452, 2454, 2456, 2458,
2460, 2462, 2464, 2466, 2468, 2498, 2500, 2502, 2504, 2506, 2508, 2510, 2512, 2514, 2516,
2518, 2520, 2522, 2544, 2550, 2552, 2578, 2580, 2582, 2584, 2586, 2588, 2590, 2592, 2594,
2596, 2598, 2600, 2602, 2604, 2606, 2620, 2622, 2624, 2626, 2628, 2630, 2632, 2634, 2636,
2638, 2640, 2642, 2644, 2646, 2648, 2650, 2652, 2654, 2656, 2658, 2660, 2662, 2664, 2666,
2668, 2670, 2672, 2674, 2676, 2678, 2680, 2682, 2684, 2686, 2688, 2690, 2700, 2702, 2736,
2738, 2752, 2754, 2756, 2758, 2760, 2762, 2764, 2766, 2768, 2774, 2778, 2780, 2782, 2784,
2786, 2788, 2810, 2812, 2844, 2846, 2862, 2864, 2866, 2868,

TABLE 5
Sense strands with cross-species
compatibility with Human and Mouse MSH3
SENSE STRAND SEQ ID NOS/SENSE OLIGO NOS
384, 492, 494, 496, 498, 500, 502, 504, 506, 832, 1052, 1084, 1086,
1088, 1090, 1092, 1094, 1096, 1098, 1100, 1102, 1104, 1138, 1140,
1266, 1268, 1270, 1450, 1452, 1584, 1586, 1588, 1590, 1592, 1594,
1630, 1632, 1634, 1636, 1638, 1640, 1642, 1702, 1704, 1706, 1724,
1906, 1908, 1910, 1912, 1914, 1916, 1952, 1954, 1956, 1958, 1960,
1962, 1964, 1966, 1968, 1970, 1972, 1974, 1976, 1978, 1980, 1982,
1984, 2188, 2190, 2192, 2194, 2196, 2198, 2200, 2202

TABLE 6
Sense strands with cross-species
compatibility with Human and Rat MSH3
SENSE STRAND SEQ ID NOS/SENSE OLIGO NOS
82, 342, 344, 346, 492, 494, 496, 498, 500, 502, 504, 506, 520, 522,
524, 526, 528, 558, 560, 562, 564, 832, 1024, 1026, 1028, 1030, 1032,
1034, 1036, 1038, 1040, 1042, 1044, 1046, 1048, 1050, 1052, 1084, 1086,
1088, 1090, 1410, 1450, 1452, 1584, 1586, 1588, 1590, 1592, 1594, 1596,
1598, 1600, 1602, 1630, 1632, 1634, 1636, 1638, 1640, 1642, 1870, 1982,
1984, 2184, 2186, 2188

TABLE 7
Sense strands with cross-species
compatibility with Human, Cyno, and Mouse MSH3
SENSE STRAND SEQ ID NOS/SENSE OLIGO NOS
384, 492, 494, 496, 498, 500, 502, 504, 506, 832, 1052, 1084, 1086,
1088, 1090, 1092, 1094, 1096, 1098, 1100, 1102, 1104, 1138, 1140, 1266,
1268, 1270, 1450, 1452, 1584, 1586, 1588, 1590, 1592, 1630, 1632, 1634,
1636, 1638, 1640, 1642, 1702, 1704, 1706, 1724, 1906, 1908, 1910, 1912,
1914, 1916, 1952, 1954, 1956, 1958, 1960, 1962, 1964, 1966, 1968, 1970,
1972, 1974, 1976, 1978, 1980, 1982, 1984, 2188, 2190, 2192, 2194, 2196,
2198, 2200, 2202

TABLE 8
Sense strands with cross-species
compatibility with Human, Cyno, and Rat MSH3
SENSE STRAND SEQ ID NOS/SENSE OLIGO NOS
82, 342, 344, 346, 492, 494, 496, 498, 500, 502, 504, 506, 520, 522,
524, 526, 528, 558, 560, 562, 564, 832, 1024, 1026, 1028, 1030, 1032,
1034, 1036, 1038, 1040, 1042, 1044, 1046, 1048, 1050, 1052, 1084, 1086,
1088, 1090, 1410, 1450, 1452, 1584, 1586, 1588, 1590, 1592, 1630, 1632,
1634, 1636, 1638, 1640, 1642, 1870, 1982, 1984, 2184, 2186, 2188

TABLE 9
Sense strands with cross-species
compatibility with Human, Mouse, and Rat MSH3
SENSE STRAND SEQ ID NOS/SENSE OLIGO NOS
492, 494, 496, 498, 500, 502, 504, 506, 832, 1052, 1084, 1086, 1088,
1090, 1450, 1452, 1584, 1586, 1588, 1590, 1592, 1594, 1630, 1632, 1634,
1636, 1638, 1640, 1642, 1982, 1984, 2188

TABLE 10
Sense strands with cross-species
compatibility with Human, Cyno, Mouse, and Rat MSH3
SENSE STRAND SEQ ID NOS/SENSE OLIGO NOS
492, 494, 496, 498, 500, 502, 504, 506, 832, 1052, 1084, 1086, 1088,
1090, 1450, 1452, 1584, 1586, 1588, 1590, 1592, 1630, 1632, 1634, 1636,
1638, 1640, 1642, 1982, 1984, 2188

### Example 2. In Vitro Screening of MSH3 Knockdown

Inhibition or knockdown of MSH3 can be demonstrated using a cell-based assay. For example, HEK293, NIH3T3, or Hela or another available mammalian cell line with dsRNA agents targeting MSH3 identified above in Example 1 using at least five different dose levels, using transfection reagents such as lipofectamine 2000 (Invitrogen) following the manufacturer's instructions. Cells are harvested at multiple time points up to 7 days post transfection for either mRNA or protein analyses. Knockdown of mRNA and protein are determined by RT-qPCR or western blot analyses respectively, using standard molecular biology techniques as previously described (see, for example, as described in Drouet et al., 2014, PLOS One 9(6): e99341). The relative levels of the MSH3 mRNA and protein at the different dsRNA levels are compared with a mock oligonucleotide control. The most potent dsRNA agents (for example, those which are capable of at least 90%, at least 95%, at least 97%, at least 98%, or at least 99% or more, reduction, in protein levels when compared with controls) are selected for subsequent studies, for example, as described in the examples below.

Some siRNA duplexes were evaluated through mRNA knockdown at 10 nM and 0.5 nM, 24 hours after transfection of HeLa cells. The extent of mRNA knockdown by the siRNA duplexes was analyzed by quantitative reverse transcription polymerase chain reaction (RT-qPCR) using TaqMan Gene Expression probes. mRNA expression was calculated via delta-delta Ct(ΔΔCT) method were target expression was doubly normalized to express of the reference gene beta-glucuronidase (GUSB) and cells treated with non-targeting control siRNA.

In Table 11 below, the 5′ U of the antisense oligonucleotide can be any nucleotide (e.g., U, A, G, C, T). In some aspects, the 5′ U of the antisense oligonucleotide in Table 11 is U. The sense and antisense oligonucleotides in Table 11 each include a dTdT overhang on the 3′ end.

Additionally, every A and G in each sense oligonucleotide in Table 11 is a ribonucleotide. Every C and U in each sense oligonucleotide is a 2′-O-Methyl ribonucleotide.

Also, every A and G in each antisense oligonucleotide in Table 11 is a ribonucleotide. Every C and U preceding an A in the antisense oligonucleotide is a 2′-O-Methyl ribonucleotide, with one exception: U is the first nucleotide of the antisense strand, and it is a ribonucleotide.

Each nucleotide in the sense and antisense oligonucleotide in Table 11 is linked by a phosphate.

TABLE 11

| SEQ ID | | | | | | | | | |
|---|---|---|---|---|---|---|---|---|---|
| NO/ | | | | | | | | mean % | |
| SENSE | | | SEQ ID NO/ | | | | | mRNA | |
| OLIGO | | | ANTISENSE | | | | | remaining | |
| NO | Sense | Antisense | OLIGO NO | Pos | Cyno | Mouse | Rat | 0.5 nM | 10 nM |
| 118 | GUCCAGACAGAAUCUCUGA | UCAGAGAUUCUGUCUGGAC | 119 | 531 | Yes | No | No | 67.25 | 44.74 |
| 140 | GCCAAAAUGUACUGAUUUA | UAAAUCAGUACAUUUUGGC | 141 | 569 | Yes | No | No | 27.44 | 23.29 |
| 156 | AUGAUAUCAGUCUUCUACA | UGUAGAAGACUGAUAUCAU | 157 | 589 | Yes | No | No | 71.33 | 45.58 |
| 234 | AGUCAGUUUGGAUCAUCAA | UUGAUGAUCCAAACUGACU | 235 | 681 | Yes | No | No | 66.41 | 40.47 |
| 240 | UUGGAUCAUCAAAUACAAA | UUUGUAUUUGAUGAUCCAA | 241 | 688 | Yes | No | No | 66.56 | 37.87 |
| 246 | AUACAAGUCAUGAAAAUUA | UAAUUUUCAUGACUUGUAU | 247 | 700 | Yes | No | No | 66.57 | 42.32 |
| 304 | CGCUAGAAUUACAAUACAA | UUGUAUUGUAAUUCUAGCG | 305 | 772 | Yes | No | No | 67.39 | 41.15 |
| 308 | UAGAAUUACAAUACAUAGA | UCUAUGUAUUGUAAUUCUA | 309 | 775 | Yes | No | No | 65.28 | 35.66 |
| 350 | GUGGAAUGUGGAUAUAAGA | UCUUAUAUCCACAUUCCAC | 351 | 828 | Yes | No | No | 52.23 | 36.69 |
| 364 | GAUAUAAGUAUAGAUUCUA | UAGAAUCUAUACUUAUAUC | 365 | 838 | Yes | No | No | 66.91 | 35.40 |
| 380 | AGCCCGAGAGCUCAAUAUA | UAUAUUGAGCUCUCGGGCU | 381 | 878 | Yes | No | No | 63.05 | 35.36 |
| 382 | GCCCGAGAGCUCAAUAUUA | UAAUAUUGAGCUCUCGGGC | 383 | 879 | Yes | No | No | 40.56 | 30.67 |
| 386 | CGAGAGCUCAAUAUUUAUA | UAUAAAUAUUGAGCUCUCG | 387 | 882 | Yes | No | No | 40.50 | 26.99 |
| 388 | GAGAGCUCAAUAUUUAUUA | UAAUAAAUAUUGAGCUCUC | 389 | 883 | Yes | No | No | 48.94 | 28.07 |
| 396 | UUUAUUGCCAUUUAGAUCA | UGAUCUAAAUGGCAAUAAA | 397 | 895 | Yes | No | No | 45.93 | 26.42 |
| 406 | GCCAUUUAGAUCACAACUA | UAGUUGUGAUCUAAAUGGC | 407 | 901 | Yes | No | No | 36.86 | 16.73 |
| 418 | AGAUCACAACUUUAUGACA | UGUCAUAAAGUUGUGAUCU | 419 | 908 | Yes | No | No | 49.30 | 30.61 |
| 464 | CACAGACUGUUUGUUCAUA | UAUGAACAAACAGUCUGUG | 465 | 942 | Yes | No | No | 48.64 | 31.30 |
| 478 | CUGGUGGCAAAAGGAUAUA | UAUAUCCUUUUGCCACCAG | 479 | 969 | Yes | No | No | 46.40 | 29.04 |
| 520 | AUUAAAGGCCAUUGGAGAA | UUCUCCAAUGGCCUUUAAU | 521 | 1022 | Yes | No | No | 49.09 | 28.53 |
| 540 | CAACAGAAGUUCACUCUUA | UAAGAGUGAACUUCUGUUG | 541 | 1040 | Yes | No | No | 45.65 | 31.32 |
| 564 | AAAUUGACUGCCCUUUAUA | UAUAAAGGGCAGUCAAUUU | 565 | 1065 | Yes | No | No | 52.13 | 29.00 |
| 568 | UUGACUGCCCUUUAUACAA | UUGUAUAAAGGGCAGUCAA | 569 | 1068 | Yes | No | No | 47.91 | 31.00 |
| 618 | CUAAUCAAGCUGGAUGAUA | UAUCAUCCAGCUUGAUUAG | 619 | 1119 | Yes | No | No | 55.64 | 39.22 |
| 660 | ACUUCUACCAGCUAUCUUA | UAAGAUAGCUGGUAGAAGU | 661 | 1167 | Yes | No | No | 59.77 | 42.59 |
| 696 | AGGCGAGGUUGUGUUUGAA | UUCAAACACAACCUCGCCU | 697 | 1271 | Yes | No | No | 71.11 | 47.44 |
| 698 | GGCGAGGUUGUGUUUGAUA | UAUCAAACACAACCUCGCC | 699 | 1272 | Yes | No | No | 66.28 | 47.74 |
| 700 | GCGAGGUUGUGUUUGAUAA | UUAUCAAACACAACCUCGC | 701 | 1273 | Yes | No | No | 65.54 | 44.44 |
| 750 | CUUGUCCGAGCAAACAGAA | UUCUGUUUGCUCGGACAAG | 751 | 1373 | Yes | No | No | 48.42 | 33.40 |
| 770 | AGAGCCACAUCUGUUAGUA | UACUAACAGAUGUGGCUCU | 771 | 1404 | Yes | No | No | 47.87 | 31.94 |
| 822 | UGAAUACAGCCAUGCUUUA | UAAAGCAUGGCUGUAUUCA | 823 | 1469 | Yes | Yes | Yes | 44.77 | 23.02 |
| 830 | GCUUUCCAGGCAGUUACAA | UUGUAACUGCCUGGAAAGC | 831 | 1482 | Yes | No | No | 52.34 | 32.63 |
| 844 | GCAGUUACAGAGUUUUAUA | UAUAAAACUCUGUAACUGC | 845 | 1491 | Yes | No | No | 55.44 | 33.99 |
| 868 | ACAGUUGACAUCAAAGGUA | UACCUUUGAUGUCAACUGU | 869 | 1518 | Yes | No | No | 57.73 | 34.30 |
| 870 | CAGUUGACAUCAAAGGUUA | UAACCUUUGAUGUCAACUG | 871 | 1519 | Yes | No | No | 61.36 | 32.08 |
| 874 | GUUGACAUCAAAGGUUCUA | UAGAACCUUUGAUGUCAAC | 875 | 1521 | Yes | No | No | 60.17 | 31.68 |
| 904 | CUGGCAUUGUUAACUUAGA | UCUAAGUUAACAAUGCCAG | 905 | 1549 | Yes | No | No | 24.78 | 18.66 |
| 972 | CUCCAAACCUGAGAAUUUA | UAAAUUCUCAGGUUUGGAG | 973 | 1637 | Yes | No | No | 27.05 | 21.76 |
| 1004 | AUGGAAUUUAUGACAAUUA | UAAUUGUCAUAAAUUCCAU | 1005 | 1674 | Yes | No | No | 50.63 | 36.16 |
| 1042 | ACAGAAUCAGACUGAUAUA | UAUAUCAGUCUGAUUCUGU | 1043 | 1724 | Yes | No | No | 60.17 | 33.01 |
| 1060 | UGCUGUGGGUUUUAGACCA | UGGUCUAAAACCCACAGCA | 1061 | 1759 | Yes | No | Vo | 51.81 | 34.96 |
| 1062 | GCUGUGGGUUUUAGACCAA | UUGGUCUAAAACCCACAGC | 1063 | 1760 | Yes | No | No | 51.99 | 31.07 |
| 1064 | UGGGUUUUAGACCACACUA | UAGUGUGGUCUAAAACCCA | 1065 | 1764 | Yes | Yes | Yes | 120.87 | 320.61 |
| 1068 | GGUUUUAGACCACACUAAA | UUUAGUGUGGUCUAAAACC | 1069 | 1766 | Yes | Yes | Yes | 65.36 | 35.69 |
| 1090 | UUGGGAGACGGAAGUUAAA | UUUAACUUCCGUCUCCCAA | 1091 | 1792 | Yes | No | No | 57.77 | 30.99 |
| 1096 | GACGGAAGUUAAAGAAGUA | UACUUCUUUAACUUCCGUC | 1097 | 1798 | Yes | No | No | 36.69 | 31.20 |
| 1098 | ACGGAAGUUAAAGAAGUGA | UCACUUCUUUAACUUCCGU | 1099 | 1799 | Yes | No | No | 50.65 | 30.92 |
| 1114 | CACUCCUUAAAUUAAGGGA | UCCCUUAAUUUAAGGAGUG | 1115 | 1828 | Yes | No | No | 54.62 | 41.66 |
| 1116 | ACUCCUUAAAUUAAGGGAA | UUCCCUUAAUUUAAGGAGU | 1117 | 1829 | Yes | No | No | 52.74 | 37.90 |
| 1166 | UCCAUUCAGAAUCUAGUGA | UCACUAGAUUCUGAAUGGA | 1167 | 1882 | Yes | No | No | 56.52 | 36.88 |
| 1168 | CCAUUCAGAAUCUAGUGUA | UACACUAGAUUCUGAAUGG | 1169 | 1883 | Yes | No | No | 55.00 | 40.41 |
| 1170 | AUUCAGAAUCUAGUGUGUA | UACACACUAGAUUCUGAAU | 1171 | 1885 | Yes | No | No | 56.34 | 36.81 |
| 1182 | AUCUAGUGUGUUUGGUCAA | UUGACCAAACACACUAGAU | 1183 | 1892 | Yes | No | No | 49.97 | 32.53 |
| 1192 | GUGUUUGGUCAGAUAGAAA | UUUCUAUCUGACCAAACAC | 1193 | 1899 | Yes | No | No | 55.26 | 34.88 |
| 1212 | UAGAAAAUCAUCUACGUAA | UUACGUAGAUGAUUUUCUA | 1213 | 1912 | Yes | No | No | 48.24 | 30.58 |
| 1214 | AGAAAAUCAUCUACGUAAA | UUUACGUAGAUGAUUUUCU | 1215 | 1913 | Yes | No | No | 54.57 | 36.51 |
| 1216 | AAAAUCAUCUACGUAAAUA | UAUUUACGUAGAUGAUUUU | 1217 | 1915 | Yes | No | No | 53.87 | 38.28 |
| 1222 | AUCAUCUACGUAAAUUGCA | UGCAAUUUACGUAGAUGAU | 1223 | 1918 | Yes | No | No | 47.43 | 34.83 |
| 1244 | GGACUCUGUAGCAUUUAUA | UAUAAAUGCUACAGAGUCC | 1245 | 1950 | Yes | No | No | 50.88 | 31.70 |
| 1258 | UACCCAAGAGUUCUUCUUA | UAAGAAGAACUCUUGGGUA | 1259 | 1982 | Yes | Yes | No | 55.68 | 47.62 |
| 1292 | UCACCUAAAGUCAGAAUUA | UAAUUCUGACUUUAGGUGA | 1293 | 2018 | Yes | No | No | 58.53 | 46.28 |
| 1358 | CCGUUAUUUUAGAAAUUCA | UGAAUUUCUAAAAUAACGG | 1359 | 2089 | Yes | No | No | 44.55 | 25.12 |
| 1360 | CGUUAUUUUAGAAAUUCCA | UGGAAUUUCUAAAAUAACG | 1361 | 2090 | Yes | No | No | 27.08 | 17.25 |
| 1374 | GUCCAGUGGAGCAUUACUA | UAGUAAUGCUCCACUGGAC | 1375 | 2119 | Yes | No | No | 51.19 | 41.54 |
| 1378 | AGUGGAGCAUUACUUAAAA | UUUUAAGUAAUGCUCCACU | 1379 | 2123 | Yes | No | No | 56.98 | 42.84 |
| 1380 | GUGGAGCAUUACUUAAAGA | UCUUUAAGUAAUGCUCCAC | 1381 | 2124 | Yes | No | No | 51.30 | 42.74 |
| 1400 | GAACAAGCUGCCAAAGUUA | UAACUUUGGCAGCUUGUUC | 1401 | 2151 | Yes | No | No | 48.03 | 34.27 |
| 1488 | UCCGAAUGCAUUUGCAAGA | UCUUGCAAAUGCAUUCGGA | 1489 | 2257 | Yes | No | No | 67.83 | 43.62 |
| 1500 | CUUCUGCACAAUAUGUGAA | UUCACAUAUUGUGCAGAAG | 1501 | 2299 | Yes | No | No | 64.86 | 38.80 |
| 1610 | AGCUAGUCCUUGACUGCAA | UUGCAGUCAAGGACUAGCU | 1611 | 2473 | Yes | Yes | Yes | 55.51 | 29.25 |
| 1682 | AAAGCAGUGCAUCACCUAA | UUAGGUGAUGCACUGCUUU | 1683 | 2547 | Yes | No | No | 59.92 | 38.41 |
| 1684 | GUGCAUCACCUAGCAACUA | UAGUUGCUAGGUGAUGCAC | 1685 | 2553 | Yes | No | No | 56.67 | 38.49 |
| 1748 | GCAGACCAACUGUACAAGA | UCUUGUACAGUUGGUCUGC | 1749 | 2620 | Yes | No | No | 56.19 | 35.33 |
| 1782 | CUGGGAGAACAGGAUCAAA | UUUGAUCCUGUUCUCCCAG | 1783 | 2691 | Yes | No | No | 57.75 | 36.78 |
| 1788 | GAGAACAGGAUCAAUAUGA | UCAUAUUGAUCCUGUUCUC | 1789 | 2695 | Yes | No | No | 73.61 | 43.21 |
| 1790 | AGAACAGGAUCAAUAUGUA | UACAUAUUGAUCCUGUUCU | 1791 | 2696 | Yes | No | No | 64.89 | 39.40 |
| 1826 | GGACUCAGAGAGAGUAAUA | UAUUACUCUCUCUGAGUCC | 1827 | 2738 | Yes | No | | 70.45 | 38.70 |
| 1866 | GAGCUCCUACAUAAAACAA | UUGUUUUAUGUAGGAGCUC | 1867 | 2786 | Yes | No | No | 55.61 | 36.30 |
| 1868 | AGCUCCUACAUAAAACAAA | UUUGUUUUAUGUAGGAGCU | 1869 | 2787 | Yes | No | No | 59.92 | 40.71 |
| 1870 | GCUCCUACAUAAAACAAGA | UCUUGUUUUAUGUAGGAGC | 1871 | 2788 | Yes | No | No | 28.05 | 23.06 |
| 1882 | AAAACAAGUUGCAUUGAUA | UAUCAAUGCAACUUGUUUU | 1883 | 2798 | Yes | No | No | 57.83 | 38.63 |
| 1892 | AGUUGCAUUGAUUACCAUA | UAUGGUAAUCAAUGCAACU | 1893 | 2804 | Yes | No | No | 49.53 | 31.05 |
| 1926 | GCAGAAGAAGCGACAAUUA | UAAUUGUCGCUUCUUCUGC | 1927 | 2850 | Yes | No | No | 46.34 | 30.70 |
| 1946 | GAUUGUGGAUGGCAUUUUA | UAAAAUGCCAUCCACAAUC | 1947 | 2870 | Yes | Yes | No | 51.01 | 29.16 |
| 1964 | GGGUGCUGCAGACAAUAUA | UAUAUUGUCUGCAGCACCC | 1965 | 2897 | Yes | Yes | Yes | 43.17 | 37.54 |
| 1970 | CUGCAGACAAUAUAUAUAA | UUAUAUAUAUUGUCUGCAG | 1971 | 2902 | Yes | No | No | 49.75 | 35.31 |
| 2022 | CAUCACAGUCCUUGGUUAA | UUAACCAAGGACUGUGAUG | 2023 | 2980 | Yes | No | No | 48.00 | 32.17 |
| 2026 | CACAGUCCUUGGUUAUCUA | UAGAUAACCAAGGACUGUG | 2027 | 2983 | Yes | No | No | 48.28 | 31.26 |
| 2030 | CAGUCCUUGGUUAUCUUGA | UCAAGAUAACCAAGGACUG | 2031 | 2985 | Yes | No | No | 50.46 | 36.22 |
| 2084 | UUGCCUAUGCUACACUUGA | UCAAGUGUAGCAUAGGCAA | 2085 | 3046 | Yes | No | No | 48.14 | 33.67 |
| 2088 | CUAUGCUACACUUGAGUAA | UUACUCAAGUGUAGCAUAG | 2089 | 3050 | Yes | No | No | 41.87 | 31.74 |
| 2090 | UAUGCUACACUUGAGUAUA | UAUACUCAAGUGUAGCAUA | 2091 | 3051 | Yes | No | No | 46.64 | 33.27 |
| 2094 | CUACACUUGAGUAUUUCAA | UUGAAAUACUCAAGUGUAG | 2095 | 3055 | Yes | No | No | 48.56 | 32.59 |
| 2124 | UGUGAAAUCCUUAACCCUA | UAGGGUUAAGGAUUUCACA | 2125 | 3080 | Yes | No | No | 52.19 | 35.19 |
| 2130 | AUCCUUAACCCUGUUUGUA | UACAAACAGGGUUAAGGAU | 2131 | 3086 | Yes | No | No | 52.13 | 32.29 |
| 2146 | UUUGUCACCCAUUAUCCGA | UCGGAUAAUGGGUGACAAA | 2147 | 3099 | Yes | No | No | 49.33 | 27.75 |
| 2194 | GAAUUACCACAUGGGAUUA | UAAUCCCAUGUGGUAAUUC | 2195 | 3158 | Yes | Yes | No | 51.85 | 35.54 |
| 2200 | UACCACAUGGGAUUCUUGA | UCAAGAAUCCCAUGUGGUA | 2201 | 3162 | Yes | Yes | No | 49.91 | 34.14 |
| 2204 | CCACAUGGGAUUCUUGGUA | UACCAAGAAUCCCAUGUGG | 2205 | 3164 | Yes | Yes | No | 49.75 | 33.43 |
| 2264 | UCCUUUACCAAAUAACUAA | UUAGUUAUUUGGUAAAGGA | 2265 | 3244 | Yes | No | No | 49.51 | 34.46 |
| 2290 | GCAAGGAGUUAUGGAUUAA | UUAAUCCAUAACUCCUUGC | 2291 | 3273 | Yes | No | No | 46.01 | 32.27 |
| 2308 | UUAAAUGUGGCUAAACUAA | UUAGUUUAGCCACAUUUAA | 2309 | 3288 | Yes | No | No | 46.98 | 33.49 |
| 2318 | GUGGCUAAACUAGCAGAUA | UAUCUGCUAGUUUAGCCAC | 2319 | 3294 | Yes | No | No | 33.99 | 28.52 |
| 2324 | AAAGCAGCUCACAAGUCAA | UUGACUUGUGAGCUGCUUU | 2325 | 3333 | Yes | No | No | 52.22 | 31.71 |
| 2338 | GAAGGAUUAAUAAAUACGA | UCGUAUUUAUUAAUCCUUC | 2339 | 3360 | Yes | No | No | 28.47 | 23.20 |
| 2386 | AGUUAUGGACGAUGCAUAA | UUAUGCAUCGUCCAUAACU | 2387 | 3406 | Yes | No | No | 63.89 | 34.90 |
| 2388 | GUUAUGGACGAUGCAUAAA | UUUAUGCAUCGUCCAUAAC | 2389 | 3407 | Yes | No | No | 53.12 | 34.65 |
| 2390 | GGACGAUGCAUAAUGCACA | UGUGCAUUAUGCAUCGUCC | 2391 | 3412 | Yes | No | No | 65.37 | 30.59 |
| 2392 | GACGAUGCAUAAUGCACAA | UUGUGCAUUAUGCAUCGUC | 2393 | 3413 | Yes | No | No | 71.34 | 51.55 |
| 2466 | ACUGUACAAAAUAACUCUA | UAGAGUUAUUUUGUACAGU | 2467 | 3542 | Yes | No | No | 73.70 | 46.89 |
| 2606 | UAGACUUCCACUUUGUAAA | UUUACAAAGUGGAAGUCUA | 2607 | 3704 | Yes | No | No | 59.07 | 40.22 |
| 2608 | GACUUCCACUUUGUAAUUA | UAAUUACAAAGUGGAAGUC | 2609 | 3706 | Yes | No | No | 62.67 | 36.70 |
| 2610 | ACUUCCACUUUGUAAUUAA | UUAAUUACAAAGUGGAAGU | 2611 | 3707 | Yes | No | No | 58.14 | 41.10 |
| 2632 | GACAGUAAGUCCAGUAAAA | UUUUACUGGACUUACUGUC | 2633 | 3737 | Yes | No | No | 56.92 | 35.95 |
| 2652 | CCAGUAAAGCCUUAAGUGA | UCACUUAAGGCUUUACUGG | 2653 | 3747 | Yes | No | No | 84.65 | 66.02 |
| 2678 | AUAAUUCCCAAGCUUUUGA | UCAAAAGCUUGGGAAUUAU | 2679 | 3772 | Yes | No | No | 59.06 | 38.31 |
| 2690 | CUUUUGGAGGGUGAUAUAA | UUAUAUCACCCUCCAAAAG | 2691 | 3784 | Yes | No | No | 58.68 | 35.99 |
| 2758 | CACCAAGAACAUAAGAAUA | UAUUCUUAUGUUCUUGGUG | 2759 | 3914 | Yes | No | No | 58.28 | 36.18 |

### Example 3. In Vitro Screen for Reduced Expansion

Expansion of DNA triplet repeats can be replicated in vitro using patient-derived cells lines and DNA-damaging agents. Human fibroblasts from Huntington's (GM04281, GM04687 and GM04212) or Friedreich's Ataxia patients (GM03816 and GM02153) or Myotonic dystrophy1 (GM04602, GM03987 and GM03989) are purchased from Coriell Cell Repositories and are maintained in medium following the manufacturer's instructions (Kovtum et al., 2007 Nature, 447(7143): 447-452; Li et al., 2016 Biopreservation and Biobanking 14(4):324-29; Zhang et al., 2013 Mol Ther 22(2): 312-320). To induce CAG-repeat expansion in vitro, fibroblast cells are treated with oxidizing agents such as hydrogen peroxide (H₂O₂), potassium chromate (K₂CrO₄) or potassium bromate (KBrO₃) for up to 2 hrs (Kovtum et al., ibid). Cells are washed, and medium replace to allow cells to recover for 3 days. The treatment is repeated up to twice more before cells are harvested and DNA isolated. CAG repeat length is determined using methods described below. The effect of dsRNA agents on altering CAG-repeat expansion is measured at different concentrations and is compared with controls (mock-transfected and/or control dsRNA at the same concentration as the experimental agent).

### Example 4. Genomic DNA Extraction and Quantitation of CAG Repeat Length by Small Pool-PCR (Sp-PCR) Analyses

Genomic DNA is purified using standard Proteinase K digestions and extracted using DNAzol (Invitrogen) following the manufacturer's instructions. CAG repeat length is determined by small pool-PCR analyses as previously described (Mario Gomes-Pereira and Darren Monckton, 2017, Front Cell Neuro 11:153). In brief, DNA is digested with HindIII, diluted to a final concentration between 1-6 pg/μl and approximately 10 pg was used in subsequent PCR reactions. Primer flanking Exon 1 of the human HTT are used to amplify the CAG alleles and the PCR product is resolved by electrophoresis. Subsequently, Southern blot hybridization is performed, and the CAG alleles are observed by autoradiography OR visualized with ethidium bromide staining. CAG length can be measured directly by sequencing on a MiSeQ or appropriate machine. The change in CAG repeat number in various treatment groups in comparison with controls is calculated using simple descriptive statistics (e.g., mean±standard deviation).

### Example 5. Mouse Studies

Mouse models recapitulating many of the features of trinucleotide repeat expansion diseases including, HD, FA and DM1, are readily available from commercial and academic institutions (Polyglutamine Disorders, Advances in Experimental Medicine and Biology, Vol 1049, 2018: Editors Clevio Nobrega and Lois Pereira de Almeida, Springer). All mouse experiments are conducted in accordance with local IACUC guidelines. Three examples of different diseased mouse models and how they could be used to investigate the usefulness of pharmacological intervention against MSH3 for somatic expansion are included below.

In Huntington's research, several transgenic and knock-in mouse models were generated to investigate the underlying pathological mechanisms involved in the disease. For example, the R2/6 transgenic mouse contains a transgene of ˜1.9 kb of human HTT containing 144 copies of the CAG repeat (Mangiarini et al., 1996 Cell 87: 493-506) while the HdhQ111 model was generated by replacing the mouse HTT exon 1 with a human exon1 containing 111 copies of the CAG repeat (Wheeler et al., 2000 Hum Mol Genet 9:503-513). Both the R2/6 and HdhQ111 models replicate many of the features of human HD including motor and behavioral dysfunctions, neuronal loss, as well as the expansion of CAG repeats in the striatum (Pouladi et al., 2013, Nature Reviews Neuroscience 14: 708-721; Mangiarini et al., 1997 Nature Genet 15: 197-200; Wheeler et al., Hum Mol Genet 8: 115-122). HD Mice are genotyped using DNA derived from tail snips at weaning and the CAG repeat size is determined. Mice are randomized into groups (n=5/group) at weaning at 4 wks old and dosed with a single ICV injection of either up to a 500 μg dose of dsRNA agents (optionally encapsulated in lipid nanoparticle (LNP)) targeting MSH3 or control dsRNA agents (also optionally encapsulated in LNP). A series of dsRNA agents targeting different regions of MSH3 are tested to identify the most efficacious oligo sequence in vivo. At 12 wks of age, mice are euthanized, and tissues extracted for analyses. The list of tissues includes, but not restricted to, striatum, cortex, cerebellum, and liver. Genomic DNA is extracted and the length of CAG repeats measured as described below, and the extent of CAG repeats compared with control mice. Additional pertinent mouse models of HD can be considered.

In Friedreich Ataxia, the YG8 FRDA transgenic mouse model is commonly used to understand the pathology (Al-Mandawi et al., 2006 Genomics 88(5)580-590; Bourn et al., 2012 PLOS One 7(10); e47085). This model was generated through the insertion of a human YAC transgenic containing in the background of a null FRDA mouse. The YG8 model demonstrates somatic expansion of the GAA triplet repeat expansion in neuronal tissues with only mild motor defects. YG8 FRDA mice are genotyped using DNA derived from tail snips at weaning and the CAG repeat size is determined using methods. To determine if MSH3 plays a role in somatic expansion of the disease allele, hemizygous YG8 FRDA animals are administered ICV with dsRNA agents targeting MSH3 or control dsRNA agents (both optionally encapsulated in LNP) targeting knockdown of MSH3 identified above.

Approximately 2 months later, animals are euthanized and tissues collected for molecular analyses. Suitable tissues are heart, quadriceps, dorsal root ganglia (DRG's), cerebellum, kidney, and liver. Genomic DNA is extracted and the length of CAG repeats compared in MSH3 and control dsRNA groups as described above in Example 4.

In Myotonic dystrophy, the DM300-328 transgenic mouse model is suitable for investigating the pathology behind DM1. This mouse model has a large human genomic sequence (˜45 kb) containing over 300 CTG repeats and displays both the somatic expansion and degenerative muscle changes observed in human DM1 (Seznec et al., 2000; Tome et al., 2009 PLOS Genetics 5(5): e1000482; Pandey et al., 2015 J Pharmacol Exp Ther 355:329-340). DM300-328 mice are genotyped using DNA derived from tail snips at weaning and the CAG repeat size is determined. To determine if MSH3 plays a role in somatic expansion of the disease allele in myotonic dystrophy, DM300-328 transgenic animals are administered ASOs targeting knockdown of MSH3 by either subcutaneous injections (sc), intraperitoneal (ip) or intravenous tail injections (iv). Mice are administered with MSH3 or control dsRNA agents (optionally encapsulated in LNP) up to 2×/week for maximum 8 weeks of treatment. Animals are euthanized at multiple time points and tissues collected for molecular analyses. Suitable tissues are quadriceps, heart, diaphragm, cortex, cerebellum, sperm, kidney, and liver. Genomic DNA is extracted and the length of CAG repeats measured and compared with parallel controls.

The Hdh^{Q111} mouse model for Huntington Disease is a heterozygous knock-in line, in which the majority of exon 1 and part of intron 1 on one allele of the huntingtin gene (i.e., HTT or Huntington Disease gene) are replaced with human DNA containing ˜111 CAG repeats. In this example, ASOs to knock down MSH3 activity or levels is administered. After a treatment period, brain tissue from treated or untreated mice is isolated (e.g., striatum tissue) and analyzed using qRT-PCR as previously described to determine RNA levels of MSH3. Huntingtin gene repeat analysis is performed using mouse tissues (e.g., striatum tissue) after a treatment period using a human-specific PCR assay that amplifies the HTT CAG repeat from the knock-in allele but does not amplify the mouse sequence (i.e., the wild type allele). In this protocol, the forward primer is fluorescently labeled (e.g., with 6-FAM as described previously, for example Pinto R M, Dragileva E, Kirby A, et al. Mismatch repair genes MLH1 and MSH3 modify CAG instability in Huntington's disease mice: genome-wide and candidate approaches. PLoS Genet. 2013; 9(10):e1003930.), and products can be resolved using an analyzer with comparison against an internal size standard to generate CAG repeat size distribution traces. Repeat size is determined from the peak with the greatest intensity from a control tissue (e.g., tail tissue in a mouse) and from an affected tissue (e.g., brain striatum tissue or brain cortex tissue). Immunohistochemistry is carried out with polyclonal anti-huntingtin antibody (e.g., EM48) on paraffin-embedded or otherwise prepared sections of brain tissue and can be quantified using a standardized staining index to capture both nuclear staining intensity and number of stained nuclei. A decrease in repeat size in affected tissue when compared with controls indicates that the agent that reduces the level and/or activity of MSH3 is capable of decreasing the repeat which are responsible for the toxic and/or defective gene products in Huntington's disease.

### Example 6. In Vitro Screening of MSH3 Knockdown

Knockdown of MSH3 in HeLa cells transfected with 10 nM dsRNA is shown in Table 12.

Two different screening protocols were utilized to screen for siRNA duplexes targeting human MSH3 to determine MSH3 knockdown, as described below.

### Screening Protocol 1

### Human Cell Lines

All human MSH3 targets have been screened in HeLa cells. HeLa cells were obtained from the ATCC (ATCC in partnership with LGC Standards, Wesel, Germany, cat. #ATCC-CRM-CCL-2) and cultured in HAM's F12 (#FG0815, Biochrom, Berlin, Germany), supplemented to contain 10% fetal calf serum (1248D, Biochrom GmbH, Berlin, Germany), and 100 U/ml Penicillin/100 μg/ml Streptomycin (A2213, Biochrom GmbH, Berlin, Germany) at 37° C. in an atmosphere with 5% CO2 in a humidified incubator. For transfection of HeLa cells with siRNAs, cells were seeded at a density of 15,000 cells/well into 96-well tissue culture plates (#655180, GBO, Germany).

PC3 cells were obtained from ATCC (ATCC in partnership with LGC Standards, Wesel, Germany, cat. #ATCC-CRL-1435) and cultured in RPMI 1640 (#FG1215, Biochrom, Berlin, Germany), supplemented to contain 10% fetal calf serum (#1248D, Biochrom GmbH, Berlin, Germany), 25 mM Hepes (#1615, Biochrom, Berlin, Germany), 1×non-essential amino acids (#K0293; Biochrom, Berlin, Germany), 1 mM Na-Pyruvate (#10473; Biochrom, Berlin, Germany) and 100 U/ml Penicillin/100 μg/ml Streptomycin (A2213, Biochrom GmbH, Berlin, Germany) at 37° C. in an atmosphere with 5% CO2 in a humidified incubator. For transfection of PC3 cells with siRNAs, cells were seeded at a density of 15,000 cells/well into 96-well tissue culture plates (#655180, GBO, Germany).

### Transfection

In all cell lines used, transfection of siRNA was carried out with Lipofectamine RNAiMax (Invitrogen/Life Technologies, Karlsruhe, Germany) according to the manufacturer's instructions for reverse transfection. The dual dose screen was performed with siRNAs in quadruplicates at 10 nM and 0.5 nM, respectively, with siRNAs targeting Aha1, Firefly-Luciferase and Factor VII as unspecific controls and a mock transfection. Dose-response experiments were done with siRNA in 10 concentrations transfected in quadruplicates, starting at 100 nM in 6-fold dilutions steps down to ˜10 fM. Mock transfected cells served as control in DRC experiments. After 24 h of incubation with siRNAs, medium was removed and cells were lysed in 150 μl Medium-Lysis Mixture (1 volume lysis mixture, 2 volumes cell culture medium) and then incubated at 53° C. for 30 minutes. bDNA assay was performed according to manufacturer's instructions. Luminescence was read using 1420 Luminescence Counter (WALLAC VICTOR Light, Perkin Elmer, Rodgau-Jügesheim, Germany) following 30 minutes incubation at RT in the dark.

The Aha1-siRNA served at the same time as an unspecific control for respective target mRNA expression and as a positive control to analyze transfection efficiency with regards to Aha1 mRNA level. By hybridization with an Aha1 probeset, the other two target-unspecific controls served as controls for Aha1 mRNA level. Transfection efficiency for each 96-well plate and both doses in the dual dose screen was calculated by relating Aha1-level in wells with Aha1-siRNA (normalized to GapDH) to Aha1-level obtained with controls.

For each well, the target mRNA level was normalized to the respective GAPDH mRNA level. The activity of a given siRNA was expressed as percent mRNA concentration of the respective target (normalized to GAPDH mRNA) in treated cells, relative to the target mRNA concentration (normalized to GAPDH mRNA) averaged across control wells.

### Protocol 2

### 1. Cell Seeding Density Evaluation

HeLa cells were optimized for growth rate over 72 h in 384 well plate format. The optimal cell seeding density was 5,000 HeLa cells per well. This allowed for efficient reverse transfection and sufficient mRNA to be measured by RTqPCR

### 2. Transfection Optimization

HeLa cells were reverse transfected with 10 nM and 25 nM siRNA for the following controls: NT2 (siGENOME Non-targeting Control siRNA #2, Dharmacon D-001210-02) and siTox (AllStars Hs Cell Death Control siRNA, Qiagen SI04381048) with concentrations of Lipofectamine RNAiMAX (Catalog #13778150, ThermoFisher Scientific) ranging from 0.03-0.25 μL per well. Transfection was performed in four replicates per control and per amount of Lipofectamine RNAiMAX. After 72 h the viability of the HeLa cells were measured using CTG2.0 Assay (CellTitre-Glo 2.0, Promega G924C) according to manufacturer's instructions. Briefly, a reagent volume equal to the amount of media was added per well, followed by a five-minute lysis reaction on an orbital shaker. Following a ten-minute incubation at room temperature, luminescence was measured. Lipofectamine RNAiMAX transfection reagent concentration was optimised at 0.12 μL per well (in a 384 well plate).

### 3. RT-qPCR Assay Optimization:

HeLa cells were reverse transfected with 10 nM and 0.5 nM siRNA for the following controls: NT2 (siGENOME Non-targeting Control siRNA #2, Dharmacon D-001210-02) and siGAPDH (siGENOME GAPDH, Dharmacon M-004253-02) using Lipofectamine RNAiMAX at 0.12 μL per well (in a 384 well plate). Twenty fours after transfection, cells were processed for RT-qPCR read-out using the Cellsto-CT 1-step TaqMan Kit (Invitrogen 4391852C and 4444436) following the manufacturer's instructions.

Briefly, cells were washed with 50 μl PBS and then lysed in 20 μl Lysis solution containing DNase I. After 5 min, lysis was stopped by addition of 2 μl STOP Solution for 2 min. Lysates were kept at −20° C. until RT-qPCR analysis or on ice for immediate RT-qPCR analysis. Cell lysates were diluted 1:1 with H2O. 3 μl of lysate was used as template in a 11 μl reaction volume.

Expression levels of GAPDH (TaqMan 4310884E) and GUSB (TaqMan 4333767F) were determined using RT-qPCR (Cells-to-CT 1-step TaqMan Kit) on a QuantStudio 6 (QS6) thermocycling instrument (Applied BioSystems). Relative quantification was determined using the ΔΔCT method, where a duplexed control GUSB was used and expression changes normalized to the reference sample (plate average of negative control siRNA transfected cells) set to 1.

### 4. RNAi Screen

All 1080 siRNA duplexes were resuspended in UltraPure DNase and RNase free distilled water (Invitrogen, 10977035) at 1000-fold their final assay concentration (10 μM or 0.5 μM). siRNA duplexes were dispensed in quadruplicates at 25 nL per well using the Echo 525 acoustic dispenser (LabCyte). These assay plates containing siRNA duplexes were stored at −80° C. until reverse transfection of siRNA duplexes were allowed to complex with 5 μL of Lipofectamine RNAiMAX for 20 minutes before HeLa cells were added at 5,000 cells per well (20 μl). Assay plates were kept in a cell culture incubator for 24 hours. RT-qPCR readout (using Cells-to-CT 1-step TaqMan protocol) was performed as described above.

The sense and antisense oligonucleotides of Table 12 each contain a dTdT overhang on the 3′ end. Additionally, every A and G in each sense oligonucleotide in Table 12 is a ribonucleotide. Every C and U in each sense oligonucleotide is a 2′-O-Methyl ribonucleotide.

Also, every A and G in each antisense oligonucleotide in Table 12 is a ribonucleotide. Every C and U preceding an A in the antisense oligonucleotide is a 2′-O-Methyl ribonucleotide, with one exception: U is the first nucleotide of the antisense strand, and it is a ribonucleotide.

Each nucleotide in the sense and antisense oligonucleotide in Table 12 is linked by a phosphate.

TABLE 12

| SEQ | | | | | | | |
|---|---|---|---|---|---|---|---|
| ID | | | | | | | |
| NO/ | | | SEQ | Coordinates | | Mean % mRNA | |
| SENSE | | | ID NO/ | in | | remaining | |
| OLIGO | | | ANTISENSE | NM_002439.4 | | | Std. |
| NO | Sense | Antisense | OLIGO NO | Begin | End | Mean | Dev. |
| 78 | ACUCUGAGCCAAAGAAAUA | UAUUUCUUUGGCUCAGAGU | 79 | 433 | 450 | 29.16% | 1.30% |
| 82 | UGAGCCAAAGAAAUGUCUA | UAGACAUUUCUUUGGCUCA | 83 | 437 | 454 | 28.48% | 3.21% |
| 104 | CUGCCCUUCCUCAAAGUAA | UUACUUUGAGGAAGGGCAG | 105 | 511 | 528 | 66.45% | 13.29% |
| 148 | AGUUCUGCCAAAAUGUACA | UGUACAUUUUGGCAGAACU | 149 | 563 | 580 | 41.92% | 0.95% |
| 158 | GUACUGAUUUUGAUGAUAA | UUAUCAUCAAAAUCAGUAC | 159 | 577 | 594 | 34.73% | 1.63% |
| 160 | ACUGAUUUUGAUGAUAUCA | UGAUAUCAUCAAAAUCAGU | 161 | 579 | 596 | 39.18% | 6.08% |
| 190 | AAGAAUGCAGUUUCUUCUA | UAGAAGAAACUGCAUUCUU | 191 | 612 | 629 | 34.71% | 1.43% |
| 240 | CAGUUUGGAUCAUCAAAUA | UAUUUGAUGAUCCAAACUG | 241 | 684 | 701 | 34.87% | 0.56% |
| 244 | GUUUGGAUCAUCAAAUACA | UGUAUUUGAUGAUCCAAAC | 245 | 686 | 703 | 30.29% | 0.25% |
| 246 | UUUGGAUCAUCAAAUACAA | UUGUAUUUGAUGAUCCAAA | 247 | 687 | 704 | 36.80% | 5.06% |
| 250 | UGGAUCAUCAAAUACAAGA | UCUUGUAUUUGAUGAUCCA | 251 | 689 | 706 | 42.39% | 1.25% |
| 252 | GGAUCAUCAAAUACAAGUA | UACUUGUAUUUGAUGAUCC | 253 | 690 | 707 | 37.57% | 4.10% |
| 260 | UACAAGUCAUGAAAAUUUA | UAAAUUUUCAUGACUUGUA | 261 | 701 | 718 | 33.20% | 3.47% |
| 308 | CCGCUAGAAUUACAAUACA | UGUAUUGUAAUUCUAGCGG | 309 | 771 | 788 | 28.38% | 2.64% |
| 314 | AGAAUUACAAUACAUAGAA | UUCUAUGUAUUGUAAUUCU | 315 | 776 | 793 | 28.83% | 0.31% |
| 316 | GAAUUACAAUACAUAGAAA | UUUCUAUGUAUUGUAAUUC | 317 | 777 | 794 | 33.30% | 2.34% |
| 354 | GUGUGGAAUGUGGAUAUAA | UUAUAUCCACAUUCCACAC | 355 | 826 | 843 | 41.99% | 10.04% |
| 356 | UGUGGAAUGUGGAUAUAAA | UUUAUAUCCACAUUCCACA | 357 | 827 | 844 | 28.56% | 4.30% |
| 360 | UGGAAUGUGGAUAUAAGUA | UACUUAUAUCCACAUUCCA | 361 | 829 | 846 | 29.53% | 4.53% |
| 362 | GGAAUGUGGAUAUAAGUAA | UUACUUAUAUCCACAUUCC | 363 | 830 | 847 | 51.52% | 3.12% |
| 364 | GAAUGUGGAUAUAAGUAUA | UAUACUUAUAUCCACAUUC | 365 | 831 | 848 | 23.70% | 0.44% |
| 368 | AUGUGGAUAUAAGUAUAGA | UCUAUACUUAUAUCCACAU | 369 | 833 | 850 | 28.60% | 3.91% |
| 370 | UGUGGAUAUAAGUAUAGAA | UUCUAUACUUAUAUCCACA | 371 | 834 | 851 | 56.11% | 7.06% |
| 372 | UGGAUAUAAGUAUAGAUUA | UAAUCUAUACUUAUAUCCA | 373 | 836 | 853 | 66.22% | 6.36% |
| 396 | GAGCUCAAUAUUUAUUGCA | UGCAAUAAAUAUUGAGCUC | 379 | 885 | 902 | 37.54% | 3.89% |
| 414 | UUUAGAUCACAACUUUAUA | UAUAAAGUUGUGAUCUAAA | 415 | 905 | 922 | 37.46% | 0.99% |
| 416 | UUAGAUCACAACUUUAUGA | UCAUAAAGUUGUGAUCUAA | 417 | 906 | 923 | 50.98% | 4.13% |
| 418 | UAGAUCACAACUUUAUGAA | UUCAUAAAGUUGUGAUCUA | 419 | 907 | 924 | 28.29% | 0.22% |
| 474 | GGUGGCAAAAGGAUAUAAA | UUUAUAUCCUUUUGCCACC | 475 | 971 | 988 | 28.32% | 2.21% |
| 476 | GUGGCAAAAGGAUAUAAGA | UCUUAUAUCCUUUUGCCAC | 477 | 972 | 989 | 33.27% | 0.44% |
| 478 | UGGCAAAAGGAUAUAAGGA | UCCUUAUAUCCUUUUGCCA | 479 | 973 | 990 | 47.43% | 20.58% |
| 480 | GGCAAAAGGAUAUAAGGUA | UACCUUAUAUCCUUUUGCC | 481 | 974 | 991 | 25.90% | 4.29% |
| 502 | GUUGUGAAGCAAACUGAAA | UUUCAGUUUGCUUCACAAC | 503 | 996 | 1013 | 28.49% | 2.40% |
| 512 | CUGAAACUGCAGCAUUAAA | UUUAAUGCUGCAGUUUCAG | 513 | 1009 | 1026 | 40.65% | 4.27% |
| 552 | ACAGAAGUUCACUCUUUUA | UAAAAGAGUGAACUUCUGU | 553 | 1042 | 1059 | 32.99% | 2.47% |
| 558 | UCUUUUCCCGGAAAUUGAA | UUCAAUUUCCGGGAAAAGA | 559 | 1054 | 1071 | 29.47% | 2.18% |
| 560 | CUUUUCCCGGAAAUUGACA | UGUCAAUUUCCGGGAAAAG | 561 | 1055 | 1072 | 41.39% | 2.43% |
| 582 | CCUUUAUACAAAAUCUACA | UGUAGAUUUUGUAUAAAGG | 583 | 1076 | 1093 | 74.65% | 11.91% |
| 616 | UGUGAAUCCCCUAAUCAAA | UUUGAUUAGGGGAUUCACA | 617 | 1109 | 1126 | 43.41% | 3.79% |
| 618 | GUGAAUCCCCUAAUCAAGA | UCUUGAUUAGGGGAUUCAC | 619 | 1110 | 1127 | 36.19% | 3.08% |
| 634 | UGGAUGAUGCUGUAAAUGA | UCAUUUACAGCAUCAUCCA | 635 | 1129 | 1146 | 61.80% | 10.95% |
| 636 | GAUGAUGCUGUAAAUGUUA | UAACAUUUACAGCAUCAUC | 637 | 1131 | 1148 | 23.42% | 1.99% |
| 642 | UAAAUGUUGAUGAGAUAAA | UUUAUCUCAUCAACAUUUA | 643 | 1141 | 1158 | 46.72% | 7.85% |
| 646 | UGUUGAUGAGAUAAUGACA | UGUCAUUAUCUCAUCAACA | 647 | 1145 | 1162 | 29.37% | 1.10% |
| 648 | GUUGAUGAGAUAAUGACUA | UAGUCAUUAUCUCAUCAAC | 649 | 1146 | 1163 | 16.93% | 2.14% |
| 656 | AGAUAAUGACUGAUACUUA | UAAGUAUCAGUCAUUAUCU | 657 | 1153 | 1170 | 22.55% | 2.42% |
| 660 | AUAAUGACUGAUACUUCUA | UAGAAGUAUCAGUCAUUAU | 661 | 1155 | 1172 | 45.26% | 4.42% |
| 690 | AUCUCUGAAAAUAAGGAAA | UUUCCUUAUUUUCAGAGAU | 691 | 1191 | 1208 | 27.95% | 1.45% |
| 692 | GAAAAUAAGGAAAAUGUUA | UAACAUUUUCCUUAUUUUC | 693 | 1197 | 1214 | 32.77% | 4.88% |
| 718 | GAGGUUGUGUUUGAUAGUA | UACUAUCAAACACAACCUC | 719 | 1275 | 1292 | 25.27% | 1.85% |
| 720 | AGGUUGUGUUUGAUAGUUA | UAACUAUCAAACACAACCU | 721 | 1276 | 1293 | 30.55% | 1.57% |
| 722 | GGUUGUGUUUGAUAGUUUA | UAAACUAUCAAACACAACC | 723 | 1277 | 1294 | 29.47% | 1.48% |
| 796 | GUGCAGGAUGACAGAAUUA | UAAUUCUGUCAUCCUGCAC | 797 | 1422 | 1439 | 33.29% | 3.25% |
| 820 | GAAAGGAUGGAUAACAUUA | UAAUGUUAUCCAUCCUUUC | 821 | 1446 | 1463 | 28.18% | 2.33% |
| 822 | AAAGGAUGGAUAACAUUUA | UAAAUGUUAUCCAUCCUUU | 823 | 1447 | 1464 | 34.97% | 4.72% |
| 826 | GGAUAACAUUUAUUUUGAA | UUCAAAAUAAAUGUUAUCC | 827 | 1454 | 1471 | 29.04% | 3.27% |
| 848 | CAGUUACAGAGUUUUAUGA | UCAUAAAACUCUGUAACUG | 849 | 1492 | 1509 | 46.08% | 2.51% |
| 852 | UUACAGAGUUUUAUGCAAA | UUUGCAUAAAACUCUGUAA | 853 | 1495 | 1512 | 35.97% | 5.19% |
| 854 | UACAGAGUUUUAUGCAAAA | UUUUGCAUAAAACUCUGUA | 855 | 1496 | 1513 | 30.67% | 1.55% |
| 900 | UUAUUUCUGGCAUUGUUAA | UUAACAAUGCCAGAAAUAA | 901 | 1543 | 1560 | 41.90% | 0.76% |
| 914 | UAGAGAAGCCUGUGAUUUA | UAAAUCACAGGCUUCUCUA | 915 | 1564 | 1581 | 32.55% | 3.19% |
| 928 | CUUUGGCUGCCAUCAUAAA | UUUAUGAUGGCAGCCAAAG | 929 | 1585 | 1602 | 37.53% | 0.23% |
| 930 | UUUGGCUGCCAUCAUAAAA | UUUUAUGAUGGCAGCCAAA | 931 | 1586 | 1603 | 62.24% | 6.30% |
| 934 | GGCUGCCAUCAUAAAAUAA | UUAUUUUAUGAUGGCAGCC | 935 | 1589 | 1606 | 75.79% | 3.96% |
| 936 | GCUGCCAUCAUAAAAUACA | UGUAUUUUAUGAUGGCAGC | 937 | 1590 | 1607 | 39.73% | 1.77% |
| 946 | UACCUCAAAGAAUUCAACA | UGUUGAAUUCUUUGAGGUA | 947 | 1605 | 1622 | 39.67% | 5.42% |
| 948 | CCUCAAAGAAUUCAACUUA | UAAGUUGAAUUCUUUGAGG | 949 | 1607 | 1624 | 38.11% | 2.55% |
| 966 | UCUCCAAACCUGAGAAUUA | UAAUUCUCAGGUUUGGAGA | 967 | 1636 | 1653 | 29.07% | 4.75% |
| 970 | AACCUGAGAAUUUUAAACA | UGUUUAAAAUUCUCAGGUU | 971 | 1642 | 1659 | 63.55% | 10.21% |
| 972 | CCUGAGAAUUUUAAACAGA | UCUGUUUAAAAUUCUCAGG | 973 | 1644 | 1661 | 36.98% | 1.63% |
| 988 | GCUAUCAAGUAAAAUGGAA | UUCCAUUUUACUUGAUAGC | 989 | 1661 | 1678 | 35.63% | 2.07% |
| 990 | CUAUCAAGUAAAAUGGAAA | UUUCCAUUUUACUUGAUAG | 991 | 1662 | 1679 | 35.08% | 2.46% |
| 992 | UAUCAAGUAAAAUGGAAUA | UAUUCCAUUUUACUUGAUA | 993 | 1663 | 1680 | 39.55% | 2.94% |
| 994 | UCAAGUAAAAUGGAAUUUA | UAAAUUCCAUUUUACUUGA | 995 | 1665 | 1682 | 41.67% | 0.76% |
| 996 | CAAGUAAAAUGGAAUUUAA | UUAAAUUCCAUUUUACUUG | 997 | 1666 | 1683 | 46.47% | 5.33% |
| 1006 | UUAUGACAAUUAAUGGAAA | UUUCCAUUAAUUGUCAUAA | 1007 | 1681 | 1698 | 33.49% | 0.60% |
| 1020 | GGAACAACAUUAAGGAAUA | UAUUCCUUAAUGUUGUUCC | 1021 | 1695 | 1712 | 38.00% | 1.90% |
| 1032 | CUGGAAAUCCUACAGAAUA | UAUUCUGUAGGAUUUCCAG | 1032 | 1713 | 1730 | 40.41% | 2.43% |
| 1054 | UCAGACUGAUAUGAAAACA | UGUUUUCAUAUCAGUCUGA | 1055 | 1730 | 1747 | 68.98% | 8.10% |
| 1056 | GACUGAUAUGAAAACCAAA | UUUGGUUUUCAUAUCAGUC | 1057 | 1733 | 1750 | 39.79% | 1.76% |
| 1058 | ACUGAUAUGAAAACCAAAA | UUUUGGUUUUCAUAUCAGU | 1059 | 1734 | 1751 | 38.68% | 2.75% |
| 1076 | UUGCUGUGGGUUUUAGACA | UGUCUAAAACCCACAGCAA | 1077 | 1758 | 1775 | 121.44% | 74.40% |
| 1088 | GUUUUAGACCACACUAAAA | UUUUAGUGUGGUCUAAAAC | 1089 | 1767 | 1784 | 86.75% | 7.70% |
| 1096 | AGACCACACUAAAACUUCA | UGAAGUUUUAGUGUGGUCU | 1097 | 1772 | 1789 | 52.58% | 5.39% |
| 1098 | CCACACUAAAACUUCAUUA | UAAUGAAGUUUUAGUGUGG | 1099 | 1775 | 1792 | 76.51% | 11.60% |
| 1110 | GGGAGACGGAAGUUAAAGA | UCUUUAACUUCCGUCUCCC | 1111 | 1794 | 1811 | 37.03% | 3.68% |
| 1112 | GGAGACGGAAGUUAAAGAA | UUCUUUAACUUCCGUCUCC | 1113 | 1795 | 1812 | 40.15% | 4.44% |
| 1126 | CCCAGCCACUCCUUAAAUA | UAUUUAAGGAGUGGCUGGG | 1127 | 1822 | 1839 | 58.33% | 3.02% |
| 1214 | UUUGGUCAGAUAGAAAAUA | UAUUUUCUAUCUGACCAAA | 1215 | 1902 | 1919 | 45.68% | 5.83% |
| 1220 | CAGAUAGAAAAUCAUCUAA | UUAGAUGAUUUUCUAUCUG | 1221 | 1908 | 1925 | 56.12% | 2.65% |
| 1230 | AAUCAUCUACGUAAAUUGA | UCAAUUUACGUAGAUGAUU | 1230 | 1917 | 1934 | 71.94% | 2.14% |
| 1306 | AGAAUUUCAAGCAAUAAUA | UAUUAUUGCUUGAAAUUCU | 1307 | 2030 | 2047 | 36.86% | 0.88% |
| 1308 | AUUUCAAGCAAUAAUACCA | UGGUAUUAUUGCUUGAAAU | 1309 | 2033 | 2050 | 43.83% | 5.54% |
| 1310 | UUUCAAGCAAUAAUACCUA | UAGGUAUUAUUGCUUGAAA | 1311 | 2034 | 2051 | 36.53% | 1.45% |
| 1318 | AUACCUGCUGUUAAUUCCA | UGGAAUUAACAGCAGGUAU | 1319 | 2046 | 2063 | 44.51% | 3.47% |
| 1326 | ACCGUUAUUUUAGAAAUUA | UAAUUUCUAAAAUAACGGU | 1363 | 2088 | 2105 | 40.01% | 3.36% |
| 1386 | UCCAGUGGAGCAUUACUUA | UAAGUAAUGCUCCACUGGA | 1387 | 2120 | 2137 | 37.78% | 4.62% |
| 1394 | GAGCAUUACUUAAAGAUAA | UUAUCUUUAAGUAAUGCUC | 1395 | 2127 | 2144 | 42.33% | 1.87% |
| 1396 | AGCAUUACUUAAAGAUACA | UGUAUCUUUAAGUAAUGCU | 1397 | 2128 | 2145 | 38.19% | 0.66% |
| 1400 | UACUUAAAGAUACUCAAUA | UAUUGAGUAUCUUUAAGUA | 1401 | 2133 | 2150 | 88.29% | 67.87% |
| 1404 | UUAAAGAUACUCAAUGAAA | UUUCAUUGAGUAUCUUUAA | 1405 | 2136 | 2153 | 34.87% | 2.77% |
| 1424 | GUUGGGGAUAAAACUGAAA | UUUCAGUUUUAUCCCCAAC | 1425 | 2166 | 2183 | 37.58% | 1.87% |
| 1426 | UUGGGGAUAAAACUGAAUA | UAUUCAGUUUUAUCCCCAA | 1427 | 2167 | 2184 | 45.94% | 2.86% |
| 1448 | UUCUGACUUCCCUUUAAUA | UAUUAAAGGGAAGUCAGAA | 1449 | 2198 | 2215 | 42.92% | 2.41% |
| 1452 | UGACUUCCCUUUAAUAAAA | UUUUAUUAAAGGGAAGUCA | 1453 | 2201 | 2218 | 34.18% | 1.53% |
| 1454 | AGAGGAAGGAUGAAAUUCA | UGAAUUUCAUCCUUCCUCU | 1455 | 2221 | 2238 | 38.71% | 4.13% |
| 1506 | AUCCUUCUGCACAAUAUGA | UCAUAUUGUGCAGAAGGAU | 1507 | 2296 | 2313 | 78.53% | 13.44% |
| 1524 | CAGGACAGGAGUUUAUGAA | UUCAUAAACUCCUGUCCUG | 1525 | 2323 | 2340 | 41.36% | 5.28% |
| 1540 | AGAACUCUGCUGUAUCUUA | UAAGAUACAGCAGAGUUCU | 1541 | 2350 | 2367 | 39.12% | 1.41% |
| 1546 | CUCUGCUGUAUCUUGUAUA | UAUACAAGAUACAGCAGAG | 1547 | 2354 | 2371 | 42.74% | 3.97% |
| 1656 | GAAUGGCUUGAUUUUCUAA | UUAGAAAAUCAAGCCAUUC | 1657 | 2496 | 2513 | 43.75% | 3.47% |
| 1666 | UUUCUAGAGAAAUUCAGUA | UACUGAAUUUCUCUAGAAA | 1667 | 2508 | 2525 | 56.85% | 11.91% |
| 1674 | AAAUUCAGUGAACAUUAUA | UAUAAUGUUCACUGAAUUU | 1675 | 2517 | 2534 | 45.05% | 5.48% |
| 1676 | AAUUCAGUGAACAUUAUCA | UGAUAAUGUUCACUGAAUU | 1677 | 2518 | 2535 | 29.77% | 2.22% |
| 1678 | AUUCAGUGAACAUUAUCAA | UUGAUAAUGUUCACUGAAU | 1679 | 2519 | 2536 | 36.74% | 3.41% |
| 1722 | UGUUGACUGCAUUUUCUCA | UGAGAAAAUGCAGUCAACA | 1723 | 2570 | 2587 | 34.29% | 3.73% |
| 1762 | AGACCAACUGUACAAGAAA | UUUCUUGUACAGUUGGUCU | 1763 | 2622 | 2639 | 37.30% | 3.16% |
| 1766 | ACUGUACAAGAAGAAAGAA | UUCUUUCUUCUUGUACAGU | 1767 | 2628 | 2645 | 54.43% | 4.31% |
| 1768 | CUGUACAAGAAGAAAGAAA | UUUCUUUCUUCUUGUACAG | 1769 | 2629 | 2646 | 33.89% | 5.86% |
| 1836 | ACUCAGAGAGAGUAAUGAA | UUCAUUACUCUCUCUGAGU | 1837 | 2740 | 2757 | 29.94% | 3.38% |
| 1838 | UCAGAGAGAGUAAUGAUAA | UUAUCAUUACUCUCUCUGA | 1839 | 2742 | 2759 | 32.69% | 1.16% |
| 1842 | AGAGAGAGUAAUGAUAAUA | UAUUAUCAUUACUCUCUCU | 1843 | 2744 | 2761 | 35.60% | 3.31% |
| 1868 | GAAAGAGCUCCUACAUAAA | UUUAUGUAGGAGCUCUUUC | 1869 | 2782 | 2799 | 36.26% | 1.38% |
| 1886 | AAACAAGUUGCAUUGAUUA | UAAUCAAUGCAACUUGUUU | 1887 | 2799 | 2816 | 48.40% | 12.84% |
| 1888 | AACAAGUUGCAUUGAUUAA | UUAAUCAAUGCAACUUGUU | 1889 | 2800 | 2817 | 63.96% | 2.64% |
| 1964 | GUGGAUGGCAUUUUCACAA | UUGUGAAAAUGCCAUCCAC | 1965 | 2874 | 2891 | 49.72% | 1.59% |
| 1990 | UGCAGACAAUAUAUAUAAA | UUUAUAUAUAUUGUCUGCA | 1991 | 2903 | 2920 | 47.35% | 2.46% |
| 2030 | GACACAGCAGAAAUAAUCA | UGAUUAUUUCUGCUGUGUC | 2031 | 2952 | 2969 | 43.67% | 8.62% |
| 2108 | AUGCUACACUUGAGUAUUA | UAAUACUCAAGUGUAGCAU | 2109 | 3052 | 3069 | 41.76% | 2.04% |
| 2128 | GAGAUGUGAAAUCCUUAAA | UUUAAGGAUUUCACAUCUC | 2129 | 3076 | 3093 | 62.62% | 4.94% |
| 2230 | AACAAGUCCCUGAUUUUGA | UCAAAAUCAGGGACUUGUU | 2231 | 3220 | 3237 | 73.43% | 10.39% |
| 2242 | UUUUGUCACCUUCCUUUAA | UUAAAGGAAGGUGACAAAA | 2243 | 3233 | 3250 | 49.17% | 4.42% |
| 2246 | UCACCUUCCUUUACCAAAA | UUUUGGUAAAGGAAGGUGA | 2247 | 3238 | 3255 | 44.74% | 5.16% |
| 2254 | CUUUACCAAAUAACUAGAA | UUCUAGUUAUUUGGUAAAG | 2255 | 3246 | 3263 | 39.87% | 3.77% |
| 2274 | AAGGAGUUAUGGAUUAAAA | UUUUAAUCCAUAACUCCUU | 2275 | 3275 | 3292 | 41.52% | 1.53% |
| 2294 | UAAAUGUGGCUAAACUAGA | UCUAGUUUAGCCACAUUUA | 2295 | 3289 | 3306 | 39.85% | 2.79% |
| 2330 | GAGCUGGAAGGAUUAAUAA | UUAUUAAUCCUUCCAGCUC | 2331 | 3354 | 3371 | 56.05% | 14.99% |
| 2334 | GGAUUAAUAAAUACGAAAA | UUUUCGUAUUUAUUAAUCC | 2335 | 3363 | 3380 | 52.40% | 5.26% |
| 2356 | ACUCAAGUAUUUUGCAAAA | UUUUGCAAAAUACUUGAGU | 2357 | 3389 | 3406 | 35.71% | 2.25% |
| 2360 | AAGUAUUUUGCAAAGUUAA | UUAACUUUGCAAAAUACUU | 2361 | 3393 | 3410 | 41.68% | 2.43% |
| 2362 | GUAUUUUGCAAAGUUAUGA | UCAUAACUUUGCAAAAUAC | 2363 | 3395 | 3412 | 45.57% | 7.28% |
| 2448 | CAACUGUACAAAAUAACUA | UAGUUAUUUUGUACAGUUG | 2449 | 3540 | 3557 | 59.64% | 10.75% |
| 2502 | ACAUGUGAGCAUAAAAUUA | UAAUUUUAUGCUCACAUGU | 2503 | 3584 | 3601 | 40.95% | 4.16% |
| 2504 | CAUGUGAGCAUAAAAUUAA | UUAAUUUUAUGCUCACAUG | 2505 | 3585 | 3602 | 56.03% | 3.39% |
| 2516 | AAUUAUGACCAUGGUAUAA | UUAUACCAUGGUCAUAAUU | 2517 | 3598 | 3615 | 53.24% | 2.72% |
| 2518 | AUUAUGACCAUGGUAUAUA | UAUAUACCAUGGUCAUAAU | 2519 | 3599 | 3616 | 67.48% | 5.75% |
| 2578 | AUAAACACUCUUGAAUAGA | UCUAUUCAAGAGUGUUUAU | 2579 | 3689 | 3706 | 55.57% | 9.42% |
| 2580 | UAAACACUCUUGAAUAGAA | UUCUAUUCAAGAGUGUUUA | 2581 | 3690 | 3707 | 42.79% | 1.54% |
| 2592 | AUAGACUUCCACUUUGUAA | UUACAAAGUGGAAGUCUAU | 2593 | 3703 | 3720 | 65.27% | 6.23% |
| 2596 | AGACUUCCACUUUGUAAUA | UAUUACAAAGUGGAAGUCU | 2597 | 3705 | 3722 | 58.85% | 9.89% |
| 2602 | CUUCCACUUUGUAAUUAGA | UCUAAUUACAAAGUGGAAG | 2603 | 3708 | 3725 | 51.93% | 7.38% |
| 2654 | CUUAAGUGGCAGAAUAUAA | UUAUAUUCUGCCACUUAAG | 2655 | 3757 | 3774 | 61.67% | 11.09% |
| 2656 | UUAAGUGGCAGAAUAUAAA | UUUAUAUUCUGCCACUUAA | 2657 | 3758 | 3775 | 48.43% | 7.02% |
| 2686 | UUUUGGAGGGUGAUAUAAA | UUUAUAUCACCCUCCAAAA | 2687 | 3785 | 3802 | 48.16% | 6.02% |
| 2762 | UCCACCAAGAACAUAAGAA | UUCUUAUGUUCUUGGUGGA | 2763 | 3912 | 3929 | 49.77% | 1.05% |
| 2768 | ACCAAGAACAUAAGAAUUA | UAAUUCUUAUGUUCUUGGU | 2769 | 3915 | 3932 | 44.17% | 3.47% |
| 2782 | UAGAAUUAUCAAGCUUUUA | UAAAAGCUUGAUAAUUCUA | 2783 | 4290 | 4307 | 53.00% | 2.15% |
| 2844 | UUGGAUGAAAUUAUUUGUA | UACAAAUAAUUUCAUCCAA | 2845 | 4389 | 4406 | 71.32% | 9.32% |
| 2846 | GAUGAAAUUAUUUGUCAUA | UAUGACAAAUAAUUUCAUC | 2847 | 4392 | 4409 | 55.83% | 6.18% |

For the chemical modifications, “2m” means 2′-O-Methyl ribonucleotides, “r” means ribonucleotide, “p” means phosphate linkage, and “d” means deoxyribonucleotide.

TABLE 13
Chemical Modifications for Sense Strands

| SEQ | | |
|---|---|---|
| ID | | |
| No./ | | |
| Sense | | |
| No. | Sense | Sense Chemical Modifications |
| 78 | ACUCUGAGCCAAAGAAAUA | [rAp\|2mCp\|2mUp\|2mCp\|2mUp\|rGp\|rAp\|rGp\|2mCp |
| | | \|2mCp\|rAp\|rAp\|rAp\|rGp\|rAp |
| | | \|rAp\|rAp\|2mUp\|rAp\|dTp\|dT] |
| 82 | UGAGCCAAAGAAAUGUCUA | [2mUp\|rGp\|rAp\|rGp\|2mCp\|2mCp\|rAp\|rAp\|rAp\|r |
| | | Gp\|rAp\|rAp\|rAp\|2mUp\|rGp\|2m |
| | | Up\|2mCp\|2mUp\|rAp\|dTp\|dT] |
| 104 | CUGCCCUUCCUCAAAGUAA | [2mCp\|2mUp\|rGp\|2mCp\|2mCp\|2mCp\|2mUp\|2mUp\|2 |
| | | mCp\|2mCp\|2mUp\|2mCp\| |
| | | rAp\|rAp\|rAp\|rGp\|2mUp\|rAp\|rAp\|dTp\|dT] |
| 148 | AGUUCUGCCAAAAUGUACA | [rAp\|rGp\|2mUp\|2mUp\|2mCp\|2mUp\|rGp\|2mCp\|2mC |
| | | p\|rAp\|r |
| | | Ap\|rAp\|rAp\|2mUp\|rGp\|2mUp\|rAp\|2mCp\|rAp\|dT |
| | | p\|dT] |
| 158 | GUACUGAUUUUGAUGAUAA | [rGp\|2mUp\|rAp\|2mCp\|2mUp\|rGp\|rAp\|2mUp\|2mUp |
| | | \|2mUp\|2mUp\|rGp\|rAp\|2mU |
| | | p\|rGp\|rAp\|2mUp\|rAp\|rAp\|dTp\|dT] |
| 160 | ACUGAUUUUGAUGAUAUCA | [rAp\|2mCp\|2mUp\|rGp\|rAp\|2mUp\|2mUp\|2mUp\|2mU |
| | | p\|rGp\|rAp\|2mUp\|rGp\|rAp\|2 |
| | | mUp\|rAp\|2mUp\|2mCp\|rAp\|dTp\|dT] |
| 190 | AAGAAUGCAGUUUCUUCUA | [rAp\|rAp\|rGp\|rAp\|rAp\|2mUp\|rGp\|2mCp\|rAp\|rG |
| | | p\|2mUp\|2mUp\|2mUp\|2mCp\|2m |
| | | Up\|2mUp\|2mCp\|2mUp\|rAp\|dTp\|dT] |
| 240 | CAGUUUGGAUCAUCAAAUA | [2mCp\|rAp\|rGp\|2mUp\|2mUp\|2mUp\|rGp\|rGp\|rAp\| |
| | | 2mUp\|2mCp\|rAp\|2mUp\|2mC |
| | | p\|rAp\|rAp\|rAp\|2mUp\|rAp\|dTp\|dT] |
| 244 | GUUUGGAUCAUCAAAUACA | [rGp\|2mUp\|2mUp\|2mUp\|rGp\|rGp\|rAp\|2mUp\|2mCp |
| | | \|rAp\|2mUp\|2mCp\|rAp\|rAp\|r |
| | | Ap\|2mUp\|rAp\|2mCp\|rAp\|dTp\|dT] |
| 246 | UUUGGAUCAUCAAAUACAA | [2mUp\|2mUp\|2mUp\|rGp\|rGp\|rAp\|2mUp\|2mCp\|rAp |
| | | \|2mUp\|2mCp\|rAp\|rAp\|rAp\|2 |
| | | mUp\|rAp\|2mCp\|rAp\|rAp\|dTp\|dT] |
| 250 | UGGAUCAUCAAAUACAAGA | [2mUp\|rGp\|rGp\|rAp\|2mUp\|2mCp\|rAp\|2mUp\|2mCp |
| | | \|rAp\|rAp\|rAp\|2mUp\|rAp\|2m |
| | | Cp\|rAp\|rAp\|rGp\|rAp\|dTp\|dT] |
| 252 | GGAUCAUCAAAUACAAGUA | [rGp\|rGp\|rAp\|2mUp\|2mCp\|rAp\|2mUp\|2mCp\|rAp\| |
| | | rAp\|rAp\|2mUp\|rAp\|2mCp\|rAp\| |
| | | rAp\|rGp\|2mUp\|rAp\|dTp\|dT] |
| 260 | UACAAGUCAUGAAAAUUUA | [2mUp\|rAp\|2mCp\|rAp\|rAp\|rGp\|2mUp\|2mCp\|rAp\| |
| | | 2mUp\|rGp\|rAp\|rAp\|rAp\|rAp\|2 |
| | | mUp\|2mUp\|2mUp\|rAp\|dTp\|dT] |
| 308 | CCGCUAGAAUUACAAUACA | [2mCp\|2mCp\|rGp\|2mCp\|2mUp\|rAp\|rGp\|rAp\|rAp\| |
| | | 2mUp\|2mUp\|rAp\|2mCp\|rAp\|r |
| | | Ap\|2mUp\|rAp\|2mCp\|rAp\|dTp\|dT] |
| 314 | AGAAUUACAAUACAUAGAA | [rAp\|rGp\|rAp\|rAp\|2mUp\|2mUp\|rAp\|2mCp\|rAp\|r |
| | | Ap\|2mUp\|rAp\|2mCp\|rAp\|2mUp\| |
| | | rAp\|rGp\|rAp\|rAp\|dTp\|dT] |
| 316 | GAAUUACAAUACAUAGAAA | [rGp\|rAp\|rAp\|2mUp\|2mUp\|rAp\|2mCp\|rAp\|rAp\|2 |
| | | mUp\|rAp\|2mCp\|rAp\|2mUp\|rAp\| |
| | | rGp\|rAp\|rAp\|rAp\|dTp\|dT] |
| 354 | GUGUGGAAUGUGGAUAUAA | [rGp\|2mUp\|rGp\|2mUp\|rGp\|rGp\|rAp\|rAp\|2mUp\|r |
| | | Gp\|2mUp\|rGp\|rGp\|rAp\|2mUp\|r |
| | | Ap\|2mUp\|rAp\|rAp\|dTp\|dT] |
| 356 | UGUGGAAUGUGGAUAUAAA | [2mUp\|rGp\|2mUp\|rGp\|rGp\|rAp\|rAp\|2mUp\|rGp\|2 |
| | | mUp\|rGp\|rGp\|rAp\|2mUp\|rAp\|2 |
| | | mUp\|rAp\|rAp\|rAp\|dTp\|dT] |
| 360 | UGGAAUGUGGAUAUAAGUA | [2mUp\|rGp\|rGp\|rAp\|rAp\|2mUp\|rGp\|2mUp\|rGp\|r |
| | | Gp\|rAp\|2mUp\|rAp\|2mUp\|rAp\|r |
| | | Ap\|rGp\|2mUp\|rAp\|dTp\|dT] |
| 362 | GGAAUGUGGAUAUAAGUAA | [rGp\|rGp\|rAp\|rAp\|2mUp\|rGp\|2mUp\|rGp\|rGp\|rA |
| | | p\|2mUp\|rAp\|2mUp\|rAp\|rAp\|rGp\| |
| | | 2mUp\|rAp\|rAp\|dTp\|dT] |
| 364 | GAAUGUGGAUAUAAGUAUA | [rGp\|rAp\|rAp\|2mUp\|rGp\|2mUp\|rGp\|rGp\|rAp\|2m |
| | | Up\|rAp\|2mUp\|rAp\|rAp\|rGp\|2m |
| | | Up\|rAp\|2mUp\|rAp\|dTp\|dT] |
| 368 | AUGUGGAUAUAAGUAUAGA | [rAp\|2mUp\|rGp\|2mUp\|rGp\|rGp\|rAp\|2mUp\|rAp\|2 |
| | | mUp\|rAp\|rAp\|rGp\|2mUp\|rAp\|2 |
| | | mUp\|rAp\|rGp\|rAp\|dTp\|dT] |
| 370 | UGUGGAUAUAAGUAUAGAA | [2mUp\|rGp\|2mUp\|rGp\|rGp\|rAp\|2mUp\|rAp\|2mUp\| |
| | | rAp\|rAp\|rGp\|2mUp\|rAp\|2mUp |
| | | \|rAp\|rGp\|rAp\|rAp\|dTp\|dT] |
| 372 | UGGAUAUAAGUAUAGAUUA | [2mUp\|rGp\|rGp\|rAp\|2mUp\|rAp\|2mUp\|rAp\|rAp\|r |
| | | Gp\|2mUp\|rAp\|2mUp\|rAp\|rGp\|r |
| | | Ap\|2mUp\|2mUp\|rAp\|dTp\|dT] |
| 396 | GAGCUCAAUAUUUAUUGCA | [rGp\|rAp\|rGp\|2mCp\|2mUp\|2mCp\|rAp\|rAp\|2mUp\| |
| | | rAp\|2mUp\|2mUp\|2mUp\|rAp\|2 |
| | | mUp\|2mUp\|rGp\|2mCp\|rAp\|dTp\|dT] |
| 414 | UUUAGAUCACAACUUUAUA | [2mUp\|2mUp\|2mUp\|rAp\|rGp\|rAp\|2mUp\|2mCp\|rAp |
| | | \|2mCp\|rAp\|rAp\|2mCp\|2mUp |
| | | 2mUp\|2mUp\|rAp\|2mUp\|rAp\|dTp\|dT] |
| 416 | UUAGAUCACAACUUUAUGA | [2mUp\|2mUp\|rAp\|rGp\|rAp\|2mUp\|2mCp\|rAp\|2mCp |
| | | \|rAp\|rAp\|2mCp\|2mUp\|2mUp |
| | | \|2mUp\|rAp\|2mUp\|rGp\|rAp\|dTp\|dT] |
| 418 | UAGAUCACAACUUUAUGAA | [2mUp\|rAp\|rGp\|rAp\|2mUp\|2mCp\|rAp\|2mCp\|rAp\| |
| | | rAp\|2mCp\|2mUp\|2mUp\|2mUp |
| | | rAp\|2mUp\|rGp\|rAp\|rAp\|dTp\|dT] |
| 474 | GGUGGCAAAAGGAUAUAAA | [rGp\|rGp\|2mUp\|rGp\|rGp\|2mCp\|rAp\|rAp\|rAp\|rA |
| | | p\|rGp\|rGp\|rAp\|2mUp\|rAp\|2mUp\| |
| | | rAp\|rAp\|rAp\|dTp\|dT] |
| 476 | GUGGCAAAAGGAUAUAAGA | [rGp\|2mUp\|rGp\|rGp\|2mCp\|rAp\|rAp\|rAp\|rAp\|rG |
| | | p\|rGp\|rAp\|2mUp\|rAp\|2mUp\|rAp\| |
| | | rAp\|rGp\|rAp\|dTp\|dT] |
| 478 | UGGCAAAAGGAUAUAAGGA | [2mUp\|rGp\|rGp\|2mCp\|rAp\|rAp\|rAp\|rAp\|rGp\|rG |
| | | p\|rAp\|2mUp\|rAp\|2mUp\|rAp\|rAp\| |
| | | rGp\|rGp\|rAp\|dTp\|dT] |
| 480 | GGCAAAAGGAUAUAAGGUA | [rGp\|rGp\|2mCp\|rAp\|rAp\|rAp\|rAp\|rGp\|rGp\|rAp |
| | | \|2mUp\|rAp\|2mUp\|rAp\|rAp\|rGp\|rG |
| | | p\|2mUp\|rAp\|dTp\|dT] |
| 502 | GUUGUGAAGCAAACUGAAA | [rGp\|2mUp\|2mUp\|rGp\|2mUp\|rGp\|rAp\|rAp\|rGp\| |
| | | 2mCp\|rAp\|rAp\|rAp\|2mCp\|2mUp |
| | | \|rGp\|rAp\|rAp\|rAp\|dTp\|dT] |
| 512 | CUGAAACUGCAGCAUUAAA | [2mCp\|2mUp\|rGp\|rAp\|rAp\|rAp\|2mCp\|2mUp\|rGp\| |
| | | 2mCp\|rAp\|rGp\|2mCp\|rAp\|2m |
| | | Up\|2mUp\|rAp\|rAp\|rAp\|dTp\|dT] |
| 552 | ACAGAAGUUCACUCUUUUA | [rAp\|2mCp\|rAp\|rGp\|rAp\|rAp\|rGp\|2mUp\|2mUp\|2 |
| | | mCp\|rAp\|2mCp\|2mUp\|2mCp\|2 |
| | | mUp\|2mUp\|2mUp\|2mUp\|rAp\|dTp\|dT] |
| 558 | UCUUUUCCCGGAAAUUGAA | [2mUp\|2mCp\|2mUp\|2mUp\|2mUp\|2mUp\|2mCp\|2mCp\| |
| | | 2mCp\|rGp\|rGp\|rAp\|rAp\|r |
| | | Ap\|2mUp\|2mUp\|rGp\|rAp\|rAp\|dTp\|dT] |
| 560 | CUUUUCCCGGAAAUUGACA | [2mCp\|2mUp\|2mUp\|2mUp\|2mUp\|2mCp\|2mCp\|2mCp\| |
| | | rGp\|rGp\|rAp\|rAp\|rAp\|2m |
| | | Up\|2mUp\|rGp\|rAp\|2mCp\|rAp\|dTp\|dT] |
| 582 | CCUUUAUACAAAAUCUACA | [2mCp\|2mCp\|2mUp\|2mUp\|2mUp\|rAp\|2mUp\|rAp\|2m |
| | | Cp\|rAp\|rAp\|rAp\|rAp\|2mUp |
| | | 2mCp\|2mUp\|rAp\|2mCp\|rAp\|dTp\|dT] |
| 616 | UGUGAAUCCCCUAAUCAAA | [2mUp\|rGp\|2mUp\|rGp\|rAp\|rAp\|2mUp\|2mCp\|2mCp |
| | | \|2mCp\|2mCp\|2mUp\|rAp\|rA |
| | | p\|2mUp\|2mCp\|rAp\|rAp\|rAp\|dTp\|dT] |
| 618 | GUGAAUCCCCUAAUCAAGA | [rGp\|2mUp\|rGp\|rAp\|rAp\|2mUp\|2mCp\|2mCp\|2mCp |
| | | \|2mCp\|2mUp\|rAp\|rAp\|2mU |
| | | p\|2mCp\|rAp\|rAp\|rGp\|rAp\|dTp\|dT] |
| 634 | UGGAUGAUGCUGUAAAUGA | [2mUp\|rGp\|rGp\|rAp\|2mUp\|rGp\|rAp\|2mUp\|rGp\|2 |
| | | mCp\|2mUp\|rGp\|2mUp\|rAp\|rAp |
| | | rAp\|2mUp\|rGp\|rAp\|dTp\|dT] |
| 636 | GAUGAUGCUGUAAAUGUUA | [rGp\|rAp\|2mUp\|rGp\|rAp\|2mUp\|rGp\|2mCp\|2mUp\| |
| | | rGp\|2mUp\|rAp\|rAp\|rAp\|2mUp |
| | | \|rGp\|2mUp\|2mUp\|rAp\|dTp\|dT] |
| 642 | UAAAUGUUGAUGAGAUAAA | [2mUp\|rAp\|rAp\|rAp\|2mUp\|rGp\|2mUp\|2mUp\|rGp\| |
| | | rAp\|2mUp\|rGp\|rAp\|rGp\|rAp\|2 |
| | | mUp\|rAp\|rAp\|rAp\|dTp\|dT] |
| 646 | UGUUGAUGAGAUAAUGACA | [2mUp\|rGp\|2mUp\|2mUp\|rGp\|rAp\|2mUp\|rGp\|rAp\| |
| | | rGp\|rAp\|2mUp\|rAp\|rAp\|2mUp |
| | | \|rGp\|rAp\|2mCp\|rAp\|dTp\|dT] |
| 648 | GUUGAUGAGAUAAUGACUA | [rGp\|2mUp\|2mUp\|rGp\|rAp\|2mUp\|rGp\|rAp\|rGp\|r |
| | | Ap\|2mUp\|rAp\|rAp\|2mUp\|rGp\|r |
| | | Ap\|2mCp\|2mUp\|rAp\|dTp\|dT] |
| 656 | AGAUAAUGACUGAUACUUA | [rAp\|rGp\|rAp\|2mUp\|rAp\|rAp\|2mUp\|rGp\|rAp\|2m |
| | | Cp\|2mUp\|rGp\|rAp\|2mUp\|rAp\|2 |
| | | mCp\|2mUp\|2mUp\|rAp\|dTp\|dT] |
| 660 | AUAAUGACUGAUACUUCUA | [rAp\|2mUp\|rAp\|rAp\|2mUp\|rGp\|rAp\|2mCp\|2mUp\| |
| | | rGp\|rAp\|2mUp\|rAp\|2mCp\|2m |
| | | Up\|2mUp\|2mCp\|2mUp\|rAp\|dTp\|dT] |
| 690 | AUCUCUGAAAAUAAGGAAA | [rAp\|2mUp\|2mCp\|2mUp\|2mCp\|2mUp\|rGp\|rAp\|rAp |
| | | \|rAp\|rAp\|2mUp\|rAp\|rAp\|rGp\| |
| | | rGp\|rAp\|rAp\|rAp\|dTp\|dT] |
| 692 | GAAAAUAAGGAAAAUGUUA | [rGp\|rAp\|rAp\|rAp\|rAp\|2mUp\|rAp\|rAp\|rGp\|rGp |
| | | \|rAp\|rAp\|rAp\|rAp\|2mUp\|rGp\|2mU |
| | | p\|2mUp\|rAp\|dTp\|dT] |
| 718 | GAGGUUGUGUUUGAUAGUA | [rGp\|rAp\|rGp\|rGp\|2mUp\|2mUp\|rGp\|2mUp\|rGp\|2 |
| | | mUp\|2mUp\|2mUp\|rGp\|rAp\|2m |
| | | Up\|rAp\|rGp\|2mUp\|rAp\|dTp\|dT] |
| 720 | AGGUUGUGUUUGAUAGUUA | [rAp\|rGp\|rGp\|2mUp\|2mUp\|rGp\|2mUp\|rGp\|2mUp\| |
| | | 2mUp\|2mUp\|rGp\|rAp\|2mUp\|r |
| | | Ap\|rGp\|2mUp\|2mUp\|rAp\|dTp\|dT] |
| 722 | GGUUGUGUUUGAUAGUUUA | [rGp\|rGp\|2mUp\|2mUp\|rGp\|2mUp\|rGp\|2mUp\|2mUp |
| | | \|2mUp\|rGp\|rAp\|2mUp\|rAp\|r |
| | | Gp\|2mUp\|2mUp\|2mUp\|rAp\|dTp\|dT] |
| 796 | GUGCAGGAUGACAGAAUUA | [rGp\|2mUp\|rGp\|2mCp\|rAp\|rGp\|rGp\|rAp\|2mUp\|r |
| | | Gp\|rAp\|2mCp\|rAp\|rGp\|rAp\|rAp\| |
| | | 2mUp\|2mUp\|rAp\|dTp\|dT] |
| 820 | GAAAGGAUGGAUAACAUUA | [rGp\|rAp\|rAp\|rAp\|rGp\|rGp\|rAp\|2mUp\|rGp\|rGp |
| | | \|rAp\|2mUp\|rAp\|rAp\|2mCp\|rAp\|2 |
| | | mUp\|2mUp\|rAp\|dTp\|dT] |
| 822 | AAAGGAUGGAUAACAUUUA | [rAp\|rAp\|rAp\|rGp\|rGp\|rAp\|2mUp\|rGp\|rGp\|rAp |
| | | \|2mUp\|rAp\|rAp\|2mCp\|rAp\|2mUp\| |
| | | 2mUp\|2mUp\|rAp\|dTp\|dT] |
| 826 | GGAUAACAUUUAUUUUGAA | [rGp\|rGp\|rAp\|2mUp\|rAp\|rAp\|2mCp\|rAp\|2mUp\|2 |
| | | mUp\|2mUp\|rAp\|2mUp\|2mUp\|2 |
| | | mUp\|2mUp\|rGp\|rAp\|rAp\|dTp\|dT] |
| 848 | CAGUUACAGAGUUUUAUGA | [2mCp\|rAp\|rGp\|2mUp\|2mUp\|rAp\|2mCp\|rAp\|rGp\| |
| | | rAp\|rGp\|2mUp\|2mUp\|2mUp\|2 |
| | | mUp\|rAp\|2mUp\|rGp\|rAp\|dTp\|dT] |
| 852 | UUACAGAGUUUUAUGCAAA | [2mUp\|2mUp\|rAp\|2mCp\|rAp\|rGp\|rAp\|rGp\|2mUp\| |
| | | 2mUp\|2mUp\|2mUp\|rAp\|2mU |
| | | p\|rGp\|2mCp\|rAp\|rAp\|rAp\|dTp\|dT] |
| 854 | UACAGAGUUUUAUGCAAAA | [2mUp\|rAp\|2mCp\|rAp\|rGp\|rAp\|rGp\|2mUp\|2mUp\| |
| | | 2mUp\|2mUp\|rAp\|2mUp\|rGp\|2 |
| | | mCp\|rAp\|rAp\|rAp\|rAp\|dTp\|dT] |
| 900 | UUAUUUCUGGCAUUGUUAA | [2mUp\|2mUp\|rAp\|2mUp\|2mUp\|2mUp\|2mCp\|2mUp\|r |
| | | Gp\|rGp\|2mCp\|rAp\|2mUp\| |
| | | 2mUp\|rGp\|2mUp\|2mUp\|rAp\|rAp\|dTp\|dT] |
| 914 | UAGAGAAGCCUGUGAUUUA | [2mUp\|rAp\|rGp\|rAp\|rGp\|rAp\|rAp\|rGp\|2mCp\|2m |
| | | Cp\|2mUp\|rGp\|2mUp\|rGp\|rAp\|2 |
| | | mUp\|2mUp\|2mUp\|rAp\|dTp\|dT] |
| 928 | CUUUGGCUGCCAUCAUAAA | [2mCp\|2mUp\|2mUp\|2mUp\|rGp\|rGp\|2mCp\|2mUp\|rG |
| | | p\|2mCp\|2mCp\|rAp\|2mUp\| |
| | | 2mCp\|rAp\|2mUp\|rAp\|rAp\|rAp\|dTp\|dT] |
| 930 | UUUGGCUGCCAUCAUAAAA | [2mUp\|2mUp\|2mUp\|rGp\|rGp\|2mCp\|2mUp\|rGp\|2mC |
| | | p\|2mCp\|rAp\|2mUp\|2mCp\| |
| | | rAp\|2mUp\|rAp\|rAp\|rAp\|rAp\|dTp\|dT] |
| 934 | GGCUGCCAUCAUAAAAUAA | [rGp\|rGp\|2mCp\|2mUp\|rGp\|2mCp\|2mCp\|rAp\|2mUp |
| | | \|2mCp\|rAp\|2mUp\|rAp\|rAp\|r |
| | | Ap\|rAp\|2mUp\|rAp\|rAp\|dTp\|dT] |
| 936 | GCUGCCAUCAUAAAAUACA | [rGp\|2mCp\|2mUp\|rGp\|2mCp\|2mCp\|rAp\|2mUp\|2mC |
| | | p\|rAp\|2mUp\|rAp\|rAp\|rAp\|r |
| | | Ap\|2mUp\|rAp\|2mCp\|rAp\|dTp\|dT] |
| 946 | UACCUCAAAGAAUUCAACA | [2mUp\|rAp\|2mCp\|2mCp\|2mUp\|2mCp\|rAp\|rAp\|rAp |
| | | \|rGp\|rAp\|rAp\|2mUp\|2mUp\|2 |
| | | mCp\|rAp\|rAp\|2mCp\|rAp\|dTp\|dT] |
| 948 | CCUCAAAGAAUUCAACUUA | [2mCp\|2mCp\|2mUp\|2mCp\|rAp\|rAp\|rAp\|rGp\|rAp\| |
| | | rAp\|2mUp\|2mUp\|2mCp\|rAp\|r |
| | | Ap\|2mCp\|2mUp\|2mUp\|rAp\|dTp\|dT] |
| 966 | UCUCCAAACCUGAGAAUUA | [2mUp\|2mCp\|2mUp\|2mCp\|2mCp\|rAp\|rAp\|rAp\|2mC |
| | | p\|2mCp\|2mUp\|rGp\|rAp\|rG |
| | | p\|rAp\|rAp\|2mUp\|2mUp\|rAp\|dTp\|dT] |
| 970 | AACCUGAGAAUUUUAAACA | [rAp\|rAp\|2mCp\|2mCp\|2mUp\|rGp\|rAp\|rGp\|rAp\|r |
| | | Ap\|2mUp\|2mUp\|2mUp\|2mUp\|r |
| | | Ap\|rAp\|rAp\|2mCp\|rAp\|dTp\|dT] |
| 972 | CCUGAGAAUUUUAAACAGA | [2mCp\|2mCp\|2mUp\|rGp\|rAp\|rGp\|rAp\|rAp\|2mUp\| |
| | | 2mUp\|2mUp\|2mUp\|rAp\|rAp\|r |
| | | Ap\|2mCp\|rAp\|rGp\|rAp\|dTp\|dT] |
| 988 | GCUAUCAAGUAAAAUGGAA | [rGp\|2mCp\|2mUp\|rAp\|2mUp\|2mCp\|rAp\|rAp\|rGp\| |
| | | 2mUp\|rAp\|rAp\|rAp\|rAp\|2mUp\| |
| | | rGp\|rGp\|rAp\|rAp\|dTp\|dT] |
| 990 | CUAUCAAGUAAAAUGGAAA | [2mCp\|2mUp\|rAp\|2mUp\|2mCp\|rAp\|rAp\|rGp\|2mUp |
| | | \|rAp\|rAp\|rAp\|rAp\|2mUp\|rGp\| |
| | | rGp\|rAp\|rAp\|rAp\|dTp\|dT] |
| 992 | UAUCAAGUAAAAUGGAAUA | [2mUp\|rAp\|2mUp\|2mCp\|rAp\|rAp\|rGp\|2mUp\|rAp\| |
| | | rAp\|rAp\|rAp\|2mUp\|rGp\|rGp\|rA |
| | | p\|rAp\|2mUp\|rAp\|dTp\|dT] |
| 994 | UCAAGUAAAAUGGAAUUUA | [2mUp\|2mCp\|rAp\|rAp\|rGp\|2mUp\|rAp\|rAp\|rAp\|r |
| | | Ap\|2mUp\|rGp\|rGp\|rAp\|rAp\|2mU |
| | | p\|2mUp\|2mUp\|rAp\|dTp\|dT] |
| 996 | CAAGUAAAAUGGAAUUUAA | [2mCp\|rAp\|rAp\|rGp\|2mUp\|rAp\|rAp\|rAp\|rAp\|2m |
| | | Up\|rGp\|rGp\|rAp\|rAp\|2mUp\|2mU |
| | | p\|2mUp\|rAp\|rAp\|dTp\|dT] |
| 1006 | UUAUGACAAUUAAUGGAAA | [2mUp\|2mUp\|rAp\|2mUp\|rGp\|rAp\|2mCp\|rAp\|rAp\| |
| | | 2mUp\|2mUp\|rAp\|rAp\|2mUp\|r |
| | | Gp\|rGp\|rAp\|rAp\|rAp\|dTp\|dT] |
| 1020 | GGAACAACAUUAAGGAAUA | [rGp\|rGp\|rAp\|rAp\|2mCp\|rAp\|rAp\|2mCp\|rAp\|2m |
| | | Up\|2mUp\|rAp\|rAp\|rGp\|rGp\|rAp\| |
| | | rAp\|2mUp\|rAp\|dTp\|dT] |
| 1032 | CUGGAAAUCCUACAGAAUA | [2mCp\|2mUp\|rGp\|rGp\|rAp\|rAp\|rAp\|2mUp\|2mCp\| |
| | | 2mCp\|2mUp\|rAp\|2mCp\|rAp\|r |
| | | Gp\|rAp\|rAp\|2mUp\|rAp\|dTp\|dT] |
| 1054 | UCAGACUGAUAUGAAAACA | [2mUp\|2mCp\|rAp\|rGp\|rAp\|2mCp\|2mUp\|rGp\|rAp\| |
| | | 2mUp\|rAp\|2mUp\|rGp\|rAp\|rAp |
| | | \|rAp\|rAp\|2mCp\|rAp\|dTp\|dT] |
| 1056 | GACUGAUAUGAAAACCAAA | [rGp\|rAp\|2mCp\|2mUp\|rGp\|rAp\|2mUp\|rAp\|2mUp\| |
| | | rGp\|rAp\|rAp\|rAp\|rAp\|2mCp\|2 |
| | | mCp\|rAp\|rAp\|rAp\|dTp\|dT] |
| 1058 | ACUGAUAUGAAAACCAAAA | [rAp\|2mCp\|2mUp\|rGp\|rAp\|2mUp\|rAp\|2mUp\|rGp\| |
| | | rAp\|rAp\|rAp\|rAp\|2mCp\|2mCp\| |
| | | rAp\|rAp\|rAp\|rAp\|dTp\|dT] |
| 1076 | UUGCUGUGGGUUUUAGACA | [2mUp\|2mUp\|rGp\|2mCp\|2mUp\|rGp\|2mUp\|rGp\|rGp |
| | | \|rGp\|2mUp\|2mUp\|2mUp\|2 |
| | | mUp\|rAp\|rGp\|rAp\|2mCp\|rAp\|dTp\|dT] |
| 1088 | GUUUUAGACCACACUAAAA | [rGp\|2mUp\|2mUp\|2mUp\|2mUp\|rAp\|rGp\|rAp\|2mCp |
| | | \|2mCp\|rAp\|2mCp\|rAp\|2mC |
| | | p\|2mUp\|rAp\|rAp\|rAp\|rAp\|dTp\|dT] |
| 1096 | AGACCACACUAAAACUUCA | [rAp\|rGp\|rAp\|2mCp\|2mCp\|rAp\|2mCp\|rAp\|2mCp\| |
| | | 2mUp\|rAp\|rAp\|rAp\|rAp\|2mCp\| |
| | | 2mUp\|2mUp\|2mCp\|rAp\|dTp\|dT] |
| 1098 | CCACACUAAAACUUCAUUA | [2mCp\|2mCp\|rAp\|2mCp\|rAp\|2mCp\|2mUp\|rAp\|rAp |
| | | \|rAp\|rAp\|2mCp\|2mUp\|2mUp |
| | | \|2mCp\|rAp\|2mUp\|2mUp\|rAp\|dTp\|dT] |
| 1110 | GGGAGACGGAAGUUAAAGA | [rGp\|rGp\|rGp\|rAp\|rGp\|rAp\|2mCp\|rGp\|rGp\|rAp |
| | | \|rAp\|rGp\|2mUp\|2mUp\|rAp\|rAp\|rA |
| | | p\|rGp\|rAp\|dTp\|dT] |
| 1112 | GGAGACGGAAGUUAAAGAA | [rGp\|rGp\|rAp\|rGp\|rAp\|2mCp\|rGp\|rGp\|rAp\|rAp |
| | | \|rGp\|2mUp\|2mUp\|rAp\|rAp\|rAp\|rG |
| | | p\|rAp\|rAp\|dTp\|dT] |
| 1126 | CCCAGCCACUCCUUAAAUA | [2mCp\|2mCp\|2mCp\|rAp\|rGp\|2mCp\|2mCp\|rAp\|2mC |
| | | p\|2mUp\|2mCp\|2mCp\|2mU |
| | | p\|2mUp\|rAp\|rAp\|rAp\|2mUp\|rAp\|dTp\|dT] |
| 1214 | UUUGGUCAGAUAGAAAAUA | [2mUp\|2mUp\|2mUp\|rGp\|rGp\|2mUp\|2mCp\|rAp\|rGp |
| | | \|rAp\|2mUp\|rAp\|rGp\|Ap\|Ap |
| | | \|rAp\|rAp\|2mUp\|rAp\|dTp\|dT] |
| 1220 | CAGAUAGAAAAUCAUCUAA | [2mCp\|rAp\|rGp\|rAp\|2mUp\|rAp\|rGp\|rAp\|rAp\|rA |
| | | p\|rAp\|2mUp\|2mCp\|rAp\|2mUp\|2 |
| | | mCp\|2mUp\|rAp\|rAp\|dTp\|dT] |
| 1230 | AAUCAUCUACGUAAAUUGA | [rAp\|rAp\|2mUp\|2mCp\|rAp\|2mUp\|2mCp\|2mUp\|rAp |
| | | \|2mCp\|rGp\|2mUp\|rAp\|rAp\|r |
| | | Ap\|2mUp\|2mUp\|rGp\|rAp\|dTp\|dT] |
| 1306 | AGAAUUUCAAGCAAUAAUA | [rAp\|rGp\|rAp\|rAp\|2mUp\|2mUp\|2mUp\|2mCp\|rAp\| |
| | | rAp\|rGp\|2mCp\|rAp\|rAp\|2mUp\| |
| | | rAp\|rAp\|2mUp\|rAp\|dTp\|dT] |
| 1308 | AUUUCAAGCAAUAAUACCA | [rAp\|2mUp\|2mUp\|2mUp\|2mCp\|rAp\|rAp\|rGp\|2mCp |
| | | \|rAp\|rAp\|2mUp\|rAp\|rAp\|2m |
| | | Up\|rAp\|2mCp\|2mCp\|rAp\|dTp\|dT] |
| 1310 | UUUCAAGCAAUAAUACCUA | [2mUp\|2mUp\|2mUp\|2mCp\|rAp\|rAp\|rGp\|2mCp\|rAp |
| | | \|rAp\|2mUp\|rAp\|rAp\|2mUp\|r |
| | | Ap\|2mCp\|2mCp\|2mUp\|rAp\|dTp\|dT] |
| 1318 | AUACCUGCUGUUAAUUCCA | [rAp\|2mUp\|rAp\|2mCp\|2mCp\|2mUp\|rGp\|2mCp\|2mU |
| | | p\|rGp\|2mUp\|2mUp\|rAp\|rA |
| | | p\|2mUp\|2mUp\|2mCp\|2mCp\|rAp\|dTp\|dT] |
| 1326 | ACCGUUAUUUUAGAAAUUA | [rAp\|2mCp\|2mCp\|rGp\|2mUp\|2mUp\|rAp\|2mUp\|2mU |
| | | p\|2mUp\|2mUp\|rAp\|rGp\|rA |
| | | p\|rAp\|rAp\|2mUp\|2mUp\|rAp\|dTp\|dT] |
| 1386 | UCCAGUGGAGCAUUACUUA | [2mUp\|2mCp\|2mCp\|rAp\|rGp\|2mUp\|rGp\|rGp\|rAp\| |
| | | rGp\|2mCp\|rAp\|2mUp\|2mUp\|r |
| | | Ap\|2mCp\|2mUp\|2mUp\|rAp\|dTp\|dT] |
| 1394 | GAGCAUUACUUAAAGAUAA | [rGp\|rAp\|rGp\|2mCp\|rAp\|2mUp\|2mUp\|rAp\|2mCp\| |
| | | 2mUp\|2mUp\|rAp\|rAp\|rAp\|rGp |
| | | \|rAp\|2mUp\|rAp\|rAp\|dTp\|dT] |
| 1396 | AGCAUUACUUAAAGAUACA | [rAp\|rGp\|2mCp\|rAp\|2mUp\|2mUp\|rAp\|2mCp\|2mUp |
| | | \|2mUp\|rAp\|rAp\|rAp\|rGp\|rAp\| |
| | | 2mUp\|rAp\|2mCp\|rAp\|dTp\|dT] |
| 1400 | UACUUAAAGAUACUCAAUA | [2mUp\|rAp\|2mCp\|2mUp\|2mUp\|rAp\|rAp\|rAp\|rGp\| |
| | | rAp\|2mUp\|rAp\|2mCp\|2mUp\|2 |
| | | mCp\|rAp\|rAp\|2mUp\|rAp\|dTp\|dT] |
| 1404 | UUAAAGAUACUCAAUGAAA | [2mUp\|2mUp\|rAp\|rAp\|rAp\|rGp\|rAp\|2mUp\|rAp\|2 |
| | | mCp\|2mUp\|2mCp\|rAp\|rAp\|2m |
| | | Up\|rGp\|rAp\|rAp\|rAp\|dTp\|dT] |
| 1424 | GUUGGGGAUAAAACUGAAA | [rGp\|2mUp\|2mUp\|rGp\|rGp\|rGp\|rGp\|rAp\|2mUp\|r |
| | | Ap\|rAp\|rAp\|rAp\|2mCp\|2mUp\|r |
| | | Gp\|rAp\|rAp\|rAp\|dTp\|dT] |
| 1426 | UUGGGGAUAAAACUGAAUA | [2mUp\|2mUp\|rGp\|rGp\|rGp\|rGp\|rAp\|2mUp\|rAp\|r |
| | | Ap\|rAp\|rAp\|2mCp\|2mUp\|rGp\|r |
| | | Ap\|rAp\|2mUp\|rAp\|dTp\|dT] |
| 1448 | UUCUGACUUCCCUUUAAUA | [2mUp\|2mUp\|2mCp\|2mUp\|rGp\|rAp\|2mCp\|2mUp\|2m |
| | | Up\|2mCp\|2mCp\|2mCp\|2 |
| | | mUp\|2mUp\|2mUp\|rAp\|rAp\|2mUp\|rAp\|dTp\|dT] |
| 1452 | UGACUUCCCUUUAAUAAAA | [2mUp\|rGp\|rAp\|2mCp\|2mUp\|2mUp\|2mCp\|2mCp\|2m |
| | | Cp\|2mUp\|2mUp\|2mUp\|rA |
| | | 2mUp\|rAp\|2mUp\|rAp\|dTp\|dT] |
| 1454 | AGAGGAAGGAUGAAAUUCA | [rAp\|rGp\|rAp\|rGp\|rGp\|rAp\|rAp\|rGp\|rGp\|rAp\| |
| | | 2mUp\|rGp\|rAp\|rAp\|rAp\|2mUp\|2mU |
| | | p\|2mCp\|rAp\|dTp\|dT] |
| 1506 | AUCCUUCUGCACAAUAUGA | [rAp\|2mUp\|2mCp\|2mCp\|2mUp\|2mUp\|2mCp\|2mUp\|r |
| | | Gp\|2mCp\|rAp\|2mCp\|rAp\|r |
| | | Ap\|2mUp\|rAp\|2mUp\|rGp\|rAp\|dTp\|dT] |
| 1524 | CAGGACAGGAGUUUAUGAA | [2mCp\|rAp\|rGp\|rGp\|rAp\|2mCp\|rAp\|rGp\|rGp\|rA |
| | | p\|rGp\|2mUp\|2mUp\|2mUp\|rAp\|2 |
| | | mUp\|rGp\|rAp\|rAp\|dTp\|dT] |
| 1540 | AGAACUCUGCUGUAUCUUA | [rAp\|rGp\|rAp\|rAp\|2mCp\|2mUp\|2mCp\|2mUp\|rGp\| |
| | | 2mCp\|2mUp\|rGp\|2mUp\|rAp\|2 |
| | | mUp\|2mCp\|2mUp\|2mUp\|rAp\|dTp\|dT] |
| 1546 | CUCUGCUGUAUCUUGUAUA | [2mCp\|2mUp\|2mCp\|2mUp\|rGp\|2mCp\|2mUp\|rGp\|2m |
| | | Up\|rAp\|2mUp\|2mCp\|2mU |
| | | p\|2mUp\|rGp\|2mUp\|rAp\|2mUp\|rAp\|dTp\|dT] |
| 1656 | GAAUGGCUUGAUUUUCUAA | [rGp\|rAp\|rAp\|2mUp\|rGp\|rGp\|2mCp\|2mUp\|2mUp\| |
| | | rGp\|rAp\|2mUp\|2mUp\|2mUp\|2 |
| | | mUp\|2mCp\|2mUp\|rAp\|rAp\|dTp\|dT] |
| 1666 | UUUCUAGAGAAAUUCAGUA | [2mUp\|2mUp\|2mUp\|2mCp\|2mUp\|rAp\|rGp\|rAp\|rGp |
| | | \|rAp\|rAp\|rAp\|2mUp\|2mUp\|2 |
| | | mCp\|rAp\|rGp\|2mUp\|rAp\|dTp\|dT] |
| 1674 | AAAUUCAGUGAACAUUAUA | [rAp\|rAp\|rAp\|2mUp\|2mUp\|2mCp\|rAp\|rGp\|2mUp\| |
| | | rGp\|rAp\|rAp\|2mCp\|rAp\|2mUp\| |
| | | 2mUp\|rAp\|2mUp\|rAp\|dTp\|dT] |
| 1676 | AAUUCAGUGAACAUUAUCA | [rAp\|rAp\|2mUp\|2mUp\|2mCp\|rAp\|rGp\|2mUp\|rGp\| |
| | | rAp\|rAp\|2mCp\|rAp\|2mUp\|2m |
| | | Up\|rAp\|2mUp\|2mCp\|rAp\|dTp\|dT] |
| 1678 | AUUCAGUGAACAUUAUCAA | [rAp\|2mUp\|2mUp\|2mCp\|rAp\|rGp\|2mUp\|rGp\|rAp\| |
| | | rAp\|2mCp\|rAp\|2mUp\|2mUp\|r |
| | | Ap\|2mUp\|2mCp\|rAp\|rAp\|dTp\|dT] |
| 1722 | UGUUGACUGCAUUUUCUCA | [2mUp\|rGp\|2mUp\|2mUp\|rGp\|rAp\|2mCp\|2mUp\|rGp |
| | | \|2mCp\|rAp\|2mUp\|2mUp\|2 |
| | | mUp\|2mUp\|2mCp\|2mUp\|2mCp\|rAp\|dTp\|dT] |
| 1762 | AGACCAACUGUACAAGAAA | [rAp\|rGp\|rAp\|2mCp\|2mCp\|rAp\|rAp\|2mCp\|2mUp\| |
| | | rGp\|2mUp\|rAp\|2mCp\|rAp\|rAp\| |
| | | rGp\|rAp\|rAp\|rAp\|dTp\|dT] |
| 1766 | ACUGUACAAGAAGAAAGAA | [rAp\|2mCp\|2mUp\|rGp\|2mUp\|rAp\|2mCp\|rAp\|rAp |
| | | \|rGp\|rAp\|rAp\|rGp\|rAp\|rAp\|rAp\|r |
| | | Gp\|rAp\|rAp\|dTp\|dT] |
| 1768 | CUGUACAAGAAGAAAGAAA | [2mCp\|2mUp\|rGp\|2mUp\|rAp\|2mCp\|rAp\|rAp\|rGp\| |
| | | rAp\|rAp\|rGp\|rAp\|rAp\|rAp\|rGp\| |
| | | rAp\|rAp\|rAp\|dTp\|dT] |
| 1836 | ACUCAGAGAGAGUAAUGAA | [rAp\|2mCp\|2mUp\|2mCp\|rAp\|rGp\|rAp\|rGp\|rAp\|r |
| | | Gp\|rAp\|rGp\|2mUp\|rAp\|rAp\|2m |
| | | Up\|rGp\|rAp\|rAp\|dTp\|dT] |
| 1838 | UCAGAGAGAGUAAUGAUAA | [2mUp\|2mCp\|rAp\|rGp\|rAp\|rGp\|rAp\|rGp\|rAp\|rG |
| | | p\|2mUp\|rAp\|rAp\|2mUp\|rGp\|rAp\| |
| | | 2mUp\|rAp\|rAp\|dTp\|dT] |
| 1842 | AGAGAGAGUAAUGAUAAUA | [rAp\|rGp\|rAp\|rGp\|rAp\|rGp\|rAp\|rGp\|2mUp\|rAp |
| | | \|rAp\|2mUp\|rGp\|rAp\|2mUp\|rAp\|rA |
| | | p\|2mUp\|rAp\|dTp\|dT] |
| 1868 | GAAAGAGCUCCUACAUAAA | [rGp\|rAp\|rAp\|rAp\|rGp\|rAp\|rGp\|2mCp\|2mUp\|2m |
| | | Cp\|2mCp\|2mUp\|rAp\|2mCp\|rAp |
| | | \|2mUp\|rAp\|rAp\|rAp\|dTp\|dT] |
| 1886 | AAACAAGUUGCAUUGAUUA | [rAp\|rAp\|rAp\|2mCp\|rAp\|rAp\|rGp\|2mUp\|2mUp\|r |
| | | Gp\|2mCp\|rAp\|2mUp\|2mUp\|rGp |
| | | \|rAp\|2mUp\|2mUp\|rAp\|dTp\|dT] |
| 1888 | AACAAGUUGCAUUGAUUAA | [rAp\|rAp\|2mCp\|rAp\|rAp\|rGp\|2mUp\|2mUp\|rGp\|2 |
| | | mCp\|rAp\|2mUp\|2mUp\|rGp\|rAp |
| | | \|2mUp\|2mUp\|rAp\|rAp\|dTp\|dT] |
| 1964 | GUGGAUGGCAUUUUCACAA | [rGp\|2mUp\|rGp\|rGp\|rAp\|2mUp\|rGp\|rGp\|2mCp\|r |
| | | Ap\|2mUp\|2mUp\|2mUp\|2mUp\| |
| | | 2mCp\|rAp\|2mCp\|rAp\|rAp\|dTp\|dT] |
| 1990 | UGCAGACAAUAUAUAUAAA | [2mUp\|rGp\|2mCp\|rAp\|rGp\|rAp\|2mCp\|rAp\|rAp\|2 |
| | | mUp\|rAp\|2mUp\|rAp\|2mUp\|rAp\| |
| | | 2mUp\|rAp\|rAp\|rAp\|dTp\|dT] |
| 2030 | GACACAGCAGAAAUAAUCA | [rGp\|rAp\|2mCp\|rAp\|2mCp\|rAp\|rGp\|2mCp\|rAp\|r |
| | | Gp\|rAp\|rAp\|rAp\|2mUp\|rAp\|rAp\| |
| | | 2mUp\|2mCp\|rAp\|dTp\|dT] |
| 2108 | AUGCUACACUUGAGUAUUA | [rAp\|2mUp\|rGp\|2mCp\|2mUp\|rAp\|2mCp\|rAp\|2mCp |
| | | \|2mUp\|2mUp\|rGp\|rAp\|rGp\|2 |
| | | mUp\|rAp\|2mUp\|2mUp\|rAp\|dTp\|dT] |
| 2128 | GAGAUGUGAAAUCCUUAAA | [rGp\|rAp\|rGp\|rAp\|2mUp\|rGp\|2mUp\|rGp\|rAp\|rA |
| | | p\|rAp\|2mUp\|2mCp\|2mCp\|2mUp |
| | | 2mUp\|rAp\|rAp\|rAp\|dTp\|dT] |
| 2230 | AACAAGUCCCUGAUUUUGA | [rAp\|rAp\|2mCp\|rAp\|rAp\|rGp\|2mUp\|2mCp\|2mCp\| |
| | | 2mCp\|2mUp\|rGp\|rAp\|2mUp\|2 |
| | | mUp\|2mUp\|2mUp\|rGp\|rAp\|dTp\|dT] |
| 2242 | UUUUGUCACCUUCCUUUAA | [2mUp\|2mUp\|2mUp\|2mUp\|rGp\|2mUp\|2mCp\|rAp\|2m |
| | | Cp\|2mCp\|2mUp\|2mUp\|2 |
| | | mCp\|2mCp\|2mUp\|2mUp\|2mUp\|rAp\|rAp\|dTp\|dT] |
| 2246 | UCACCUUCCUUUACCAAAA | [2mUp\|2mCp\|rAp\|2mCp\|2mCp\|2mUp\|2mUp\|2mCp\|2 |
| | | mCp\|2mUp\|2mUp\|2mUp\| |
| | | rAp\|2mCp\|2mCp\|rAp\|rAp\|rAp\|rAp\|dTp\|dT] |
| 2254 | CUUUACCAAAUAACUAGAA | [2mCp\|2mUp\|2mUp\|2mUp\|rAp\|2mCp\|2mCp\|rAp\|rA |
| | | p\|rAp\|2mUp\|rAp\|rAp\|2mCp |
| | | \|2mUp\|rAp\|rGp\|rAp\|rAp\|dTp\|dT] |
| 2274 | AAGGAGUUAUGGAUUAAAA | [rAp\|rAp\|rGp\|rGp\|rAp\|rGp\|2mUp\|2mUp\|rAp\|2m |
| | | Up\|rGp\|rGp\|rAp\|2mUp\|2mUp\|r |
| | | Ap\|rAp\|rAp\|rAp\|dTp\|dT] |
| 2294 | UAAAUGUGGCUAAACUAGA | [2mUp\|rAp\|rAp\|rAp\|2mUp\|rGp\|2mUp\|rGp\|rGp\|2 |
| | | mCp\|2mUp\|rAp\|rAp\|rAp\|2mCp |
| | | \|2mUp\|rAp\|rGp\|rAp\|dTp\|dT] |
| 2330 | GAGCUGGAAGGAUUAAUAA | [rGp\|rAp\|rGp\|2mCp\|2mUp\|rGp\|rGp\|rAp\|rAp\|rG |
| | | p\|rGp\|rAp\|2mUp\|2mUp\|rAp\|rAp\| |
| | | 2mUp\|rAp\|rAp\|dTp\|dT] |
| 2334 | GGAUUAAUAAAUACGAAAA | [rGp\|rGp\|rAp\|2mUp\|2mUp\|rAp\|rAp\|2mUp\|rAp\|r |
| | | Ap\|rAp\|2mUp\|rAp\|2mCp\|rGp\|rA |
| | | p\|rAp\|rAp\|rAp\|dTp\|dT] |
| 2356 | ACUCAAGUAUUUUGCAAAA | [rAp\|2mCp\|2mUp\|2mCp\|rAp\|rAp\|rGp\|2mUp\|rAp\| |
| | | 2mUp\|2mUp\|2mUp\|2mUp\|rG |
| | | p\|2mCp\|rAp\|rAp\|rAp\|rAp\|dTp\|dT] |
| 2360 | AAGUAUUUUGCAAAGUUAA | [rAp\|rAp\|rGp\|2mUp\|rAp\|2mUp\|2mUp\|2mUp\|2mUp |
| | | \|rGp\|2mCp\|rAp\|rAp\|rAp\|rGp |
| | | \|2mUp\|2mUp\|rAp\|rAp\|dTp\|dT] |
| 2362 | GUAUUUUGCAAAGUUAUGA | [rGp\|2mUp\|rAp\|2mUp\|2mUp\|2mUp\|2mUp\|rGp\|2mC |
| | | p\|rAp\|rAp\|rAp\|rGp\|2mUp\|2 |
| | | mUp\|rAp\|2mUp\|rGp\|rAp\|dTp\|dT] |
| 2448 | CAACUGUACAAAAUAACUA | [2mCp\|rAp\|rAp\|2mCp\|2mUp\|rGp\|2mUp\|rAp\|2mCp |
| | | \|rAp\|rAp\|rAp\|rAp\|2mUp\|rAp\| |
| | | rAp\|2mCp\|2mUp\|rAp\|dTp\|dT] |
| 2502 | ACAUGUGAGCAUAAAAUUA | [rAp\|2mCp\|rAp\|2mUp\|rGp\|2mUp\|rGp\|rAp\|rGp\|2 |
| | | mCp\|rAp\|2mUp\|rAp\|rAp\|rAp\|rA |
| | | p\|2mUp\|2mUp\|rAp\|dTp\|dT] |
| 2504 | CAUGUGAGCAUAAAAUUAA | [2mCp\|rAp\|2mUp\|rGp\|2mUp\|rGp\|rAp\|rGp\|2mCp\| |
| | | rAp\|2mUp\|rAp\|rAp\|rAp\|rAp\|2 |
| | | mUp\|2mUp\|rAp\|rAp\|dTp\|dT] |
| 2516 | AAUUAUGACCAUGGUAUAA | [rAp\|rAp\|2mUp\|2mUp\|rAp\|2mUp\|rGp\|rAp\|2mCp\| |
| | | 2mCp\|rAp\|2mUp\|rGp\|rGp\|2m |
| | | Up\|rAp\|2mUp\|rAp\|rAp\|dTp\|dT] |
| 2518 | AUUAUGACCAUGGUAUAUA | [rAp\|2mUp\|2mUp\|rAp\|2mUp\|rGp\|rAp\|2mCp\|2mCp |
| | | \|rAp\|2mUp\|rGp\|rGp\|2mUp\|r |
| | | Ap\|2mUp\|rAp\|2mUp\|rAp\|dTp\|dT] |
| 2578 | AUAAACACUCUUGAAUAGA | [rAp\|2mUp\|rAp\|rAp\|rAp\|2mCp\|rAp\|2mCp\|2mUp\| |
| | | 2mCp\|2mUp\|2mUp\|rGp\|rAp\|r |
| | | Ap\|2mUp\|rAp\|rGp\|rAp\|dTp\|dT] |
| 2580 | UAAACACUCUUGAAUAGAA | [2mUp\|rAp\|rAp\|rAp\|2mCp\|rAp\|2mCp\|2mUp\|2mCp |
| | | \|2mUp\|2mUp\|rGp\|rAp\|rAp\|2 |
| | | mUp\|rAp\|rGp\|rAp\|rAp\|dTp\|dT] |
| 2592 | AUAGACUUCCACUUUGUAA | [rAp\|2mUp\|rAp\|rGp\|rAp\|2mCp\|2mUp\|2mUp\|2mCp |
| | | \|2mCp\|rAp\|2mCp\|2mUp\|2m |
| | | Up\|2mUp\|rGp\|2mUp\|rAp\|rAp\|dTp\|dT] |
| 2596 | AGACUUCCACUUUGUAAUA | [rAp\|rGp\|rAp\|2mCp\|2mUp\|2mUp\|2mCp\|2mCp\|rAp |
| | | \|2mCp\|2mUp\|2mUp\|2mUp\|r |
| | | Gp\|2mUp\|rAp\|rAp\|2mUp\|rAp\|dTp\|dT] |
| 2602 | CUUCCACUUUGUAAUUAGA | [2mCp\|2mUp\|2mUp\|2mCp\|2mCp\|rAp\|2mCp\|2mUp\|2 |
| | | mUp\|2mUp\|rGp\|2mUp\|rA |
| | | p\|rAp\|2mUp\|2mUp\|rAp\|rGp\|Ap\|dTp\|dT] |
| 2654 | CUUAAGUGGCAGAAUAUAA | [2mCp\|2mUp\|2mUp\|rAp\|rAp\|rGp\|2mUp\|rGp\|rGp\| |
| | | 2mCp\|rAp\|rGp\|rAp\|rAp\|2mUp |
| | | \|rAp\|2mUp\|rAp\|rAp\|dTp\|dT] |
| 2656 | UUAAGUGGCAGAAUAUAAA | [2mUp\|2mUp\|rAp\|rAp\|rGp\|2mUp\|rGp\|rGp\|2mCp\| |
| | | rAp\|rGp\|rAp\|rAp\|2mUp\|rAp\|2 |
| | | mUp\|rAp\|rAp\|rAp\|dTp\|dT] |
| 2686 | UUUUGGAGGGUGAUAUAAA | [2mUp\|2mUp\|2mUp\|2mUp\|rGp\|rGp\|rAp\|rGp\|rGp\| |
| | | rGp\|2mUp\|rGp\|rAp\|2mUp\|rA |
| | | p\|2mUp\|rAp\|rAp\|rAp\|dTp\|dT] |
| 2762 | UCCACCAAGAACAUAAGAA | [2mUp\|2mCp\|2mCp\|rAp\|2mCp\|2mCp\|rAp\|rAp\|rGp |
| | | \|rAp\|rAp\|2mCp\|rAp\|2mUp\|r |
| | | Ap\|rAp\|rGp\|rAp\|rAp\|dTp\|dT] |
| 2768 | ACCAAGAACAUAAGAAUUA | [rAp\|2mCp\|2mCp\|rAp\|rAp\|rGp\|rAp\|rAp\|2mCp\| |
| | | rAp\|2mUp\|rAp\|rAp\|rGp\|rAp\|rAp\| |
| | | 2mUp\|2mUp\|rAp\|dTp\|dT] |
| 2782 | UAGAAUUAUCAAGCUUUUA | [2mUp\|rAp\|rGp\|rAp\|rAp\|2mUp\|2mUp\|rAp\|2mUp\| |
| | | 2mCp\|rAp\|rAp\|rGp\|2mCp\|2m |
| | | Up\|2mUp\|2mUp\|2mUp\|rAp\|dTp\|dT] |
| 2844 | UUGGAUGAAAUUAUUUGUA | [2mUp\|2mUp\|rGp\|rGp\|rAp\|2mUp\|rGp\|rAp\|rAp\|r |
| | | Ap\|2mUp\|2mUp\|rAp\|2mUp\|2m |
| | | Up\|2mUp\|rGp\|2mUp\|rAp\|dTp\|dT] |
| 2846 | GAUGAAAUUAUUUGUCAUA | [rGp\|rAp\|2mUp\|rGp\|rAp\|rAp\|rAp\|2mUp\|2mUp\|r |
| | | Ap\|2mUp\|2mUp\|2mUp\|rGp\|2m |
| | | Up\|2mCp\|rAp\|2mUp\|rAp\|dTp\|dT] |

For the chemical modifications, “2m” means 2′-O-Methyl ribonucleotides, “r” means ribonucleotide, “p” means phosphate linkage, and “d” means deoxyribonucleotide.

TABLE 14
Chemical Modifications for Antisense Strands

| SEQ ID | | |
|---|---|---|
| No./ | | |
| Antisense | | |
| No. | Antisense | Antisense Chemical Modifications |
| 79 | UAUUUCUUUGGCUCAGAGU | [rUp\|rAp\|rUp\|rUp\|rUp\|rCp\|rUp\|rUp\|rUp\| |
| | | rGp\|rGp\|rCp\|rUp\|2mCp\|rAp\|rGp\|rAp\|rG |
| | | p\|rUp\|dTp\|dT] |
| 83 | UAGACAUUUCUUUGGCUCA | [rUp\|rAp\|rGp\|rAp\|2mCp\|rAp\|rUp\|rUp\|rUp |
| | | \|rCp\|rUp\|rUp\|rUp\|rGp\|rGp\|rCp\|rUp\|2 |
| | | mCp\|rAp\|dTp\|dT] |
| 105 | UUACUUUGAGGAAGGGCAG | [rUp\|2mUp\|rAp\|rCp\|rUp\|rUp\|rUp\|rGp\|rAp |
| | | \|rGp\|rGp\|rAp\|rAp\|rGp\|rGp\|rGp\|2mCp\| |
| | | rAp\|rGp\|dTp\|dT] |
| 149 | UGUACAUUUUGGCAGAACU | [rUp\|rGp\|2mUp\|rAp\|2mCp\|rAp\|rUp\|rUp\|rU |
| | | p\|rUp\|rGp\|rGp\|2mCp\|rAp\|rGp\|rAp\|rA |
| | | p\|rCp\|rUp\|dTp\|dT] |
| 159 | UUAUCAUCAAAAUCAGUAC | [rUp\|2mUp\|rAp\|rUp\|2mCp\|rAp\|rUp\|2mCp\|r |
| | | Ap\|rAp\|rAp\|rAp\|rUp\|2mCp\|rAp\|rGp\| |
| | | 2mUp\|rAp\|rCp\|dTp\|dT] |
| 161 | UGAUAUCAUCAAAAUCAGU | [rUp\|rGp\|rAp\|2mUp\|rAp\|rUp\|2mCp\|rAp\|rU |
| | | p\|2mCp\|rAp\|rAp\|rAp\|rAp\|rUp\|2mCp\|r |
| | | Ap\|rGp\|rUp\|dTp\|dT] |
| 191 | UAGAAGAAACUGCAUUCUU | [rUp\|rAp\|rGp\|rAp\|rAp\|rGp\|rAp\|rAp\|rAp\| |
| | | rCp\|rUp\|rGp\|2mCp\|rAp\|rUp\|rUp\|rCp\|rU |
| | | p\|rUp\|dTp\|dT] |
| 241 | UAUUUGAUGAUCCAAACUG | [rUp\|rAp\|rUp\|rUp\|rUp\|rGp\|rAp\|rUp\|rGp\| |
| | | rAp\|rUp\|rCp\|2mCp\|rAp\|rAp\|rAp\|rCp\|rU |
| | | p\|rGp\|dTp\|dT] |
| 245 | UGUAUUUGAUGAUCCAAAC | [rUp\|rGp\|2mUp\|rAp\|rUp\|rUp\|rUp\|rGp\|rAp |
| | | \|rUp\|rGp\|rAp\|rUp\|rCp\|2mCp\|rAp\|rAp\|r |
| | | Ap\|rCp\|dTp\|dT] |
| 247 | UUGUAUUUGAUGAUCCAAA | [rUp\|rUp\|rGp\|2mUp\|rAp\|rUp\|rUp\|rUp\|rGp |
| | | \|rAp\|rUp\|rGp\|rAp\|rUp\|rCp\|2mCp\|rAp\|r |
| | | Ap\|rAp\|dTp\|dT] |
| 251 | UCUUGUAUUUGAUGAUCCA | [rUp\|rCp\|rUp\|rUp\|rGp\|2mUp\|rAp\|rUp\|rUp |
| | | \|rUp\|rGp\|rAp\|rUp\|rGp\|rAp\|rUp\|rCp\|2 |
| | | mCp\|rAp\|dTp\|dT] |
| 253 | UACUUGUAUUUGAUGAUCC | [rUp\|rAp\|rCp\|rUp\|rUp\|rGp\|2mUp\|rAp\|rUp |
| | | \|rUp\|rUp\|rGp\|rAp\|rUp\|rGp\|rAp\|rUp\|rC |
| | | p\|rCp\|dTp\|dT] |
| 261 | UAAAUUUUCAUGACUUGUA | [rUp\|rAp\|rAp\|rAp\|rUp\|rUp\|rUp\|rUp\|2mCp |
| | | \|rAp\|rUp\|rGp\|rAp\|rCp\|rUp\|rUp\|rGp\|2m |
| | | Up\|rAp\|dTp\|dT] |
| 309 | UGUAUUGUAAUUCUAGCGG | [rUp\|rGp\|2mUp\|rAp\|rUp\|rUp\|rGp\|2mUp\|rAp |
| | | \|rAp\|rUp\|rUp\|rCp\|2mUp\|rAp\|rGp\|rC |
| | | p\|rGp\|rGp\|dTp\|dT] |
| 315 | UUCUAUGUAUUGUAAUUCU | [rUp\|rUp\|rCp\|2mUp\|rAp\|rUp\|rGp\|2mUp\|rA |
| | | p\|rUp\|rUp\|rGp\|2mUp\|rAp\|rAp\|rUp\|rU |
| | | p\|rCp\|rUp\|dTp\|dT] |
| 317 | UUUCUAUGUAUUGUAAUUC | [rUp\|rUp\|rUp\|rCp\|2mUp\|rAp\|rUp\|rGp\|2mU |
| | | p\|rAp\|rUp\|rUp\|rGp\|2mUp\|rAp\|rAp\|rU |
| | | p\|rUp\|rCp\|dTp\|dT] |
| 355 | UUAUAUCCACAUUCCACAC | [rUp\|2mUp\|rAp\|2mUp\|rAp\|rUp\|rCp\|2mCp\|r |
| | | Ap\|2mCp\|rAp\|rUp\|rUp\|rCp\|2mCp\|rA |
| | | p\|2mCp\|rAp\|rCp\|dTp\|dT] |
| 357 | UUUAUAUCCACAUUCCACA | [rUp\|rUp\|2mUp\|rAp\|2mUp\|rAp\|rUp\|rCp\|2m |
| | | Cp\|rAp\|2mCp\|rAp\|rUp\|rUp\|rCp\|2mC |
| | | p\|rAp\|2mCp\|rAp\|dTp\|dT] |
| 361 | UACUUAUAUCCACAUUCCA | [rUp\|rAp\|rCp\|rUp\|2mUp\|rAp\|2mUp\|rAp\|rU |
| | | p\|rCp\|2mCp\|rAp\|2mCp\|rAp\|rUp\|rUp\|r |
| | | Cp\|2mCp\|rAp\|dTp\|dT] |
| 363 | UUACUUAUAUCCACAUUCC | [rUp\|2mUp\|rAp\|rCp\|rUp\|2mUp\|rAp\|2mUp\|r |
| | | Ap\|rUp\|rCp\|2mCp\|rAp\|2mCp\|rAp\|rU |
| | | p\|rUp\|rCp\|rCp\|dTp\|dT] |
| 365 | UAUACUUAUAUCCACAUUC | [rUp\|rAp\|2mUp\|rAp\|rCp\|rUp\|2mUp\|rAp\|2m |
| | | Up\|rAp\|rUp\|rCp\|2mCp\|rAp\|2mCp\|rA |
| | | p\|rUp\|rUp\|rCp\|dTp\|dT] |
| 369 | UCUAUACUUAUAUCCACAU | [rUp\|rCp\|2mUp\|rAp\|2mUp\|rAp\|rCp\|rUp\|2m |
| | | Up\|rAp\|2mUp\|rAp\|rUp\|rCp\|2mCp\|rA |
| | | p\|2mCp\|rAp\|rUp\|dTp\|dT] |
| 371 | UUCUAUACUUAUAUCCACA | [rUp\|rUp\|rCp\|2mUp\|rAp\|2mUp\|rAp\|rCp\|rU |
| | | p\|2mUp\|rAp\|2mUp\|rAp\|rUp\|rCp\|2mC |
| | | p\|rAp\|2mCp\|rAp\|dTp\|dT] |
| 373 | UAAUCUAUACUUAUAUCCA | [rUp\|rAp\|rAp\|rUp\|rCp\|2mUp\|rAp\|2mUp\|rA |
| | | p\|rCp\|rUp\|2mUp\|rAp\|2mUp\|rAp\|rUp\|r |
| | | Cp\|2mCp\|rAp\|dTp\|dT] |
| 379 | UGCAAUAAAUAUUGAGCUC | [rUp\|rGp\|2mCp\|rAp\|rAp\|2mUp\|rAp\|rAp\|rA |
| | | p\|2mUp\|rAp\|rUp\|rUp\|rGp\|rAp\|rGp\|rC |
| | | p\|rUp\|rCp\|dTp\|dT] |
| 415 | UAUAAAGUUGUGAUCUAAA | [rUp\|rAp\|2mUp\|rAp\|rAp\|rAp\|rGp\|rUp\|rUp |
| | | \|rGp\|rUp\|rGp\|rAp\|rUp\|rCp\|2mUp\|rAp\|r |
| | | Ap\|rAp\|dTp\|dT] |
| 417 | UCAUAAAGUUGUGAUCUAA | [rUp\|2mCp\|rAp\|2mUp\|rAp\|rAp\|rAp\|rGp\|rU |
| | | p\|rUp\|rGp\|rUp\|rGp\|rAp\|rUp\|rCp\|2mU |
| | | p\|rAp\|rAp\|dTp\|dT] |
| 419 | UUCAUAAAGUUGUGAUCUA | [rUp\|rUp\|2mCp\|rAp\|2mUp\|rAp\|rAp\|rAp\|rG |
| | | p\|rUp\|rUp\|rGp\|rUp\|rGp\|rAp\|rUp\|rCp\| |
| | | 2mUp\|rAp\|dTp\|dT] |
| 475 | UUUAUAUCCUUUUGCCACC | [rUp\|rUp\|2mUp\|rAp\|2mUp\|rAp\|rUp\|rCp\|rC |
| | | p\|rUp\|rUp\|rUp\|rUp\|rGp\|rCp\|2mCp\|rA |
| | | p\|rCp\|rCp\|dTp\|dT] |
| 477 | UCUUAUAUCCUUUUGCCAC | [rUp\|rCp\|rUp\|2mUp\|rAp\|2mUp\|rAp\|rUp\|r |
| | | Cp\|rCp\|rUp\|rUp\|rUp\|rUp\|rGp\|rCp\|2mC |
| | | p\|rAp\|rCp\|dTp\|dT] |
| 479 | UCCUUAUAUCCUUUUGCCA | [rUp\|rCp\|rCp\|rUp\|2mUp\|rAp\|2mUp\|rAp\|rU |
| | | p\|rCp\|rCp\|rUp\|rUp\|rUp\|rUp\|rGp\|rCp\| |
| | | 2mCp\|rAp\|dTp\|dT] |
| 481 | UACCUUAUAUCCUUUUGCC | [rUp\|rAp\|rCp\|rCp\|rUp\|2mUp\|rAp\|2mUp\|rA |
| | | p\|rUp\|rCp\|rCp\|rUp\|rUp\|rUp\|rUp\|rGp\|r |
| | | Cp\|rCp\|dTp\|dT] |
| 503 | UUUCAGUUUGCUUCACAAC | [rUp\|rUp\|rUp\|2mCp\|rAp\|rGp\|rUp\|rUp\|rUp |
| | | \|rGp\|rCp\|rUp\|rUp\|2mCp\|rAp\|2mCp\|rA |
| | | p\|rAp\|rCp\|dTp\|dT] |
| 513 | UUUAAUGCUGCAGUUUCAG | [rUp\|rUp\|2mUp\|rAp\|rAp\|rUp\|rGp\|rCp\|rUp |
| | | \|rGp\|2mCp\|rAp\|rGp\|rUp\|rUp\|rUp\|2mC |
| | | p\|rAp\|rGp\|dTp\|dT] |
| 553 | UAAAAGAGUGAACUUCUGU | [rUp\|rAp\|rAp\|rAp\|rAp\|rGp\|rAp\|rGp\|rUp\| |
| | | rGp\|rAp\|rAp\|rCp\|rUp\|rUp\|rCp\|rUp\|rGp\| |
| | | rUp\|dTp\|dT] |
| 559 | UUCAAUUUCCGGGAAAAGA | [rUp\|rUp\|2mCp\|rAp\|rAp\|rUp\|rUp\|rUp\|rCp |
| | | \|rCp\|rGp\|rGp\|rGp\|rAp\|rAp\|rAp\|rAp\|rG |
| | | p\|rAp\|dTp\|dT] |
| 561 | UGUCAAUUUCCGGGAAAAG | [rUp\|rGp\|rUp\|2mCp\|rAp\|rAp\|rUp\|rUp\|rUp |
| | | \|rCp\|rCp\|rGp\|rGp\|rGp\|rAp\|rAp\|rAp\|rA |
| | | p\|rGp\|dTp\|dT] |
| 583 | UGUAGAUUUUGUAUAAAGG | [rUp\|rGp\|2mUp\|rAp\|rGp\|rAp\|rUp\|rUp\|rUp |
| | | \|rUp\|rGp\|2mUp\|rAp\|2mUp\|rAp\|rAp\|rA |
| | | p\|rGp\|rGp\|dTp\|dT] |
| 617 | UUUGAUUAGGGGAUUCACA | [rUp\|rUp\|rUp\|rGp\|rAp\|rUp\|2mUp\|rAp\|rGp |
| | | \|rGp\|rGp\|rGp\|rAp\|rUp\|rUp\|2mCp\|rAp\| |
| | | 2mCp\|rAp\|dTp\|dT] |
| 619 | UCUUGAUUAGGGGAUUCAC | [rUp\|rCp\|rUp\|rUp\|rGp\|rAp\|rUp\|2mUp\|rAp |
| | | \|rGp\|rGp\|rGp\|rGp\|rAp\|rUp\|rUp\|2mCp\| |
| | | rAp\|rCp\|dTp\|dT] |
| 635 | UCAUUUACAGCAUCAUCCA | [rUp\|2mCp\|rAp\|rUp\|rUp\|2mUp\|rAp\|2mCp\|r |
| | | Ap\|rGp\|2mCp\|rAp\|rUp\|2mCp\|rAp\|rU |
| | | p\|rCp\|2mCp\|rAp\|dTp\|dT] |
| 637 | UAACAUUUACAGCAUCAUC | [rUp\|rAp\|rAp\|2mCp\|rAp\|rUp\|rUp\|2mUp\|rA |
| | | p\|2mCp\|rAp\|rGp\|2mCp\|rAp\|rUp\|2mC |
| | | p\|rAp\|rUp\|rCp\|dTp\|dT] |
| 643 | UUUAUCUCAUCAACAUUUA | [rUp\|rUp\|2mUp\|rAp\|rUp\|rCp\|rUp\|2mCp\|rA |
| | | p\|rUp\|2mCp\|rAp\|rAp\|2mCp\|rAp\|rUp\|r |
| | | Up\|2mUp\|rAp\|dTp\|dT] |
| 647 | UGUCAUUAUCUCAUCAACA | [rUp\|rGp\|rUp\|2mCp\|rAp\|rUp\|2mUp\|rAp\|rU |
| | | p\|rCp\|rUp\|2mCp\|rAp\|rUp\|2mCp\|rAp\| |
| | | rAp\|2mCp\|rAp\|dTp\|dT] |
| 649 | UAGUCAUUAUCUCAUCAAC | [rUp\|rAp\|rGp\|rUp\|2mCp\|rAp\|rUp\|2mUp\|rA |
| | | p\|rUp\|rCp\|rUp\|2mCp\|rAp\|rUp\|2mCp\| |
| | | rAp\|rAp\|rCp\|dTp\|dT] |
| 657 | UAAGUAUCAGUCAUUAUCU | [rUp\|rAp\|rAp\|rGp\|2mUp\|rAp\|rUp\|2mCp\|rA |
| | | p\|rGp\|rUp\|2mCp\|rAp\|rUp\|2mUp\|rAp\| |
| | | rUp\|rCp\|rUp\|dTp\|dT] |
| 661 | UAGAAGUAUCAGUCAUUAU | [rUp\|rAp\|rGp\|rAp\|rAp\|rGp\|2mUp\|rAp\|rUp |
| | | \|2mCp\|rAp\|rGp\|rUp\|2mCp\|rAp\|rUp\|2 |
| | | mUp\|rAp\|rUp\|dTp\|dT] |
| 691 | UUUCCUUAUUUUCAGAGAU | [rUp\|rUp\|rUp\|rCp\|rCp\|rUp\|2mUp\|rAp\|rUp |
| | | \|rUp\|rUp\|rUp\|2mCp\|rAp\|rGp\|rAp\|rGp\|r |
| | | Ap\|rUp\|dTp\|dT] |
| 693 | UAACAUUUUCCUUAUUUUC | [rUp\|rAp\|rAp\|2mCp\|rAp\|rUp\|rUp\|rUp\|rUp |
| | | \|rCp\|rCp\|rUp\|2mUp\|rAp\|rUp\|rUp\|rUp\|r |
| | | Up\|rCp\|dTp\|dT] |
| 719 | UACUAUCAAACACAACCUC | [rUp\|rAp\|rCp\|2mUp\|rAp\|rUp\|2mCp\|rAp\|rA |
| | | p\|rAp\|2mCp\|rAp\|2mCp\|rAp\|rAp\|rCp\|r |
| | | Cp\|rUp\|rCp\|dTp\|dT] |
| 721 | UAACUAUCAAACACAACCU | [rUp\|rAp\|rAp\|rCp\|2mUp\|rAp\|rUp\|2mCp\|rA |
| | | p\|rAp\|rAp\|2mCp\|rAp\|2mCp\|rAp\|rAp\|r |
| | | Cp\|rCp\|rUp\|dTp\|dT] |
| 723 | UAAACUAUCAAACACAACC | [rUp\|rAp\|rAp\|rAp\|rCp\|2mUp\|rAp\|rUp\|2mC |
| | | p\|rAp\|rAp\|rAp\|2mCp\|rAp\|2mCp\|rAp\|r |
| | | Ap\|rCp\|rCp\|dTp\|dT] |
| 797 | UAAUUCUGUCAUCCUGCAC | [rUp\|rAp\|rAp\|rUp\|rUp\|rCp\|rUp\|rGp\|rUp\| |
| | | 2mCp\|rAp\|rUp\|rCp\|rCp\|rUp\|rGp\|2mCp\|r |
| | | Ap\|rCp\|dTp\|dT] |
| 821 | UAAUGUUAUCCAUCCUUUC | [rUp\|rAp\|rAp\|rUp\|rGp\|rUp\|2mUp\|rAp\|rUp |
| | | \|rCp\|2mCp\|rAp\|rUp\|rCp\|rCp\|rUp\|rUp\|r |
| | | Up\|rCp\|dTp\|dT] |
| 823 | UAAAUGUUAUCCAUCCUUU | [rUp\|rAp\|rAp\|rAp\|rUp\|rGp\|rUp\|2mUp\|rAp |
| | | \|rUp\|rCp\|2mCp\|rAp\|rUp\|rCp\|rCp\|rUp\|r |
| | | Up\|rUp\|dTp\|dT] |
| 827 | UUCAAAAUAAAUGUUAUCC | [rUp\|rUp\|2mCp\|rAp\|rAp\|rAp\|rAp\|2mUp\|rA |
| | | p\|rAp\|rAp\|rUp\|rGp\|rUp\|2mUp\|rAp\|rU |
| | | p\|rCp\|rCp\|dTp\|dT] |
| 849 | UCAUAAAACUCUGUAACUG | [rUp\|2mCp\|rAp\|2mUp\|rAp\|rAp\|rAp\|rAp\|rC |
| | | p\|rUp\|rCp\|rUp\|rGp\|2mUp\|rAp\|rAp\|rC |
| | | p\|rUp\|rGp\|dTp\|dT] |
| 853 | UUUGCAUAAAACUCUGUAA | [rUp\|rUp\|rUp\|rGp\|2mCp\|rAp\|2mUp\|rAp\|rA |
| | | p\|rAp\|rAp\|rCp\|rUp\|rCp\|rUp\|rGp\|2mu |
| | | p\|rAp\|rAp\|dTp\|dT] |
| 855 | UUUUGCAUAAAACUCUGUA | [rUp\|rUp\|rUp\|rUp\|rGp\|2mCp\|rAp\|2mUp\|rA |
| | | p\|rAp\|rAp\|rAp\|rCp\|rUp\|rCp\|rUp\|rGp\| |
| | | 2mUp\|rAp\|dTp\|dT] |
| 901 | UUAACAAUGCCAGAAAUAA | [rUp\|2mUp\|rAp\|rAp\|2mCp\|rAp\|rAp\|rUp\|rG |
| | | p\|rCp\|2mCp\|rAp\|rGp\|rAp\|rAp\|rAp\|2m |
| | | Up\|rAp\|rAp\|dTp\|dT] |
| 915 | UAAAUCACAGGCUUCUCUA | [rUp\|rAp\|rAp\|rAp\|rUp\|2mCp\|rAp\|2mCp\|rA |
| | | p\|rGp\|rGp\|rCp\|rUp\|rUp\|rCp\|rUp\|rCp\| |
| | | 2mUp\|rAp\|dTp\|dT] |
| 929 | UUUAUGAUGGCAGCCAAAG | [rUp\|rUp\|2mUp\|rAp\|rUp\|rGp\|rAp\|rUp\|rGp |
| | | \|rGp\|2mCp\|rAp\|rGp\|rCp\|2mCp\|rAp\|rA |
| | | p\|rAp\|rGp\|dTp\|dT] |
| 931 | UUUUAUGAUGGCAGCCAAA | [rUp\|rUp\|rUp\|2mUp\|rAp\|rUp\|rGp\|rAp\|rUp |
| | | \|rGp\|rGp\|2mCp\|rAp\|rGp\|rCp\|2mCp\|rA |
| | | p\|rAp\|rAp\|dTp\|dT] |
| 935 | UUAUUUUAUGAUGGCAGCC | [rUp\|2mUp\|rAp\|rUp\|rUp\|rUp\|2mUp\|rAp\|rU |
| | | p\|rGp\|rAp\|rUp\|rGp\|rGp\|2mCp\|rAp\|rG |
| | | p\|rCp\|rCp\|dTp\|dT] |
| 937 | UGUAUUUUAUGAUGGCAGC | [rUp\|rGp\|2mUp\|rAp\|rUp\|rUp\|rUp\|2mUp\|rA |
| | | p\|rUp\|rGp\|rAp\|rUp\|rGp\|rGp\|2mCp\|rA |
| | | p\|rGp\|rCp\|dTp\|dT] |
| 947 | UGUUGAAUUCUUUGAGGUA | [rUp\|rGp\|rUp\|rUp\|rGp\|rAp\|rAp\|rUp\|rUp\| |
| | | rCp\|rUp\|rUp\|rUp\|rGp\|rAp\|rGp\|rGp\|2mU |
| | | p\|rAp\|dTp\|dT] |
| 949 | UAAGUUGAAUUCUUUGAGG | [rUp\|rAp\|rAp\|rGp\|rUp\|rUp\|rGp\|rAp\|rAp\| |
| | | rUp\|rUp\|rCp\|rUp\|rUp\|rUp\|rGp\|rAp\|rGp\| |
| | | rGp\|dTp\|dT] |
| 967 | UAAUUCUCAGGUUUGGAGA | [rUp\|rAp\|rAp\|rUp\|rUp\|rCp\|rUp\|2mCp\|rAp |
| | | \|rGp\|rGp\|rUp\|rUp\|rUp\|rGp\|rGp\|rAp\|rG |
| | | p\|rAp\|dTp\|dT] |
| 971 | UGUUUAAAAUUCUCAGGUU | [rUp\|rGp\|rUp\|rUp\|2mUp\|rAp\|rAp\|rAp\|rAp |
| | | \|rUp\|rUp\|rCp\|rUp\|2mCp\|rAp\|rGp\|rGp\|r |
| | | Up\|rUp\|dTp\|dT] |
| 973 | UCUGUUUAAAAUUCUCAGG | [rUp\|rCp\|rUp\|rGp\|rUp\|rUp\|2mUp\|rAp\|rA |
| | | p\|rAp\|rAp\|rUp\|rUp\|rCp\|rUp\|2mCp\|rAp\|r |
| | | Gp\|rGp\|dTp\|dT] |
| 989 | UUCCAUUUUACUUGAUAGC | [rUp\|rUp\|rCp\|2mCp\|rAp\|rUp\|rUp\|rUp\|2mU |
| | | p\|rAp\|rCp\|rUp\|rUp\|rGp\|rAp\|2mUp\|rA |
| | | p\|rGp\|rCp\|dTp\|dT] |
| 991 | UUUCCAUUUUACUUGAUAG | [rUp\|rUp\|rUp\|rCp\|2mCp\|rAp\|rUp\|rUp\|rUp |
| | | \|2mUp\|rAp\|rCp\|rUp\|rUp\|rGp\|rAp\|2mU |
| | | p\|rAp\|rGp\|dTp\|dT] |
| 993 | UAUUCCAUUUUACUUGAUA | [rUp\|rAp\|rUp\|rUp\|rCp\|2mCp\|rAp\|rUp\|rUp |
| | | \|rUp\|2mUp\|rAp\|rCp\|rUp\|rUp\|rGp\|rAp\| |
| | | 2mUp\|rAp\|dTp\|dT] |
| 995 | UAAAUUCCAUUUUACUUGA | [rUp\|rAp\|rAp\|rAp\|rUp\|rUp\|rCp\|2mCp\|rAp |
| | | \|rUp\|rUp\|rUp\|2mUp\|rAp\|rCp\|rUp\|rUp\|r |
| | | Gp\|rAp\|dTp\|dT] |
| 997 | UUAAAUUCCAUUUUACUUG | [rUp\|2mUp\|rAp\|rAp\|rAp\|rUp\|rUp\|rCp\|2mC |
| | | p\|rAp\|rUp\|rUp\|rUp\|2mUp\|rAp\|rCp\|rU |
| | | p\|rUp\|rGp\|dTp\|dT] |
| 1007 | UUUCCAUUAAUUGUCAUAA | [rUp\|rUp\|rUp\|rCp\|2mCp\|rAp\|rUp\|2mUp\|rA |
| | | p\|rAp\|rUp\|rUp\|rGp\|rUp\|2mCp\|rAp\|2 |
| | | mUp\|rAp\|rAp\|dTp\|dT] |
| 1021 | UAUUCCUUAAUGUUGUUCC | [rUp\|rAp\|rUp\|rUp\|rCp\|rCp\|rUp\|2mUp\|rAp |
| | | \|rAp\|rUp\|rGp\|rUp\|rUp\|rGp\|rUp\|rUp\|rC |
| | | p\|rCp\|dTp\|dT] |
| 1032 | UAUUCUGUAGGAUUUCCAG | [rUp\|rAp\|rUp\|rUp\|rCp\|rUp\|rGp\|2mUp\|rA |
| | | p\|rGp\|rGp\|rAp\|rUp\|rUp\|rUp\|rCp\|2mCp\| |
| | | rAp\|rGp\|dTp\|dT] |
| 1055 | UGUUUUCAUAUCAGUCUGA | [rUp\|rGp\|rUp\|rUp\|rUp\|rUp\|2mCp\|rAp\|2mU |
| | | p\|rAp\|rUp\|2mCp\|rAp\|rGp\|rUp\|rCp\|rU |
| | | p\|rGp\|rAp\|dTp\|dT] |
| 1057 | UUUGGUUUUCAUAUCAGUC | [rUp\|rUp\|rUp\|rGp\|rGp\|rUp\|rUp\|rUp\|rUp |
| | | \|2mCp\|rAp\|2mUp\|rAp\|rUp\|2mCp\|rAp\|rG |
| | | p\|rUp\|rCp\|dTp\|dT] |
| 1059 | UUUUGGUUUUCAUAUCAGU | [rUp\|rUp\|rUp\|rUp\|rGp\|rGp\|rUp\|rUp\|rUp\| |
| | | rUp\|2mCp\|rAp\|2mUp\|rAp\|rUp\|2mCp\|rA |
| | | p\|rGp\|rUp\|dTp\|dT] |
| 1077 | UGUCUAAAACCCACAGCAA | [rUp\|rGp\|rUp\|rCp\|2mUp\|rAp\|rAp\|rAp\|rAp |
| | | \|rCp\|rCp\|2mCp\|rAp\|2mCp\|rAp\|rGp\|2 |
| | | mCp\|rAp\|rAp\|dTp\|dT] |
| 1089 | UUUUAGUGUGGUCUAAAAC | [rUp\|rUp\|rUp\|2mUp\|rAp\|rGp\|rUp\|rGp\|rUp |
| | | \|rGp\|rGp\|rUp\|rCp\|2mUp\|rAp\|rAp\|rAp\| |
| | | rAp\|rCp\|dTp\|dT] |
| 1097 | UGAAGUUUUAGUGUGGUCU | [rUp\|rGp\|rAp\|rAp\|rGp\|rUp\|rUp\|rUp\|2mUp |
| | | \|rAp\|rGp\|rUp\|rGp\|rUp\|rGp\|rGp\|rUp\|rC |
| | | p\|rUp\|dTp\|dT] |
| 1099 | UAAUGAAGUUUUAGUGUGG | [rUp\|rAp\|rAp\|rUp\|rGp\|rAp\|rAp\|rGp\|rUp\| |
| | | rUp\|rUp\|2mUp\|rAp\|rGp\|rUp\|rGp\|rUp\|rG |
| | | p\|rGp\|dTp\|dT] |
| 1111 | UCUUUAACUUCCGUCUCCC | [rUp\|rCp\|rUp\|rUp\|2mUp\|rAp\|rAp\|rCp\|rUp |
| | | \|rUp\|rCp\|rCp\|rGp\|rUp\|rCp\|rUp\|rCp\|rC |
| | | p\|rCp\|dTp\|dT] |
| 1113 | UUCUUUAACUUCCGUCUCC | [rUp\|rUp\|rCp\|rUp\|rUp\|2mUp\|rAp\|rAp\|rCp |
| | | \|rUp\|rUp\|rCp\|rCp\|rGp\|rUp\|rCp\|rUp\|rC |
| | | p\|rCp\|dTp\|dT] |
| 1127 | UAUUUAAGGAGUGGCUGGG | [rUp\|rAp\|rUp\|rUp\|2mUp\|rAp\|rAp\|rGp\|rGp |
| | | \|rAp\|rGp\|rUp\|rGp\|rGp\|rCp\|rUp\|rGp\|rG |
| | | p\|rGp\|dTp\|dT] |
| 1215 | UAUUUUCUAUCUGACCAAA | [rUp\|rAp\|rUp\|rUp\|rUp\|rUp\|rCp\|2mUp\|rAp |
| | | \|rUp\|rCp\|rUp\|rGp\|rAp\|rCp\|2mCp\|rAp\|r |
| | | Ap\|rAp\|dTp\|dT] |
| 1221 | UUAGAUGAUUUUCUAUCUG | [rUp\|2mUp\|rAp\|rGp\|rAp\|rUp\|rGp\|rAp\|rUp |
| | | \|rUp\|rUp\|rUp\|rCp\|2mUp\|rAp\|rUp\|rCp\|r |
| | | Up\|rGp\|dTp\|dT] |
| 1230 | UCAAUUUACGUAGAUGAUU | [rUp\|2mCp\|rAp\|rAp\|rUp\|rUp\|2mUp\|rAp\|rC |
| | | p\|rGp\|2mUp\|rAp\|rGp\|rAp\|rUp\|rGp\|rA |
| | | prUp\|rUp\|dTp\|dT] |
| 1307 | UAUUAUUGCUUGAAAUUCU | [rUp\|rAp\|rUp\|2mUp\|rAp\|rUp\|rUp\|rGp\|rCp |
| | | \|rUp\|rUp\|rGp\|rAp\|rAp\|rAp\|rUp\|rUp\|rC |
| | | p\|rUp\|dTp\|dT] |
| 1309 | UGGUAUUAUUGCUUGAAAU | [rUp\|rGp\|rGp\|2mUp\|rAp\|rUp\|2mUp\|rAp\|rU |
| | | p\|rUp\|rGp\|rCp\|rUp\|rUp\|rGp\|rAp\|rAp\| |
| | | rAp\|rUp\|dTp\|dT] |
| 1311 | UAGGUAUUAUUGCUUGAAA | [rUp\|rAp\|rGp\|rGp\|2mUp\|rAp\|rUp\|2mUp\|rA |
| | | p\|rUp\|rUp\|rGp\|rCp\|rUp\|rUp\|rGp\|rAp\| |
| | | rAp\|rAp\|dTp\|dT] |
| 1319 | UGGAAUUAACAGCAGGUAU | [rUp\|rGp\|rGp\|rAp\|rAp\|rUp\|2mUp\|rAp\|rAp |
| | | \|2mCp\|rAp\|rGp\|2mCp\|rAp\|rGp\|rGp\|2 |
| | | mUp\|rAp\|rUp\|dTp\|dT] |
| 1363 | UAAUUUCUAAAAUAACGGU | [rUp\|rAp\|rAp\|rUp\|rUp\|rUp\|rCp\|2mUp\|rAp |
| | | \|rAp\|rAp\|rAp\|2mUp\|rAp\|rAp\|rCp\|rGp\|r |
| | | Gp\|rUp\|dTp\|dT] |
| 1387 | UAAGUAAUGCUCCACUGGA | [rUp\|rAp\|rAp\|rGp\|2mUp\|rAp\|rAp\|rUp\|rGp |
| | | \|rCp\|rUp\|rCp\|2mCp\|rAp\|rCp\|rUp\|rGp\|r |
| | | Gp\|rAp\|dTp\|dT] |
| 1395 | UUAUCUUUAAGUAAUGCUC | [rUp\|2mUp\|rAp\|rUp\|rCp\|rUp\|rUp\|2mUp\|rA |
| | | p\|rAp\|rGp\|2mUp\|rAp\|rAp\|rUp\|rGp\|rC |
| | | p\|rUp\|rCp\|dTp\|dT] |
| 1397 | UGUAUCUUUAAGUAAUGCU | [rUp\|rGp\|2mUp\|rAp\|rUp\|rCp\|rUp\|rUp\|2mU |
| | | p\|rAp\|rAp\|rGp\|2mUp\|rAp\|rAp\|rUp\|rG |
| | | p\|rCp\|rUp\|dTp\|dT] |
| 1401 | UAUUGAGUAUCUUUAAGUA | [rUp\|rAp\|rUp\|rUp\|rGp\|rAp\|rGp\|2mUp\|rAp |
| | | \|rUp\|rCp\|rUp\|rUp\|2mUp\|rAp\|rAp\|rGp\| |
| | | 2mUp\|rAp\|dTp\|dT] |
| 1405 | UUUCAUUGAGUAUCUUUAA | [rUp\|rUp\|rUp\|2mCp\|rAp\|rUp\|rUp\|rGp\|rAp |
| | | \|rGp\|2mUp\|rAp\|rUp\|rCp\|rUp\|rUp\|2mU |
| | | p\|rAp\|rAp\|dTp\|dT] |
| 1425 | UUUCAGUUUUAUCCCCAAC | [rUp\|rUp\|rUp\|2mCp\|rAp\|rGp\|rUp\|rUp\|rUp |
| | | \|2mUp\|rAp\|rUp\|rCp\|rCp\|rCp\|2mCp\|rA |
| | | p\|rAp\|rCp\|dTp\|dT] |
| 1427 | UAUUCAGUUUUAUCCCCAA | [rUp\|rAp\|rUp\|rUp\|2mCp\|rAp\|rGp\|rUp\|rUp |
| | | \|rUp\|2mUp\|rAp\|rUp\|rCp\|rCp\|rCp\|2mC |
| | | p\|rAp\|rAp\|dTp\|dT] |
| 1449 | UAUUAAAGGGAAGUCAGAA | [rUp\|rAp\|rUp\|2mUp\|rAp\|rAp\|rAp\|rGp\|rGp |
| | | \|rGp\|rAp\|rAp\|rGp\|rUp\|2mCp\|rAp\|rGp\|r |
| | | Ap\|rAp\|dTp\|dT] |
| 1453 | UUUUAUUAAAGGGAAGUCA | [rUp\|rUp\|rUp\|2mUp\|rAp\|rUp\|2mUp\|rAp\|rA |
| | | p\|rAp\|rGp\|rGp\|rGp\|rAp\|rAp\|rGp\|rUp\| |
| | | 2mCp\|rAp\|dTp\|dT] |
| 1455 | UGAAUUUCAUCCUUCCUCU | [rUp\|rGp\|rAp\|rAp\|rUp\|rUp\|rUp\|2mCp\|rAp |
| | | \|rUp\|rCp\|rCp\|rUp\|rUp\|rCp\|rCp\|rUp\|rC |
| | | p\|rUp\|dTp\|dT] |
| 1507 | UCAUAUUGUGCAGAAGGAU | [rUp\|2mCp\|rAp\|2mUp\|rAp\|rUp\|rUp\|rGp\|rU |
| | | p\|rGp\|2mCp\|rAp\|rGp\|rAp\|rAp\|rGp\|rG |
| | | p\|rAp\|rUp\|dTp\|dT] |
| 1525 | UUCAUAAACUCCUGUCCUG | [rUp\|rUp\|2mCp\|rAp\|2mUp\|rAp\|rAp\|rAp\|rC |
| | | p\|rUp\|rCp\|rCp\|rUp\|rGp\|rUp\|rCp\|rCp\|r |
| | | Up\|rGp\|dTp\|dT] |
| 1541 | UAAGAUACAGCAGAGUUCU | [rUp\|rAp\|rAp\|rGp\|rAp\|2mUp\|rAp\|2mCp\|rA |
| | | p\|rGp\|2mCp\|rAp\|rGp\|rAp\|rGp\|rUp\|rU |
| | | p\|rCp\|rUp\|dTp\|dT] |
| 1547 | UAUACAAGAUACAGCAGAG | [rUp\|rAp\|2mUp\|rAp\|2mCp\|rAp\|rAp\|rGp\|rA |
| | | p\|2mUp\|rAp\|2mCp\|rAp\|rGp\|2mCp\|rA |
| | | p\|rGp\|rAp\|rGp\|dTp\|dT] |
| 1657 | UUAGAAAAUCAAGCCAUUC | [rUp\|2mUp\|rAp\|rGp\|rAp\|rAp\|rAp\|rAp\|rUp |
| | | \|2mCp\|rAp\|rAp\|rGp\|rCp\|2mCp\|rAp\|rU |
| | | p\|rUp\|rCp\|dTp\|dT] |
| 1667 | UACUGAAUUUCUCUAGAAA | [rUp\|rAp\|rCp\|rUp\|rGp\|rAp\|rAp\|rUp\|rUp\| |
| | | rUp\|rCp\|rUp\|rCp\|2mUp\|rAp\|rGp\|rAp\|rA |
| | | p\|rAp\|dTp\|dT] |
| 1675 | UAUAAUGUUCACUGAAUUU | [rUp\|rAp\|2mUp\|rAp\|rAp\|rUp\|rGp\|rUp\|rUp |
| | | \|2mCp\|rAp\|rCp\|rUp\|rGp\|rAp\|rAp\|rUp\|r |
| | | Up\|rUp\|dTp\|dT] |
| 1677 | UGAUAAUGUUCACUGAAUU | [rUp\|rGp\|rAp\|2mUp\|rAp\|rAp\|rUp\|rGp\|rUp |
| | | \|rUp\|2mCp\|rAp\|rCp\|rUp\|rGp\|rAp\|rAp\|r |
| | | Up\|rUp\|dTp\|dT] |
| 1679 | UUGAUAAUGUUCACUGAAU | [rUp\|rUp\|rGp\|rAp\|2mUp\|rAp\|rAp\|rUp\|rGp |
| | | \|rUp\|rUp\|2mCp\|rAp\|rCp\|rUp\|rGp\|rAp\|r |
| | | Ap\|rUp\|dTp\|dT] |
| 1723 | UGAGAAAAUGCAGUCAACA | [rUp\|rGp\|rAp\|rGp\|rAp\|rAp\|rAp\|rAp\|rUp\| |
| | | rGp\|2mCp\|rAp\|rGp\|rUp\|2mCp\|rAp\|rAp\| |
| | | 2mCp\|rAp\|dTp\|dT] |
| 1763 | UUUCUUGUACAGUUGGUCU | [rUp\|rUp\|rUp\|rCp\|rUp\|rUp\|rGp\|2mUp\|rAp |
| | | \|2mCp\|rAp\|rGp\|rUp\|rUp\|rGp\|rGp\|rUp\| |
| | | rCp\|rUp\|dTp\|dT] |
| 1767 | UUCUUUCUUCUUGUACAGU | [rUp\|rUp\|rCp\|rUp\|rUp\|rUp\|rCp\|rUp\|rUp\| |
| | | rCp\|rUp\|rUp\|rGp\|2mUp\|rAp\|2mCp\|rAp\|r |
| | | Gp\|rUp\|dTp\|dT] |
| 1769 | UUUCUUUCUUCUUGUACAG | [rUp\|rUp\|rUp\|rCp\|rUp\|rUp\|rUp\|rCp\|rUp\| |
| | | rUp\|rCp\|rUp\|rUp\|rGp\|2mUp\|rAp\|2mCp\| |
| | | rAp\|rGp\|dTp\|dT] |
| 1837 | UUCAUUACUCUCUCUGAGU | [rUp\|rUp\|2mCp\|rAp\|rUp\|2mUp\|rAp\|rCp\|rU |
| | | p\|rCp\|rUp\|rCp\|rUp\|rCp\|rUp\|rGp\|rAp\|r |
| | | Gp\|rUp\|dTp\|dT] |
| 1839 | UUAUCAUUACUCUCUCUGA | [rUp\|2mUp\|rAp\|rUp\|2mCp\|rAp\|rUp\|2mUp\|r |
| | | Ap\|rCp\|rUp\|rCp\|rUp\|rCp\|rUp\|rCp\|ru |
| | | p\|rGp\|rAp\|dTp\|dT] |
| 1843 | UAUUAUCAUUACUCUCUCU | [rUp\|rAp\|rUp\|2mUp\|rAp\|rUp\|2mCp\|rAp\|rU |
| | | p\|2mUp\|rAp\|rCp\|rUp\|rCp\|rUp\|rCp\|rU |
| | | p\|rCp\|rUp\|dTp\|dT] |
| 1869 | UUUAUGUAGGAGCUCUUUC | [rUp\|rUp\|2mUp\|rAp\|rUp\|rGp\|2mUp\|rAp\|rG |
| | | p\|rGp\|rAp\|rGp\|rCp\|rUp\|rCp\|rUp\|rUp\| |
| | | rUp\|rCp\|dTp\|dT] |
| 1887 | UAAUCAAUGCAACUUGUUU | [rUp\|rAp\|rAp\|rUp\|2mCp\|rAp\|rAp\|rUp\|rGp |
| | | \|2mCp\|rAp\|rAp\|rCp\|rUp\|rUp\|rGp\|rUp\|r |
| | | Up\|rUp\|dTp\|dT] |
| 1889 | UUAAUCAAUGCAACUUGUU | [rUp\|2mUp\|rAp\|rAp\|rUp\|2mCp\|rAp\|rAp\|rU |
| | | p\|rGp\|2mCp\|rAp\|rAp\|rCp\|rUp\|rUp\|rG |
| | | p\|rUp\|rUp\|dTp\|dT] |
| 1965 | UUGUGAAAAUGCCAUCCAC | [rUp\|rUp\|rGp\|rUp\|rGp\|rAp\|rAp\|rAp\|rAp\| |
| | | rUp\|rGp\|rCp\|2mCp\|rAp\|rUp\|rCp\|2mCp\|r |
| | | Ap\|rCp\|dTp\|dT] |
| 1991 | UUUAUAUAUAUUGUCUGCA | [rUp\|rUp\|2mUp\|rAp\|2mUp\|rAp\|2mUp\|rAp\|2 |
| | | mUp\|rAp\|rUp\|rUp\|rGp\|rUp\|rCp\|rUp\| |
| | | rGp\|2mCp\|rAp\|dTp\|dT] |
| 2031 | UGAUUAUUUCUGCUGUGUC | [rUp\|rGp\|rAp\|rUp\|2mUp\|rAp\|rUp\|rUp\|rUp |
| | | \|rCp\|rUp\|rGp\|rCp\|rUp\|rGp\|rUp\|rGp\|rU |
| | | p\|rCp\|dTp\|dT] |
| 2109 | UAAUACUCAAGUGUAGCAU | [rUp\|rAp\|rAp\|2mUp\|rAp\|rCp\|rUp\|2mCp\|rA |
| | | p\|rAp\|rGp\|rUp\|rGp\|2mUp\|rAp\|rGp\|2 |
| | | mCp\|rAp\|rUp\|dTp\|dT] |
| 2129 | UUUAAGGAUUUCACAUCUC | [rUp\|rUp\|2mUp\|rAp\|rAp\|rGp\|rGp\|rAp\|rUp |
| | | \|rUp\|rUp\|2mCp\|rAp\|2mCp\|rAp\|rUp\|rC |
| | | p\|rUp\|rCp\|dTp\|dT] |
| 2231 | UCAAAAUCAGGGACUUGUU | [rUp\|2mCp\|rAp\|rAp\|rAp\|rAp\|rUp\|2mCp\|rA |
| | | p\|rGp\|rGp\|rGp\|rAp\|rCp\|rUp\|rUp\|rGp\|r |
| | | Up\|rUp\|dTp\|dT] |
| 2243 | UUAAAGGAAGGUGACAAAA | [rUp\|2mUp\|rAp\|rAp\|rAp\|rGp\|rGp\|rAp\|rAp |
| | | \|rGp\|rGp\|rUp\|rGp\|rAp\|2mCp\|rAp\|rAp\|r |
| | | Ap\|rAp\|dTp\|dT] |
| 2247 | UUUUGGUAAAGGAAGGUGA | [rUp\|rUp\|rUp\|rUp\|rGp\|rGp\|2mUp\|rAp\|rA |
| | | p\|rAp\|rGp\|rGp\|rAp\|rAp\|rGp\|rGp\|rUp\|rG |
| | | p\|rAp\|dTp\|dT] |
| 2255 | UUCUAGUUAUUUGGUAAAG | [rUp\|rUp\|rCp\|2mUp\|rAp\|rGp\|rUp\|2mUp\|rA |
| | | p\|rUp\|rUp\|rUp\|rGp\|rGp\|2mUp\|rAp\|rA |
| | | p\|rAp\|rGp\|dTp\|dT] |
| 2275 | UUUUAAUCCAUAACUCCUU | [rUp\|rUp\|rUp\|2mUp\|rAp\|rAp\|rUp\|rCp\|2m |
| | | Cp\|rAp\|2mUp\|rAp\|rAp\|rCp\|rUp\|rCp\|rC |
| | | p\|rUp\|rUp\|dTp\|dT] |
| 2295 | UCUAGUUUAGCCACAUUUA | [rUp\|rCp\|2mUp\|rAp\|rGp\|rUp\|rUp\|2mUp\|rA |
| | | p\|rGp\|rCp\|2mCp\|rAp\|2mCp\|rAp\|rUp\| |
| | | rUp\|2mUp\|rAp\|dTp\|dT] |
| 2331 | UUAUUAAUCCUUCCAGCUC | [rUp\|2mUp\|rAp\|rUp\|2mUp\|rAp\|rAp\|rUp\|rC |
| | | p\|rCp\|rUp\|rUp\|rCp\|2mCp\|rAp\|rGp\|rC |
| | | p\|rUp\|rCp\|dTp\|dT] |
| 2335 | UUUUCGUAUUUAUUAAUCC | [rUp\|rUp\|rUp\|rUp\|rCp\|rGp\|2mUp\|rAp\|rUp |
| | | \|rUp\|2mUp\|rAp\|rUp\|2mUp\|rAp\|rAp\|rU |
| | | p\|rCp\|rCp\|dTp\|dT] |
| 2357 | UUUUGCAAAAUACUUGAGU | [rUp\|rUp\|rUp\|rUp\|rGp\|2mCp\|rAp\|rAp\|rAp |
| | | \|rAp\|2mUp\|rAp\|rCp\|rUp\|rUp\|rGp\|rAp\|r |
| | | Gp\|rUp\|dTp\|dT] |
| 2361 | UUAACUUUGCAAAAUACUU | [rUp\|2mUp\|rAp\|rAp\|rCp\|rUp\|rUp\|rUp\|rGp |
| | | \|2mCp\|rAp\|rAp\|rAp\|rAp\|2mUp\|rAp\|rC |
| | | p\|rUp\|rUp\|dTp\|dT] |
| 2363 | UCAUAACUUUGCAAAAUAC | [rUp\|2mCp\|rAp\|2mUp\|rAp\|rAp\|rCp\|rUp\|rU |
| | | p\|rUp\|rGp\|2mCp\|rAp\|rAp\|rAp\|rAp\|2m |
| | | Up\|rAp\|rCp\|dTp\|dT] |
| 2449 | UAGUUAUUUUGUACAGUUG | [rUp\|rAp\|rGp\|rUp\|2mUp\|rAp\|rUp\|rUp\|rUp |
| | | \|rUp\|rGp\|2mUp\|rAp\|2mCp\|rAp\|rGp\|rU |
| | | p\|rUp\|rGp\|dTp\|dT] |
| 2503 | UAAUUUUAUGCUCACAUGU | [rUp\|rAp\|rAp\|rUp\|rUp\|rUp\|2mUp\|rAp\|rUp |
| | | \|rGp\|rCp\|rUp\|2mCp\|rAp\|2mCp\|rAp\|rU |
| | | p\|rGp\|rUp\|dTp\|dT] |
| 2505 | UUAAUUUUAUGCUCACAUG | [rUp\|2mUp\|rAp\|rAp\|rUp\|rUp\|rUp\|2mUp\|rA |
| | | p\|rUp\|rGp\|rCp\|rUp\|2mCp\|rAp\|2mCp\| |
| | | rAp\|rUp\|rGp\|dTp\|dT] |
| 2517 | UUAUACCAUGGUCAUAAUU | [rUp\|2mUp\|rAp\|2mUp\|rAp\|rCp\|2mCp\|rAp\|r |
| | | Up\|rGp\|rGp\|rUp\|2mCp\|rAp\|2mUp\|r |
| | | Ap\|rAp\|rUp\|rUp\|dTp\|dT] |
| 2519 | UAUAUACCAUGGUCAUAAU | [rUp\|rAp\|2mUp\|rAp\|2mUp\|rAp\|rCp\|2mCp\|r |
| | | Ap\|rUp\|rGp\|rGp\|rUp\|2mCp\|rAp\|2m |
| | | Up\|rAp\|rAp\|rUp\|dTp\|dT] |
| 2579 | UCUAUUCAAGAGUGUUUAU | [rUp\|rCp\|2mUp\|rAp\|rUp\|rUp\|2mCp\|rAp\|rA |
| | | p\|rGp\|rAp\|rGp\|rUp\|rGp\|rUp\|rUp\|2mU |
| | | p\|rAp\|rUp\|dTp\|dT] |
| 2581 | UUCUAUUCAAGAGUGUUUA | [rUp\|rUp\|rCp\|2mUp\|rAp\|rUp\|rUp\|2mCp\|rA |
| | | p\|rAp\|rGp\|rAp\|rGp\|rUp\|rGp\|rUp\|rUp |
| | | 2mUp\|rAp\|dTp\|dT] |
| 2593 | UUACAAAGUGGAAGUCUAU | [rUp\|2mUp\|rAp\|2mCp\|rAp\|rAp\|rAp\|rGp\|rU |
| | | p\|rGp\|rGp\|rAp\|rAp\|rGp\|rUp\|rCp\|2mU |
| | | p\|rAp\|rUp\|dTp\|dT] |
| 2597 | UAUUACAAAGUGGAAGUCU | [rUp\|rAp\|rUp\|2mUp\|rAp\|2mCp\|rAp\|rAp\|rA |
| | | p\|rGp\|rUp\|rGp\|rGp\|rAp\|rAp\|rGp\|rUp\|r |
| | | Cp\|rUp\|dTp\|dT] |
| 2603 | UCUAAUUACAAAGUGGAAG | [rUp\|rCp\|2mUp\|rAp\|rAp\|rUp\|2mUp\|rAp\|2m |
| | | Cp\|rAp\|rAp\|rAp\|rGp\|rUp\|rGp\|rGp\|rA |
| | | p\|rAp\|rGp\|dTp\|dT] |
| 2655 | UUAUAUUCUGCCACUUAAG | [rUp\|2mUp\|rAp\|2mUp\|rAp\|rUp\|rUp\|rCp\|rU |
| | | p\|rGp\|rCp\|2mCp\|rAp\|rCp\|rUp\|2mUp\| |
| | | rAp\|rAp\|rGp\|dTp\|dT] |
| 2657 | UUUAUAUUCUGCCACUUAA | [rUp\|rUp\|2mUp\|rAp\|2mUp\|rAp\|rUp\|rUp\|rC |
| | | p\|rUp\|rGp\|rCp\|2mCp\|rAp\|rCp\|rUp\|2 |
| | | mUp\|rAp\|rAp\|dTp\|dT] |
| 2687 | UUUAUAUCACCCUCCAAAA | [rUp\|rUp\|2mUp\|rAp\|2mUp\|rAp\|rUp\|2mCp\|r |
| | | Ap\|rCp\|rCp\|rCp\|rUp\|rCp\|2mCp\|rAp\| |
| | | rAp\|rAp\|rAp\|dTp\|dT] |
| 2763 | UUCUUAUGUUCUUGGUGGA | [rUp\|rUp\|rCp\|rUp\|2mUp\|rAp\|rUp\|rGp\|rUp |
| | | \|rUp\|rCp\|rUp\|rUp\|rGp\|rGp\|rUp\|rGp\|rG |
| | | p\|rAp\|dTp\|dT] |
| 2769 | UAAUUCUUAUGUUCUUGGU | [rUp\|rAp\|rAp\|rUp\|rUp\|rCp\|rUp\|2mUp\|rAp |
| | | \|rUp\|rGp\|rUp\|rUp\|rCp\|rUp\|rUp\|rGp\|rG |
| | | p\|rUp\|dTp\|dT] |
| 2783 | UAAAAGCUUGAUAAUUCUA | [rUp\|rAp\|rAp\|rAp\|rAp\|rGp\|rCp\|rUp\|rUp\| |
| | | rGp\|rAp\|2mUp\|rAp\|rAp\|rUp\|rUp\|rCp\|2m |
| | | Up\|rAp\|dTp\|dT] |
| 2845 | UACAAAUAAUUUCAUCCAA | [rUp\|rAp\|2mCp\|rAp\|rAp\|rAp\|2mUp\|rAp\|rA |
| | | p\|rUp\|rUp\|rUp\|2mCp\|rAp\|rUp\|rCp\|2m |
| | | Cp\|rAp\|rAp\|dTp\|dT] |
| 2847 | UAUGACAAAUAAUUUCAUC | [rUp\|rAp\|rUp\|rGp\|rAp\|2mCp\|rAp\|rAp\|rAp |
| | | \|2mUp\|rAp\|rAp\|rUp\|rUp\|rUp\|2mCprA |
| | | p\|rUp\|rCp\|dTp\|dT] |

### Example 7. MSH3 siRNA Screening Data and Knockdown Data for Selected Duplexes

Various screenings were implemented to determine potential lead siRNA duplex candidates.

Selected siRNA duplexes targeting human MSH3 were screened in Hela cells at 10 nM and 0.5 nM doses to determine potential lead siRNA candidates. The screening results are provided below in Table 15. The sense and antisense oligonucleotides in Table 15 each include a dTdT overhang on the 3′ end.

Additionally, every A and G in each sense oligonucleotide in Table 15 is a ribonucleotide. Every C and U in each sense oligonucleotide is a 2′-O-Methyl ribonucleotide.

Also, every A and Gin each antisense oligonucleotide in Table 15 is a ribonucleotide. Every C and U preceding an A in the antisense oligonucleotide is a 2′-O-Methyl ribonucleotide, with one exception: U is the first nucleotide of the antisense strand, and it is a ribonucleotide.

Each nucleotide in the sense and antisense oligonucleotide in Table 15 is linked by a phosphate.

TABLE 15

| | SEQ ID | | SEQ ID NO./ |
|---|---|---|---|
| | NO./Sense | | Antisense |
| Sense | No. | antisense | No. |
| GCCAAAAUGUACUGAUUUA | 156 | UAAAUCAGUACAUUUUGGC | 157 |
| GUGGAAUGUGGAUAUAAGA | 358 | UCUUAUAUCCACAUUCCAC | 359 |
| GAUAUAAGUAUAGAUUCUA | 374 | UAGAAUCUAUACUUAUAUC | 375 |
| GCCAUUUAGAUCACAACUA | 412 | UAGUUGUGAUCUAAAUGGC | 413 |
| CGAGGUUGUGUUUGAUAGA | 716 | UCUAUCAAACACAACCUCG | 717 |
| UGAAUACAGCCAUGCUUUA | 832 | UAAAGCAUGGCUGUAUUCA | 833 |
| CUGGCAUUGUUAACUUAGA | 906 | UCUAAGUUAACAAUGCCAG | 907 |
| CUCCAAACCUGAGAAUUUA | 968 | UAAAUUCUCAGGUUUGGAG | 969 |
| UGGGUUUUAGACCACACUA | 1084 | UAGUGUGGUCUAAAACCCA | 1085 |
| GGUUUUAGACCACACUAAA | 1086 | UUUAGUGUGGUCUAAAACC | 1087 |
| GACGGAAGUUAAAGAAGUA | 1114 | UACUUCUUUAACUUCCGUC | 1115 |
| CUCCAUUCAGAAUCUAGUA | 1186 | UACUAGAUUCUGAAUGGAG | 1187 |
| CCGUUAUUUUAGAAAUUCA | 1364 | UGAAUUUCUAAAAUAACGG | 1365 |
| CGUUAUUUUAGAAAUUCCA | 1366 | UGGAAUUUCUAAAAUAACG | 1367 |
| GGAGCAUUACUUAAAGAUA | 1392 | UAUCUUUAAGUAAUGCUCC | 1393 |
| AGCUAGUCCUUGACUGCAA | 1642 | UUGCAGUCAAGGACUAGCU | 1643 |
| GCUCCUACAUAAAACAAGA | 1874 | UCUUGUUUUAUGUAGGAGC | 1875 |
| GGGUGCUGCAGACAAUAUA | 1982 | UAUAUUGUCUGCAGCACCC | 1983 |
| GACAAUAUAUAUAAAGGAA | 1992 | UUCCUUUAUAUAUAUUGUC | 1993 |
| GUGGCUAAACUAGCAGAUA | 2302 | UAUCUGCUAGUUUAGCCAC | 2303 |
| GAAGGAUUAAUAAAUACGA | 2332 | UCGUAUUUAUUAAUCCUUC | 2333 |
| GGACGAUGCAUAAUGCACA | 2386 | UGUGCAUUAUGCAUCGUCC | 2387 |
| CUGUCUUCCUAACUUUUCA | 2550 | UGAAAAGUUAGGAAGACAG | 2551 |
| CCAGUAAAGCCUUAAGUGA | 2642 | UCACUUAAGGCUUUACUGG | 2643 |

Based on the results of Table 15, the following siRNA duplex candidates were selected for further testing and characterization.
dsRNA duplex of SENSE OLIGO NO: 156/ANTISENSE OLIGO: 157
dsRNA duplex of SENSE OLIGO NO: 906/ANTISENSE OLIGO NO: 907
dsRNA duplex of SENSE OLIGO NO: 968/ANTISENSE OLIGO NO: 1969
dsRNA duplex of SENSE OLIGO NO: 1392/ANTISENSE OLIGO NO: 1393
dsRNA duplex of SENSE OLIGO NO: 1366/ANTISENSE OLIGO NO: 1367
dsRNA duplex of SENSE OLIGO NO: 1874/ANTISENSE OLIGO NO: 1875

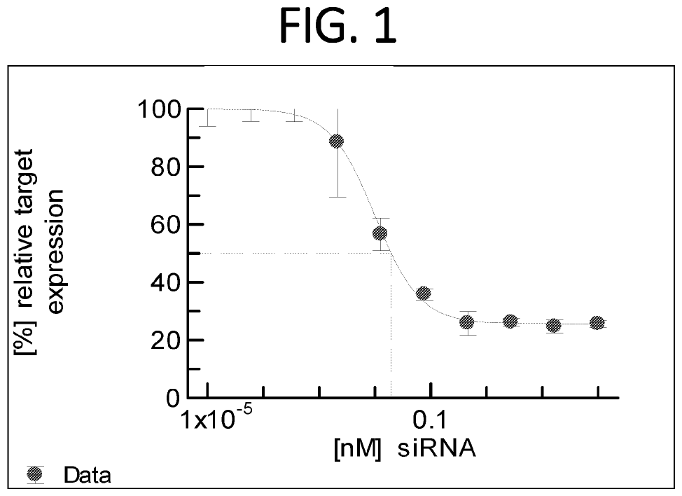

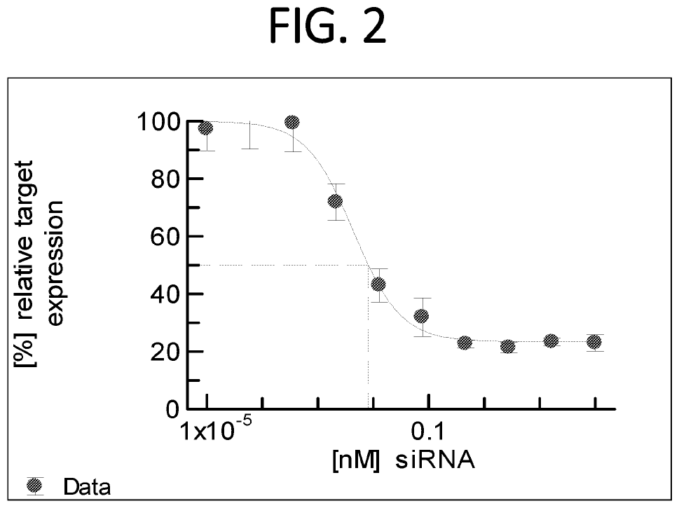

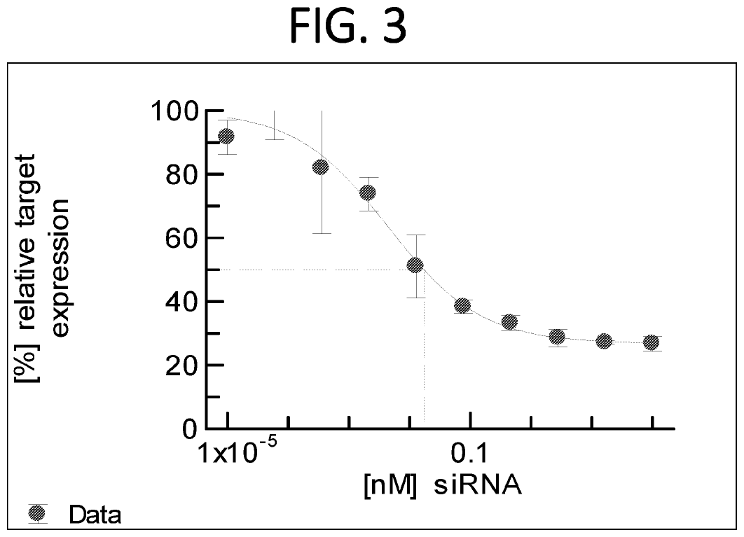

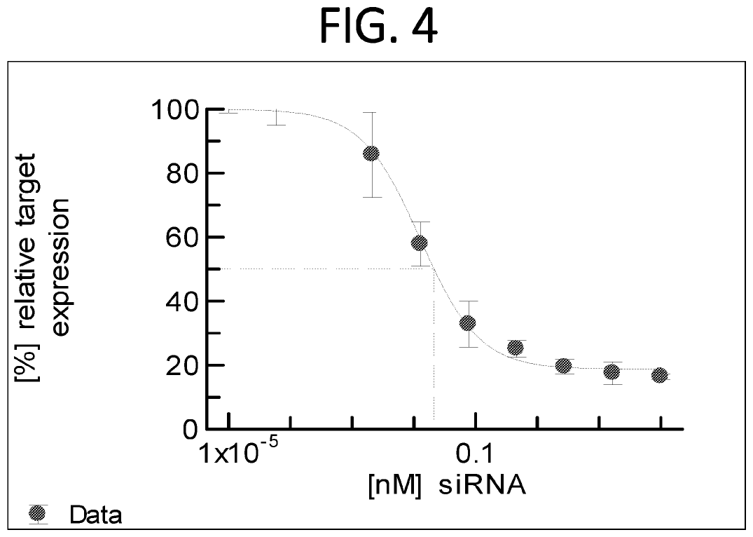

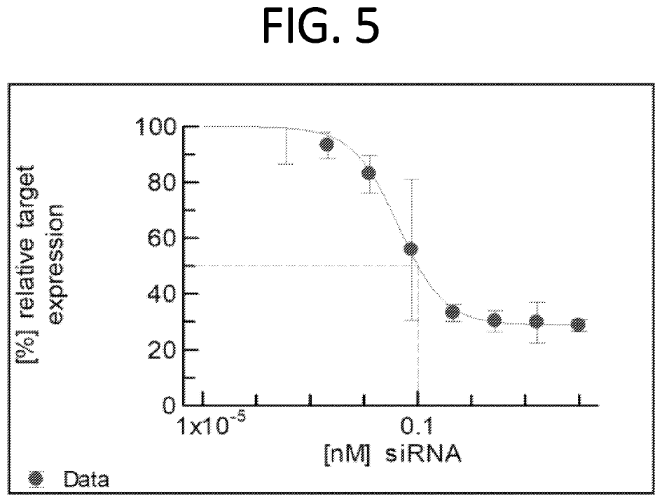

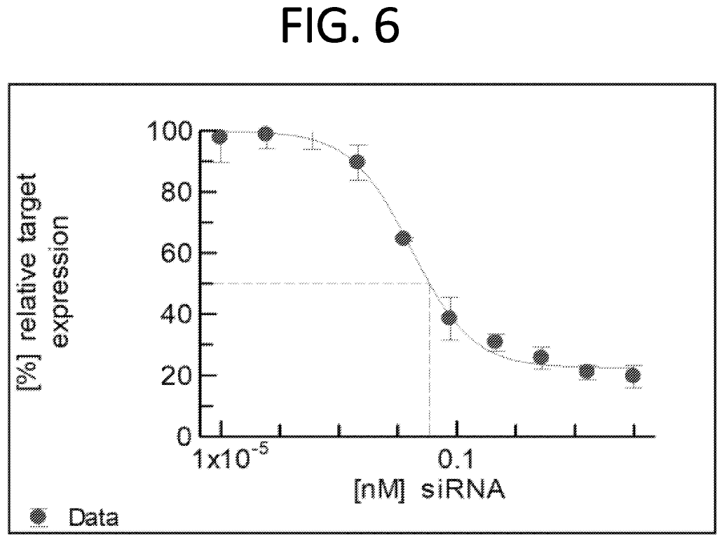

The dose response curves for the selected candidates are shown in FIGS. 1-6. The IC₂₀, IC₅₀, and IC₅₀ values for the candidates are shown below in Table 16. The sense and antisense oligonucleotides of Table 16 each contain a dTdT overhang on the 3′ end. Additionally, every A and Gin each sense oligonucleotide in Table 16 is a ribonucleotide. Every C and U in each sense oligonucleotide is a 2′-O-Methyl ribonucleotide.

Also, every A and G in each antisense oligonucleotide in Table 16 is a ribonucleotide. Every C and U preceding an A in the antisense oligonucleotide is a 2′-O-Methyl ribonucleotide, with one exception: U is the first nucleotide of the antisense strand, and it is a ribonucleotide.

Each nucleotide in the sense and antisense oligonucleotide in Table 16 is linked by a phosphate.

TABLE 16

| | | SEQ ID No. | | | |
|---|---|---|---|---|---|
| | | Sense | | | |
| | | No./ | | | |
| | | Antisense | IC₂₀ @ | IC₅₀ @ | IC₈₀ @ |
| Sense | Antisense | No. | [nM] | [nM] | [nM] |
| CUGGCAUUGUUAACUUAGA | UCUAAGUUAACAAUGCCAG | 968/969 | 0.0016 | 0.0081 | #N/A |
| GGAGCAUUACUUAAAGAUA | UAUCUUUAAGUAAUGCUCC | 1392/1393 | 0.0037 | 0.0213 | 1.1670 |
| CUCCAAACCUGAGAAUUUA | UAAAUUCUCAGGUUUGGAG | 1084/1085 | 0.0008 | 0.0172 | #N/A |
| CGUUAUUUUAGAAAUUCCA | UGGAAUUUCUAAAAUAACG | 1366/1367 | 0.0049 | 0.0343 | #N/A |
| GCCAAAAUGUACUGAUUUA | UAAAUCAGUACAUUUUGGC | 156/157 | 0.0040 | 0.0196 | #N/A |
| GCUCCUACAUAAAACAAGA | UCUUGUUUUAUGUAGGAGC | 1874/1875 | 0.0173 | 0.1013 | #N/A |

### RNAi Dose Response Screen

Twelve siRNA duplexes targeting MSH3 were tested using a Hela cell-based assay. The siRNA duplex strands and the EC₅₀ results are provided below in Table 17. The sense and antisense oligonucleotides of Table 18 each contain a dTdT overhang on the 3′ end. Additionally, every A and Gin each sense oligonucleotide in Table 17 is a ribonucleotide. Every C and U in each sense oligonucleotide is a 2′-O-Methyl ribonucleotide.

Also, every A and G in each antisense oligonucleotide in Table 17 is a ribonucleotide. Every C and U preceding an A in the antisense oligonucleotide is a 2′-O-Methyl ribonucleotide, with one exception: U is the first nucleotide of the antisense strand, and it is a ribonucleotide.

Each nucleotide in the sense and antisense oligonucleotide in Table 17 is linked by a phosphate.

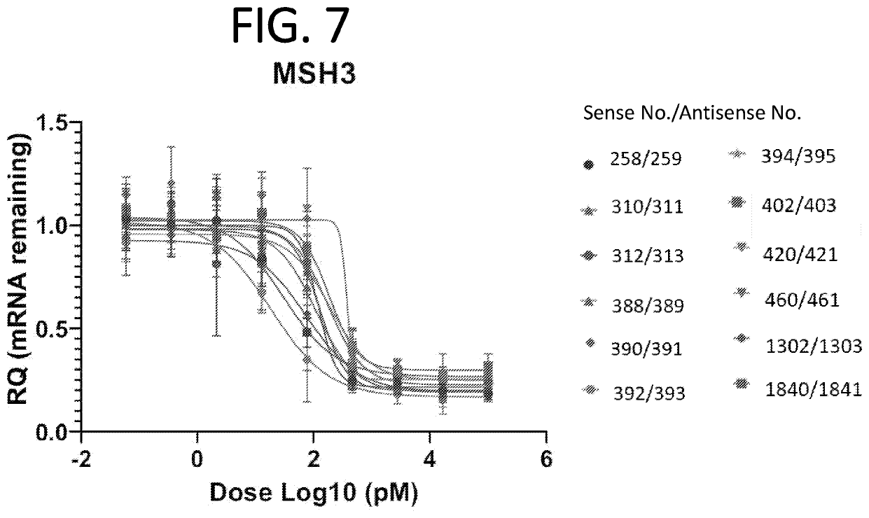

The non linear regression curves depicting the mean, the standard deviation, and the RQ values for each of the tested siRNAs at ten concentrations are plotted together in FIG. 7.

TABLE 17

| | | SEQ ID | |
|---|---|---|---|
| | | NO. | |
| | | Sense | |
| | | No./ | |
| | | Anti- | |
| | | sense | EC50 |
| Sense | Antisense | No. | (pM) |
| AUACAAGUCAUGAAAAUUA | UAAUUUUCAUGACUUGUAU | 258/259 | 41.09 |
| CGCUAGAAUUACAAUACAA | UUGUAUUGUAAUUCUAGCG | 310/311 | 129.2 |
| UAGAAUUACAAUACAUAGA | UCUAUGUAUUGUAAUUCUA | 312/313 | 120 |
| AGCCCGAGAGCUCAAUAUA | UAUAUUGAGCUCUCGGGCU | 388/389 | 109.9 |
| GCCCGAGAGCUCAAUAUUA | UAAUAUUGAGCUCUCGGGC | 390/391 | 179.9 |
| CGAGAGCUCAAUAUUUAUA | UAUAAAUAUUGAGCUCUCG | 392/393 | 18.49 |
| GAGAGCUCAAUAUUUAUUA | UAAUAAAUAUUGAGCUCUC | 394/395 | 191.7 |
| UUUAUUGCCAUUUAGAUCA | UGAUCUAAAUGGCAAUAAA | 402/403 | 184.4 |
| AGAUCACAACUUUAUGACA | UGUCAUAAAGUUGUGAUCU | 420/421 | 374.3 |
| CACAGACUGUUUGUUCAUA | UAUGAACAAACAGUCUGUG | 460/461 | 176.4 |
| UCACCUAAAGUCAGAAUUA | UAAUUCUGACUUUAGGUGA | 1302/1303 | 74.96 |
| CAGAGAGAGUAAUGAUAAA | UUUAUCAUUACUCUCUCUG | 1840/1841 | 30.16 |

The 108 siRNA duplexes selected for dose response validation had new source plates created to allow each dose to be diluted 1000-fold in the final assay plate. The final siRNA concentrations in the assay plates were as follows:
100 nM
16.67 nM
2.77 nM
462 pM
77.1 pM
12.9 pM
2.14 pM
357 fM
59.5 fM
UltraPure H2O Control

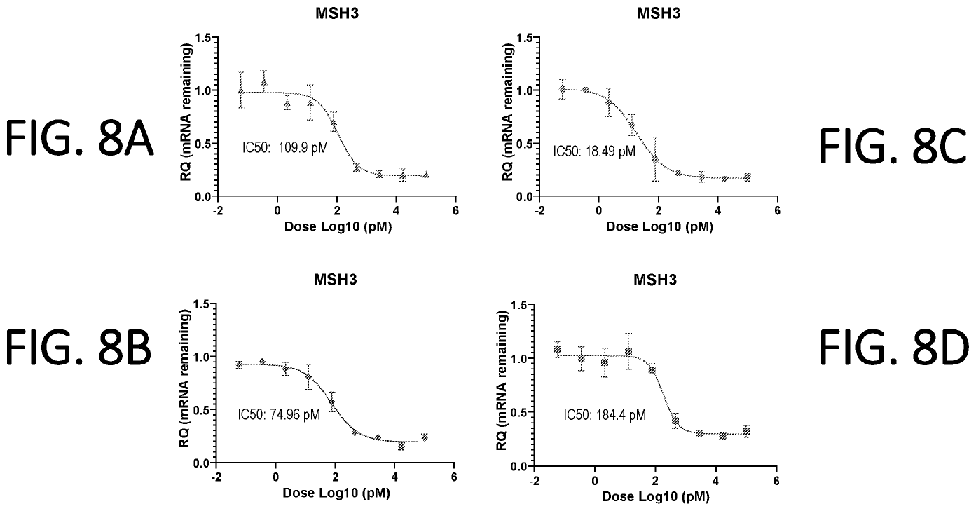

After this initial dispense the RNAi screen protocol was followed. The screening results for the top four candidates are provided in FIGS. 8A-8D.

### Example 8: Confirmatory Screen of Top Sequences in Primary Human Hepatocytes (PHH)

Primary Human Hepatocytes (PHH) were used to select potent siRNAs targeting the human MSH3 transcript for further in vivo testing. The siRNAs used in this study were selected from in vitro activity screens. Twenty MSH3 siRNA from the primary screens were screened by transient transfection at 0.1 nM and 2 nM in PHH. The eight siRNAs that showed significant KD (>75% at 2 nM) activity were further evaluated by dose response curves (DRC) in PHH. A mouse/cyno/human cross-reactive siRNA (SENSE OLIGO NO. 832/ANTISENSE OLIGO NO. 833) with known MSH3 knockdown activity in mouse was included as a reference.

Testing Top Eight siRNAs for DRCs by Transient Transfection in PHH

PHH cat. #Hu8350 were obtained from (Thermo Scientific; Cambridge, MA, USA). Cells were plated at 35K per well in Hepatocyte plating media cat. #CM3000 and maintained in Hepatocyte maintenance media cat. #CM4000 (Thermo Scientific; Cambridge, MA, USA) in a 96 well collagen coated plate cat. #12-565-909 (Fisher Scientific; Cambridge, MA, USA) and incubated at 37° C. with 5% CO2 in a humidified incubator for each experiment.

### Transfection

Five to six hours post plating, the cells were washed with maintenance media and incubated at 37° C. Twenty-four hours post-thaw, siRNAs were diluted starting at 100 nM with a 4-fold dilution to 0.000154 nM, complexed with 0.3 μl of RNAi Max cat. #13-778-150 (Fisher Scientific; Cambridge, MA, USA) and added to cells. Twenty-four hours post transfection, RNA was extracted using Quick-RNA 96 kit cat. #R1053 (Zymo Research; Irvine, CA, USA) according to manufacturer's instructions and samples were eluted in 22 μl nuclease-free water. Total RNA was quantified using Quant-iT RiboGreen RNA assay kit cat. #R11490 (Invitrogen; Carlsbad, CA, USA) and 80 ngs was used to generate cDNA with SuperScript IV VILO Master Mix cat. #11-756-500 (Thermo Scientific; Cambridge, MA, USA) according to manufacturer's instructions. The RNA was treated with DNAse I to remove any genomic DNA during RNA extraction and any remainder of it was accounted for with the addition of a cDNA reaction lacking reverse transcriptase (−RT control). 10% of the cDNA reaction was used for qPCR using PrimeTime Gene Expression Master Mix cat. #1055772 (Integrated DNA Technologies (IDT); Coralville, IA, USA) along with the hydrolysis probes for the gene of interest and housekeeper GUSB or TBP (IDT; Coralville, IA, USA) on a LightCycler 480 II (Roche; Basel, Switzerland).

### Data Analysis

The GUSB and TBP genes were used as housekeeping controls. The signal threshold for target MSH3 was set based on background signal from −RT control samples and crossing point (Cp) determined for each probe and sample. ΔCp was calculated as Cp[target]−Cp[housekeeping]. The average ΔCp for Mock treated samples was established for each target gene. The ΔΔCp was calculated as the ΔCp−Average [Mock] ΔCp, and relative expression as 2\hat{ }−(ΔΔCp). Relative fold-change in target expression calculated by 2−ΔΔCp, was averaged between the two housekeepers and analyzed in GraphPad Prism. For generating DRCs, the concentration of each treatment was converted to the Log values, IC50 for each target was calculated by analyzing the relative expression using the equation for “Non-linear regression curve fit” with Prism. The equation was fit to Log (Inhibitor) Vs response-variable slope using (Four parameters).

### DRCs and IC₅₀ Graphs

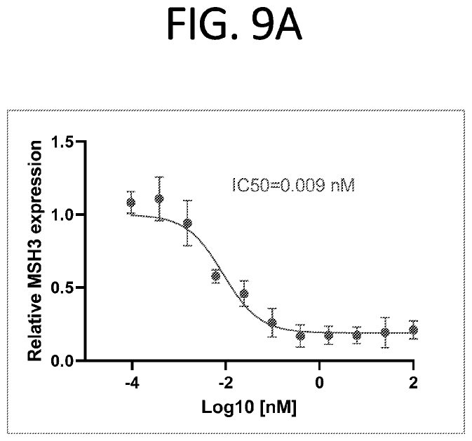

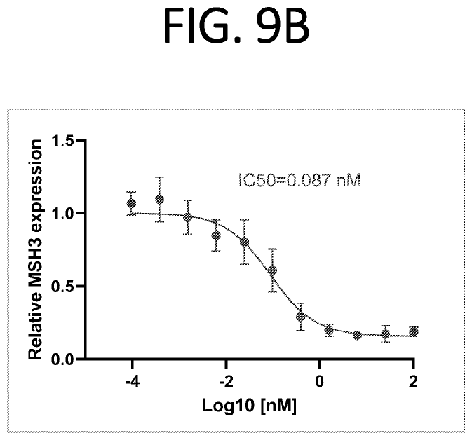

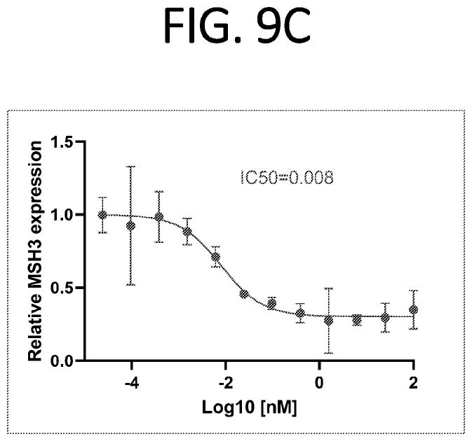

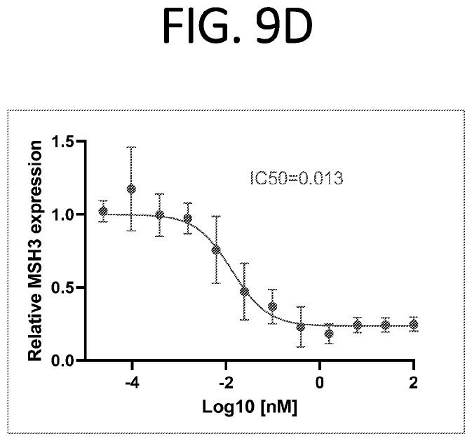

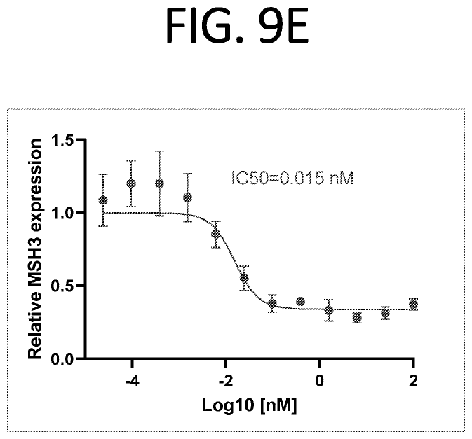

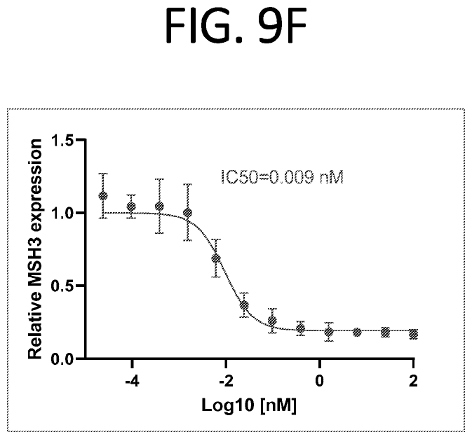

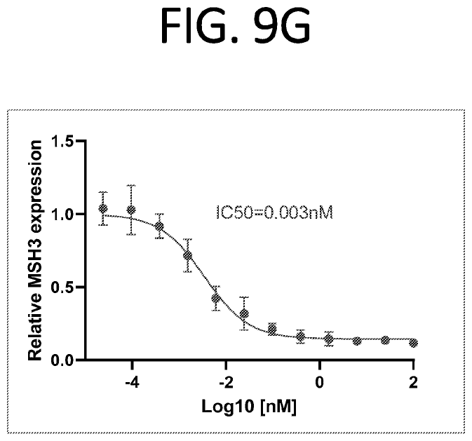

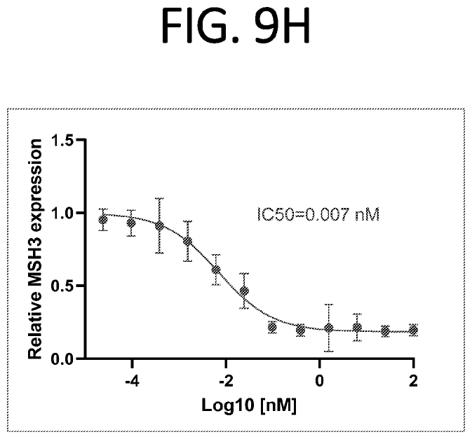

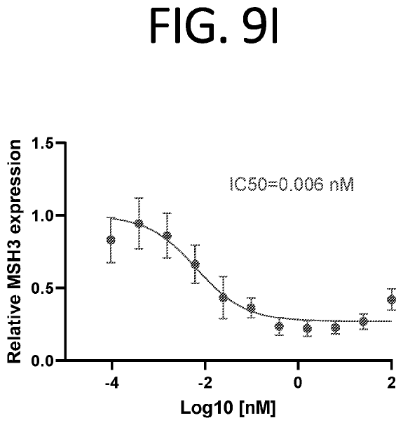

The siRNAs with highest activity in the dual-dose screen were used to generate the dose response curves. The graphs in FIGS. 9A-9I shows IC₅₀ analysis for the target knock down measured by qPCR. The X-axis represents the concentration of siRNA transfected and the Y-axis represents the relative MSH3 target remaining. The IC₅₀ for each target was calculated using the equation for “Non-linear regression curve fit” with Prism. The equation was fit to Log (Inhibitor) Vs response-variable slope using (Four parameters).

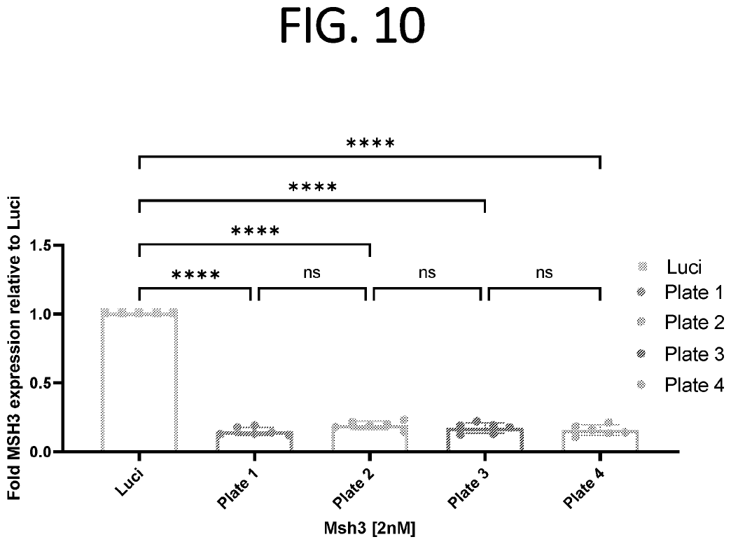

FIGS. 9A-9I show that there was good transfection efficiency between plates by the control MSH3 siRNA knock down in different plates. The DRCs for siRNA knock down were generated in four different plates. A commercially available SMART pool MSH3 siRNA was used to compare the transfection efficiency between different plates at 2 nM. The X-axis represents the concentration of siRNA transfection on different plates and Y-axis represents the percentage of target remaining. The fold change in MSH3 expression from the four plates is shown in FIG. 10. The siRNAs in each plate are provided in Table 18. The IC₅₀ with R² values and max KD for the top eight siRNAs are shown in Table 19.

The sense and antisense oligonucleotides of Tables 18 and 19 each contain a dTdT overhang on the 3′ end. Additionally, every A and G in each sense oligonucleotide in Tables 18 and 19 is a ribonucleotide. Every C and U in each sense oligonucleotide is a 2′-O-Methyl ribonucleotide.

Also, every A and Gin each antisense oligonucleotide in Tables 18 and 19 is a ribonucleotide. Every C and U preceding an A in the antisense oligonucleotide is a 2′-O-Methyl ribonucleotide, with one exception: U is the first nucleotide of the antisense strand, and it is a ribonucleotide.

Each nucleotide in the sense and antisense oligonucleotide in Tables 18 and 19 is linked by a phosphate.

TABLE 18

| | | Seq id/ | | Seq id/ |
|---|---|---|---|---|
| | | SENSE | | ANTISENSE |
| | | OLIGO | | OLIGO |
| Plates | Sense | NO | Antisense | NO |
| Plate 1 | CGUUAUUUUA | 1366 | UGGAAUUUCU | 1367 |
| | GAAAUUCCA | | AAAAUAACG | |
| Plate 1 | GCUCCUACAU | 1874 | UCUUGUUUUA | 1875 |
| | AAAACAAGA | | UGUAGGAGC | |
| Plate 2 | CAACAGAAGU | 550 | UAAGAGUGAA | 551 |
| | UCACUCUUA | | CUUCUGUUG | |
| Plate 2 | UCACCUAAAG | 1302 | UAAUUCUGAC | 1303 |
| | UCAGAAUUA | | UUUAGGUGA | |
| Plate 3 | AGCCCGAGAG | 388 | UAUAUUGAGC | 389 |
| | CUCAAUAUA | | UCUCGGGCU | |
| Plate 3 | UUUAUUGCCA | 402 | UGAUCUAAAU | 403 |
| | UUUAGAUCA | | GGCAAUAAA | |
| Plate 4 | GUUGAUGAGA | 7450 | UAGUCAUUAU | 745 |
| | UAAUGACUA | | CUCAUCAAC | |
| Plate 4 | AGAUAAUGAC | 656 | UAAGUAUCAG | 657 |
| | UGAUACUUA | | UCAUUAUCU | |

TABLE 19

| | | SEQ ID | | | | |
|---|---|---|---|---|---|---|
| | | NO. | | | | |
| | | Sense | | | | |
| | | No./ | | | | |
| | | Anti- | | | Max | |
| | | sense | IC50 | | KD | Rank- |
| Sense | Antisense | No. | [nM] | R² | (%) | ing |
| GUUGAUGAGA | UAGUCAUUAU | 648/ | 0.003 | 0.95 | 88 | 1 |
| UAAUGACUA | CUCAUCAAC | 649 | | | | |
| AGAUAAUGAC | UAAGUAUCAG | 656/ | 0.007 | 0.84 | 80 | 2 |
| UGAUACUUA | UCAUUAUCU | 657 | | | | |
| UGAAUACAGC | UAAAGCAUGG | 832/ | 0.006498 | 0.92 | 80 | 3 |
| CAUGCUUUA | CUGUAUUCA | 833 | | | | |
| AGAUCACAAC | UGUCAUAAAG | 420/ | 0.007 | 0.78 | 72 | 4 |
| UUUAUGACA | UUGUGAUCU | 421 | | | | |
| CGUUAUUUUA | UGGAAUUUCU | 1366/ | 0.009 | 0.92 | 80 | 5 |
| GAAAUUCCA | AAAAUAACG | 1367 | | | | |
| UCACCUAAAG | UAAUUCUGAC | 1302/ | 0.009 | 0.93 | 83 | 6 |
| UCAGAAUUA | UUUAGGUGA | 1303 | | | | |
| CAACAGAAGU | UAAGAGUGAA | 550/ | 0.013 | 0.85 | 75 | 7 |
| UCACUCUUA | CUUCUGUUG | 551 | | | | |
| ACUUCUACCA | UAAGAUAGCU | 672/ | 0.015 | 0.86 | 69 | 8 |
| GCUAUCUUA | GGUAGAAGU | 673 | | | | |
| GCUCCUACAU | UCUUGUUUUA | 1874/ | 0.087 | 0.92 | 79 | 9 |
| AAAACAAGA | UGUAGGAGC | 1875 | | | | |

This study identified eight siRNAs targeting MSH3 with half maximal inhibitory concentration (IC₅₀) by transfection in PHH in the low pM range. The results of the eight candidates selected from the siRNA screening at 0.5 nM and 10 nM doses and the resulting IC₅₀ are provided below in Table 20. The siRNA of Sense No. 656/Antisense No. 657 was used as a control.

TABLE 20
dsRNA

| SENSE OLIGO No./ | % target remaining | | |
|---|---|---|---|
| ANTISENSE OLIGO No. | 0.5 nM | 10 nM | IC₅₀ (nM) |
| SENSE OLIGO No. 832/ | 44.7735672 | 23.0173288 | N/A |
| ANTISENSE OLIGO No. | | | |
| 833 | | | |
| SENSE OLIGO No. 1366/ | 27.0833733 | 17.2481438 | 34.349054 |
| ANTISENSE OLIGO No. | | | |
| 1367 | | | |
| SENSE OLIGO No. 1874/ | 28.0472485 | 23.0624968 | 101.305208 |
| ANTISENSE OLIGO No. | | | |
| 1875 | | | |
| SENSE OLIGO No. 550/ | 34.4598214 | 24.5834182 | 18.49 |
| ANTISENSE OLIGO No. | | | |
| 551 | | | |
| SENSE OLIGO No. 1302/ | 36.1222642 | 25.7249608 | 74.96 |
| ANTISENSE OLIGO No. | | | |
| 1303 | | | |
| SENSE OLIGO No. 420/ | 37.1536281 | 25.4957144 | 109.9 |
| ANTISENSE OLIGO No. | | | |
| 421 | | | |
| SENSE OLIGO No. 672/ | 33.8159421 | 24.8627536 | 184.4 |
| ANTISENSE OLIGO No. | | | |
| 673 | | | |
| SENSE OLIGO No. 648/ | N/A | 16.9266119 | N/A |
| ANTISENSE OLIGO No. | | | |
| 649 | | | |
| SENSE OLIGO No. 656/ | N/A | 22.5483683 | N/A |
| ANTISENSE OLIGO No. | | | |
| 657 | | | |

This example demonstrates that the siRNAs targeting MSH3 identified in the screening studies showed confirmed MSH3 knockdown in vitro.

### OTHER ASPECTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations following, in general, the principles and including such departures from the present disclosure that come within known or customary practice within the art to which the invention pertains and can be applied to the essential features hereinbefore set forth, and follows in the scope of the claimed.

In addition to the various aspects described herein, the present disclosure includes the following aspects numbered E1 through E108. This list of aspects is presented as an exemplary list and the application is not limited to these particular aspects.
E1. A double-stranded ribonucleic acid (dsRNA), wherein the dsRNA comprises a sense strand and an antisense strand, wherein the antisense strand is complementary to at least 15 contiguous nucleobases of an MSH3 gene, and wherein the dsRNA comprises a duplex structure of between 15 and 30 linked nucleosides in length.
E2. A dsRNA for reducing expression of MSH3 in a cell, wherein the dsRNA comprises a sense strand and an antisense strand, wherein the antisense strand is complementary to at least 15 contiguous nucleobases of an MSH3 gene, and wherein the dsRNA comprises a duplex structure of between 15 and 30 linked nucleosides in length.
E3. The dsRNA of E1 or E2 comprising a duplex structure of between 19 and 23 linked nucleosides in length.
E4. The dsRNA of any one of E1-E3, further comprising a loop region joining the sense strand and antisense strand, wherein the loop region is characterized by a lack of base pairing between nucleobases within the loop region.
E5. The dsRNA of any one of E1-E4, wherein the region the sense or antisense strand is complementary to is at least 15 contiguous nucleotides of an MSH3 gene corresponding to a sequence of reference mRNA NM_002439.4 at one or more of positions 566-589, 678-701, 875-989, 1019-1088, 1370-1393, 1466-1569, 1721-1849, 1879-2038, 2086-2171, 2783-2806, 2847-2922, 3043-3119, 3241-3314, 3330-3353, or 3701-3792 of the MSH3 gene.
E6. The dsRNA of any one of E1-E4, wherein the region the sense or antisense strand is complementary to is at least 15 contiguous nucleotides of an MSH3 gene corresponding to a sequence of reference mRNA NM_002439.4 at one or more of positions 876-989, 1019-1088, 1370-1393, 1889-1938, or 3241-3314 of the MSH3 gene.
E7. The dsRNA of any one of E1-E4, wherein the region the sense or antisense strand is complementary to is at least 15 contiguous nucleotides of an MSH3 gene corresponding to a sequence of reference mRNA NM_002439.4 at one or more of positions 876-989, 1019-1088, 1370-1393, 1466-1569, 1756-1849, 1879-2038, 2086-2171, 2783-2806, 2847-2922, 3043-3119, 3241-3314, 3330-3353, or 3701-3792 of the MSH3 gene.
E8. The dsRNA of any one of E1-E4, wherein the region the sense or antisense strand is complementary to is at least 15 contiguous nucleotides of an MSH3 gene corresponding to a sequence of reference mRNA NM_002439.4 at one or more of positions 678-701, 875-989, 1019-1088, 1370-1393, 1466-1569, 1721-1849, 1879-2038, 2086-2171, 2783-2806, 2847-2922, 3043-3119, 3241-3314, 3330-3353, and 3701-3792 of the MSH3 gene.
E9. The dsRNA of any one of E1-E4, wherein the region the sense or antisense strand is complementary to is at least 15 contiguous nucleotides of an MSH3 gene corresponding to a sequence of reference mRNA NM_002439.4 at positions 879-921 of the MSH3 gene.
E10. The dsRNA of any one of E1-E4, wherein the region the sense or antisense strand is complementary to is at least 15 contiguous nucleotides of an MSH3 gene corresponding to a sequence of reference mRNA NM_002439.4 at one or more of positions 875-989, 1019-1088, 1370-1393, 1466-1569, 1721-1849, 1879-1970, 2783-2806, 2847-2922, 3043-3119, 3241-3314, 3330-3353, or 3703-3792 of the MSH3 gene.
E11. The dsRNA of any one of E1-E4, wherein the region the sense or antisense strand is complementary to is at least 15 contiguous nucleotides of an MSH3 gene corresponding to a sequence of reference mRNA NM_002439.4 at one or more of positions 566-589, 678-701, 875-989, 1019-1088, 1370-1393, 1466-1569, 1721-1849, 1879-2038, 2086-2171, 2783-2806, 2847-2922, 3043-3119, 3241-3314, 3330-3353, or 3701-3792 of the MSH3 gene.
E12. The dsRNA of any one of E1-E4, wherein the antisense strand comprises an antisense nucleobase sequence selected from Table 3, wherein the 5′ nucleotide represented by U can be any nucleotide (e.g., U, A, C, G), and the sense strand comprises a sense nucleobase sequence complementary to the antisense nucleobase sequence.
E13. The dsRNA of any one of E1-E4, wherein the antisense nucleobase sequence consists of an antisense strand in Table 3, wherein the 5′ nucleotide represented by U can be any nucleotide (e.g., U, A, C, G), and the sense nucleobase sequence consists of a sequence complementary to the antisense nucleobase sequence.
E14. The dsRNA of any one of E1-E4, wherein the sense strand comprises a sense nucleobase sequence selected from Table 3, and the antisense strand comprises an antisense nucleobase sequence complementary to the sense nucleobase sequence.
E15. The dsRNA of any one of E1-E4, wherein the sense nucleobase sequence consists of a sense strand in Table 3, and the antisense nucleobase sequence consists of a sequence complementary to the sense nucleobase sequence.
E16. The dsRNA of any one of E1-E4, wherein the sense strand comprises a sense nucleobase sequence selected from Tables 4-10, and the antisense strand comprises an antisense nucleobase sequence complementary to the sense nucleobase sequence.
E17. The dsRNA of any one of E1-E4, wherein the sense nucleobase sequence consists of a sense strand in any one of Tables 4-10, and the antisense nucleobase sequence consists of a sequence complementary to the sense nucleobase sequence.
E18. The dsRNA of any one of E1-E4, wherein the antisense strand comprises an antisense nucleobase sequence selected from Table 11, wherein the 5′ nucleotide represented by U can be any nucleotide (e.g., U, A, C, G), and the sense strand comprises a sense nucleobase sequence complementary to the antisense nucleobase sequence.
E19. The dsRNA of any one of E1-E4, wherein the antisense nucleobase sequence consists of an antisense strand in Table 11, wherein the 5′ nucleotide represented by U can be any nucleotide (e.g., U, A, C, G), and the sense nucleobase sequence consists of a sequence complementary to the antisense nucleobase sequence
E20. The dsRNA of any one of E1-E4, wherein the sense strand comprises a sense nucleobase sequence selected from Table 11, and the antisense strand comprises an antisense nucleobase sequence complementary to the sense nucleobase sequence.
E21. The dsRNA of any one of E1-E4, wherein the sense nucleobase sequence consists of a sense strand in Table 11, and the antisense nucleobase sequence consists of a sequence complementary to the sense nucleobase sequence.
E22. The dsRNA of any one of E1-E21, wherein the dsRNA comprises at least one alternative nucleobase, at least one alternative internucleoside linkage, and/or at least one alternative sugar moiety.
E23. The dsRNA of E22, wherein at least one alternative internucleoside linkage is a phosphorothioate internucleoside linkage.
E24. The dsRNA of E22, wherein at least one alternative internucleoside linkage is a 2′-alkoxy internucleoside linkage.
E25. The dsRNA of E22, wherein at least one alternative internucleoside linkage is an alkyl phosphate internucleoside linkage.
E26. The dsRNA of E22, wherein at least one alternative nucleobase is 5′-methylcytosine, pseudouridine, or 5-methoxyuridine.
E27. The dsRNA of E22, wherein at least one alternative sugar moiety is 2′-OMe or a bicyclic nucleic acid.
E28. The dsRNA of E22, wherein the dsRNA comprises at least one 2′-OMe sugar moiety and at least one phosphorothioate internucleoside linkage.
E29. The dsRNA of any one of E1-E28, wherein the dsRNA further comprises a ligand conjugated to the 3′ end of the sense strand through a monovalent or branched bivalent or trivalent linker.
E30. The dsRNA of any one of E1-E29, wherein the sense strand comprises a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.
E31. The dsRNA of any one of E1-E29, wherein the sense strand comprises a nucleobase sequence of any one of SEQ ID NOs: 380, 382, 386, 388, 396, 406, 418, 464, 478, 520, 540, 564, 568, 750, 1166, 1168, 1170, 1182, 1192, 1212, 1214, 1216, 1222, 2264, 2290, 2308, or 2318.
E32. The dsRNA of any one of E1-E29, wherein the sense strand comprises a nucleobase sequence of any one of SEQ ID NOs: 380, 382, 386, 388, 396, 406, 418, 464, 478, 520, 540, 564, 568, 750, 822, 830, 844, 868, 870, 874, 904, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.
E33. The dsRNA of any one of E1-E29, wherein the sense strand comprises a nucleobase sequence of any one of SEQ ID NOs: 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.
E34. The dsRNA of any one of E1-E29, wherein the sense strand comprises a nucleobase sequence of any one of SEQ ID NOs: 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1866, 1868, 1870, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.
E35. The dsRNA of any one of E1-E29, wherein the sense strand comprises a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.
E36. The dsRNA of any one of E1-E29, wherein the antisense strand comprises a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).
E37. The dsRNA of any one of E1-E29, wherein the antisense strand comprises a nucleobase sequence of any one of SEQ ID NOs: 381, 383, 387, 389, 397, 407, 419, 465, 479, 521, 541, 565, 569, 751, 1167, 1169, 1171, 1183, 1193, 1213, 1215, 1217, 1223, 2265, 2291, 2309, or 2319, wherein the 5′ nucleotide represented by U can be any nucleotide (e.g., U, A, C, G).
E38. The dsRNA of any one of E1-E29, wherein the antisense strand comprises a nucleobase sequence of any one of SEQ ID NOs: 381, 383, 387, 389, 397, 407, 419, 465, 479, 521, 541, 565, 569, 751, 823, 831, 845, 869, 871, 875, 905, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).
E39. The dsRNA of any one of E1-E29, wherein the antisense strand comprises a nucleobase sequence of any one of SEQ ID NOs: 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).
E40. The dsRNA of any one of E1-E29, wherein the antisense strand comprises a nucleobase sequence of any one of SEQ ID NOs: 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).
E41. The dsRNA of any one of E1-E29, wherein the antisense strand comprises a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).
E42. The dsRNA of any one of E1-E29, wherein the sense strand consists of a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.
E43. The dsRNA of any one of E1-E29, wherein the sense strand consists of a nucleobase sequence of any one of SEQ ID NOs: 380, 382, 386, 388, 396, 406, 418, 464, 478, 520, 540, 564, 568, 750, 1166, 1168, 1170, 1182, 1192, 1212, 1214, 1216, 1222, 2264, 2290, 2308, or 2318.
E44. The dsRNA of any one of E1-E29, wherein the sense strand consists of a nucleobase sequence of any one of SEQ ID NOs: 380, 382, 386, 388, 396, 406, 418, 464, 478, 520, 540, 564, 568, 750, 822, 830, 844, 868, 870, 874, 904, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.
E45. The dsRNA of any one of E1-E29, wherein the sense strand consists of a nucleobase sequence of any one of SEQ ID NOs: 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.
E46. The dsRNA of any one of E1-E29, wherein the sense strand consists of a nucleobase sequence of any one of SEQ ID NOs: 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.
E47. The dsRNA of any one of E1-E29, wherein the sense strand consists of a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.
E48. The dsRNA of any one of E1-E29, wherein the sense strand consists of a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691.
E49. The dsRNA of any one of E1-E29, wherein the antisense strand consists of a nucleobase sequence of any one of SEQ ID NOs: 381, 383, 387, 389, 397, 407, 419, 465, 479, 521, 541, 565, 569, 751, 1167, 1169, 1171, 1183, 1193, 1213, 1215, 1217, 1223, 2265, 2291, 2309, or 2319, wherein the 5′ nucleotide represented by U can be any nucleotide (e.g., U, A, C, G).
E50. The dsRNA of any one of E1-E29, wherein the antisense strand consists of a nucleobase sequence of any one of SEQ ID NOs: 381, 383, 387, 389, 397, 407, 419, 465, 479, 521, 541, 565, 569, 751, 823, 831, 845, 869, 871, 875, 905, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).
E51. The dsRNA of any one of E1-E29, wherein the antisense strand consists of a nucleobase sequence of any one of SEQ ID NOs: 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).
E52. The dsRNA of any one of E1-E29, wherein the antisense strand consists of a nucleobase sequence of any one of SEQ ID NOs: 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).
E53. The dsRNA of any one of E1-E29, wherein the antisense strand consists of a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).
E54. The dsRNA of any one of E1-E53, wherein the dsRNA exhibits at least 50% mRNA inhibition at a 0.5 nM dsRNA concentration when determined using a cell assay when compared with a control cell.
E55. The dsRNA of any one of E1-E53, wherein the dsRNA exhibits at least 40% mRNA inhibition at a 0.5 nM dsRNA concentration when determined using a cell assay when compared with a control cell.
E56. The dsRNA of any one of E1-E53, wherein the dsRNA exhibits at least 30% mRNA inhibition at a 0.5 nM dsRNA concentration when determined using a cell assay when compared with a control cell.
E57. The dsRNA of any one of E1-E53, wherein the dsRNA exhibits at least 70% mRNA inhibition at a 10 nM dsRNA concentration when determined using a cell assay when compared with a control cell.
E58. The dsRNA of any one of E1-E53, wherein the dsRNA exhibits at least 60% mRNA inhibition at a 10 nM dsRNA concentration when determined using a cell assay when compared with a control cell.
E59. The dsRNA of any one of E1-E53, wherein the dsRNA exhibits at least 50% mRNA inhibition at a 10 nM dsRNA concentration when determined using a cell assay when compared with a control cell.
E60. The dsRNA of any one of E1-E59, wherein the antisense strand is complementary to at least 17 contiguous nucleotides of an MSH3 gene.
E61. The dsRNA of any one of E1-E59, wherein the antisense strand is complementary to at least 19 contiguous nucleotides of an MSH3 gene.
E62. The dsRNA of any one of E1-E59, wherein the antisense strand is complementary to 19 contiguous nucleotides of an MSH3 gene.
E63. The dsRNA of any one of E1-E59, wherein the sense strand is complementary to at least 17 contiguous nucleotides of an MSH3 gene.
E64. The dsRNA of any one of E1-E59, wherein the sense strand is complementary to at least 19 contiguous nucleotides of an MSH3 gene.
E65. The dsRNA of any one of E1-E59, wherein the sense strand is complementary to 19 contiguous nucleotides of an MSH3 gene.
E66. The dsRNA of any one of E1-E65, wherein the antisense strand and/or the sense strand comprises a 3′ overhang of at least 1 linked nucleoside; or a 3′ overhang of at least 2 linked nucleosides.
E67. A pharmaceutical composition comprising one or more dsRNAs of any one of E1-E66 and a pharmaceutically acceptable carrier.
E68. A composition comprising one or more dsRNAs of any one of E1-E66 and a lipid nanoparticle, a polyplex nanoparticle, a lipoplex nanoparticle, or a liposome.
E69. A vector encoding at least one strand of the dsRNA of any one of E1-E66.
E70. A cell comprising the vector of E69.
E71. A method of reducing transcription of MSH3 in a cell, the method comprising contacting the cell with the dsRNA of any one of E1-E66, the pharmaceutical composition of E67, the composition of E68, the vector of E69, or the cell of E70 for a time sufficient to obtain degradation of an mRNA transcript of MSH3, thereby reducing expression of MSH3 in the cell.
E72. A method of treating, preventing, or delaying progression of a trinucleotide repeat expansion disorder in a subject in need thereof, the method comprising administering to the subject the dsRNA of any one of E1-E66, the pharmaceutical composition of E67, the composition of E68, the vector of E69, or the cell of E70.
E73. A method of reducing the level and/or activity of MSH3 in a cell of a subject identified as having a nucleotide repeat expansion disorder, the method comprising contacting the cell with the dsRNA of any one of E1-E66, the pharmaceutical composition of E67, the composition of E68, the vector of E69, or the cell of E70.
E74. A method for reducing expression of MSH3 in a cell comprising contacting the cell with the dsRNA of any one of E1-E66, the pharmaceutical composition of E67, the composition of E68, the vector of E69, or the cell of E70 and maintaining the cell for a time sufficient to obtain degradation of an mRNA transcript of MSH3, thereby reducing expression of MSH3 in the cell.
E75. A method of decreasing nucleotide repeat expansion in a cell, the method comprising contacting the cell with the dsRNA of any one of E1-E66, the pharmaceutical composition of E67, the composition of E68, the vector of E69, or the cell of E70.
E76. The method of E74 or E75, wherein the cell is in a subject.
E77. The method of any one of E72, E73, and E76, wherein the subject is a human.
E78. The method of any one of E71 and 73-E76, wherein the cell is a cell of the central nervous system or a muscle cell.
E79. The method of any one of E72, E73, and E76-E78, wherein the subject is identified as having a nucleotide repeat expansion disorder.
E80. The method of any one of E72, E73, and E75-E79 wherein the nucleotide repeat expansion disorder is a polyglutamine disease.
E81. The method of E80, wherein the polyglutamine disease is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, and Huntington's disease-like 2.
E82. The method of any one of E72, E73, and E75-E79, wherein the nucleotide repeat expansion disorder is a non-polyglutamine disease.
E83. The method of E82, wherein the non-polyglutamine disease is selected from the group consisting of fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.
E84. A dsRNA of any one of E1-E66, the pharmaceutical composition of E67, the composition of E68, the vector of E69, or the cell of E70 for use in prevention or treatment of a nucleotide repeat expansion disorder.
E85. The dsRNA, the pharmaceutical composition, the composition, the vector, or the cell of E84, wherein the nucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.
E86. The dsRNA, the pharmaceutical composition, the composition, the vector, or the cell of E84 or E85, wherein the nucleotide repeat expansion disorder is Huntington's disease.
E87. The dsRNA, the pharmaceutical composition, the composition, the vector, or the cell of E84 or E85, wherein the nucleotide repeat expansion disorder is Friedreich's ataxia.
E88. The dsRNA, the pharmaceutical composition, the composition, the vector, or the cell of E84 or E85, wherein the nucleotide repeat expansion disorder is myotonic dystrophy type 1.
E89. The dsRNA, the pharmaceutical composition, the composition, the vector, or the cell of any of E84-E88, wherein the dsRNA, pharmaceutical composition, composition, vector, or cell is administered intrathecally.
E90. The dsRNA, the pharmaceutical composition, the composition, the vector, or the cell of any of E84-E88, wherein the dsRNA, pharmaceutical composition, composition, vector, or cell is administered intraventricularly.
E91. The dsRNA, the pharmaceutical composition, the composition, the vector, or the cell of any of E84-E88, wherein the dsRNA, pharmaceutical composition, composition, vector, or cell is administered intramuscularly.
E92. A method of treating, preventing, or delaying progression of a disorder in a subject in need thereof wherein the subject is suffering from nucleotide repeat expansion disorder, comprising administering to said subject the dsRNA of any one of E1-E66, the pharmaceutical composition of E67, the composition of E68, the vector of E69, or the cell of E70.
E93. The method of E92, further comprising administering at least one additional therapeutic agent.
E94. The method of E93, wherein at least one additional therapeutic agent is an antisense oligonucleotide that hybridizes to an mRNA encoding the Huntingtin gene.
E95. A method of preventing or delaying progression of a nucleotide repeat expansion disorder in a subject, the method comprising administering to the subject the dsRNA of any one of E1-E66, the pharmaceutical composition of E67, the composition of E68, the vector of E69, or the cell of E70 in an amount effective to delay progression of a nucleotide repeat expansion disorder of the subject.
E96. The method of E95, wherein the nucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.
E97. The method of E95 or E96, wherein the nucleotide repeat expansion disorder is Huntington's disease.
E98. The method of E95 or E96, wherein the nucleotide repeat expansion disorder is Friedrich's ataxia.
E99. The method of E95 or E96, wherein the nucleotide repeat expansion disorder is myotonic dystrophy type 1.
E100. The method of any of E95 or E96, further comprising administering at least one additional therapeutic agent.
E101. The method of E100, wherein at least one additional therapeutic agent is an antisense oligonucleotide that hybridizes to an mRNA encoding the Huntingtin gene.
E102. The method of any of E94-E101, wherein progression of the nucleotide repeat expansion disorder is delayed by at least 120 days, for example, at least 6 months, at least 12 months, at least 2 years, at least 3 years, at least 4 years, at least 5 years, at least 10 years or more, when compared with a predicted progression.
E103. A dsRNA of any one of E1-E66, the pharmaceutical composition of E67, the composition of E68, the vector of E69, or the cell of E70, for use in preventing or delaying progression of a nucleotide repeat expansion disorder in a subject.
E104. The dsRNA, the pharmaceutical composition, the composition, the vector, or the cell of E103, wherein the nucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.
E105. The dsRNA, the pharmaceutical composition, the composition, the vector, or the cell of E103 or E104, wherein the nucleotide repeat expansion disorder is Huntington's disease.
E106. The dsRNA, the pharmaceutical composition, the composition, the vector, or the cell of E103 or E104, wherein the nucleotide repeat expansion disorder is Friedrich's ataxia.
E107. The dsRNA, the pharmaceutical composition, the composition, the vector, or the cell of E103 or E104, wherein the nucleotide repeat expansion disorder is myotonic dystrophy type 1.
E108. The dsRNA, the pharmaceutical composition, the composition, the vector, or the cell of any one of E103-E107, wherein progression of the nucleotide repeat expansion disorder is delayed by at least 120 days, for example, at least 6 months, at least 12 months, at least 2 years, at least 3 years, at least 4 years, at least 5 years, at least 10 years or more, when compared with a predicted progression.
E109. The dsRNA, the pharmaceutical composition, the composition, the vector, or the cell of any one of claims E103-E107, wherein progression of the nucleotide repeat expansion disorder is delayed by at least 120 days, for example, at least 6 months, at least 12 months, at least 2 years, at least 3 years, at least 4 years, at least 5 years, at least 10 years or at least 20 years or more, when compared with a predicted progression.
E110. The dsRNA of any one of E1-E4, wherein the region the sense or antisense strand is complementary to is at least 15 contiguous nucleotides of an MSH3 gene corresponding to a sequence of reference mRNA NM_002439.4 at one or more of positions 430-453, 508-531, 560-599, 609-632, 681-721, 768-797, 823-856, 882-927, 968-1029, 1039-1096, 1106-1175, 1188-1217, 1272-1297, 1419-1474, 1489-1516, 1540-1627, 1633-1815, 1819-1842, 1899-1937, 2027-2066, 2085-2108, 2117-2156, 2163-2187, 2195-2241, 2293-2343, 2347-2374, 2493-2539, 2567-2590, 2619-2649, 2737-2764, 2779-2820, 2871-2894, 2900-2923, 2949-2972, 3049-3096, 3217-3266, 3272-3309, 3351-3383, 3386-3415, 3537-3560, 3581-3619, 3686-3728, 3754-3778, 3782-3805, 3909-3935, 4287-4310, or 4386-4412 of the MSH3 gene.
E111. The dsRNA of any one of E1-E4, wherein the region the sense or antisense strand is complementary to is at least 15 contiguous nucleotides of an MSH3 gene corresponding to a sequence of reference mRNA NM_002439.4 at one or more of positions 508-531, 827-856, 903-926, 1073-1096, 1126-1149, 1583-1609, 1639-1662, 1727-1750, 1755-1795, 1819-1842, 1905-1937, 2130-2153, 2293-2316, 2505-2528, 2625-2648, 2797-2820, 3073-3096, 3217-3240, 3351-3383, 3686-3728, 3754-3777, 4287-4310, or 4386-4412 of the MSH3 gene.
E112. The dsRNA of any one of E1-E4, wherein the region the sense or antisense strand is complementary to is at least 15 contiguous nucleotides of an MSH3 gene corresponding to a sequence of reference mRNA NM_002439.4 at one or more of positions 508-531, 833-856, 1073-1096, 1126-1149, 1583-1609, 1639-1662, 1727-1750, 1755-1795, 1914-1937, 2130-2153, 2293-2316, 2797-2820, 3073-3096, 3217-3240, 3596-3619, 3700-3723, 3754-3777, or 4386-4409 of the MSH3 gene.
E113. The dsRNA of any one of E1-E4, wherein the region the sense or antisense strand is complementary to is at least 15 contiguous nucleotides of an MSH3 gene corresponding to a sequence of reference mRNA NM_002439.4 at one or more of positions 1073-1096, 1586-1609, 1755-1795, 1914-1937, 2130-2153, 2293-2316, 3217-3240, or 4386-4409 of the MSH3 gene
E114. The dsRNA of any one of E1-E4, wherein the region the sense or antisense strand is complementary to is at least 15 contiguous nucleotides of an MSH3 gene corresponding to a sequence of reference mRNA NM_002439.4 at positions 908-925 of the MSH3 gene.
E115. The dsRNA of any one of E1-E4, wherein the region the sense or antisense strand is complementary to is at least 15 contiguous nucleotides of an MSH3 gene corresponding to a sequence of reference mRNA NM_002439.4 at positions 1167-1184 of the MSH3 gene.
E116. The dsRNA of any one of E1-E4, wherein the region the sense or antisense strand is complementary to is at least 15 contiguous nucleotides of an MSH3 gene corresponding to a sequence of reference mRNA NM_002439.4 at positions 1143-1166 of the MSH3 gene.
E117. The dsRNA of any one of E1-E4, wherein the region the sense or antisense strand is complementary to is at least 15 contiguous nucleotides of an MSH3 gene corresponding to a sequence of reference mRNA NM_002439.4 at positions 1150-1173 of the MSH3 gene
E118. The dsRNA of any one of E1-E4, wherein the region the sense or antisense strand is complementary to is at least 15 contiguous nucleotides of an MSH3 gene corresponding to a sequence of reference mRNA NM_002439.4 at positions 2090-2107 of the MSH3 gene
E119. The dsRNA of any one of E1-E4, wherein the region the sense or antisense strand is complementary to is at least 15 contiguous nucleotides of an MSH3 gene corresponding to a sequence of reference mRNA NM_002439.4 at positions 1040-1057 of the MSH3 gene
E120. The dsRNA of any one of E1-E4, wherein the region the sense or antisense strand is complementary to is at least 15 contiguous nucleotides of an MSH3 gene corresponding to a sequence of reference mRNA NM_002439.4 at positions 2018-2035 of the MSH3 gene
E121. The dsRNA of any one of E1-E4, wherein the region the sense or antisense strand is complementary to is at least 15 contiguous nucleotides of an MSH3 gene corresponding to a sequence of reference mRNA NM_002439.4 at positions 1469-1486 of the MSH3 gene
E122. The dsRNA of any one of E1-E4, wherein the region the sense or antisense strand is complementary to is at least 15 contiguous nucleotides of an MSH3 gene corresponding to a sequence of reference mRNA NM_002439.4 at positions 1128-1151 of the MSH3 gene.
E123. The dsRNA of any one of E1-E4, wherein the region the sense or antisense strand is complementary to is at least 15 contiguous nucleotides of an MSH3 gene corresponding to a sequence of reference mRNA NM_002439.4 at positions 828-851 of the MSH3 gene.
E124. The dsRNA of any one of E1-E4, wherein the antisense strand comprises an antisense nucleobase sequence selected from Table 12, wherein the 5′ nucleotide represented by U can be any nucleotide (e.g., U, A, C, G), and the sense strand comprises a sense nucleobase sequence complementary to the antisense nucleobase sequence.
E125. The dsRNA of any one of E1-E4, wherein the antisense nucleobase sequence consists of an antisense strand in Table 12, wherein the 5′ nucleotide represented by U can be any nucleotide (e.g., U, A, C, G), and the sense nucleobase sequence consists of a sequence complementary to the antisense nucleobase sequence.
E126. The dsRNA of any one of E1-E4, wherein the sense strand comprises a sense nucleobase sequence selected from Table 12, and the antisense strand comprises an antisense nucleobase sequence complementary to the sense nucleobase sequence.
E127. The dsRNA of any one of E1-E4, wherein the sense nucleobase sequence consists of a sense strand in Table 12, and the antisense nucleobase sequence consists of a sequence complementary to the sense nucleobase sequence.
E128. The dsRNA of any one of E109-E127, wherein the dsRNA comprises at least one alternative nucleobase, at least one alternative internucleoside linkage, at least one alternative sugar moiety, or a combination thereof, optionally wherein the sense strand is selected from Table 13 and the antisense strand is selected from Table 14.
E129. The dsRNA of E127, wherein at least one alternative internucleoside linkage is a phosphorothioate internucleoside linkage.
E130. The dsRNA of E127, wherein at least one alternative internucleoside linkage is a 2′-alkoxy internucleoside linkage.
E131. The dsRNA of E127, wherein at least one alternative internucleoside linkage is an alkyl phosphate internucleoside linkage.
E132. The dsRNA of E127, wherein at least one alternative nucleobase is 5′-methylcytosine, pseudouridine, or 5-methoxyuridine.
E133. The dsRNA of E127, wherein at least one alternative sugar moiety is 2′-OMe or a bicyclic nucleic acid.
E134. The dsRNA of E127, wherein the dsRNA comprises at least one 2′-OMe sugar moiety and at least one phosphorothioate internucleoside linkage.
E135. The dsRNA of any one of E110-E134, wherein the dsRNA further comprises a ligand conjugated to the 3′ end of the sense strand through a monovalent or branched bivalent or trivalent linker.
E136. The dsRNA of any one of E110-135, wherein the sense strand comprises a nucleobase sequence of any one of SEQ ID NOs: 78, 82, 104, 148, 158, 160, 190, 240, 244, 246, 250, 252, 260, 308, 314, 316, 354,356, 360, 362, 364, 368, 370, 372, 396, 414, 416, 418, 474, 476,478, 480, 502, 512, 552 558, 560, 582, 616, 618, 634, 636, 642, 646, 648, 656, 660, 690, 692, 718, 720, 722, 796, 820, 822, 826, 848, 852, 854, 900, 914, 928, 930, 934,936, 946, 948, 966, 970,972, 988, 990, 992, 994, 996, 1006, 1020, 1032, 1054, 1056, 1058, 1076, 1088, 1096, 1098, 1110, 1112, 1126, 1214, 1220, 1230, 1306, 1308, 1310, 1318, 1326, 1386, 1394, 1396, 1400, 1404, 1424, 1426, 1448, 1452, 1454, 1506, 1524, 1540, 1546, 1656, 1666, 1674, 1676, 1678, 1722, 1762, 1766, 1768, 1836, 1838, 1842, 1868, 1886, 1888, 1964, 1990, 2030, 2108, 2128, 2230, 2242, 2246, 2254, 2274, 2294, 2330, 2334, 2356, 2360, 2362, 2448, 2502, 2504, 2516, 2518, 2578, 2580, 2592, 2596, 2602, 2654, 2656, 2686, 2762, 2768, 2782, 2844, or 2846.
E137. The dsRNA of any one of E110-E135, wherein the sense strand comprises a nucleobase sequence of any one of SEQ ID NOs: 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516, 2518, 2578, 2592, 2596, 2602, 2654, 2782, 2844, or 2846.
E138. The dsRNA of any one of E110-E135, wherein the sense strand comprises a nucleobase sequence of any one of SEQ ID NOs: 104, 372, 582, 634, 930, 934, 970, 1054, 1076, 1088, 1098, 1230, 1400, 1506, 1888, 2128, E2230, 2518, 2592, 2654, or 2844.
E139. The dsRNA of any one of E110-E135, wherein the sense strand comprises a nucleobase sequence of any one of SEQ ID NOs: 582, 934, 1076, 1088, 1098, 1230, 1400, 1506, 2230, or 2844.
E140. The dsRNA of any one of E110-E135, wherein the sense strand consists of a nucleobase sequence of any one of SEQ ID NOs: 78, 82, 104, 148, 158, 160, 190, 240, 244, 246, 250, 252, 260, 308, 314, 316, 354, 356, 360, 362, 364, 368, 370, 372, 396, 414, 416, 418, 474, 476, 478, 480, 502, 512, 552, 558, 560, 582, 616, 618, 634, 636, 642, 646, 648, 656, 660, 690, 692, 718, 720, 722, 796, 820, 822, 826, 848, 852, 854, 900, 914, 928, 930, 934, 936, 946, 948, 966, 970, 972, 988, 990, 992, 994, 996, 1006, 1020, 1032, 1054, 1056, 1058, 1076, 1088, 1096, 1098, 1110, 1112, 1126, 1214, 1220, 1230, 1306, 1308, 1310, 1318, 1326, 1386, 1394, 1396, 1400, 1404, 1424, 1426, 1448, 1452, 1454, 1506, 1524, 1540, 1546, 1656, 1666, 1674, 1676, 1678, 1722, 1762, 1766, 1768, 1836, 1838, 1842, 1868, 1886, 1888, 1964, 1990, 2030, 2108, 2128, 2230, 2242, 2246, 2254, 2274, 2294, 2330, 2334, 2356, 2360, 2362, 2448, 2502, 2504, 2516, 2518, 2578, 2580, 2592, 2596, 2602, 2654, 2656, 2686, 2762, 2768, 2782, 2844, or 2846.
E141. The dsRNA of any one of E110-E135, wherein the sense strand consists of a nucleobase sequence of any one of SEQ ID NOs: 104, 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516 2518, 2578, 2592, 2596, 2602, 2654, 2782, 2844, or 2846.
E142. The dsRNA of any one of E110-E135, wherein the sense strand consists of a nucleobase sequence of any one of SEQ ID NOs: 104, 372, 582, 634, 930, 934, 970, 1054, 1076, 1088, 1098, 1230, 1400, 1506, 1888, 2128, 2230, 2518, 2592, 2654, or 2844.
E143. The dsRNA of any one of E110-E135, wherein the sense strand consists of a nucleobase sequence of any one of SEQ ID NOs: 582, 934, 1076, 1088, 1098, 1230, 1400, 1506, 2230, or 2844
E144. The dsRNA of any one of E110-E135, wherein the sense strand consists of a nucleobase sequence of any one of SEQ ID NOs: 78, 82, 104, 148, 158, 160, 190, 240, 244, 246, 250, 252, 260, 308, 314, 316, 354, 356, 360, 362, 364, 368, 370, 372, 396, 414, 416, 418, 474, 476,478, 480, 502, 512, 552 558, 560, 582, 616, 618, 634, 636, 642, 646, 648, 656, 660, 690, 692, 718, 720, 722, 796, 820, 822, 826, 848, 852, 854, 900, 914, 928, 930, 934,936, 946, 948, 966, 970,972, 988, 990, 992, 994, 996, 1006, 1020, 1032, 1054, 1056, 1058, 1076, 1088, 1096, 1098, 1110, 1112, 1126, 1214, 1220, 1230, 1306, 1308, 1310, 1318, 1326, 1386, 1394, 1396, 1400, 1404, 1424, 1426, 1448, 1452, 1454, 1506, 1524, 1540, 1546, 1656, 1666, 1674, 1676, 1678, 1722, 1762, 1766, 1768, 1836, 1838, 1842, 1868, 1886, 1888, 1964, 1990, 2030, 2108, 2128, 2230, 2242, 2246, 2254, 2274, 2294, 2330, 2334, 2356, 2360, 2362, 2448, 2502, 2504, 2516, 2518, 2578, 2580, 2592, 2596, 2602, 2654, 2656, 2686, 2762, 2768, 2782, 2844, or 2846 and an overhang of 1-4 nucleotides.
E145. The dsRNA of any one of E110-E135, wherein the sense strand consists of a nucleobase sequence of any one of SEQ ID NOs: 104, 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516, 2518, 2578, 2592, 2596, 2602, 2654, 2782, 2844, or 2846 and an overhang of 1-4 nucleotides.
E146. The dsRNA of any one of E110-E135, wherein the sense strand consists of a nucleobase sequence of any one of SEQ ID NOs: 104, 372, 582, 634,930, 934, 970, 1054, 1076, 1088, 1098, 1230, 1400, 1506, 1888, 2128, 2230, 2518, 2592, 2654, or 2844 and an overhang of 1-4 nucleotides.
E147. The dsRNA of any one of E110-E135, wherein the sense strand consists of a nucleobase sequence of any one of SEQ ID NOs: 582, 934, 1076, 1088, 1098, 1230, 1400, 1506, 2230, or 2844 and an overhang of 1-4 nucleotides.
E148. The dsRNA of any one of E110-E135, wherein the antisense strand comprises a nucleobase sequence of any one of SEQ ID NOs: 79, 83, 105, 149, 159, 161, 191, 241, 245, 247, 251, 253, 261, 309, 315, 317, 355, 357, 361, 363, 365, 369, 371, 373, 379, 415, 417, 419, 475, 477, 479, 481, 503, 513, 553, 559, 561, 583, 617, 619, 635, 637, 643, 647, 649, 657, 661, 691, 693, 719, 721, 723, 797, 821, 823, 827, 849, 853, 855, 901, 915, 929, 931, 935, 937, 947, 949, 967, 971, 973, 989, 991, 993, 995, 997, 1007, 1021, 1032, 1055, 1057, 1059, 1077, 1089, 1097, 1099, 1111, 1113, 1127, 1215, 1221, 1230, 1307, 1309, 1311, 1319, 1363, 1387, 1395, 1397, 1401, 1405, 1425, 1427, 1449, 1453, 1455, 1507, 1525, 1541, 1547, 1657, 1667, 1675, 1677, 1679, 1723, 1763, 1767, 1769, 1837, 1839, 1843, 1869, 1887, 1889, 1965, 1991, 2031, 2109, 2129, 2231, 2243, 2247, 2255, 2275, 2295, 2331, 2335, 2357, 2361, 2363, 2449, 2503, 2505, 2517, 2519, 2579, 2581, 2593, 2597, 2603, 2655, 2657, 2687, 2763, 2769, 2783, 2845, or 2847, wherein the 5′ nucleotide represented by U can be any nucleotide (e.g., U, A, C, G).
E149. The dsRNA of any one of E110-E135, wherein the antisense strand comprises a nucleobase sequence of any one of SEQ ID NOs: 104, 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516, 2518, 2578, 2592, 2596, 2602, 2654, 278, 2844, or 2846, wherein the 5′ nucleotide represented by U can be any nucleotide (e.g., U, A, C, G).
E150. The dsRNA of any one of E110-E135, wherein the antisense strand comprises a nucleobase sequence of any one of SEQ ID NOs: 104, 372, 582, 634, 930, 934, 970, 1054, 1076, 1088, 1098, 1230, 1400, 1506, 1888, 2128, 2230, 2518, 2592, 2654, or 2844, wherein the 5′ nucleotide represented by U can be any nucleotide (e.g., U, A, C, G).
E151. The dsRNA of any one of E110-E135, wherein the antisense strand comprises a nucleobase sequence of any one of SEQ ID NOs: 582, 934, 1076, 1088, 1098, 1230, 1400, 1506, 2230, or 2844, wherein the 5′ nucleotide represented by U can be any nucleotide (e.g., U, A, C, G)
E152. The dsRNA of any one of E110-E135, wherein the antisense strand consists of a nucleobase sequence of any one of SEQ ID NOs: 79, 83, 105, 149, 159, 161, 191, 241, 245, 247, 251, 253, 261, 309, 315, 317, 355, 357, 361, 363, 365, 369, 371, 373, 379, 415, 417, 419, 475, 477, 479, 481, 503, 513, 553, 559, 561, 583, 617, 619, 635, 637, 643, 647, 649, 657, 661, 691, 693, 719, 721, 723, 797, 821, 823, 827, 849, 853, 855, 901, 915, 929, 931, 935, 937, 947, 949, 967, 971, 973, 989, 991, 993, 995, 997, 1007, 1021, 1032, 1055, 1057, 1059, 1077, 1089, 1097, 1099, 1111, 1113, 1127, 1215, 1221, 1230, 1307, 1309, 1311, 1319, 1363, 1387, 1395, 1397, 1401, 1405, 1425, 1427, 1449, 1453, 1455, 1507, 1525, 1541, 1547, 1657, 1667, 1675, 1677, 1679, 1723, 1763, 1767, 1769, 1837, 1839, 1843, 1869, 1887, 1889, 1965, 1991, 2031, 2109, 2129, 2231, 2243, 2247, 2255, 2275, 2295, 2331, 2335, 2357, 2361, 2363, 2449, 2503, 2505, 2517, 2519, 2579, 2581, 2593, 2597, 2603, 2655, 2657, 2687, 2763, 2769, 2783, 2845, or 2847, wherein the 5′ nucleotide represented by U can be any nucleotide (e.g., U, A, C, G).
E153. The dsRNA of any one of E110-E135, wherein the antisense strand consists of a nucleobase sequence of any one of SEQ ID NOs: 104, 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516, 2518, 2578, 2592, 2596, 2602, 2654, 2782, 2844, or 2846, wherein the 5′ nucleotide represented by U can be any nucleotide (e.g., U, A, C, G).
E154. The dsRNA of any one of E110-E135, wherein the antisense strand consists of a nucleobase sequence of any one of SEQ ID NOs: 104, 372, 582, 634, 930, 934, 970, 1054, 1076, 1088, 1098, 1230, 1400, 1506, 1888, 2128, 2230, 2518, 2592, 2654, or 2844, wherein the 5′ nucleotide represented by U can be any nucleotide (e.g., U, A, C, G).
E155. The dsRNA of any one of E110-E135, wherein the antisense strand consists of a nucleobase sequence of any one of SEQ ID NOs: 582, 934, 1076, 1088, 1098, 1230, 1400, 1506, 2230, or 2844, wherein the 5′ nucleotide represented by U can be any nucleotide (e.g., U, A, C, G)
E156. The dsRNA of any one of E110-E135, wherein the antisense strand consists of a nucleobase sequence of any one of SEQ ID NOs: 79, 83, 105, 149, 159, 161, 191, 241, 245, 247, 251, 253, 261, 309, 315, 317, 355, 357, 361, 363, 365, 369, 371, 373, 379, 415, 417, 419, 475, 477, 479, 481, 503, 513, 553, 559, 561, 583, 617, 619, 635, 637, 643, 647, 649, 657, 661, 691, 693, 719, 721, 723, 797, 821, 823, 827, 849, 853, 855, 901, 915, 929, 931, 935, 937, 947, 949, 967, 971, 973, 989, 991, 993, 995, 997, 1007, 1021, 1032, 1055, 1057, 1059, 1077, 1089, 1097, 1099, 1111, 1113, 1127, 1215, 1221, 1230, 1307, 1309, 1311, 1319, 1363, 1387, 1395, 1397, 1401, 1405, 1425, 1427, 1449, 1453, 1455, 1507, 1525, 1541, 1547, 1657, 1667, 1675, 1677, 1679, 1723, 1763, 1767, 1769, 1837, 1839, 1843, 1869, 1887, 1889, 1965, 1991, 2031, 2109, 2129, 2231, 2243, 2247, 2255, 2275, 2295, 2331, 2335, 2357, 2361, 2363, 2449, 2503, 2505, 2517, 2519, 2579, 2581, 2593, 2597, 2603, 2655, 2657, 2687, 2763, 2769, 2783, 2845, or 2847, wherein the 5′ nucleotide represented by U can be any nucleotide (e.g., U, A, C, G), and an overhang of 1-4 nucleotides.
E157. The dsRNA of any one of E110-E135, wherein the antisense strand consists of a nucleobase sequence of any one of SEQ ID NOs: 104, 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516, 2518, 2578, 2592, 2596, 2602, 2654, 2782, 2844, or 2846, wherein the 5′ nucleotide represented by U can be any nucleotide (e.g., U, A, C, G), and an overhang of 1-4 nucleotides.
E158. The dsRNA of any one of E110-E135, wherein the antisense strand consists of a nucleobase sequence of any one of SEQ ID NOs: 104, 372, 582, 634, 930, 934, 970, 1054, 1076, 1088, 1098, 1230, 1400, 1506, 1888, 2128, E2230, 2518, 2592, 2654, or 2844, wherein the 5′ nucleotide represented by U can be any nucleotide (e.g., U, A, C, G), and an overhang of 1-4 nucleotides.
E159. The dsRNA of any one of E110-E135, wherein the antisense strand consists of a nucleobase sequence of any one of SEQ ID NOs: 582, 934, 1076, 1088, 1098, 1230, 1400, 1506, 2230, or 2844, wherein the 5′ nucleotide represented by U can be any nucleotide (e.g., U, A, C, G), and an overhang of 1-4 nucleotides.
E160. The dsRNA of E136, wherein the sense strand comprises a nucleobase sequence of SEQ ID NO: 656.
E161. The dsRNA of E136, wherein the sense strand comprises a nucleobase sequence of SEQ ID NO: 636.
E162. The dsRNA of E136, wherein the sense strand comprises a nucleobase sequence of SEQ ID NO: 364.
E163. The dsRNA of E136, wherein the sense strand comprises a nucleobase sequence of SEQ ID NO: 648.
E164. The dsRNA of E136, wherein the sense strand comprises a nucleobase sequence of SEQ ID NO: E1366.
E165. The dsRNA of E136, wherein the sense strand comprises a nucleobase sequence of SEQ ID NO: 550.
E166. The dsRNA of E136, wherein the sense strand comprises a nucleobase sequence of SEQ ID NO: 1874.
E167. The dsRNA of E136, wherein the sense strand comprises a nucleobase sequence of SEQ ID NO: E1302.
E168. The dsRNA of E136, wherein the sense strand comprises a nucleobase sequence of SEQ ID NO: 420.
E169. The dsRNA of E136, wherein the sense strand comprises a nucleobase sequence of SEQ ID NO: 672.
E170. The dsRNA of E136, wherein the sense strand comprises a nucleobase sequence of SEQ ID NO: 832.
E171. The dsRNA of E148, wherein the antisense strand comprises a nucleobase sequence of SEQ ID NO: 649, wherein the 5′ nucleotide represented by U can be any nucleotide (e.g., U, A, C, G).
E172. The dsRNA of E148, wherein the antisense strand comprises a nucleobase sequence of SEQ ID NO: 657, wherein the 5′ nucleotide represented by U can be any nucleotide (e.g., U, A, C, G).
E173. The dsRNA of E148, wherein the antisense strand comprises a nucleobase sequence of SEQ ID NO: 637, wherein the 5′ nucleotide represented by U can be any nucleotide (e.g., U, A, C, G).
E174. The dsRNA of E148, wherein the antisense strand comprises a nucleobase sequence of SEQ ID NO: 365, wherein the 5′ nucleotide represented by U can be any nucleotide (e.g., U, A, C, G).
E175. The dsRNA of E148, wherein the antisense strand comprises a nucleobase sequence of SEQ ID NO: E1367, wherein the 5′ nucleotide represented by U can be any nucleotide (e.g., U, A, C, G).
E176. The dsRNA of E148, wherein the antisense strand comprises a nucleobase sequence of SEQ ID NO: 551, wherein the 5′ nucleotide represented by U can be any nucleotide (e.g., U, A, C, G).
E177. The dsRNA of E148, wherein the antisense strand comprises a nucleobase sequence of SEQ ID NO: 1875, wherein the 5′ nucleotide represented by U can be any nucleotide (e.g., U, A, C, G).
E178. The dsRNA of E148, wherein the antisense strand comprises a nucleobase sequence of SEQ ID NO: 1303, wherein the 5′ nucleotide represented by U can be any nucleotide (e.g., U, A, C, G).
E179. The dsRNA of E148, wherein the antisense strand comprises a nucleobase sequence of SEQ ID NO: 421, wherein the 5′ nucleotide represented by U can be any nucleotide (e.g., U, A, C, G).
E180. The dsRNA of E148, wherein the antisense strand comprises a nucleobase sequence of SEQ ID NO: 673, wherein the 5′ nucleotide represented by U can be any nucleotide (e.g., U, A, C, G).
E181. The dsRNA of E148, wherein the antisense strand comprises a nucleobase sequence of SEQ ID NO: 833, wherein the 5′ nucleotide represented by U can be any nucleotide (e.g., U, A, C, G).
E182. The dsRNA of any one of E110-E135, wherein the sense strand is any one of Sense Oligo Nos: 78, 82, 104, 148, 158, 160, 190, 240, 244, 246, 250, 252, 260, 308, 314, 316, 354, 356, 360, 362, 364, 368, 370, 372, 396, 414, 416, 418, 474, 476,478, 480, 502, 512, 552 558, 560, 582, 616, 618, 634, 636, 642, 646, 648, 656, 660, 690, 692, 718, 720, 722, 796, 820, 822, 826, 848, 852, 854, 900, 914, 928, 930, 934,936, 946, 948, 966, 970,972, 988, 990, 992, 994, 996, 1006, 1020, 1032, 1054, 1056, 1058, 1076, 1088, 1096, 1098, 1110, 1112, 1126, 1214, 1220, 1230, 1306, 1308, 1310, 1318, 1326, 1386, 1394, 1396, 1400, 1404, 1424, 1426, 1448, 1452, 1454, 1506, 1524, 1540, 1546, 1656, 1666, 1674, 1676, 1678, 1722, 1762, 1766, 1768, 1836, 1838, 1842, 1868, 1886, 1888, 1964, 1990, 2030, 2108, 2128, 2230, 2242, 2246, 2254, 2274, 2294, 2330, 2334, 2356, 2360, 2362, 2448, 2502, 2504, 2516, 2518, 2578, 2580, 2592, 2596, 2602, 2654, 2656, 2686, 2762, 2768, 2782, 2844, or 2846.
E183. The dsRNA of any one of E110-E135, wherein the sense strand is any one of Sense Oligo Nos: 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516 2518, 2578, 2592, 2596, 2602, 2654, 2782, 2844, or 2846.
E184. The dsRNA of any one of E110-E135, wherein the sense strand is any one of Sense Oligo Nos: 104, 372, 582, 634, 930, 934, 970, 1054, 1076, 1088, 1098, 1230, 1400, 1506, 1888, 2128, 2230, 2518, 2592, 2654, or 2844.
E185. The dsRNA of any one of E110-E135, wherein the sense strand is any one of Sense Oligo Nos: 582, 934, 1076, 1088, 1098, 1230, 1400, 1506, 2230, or 2844.
E186. The dsRNA of any one of E110-E135, wherein the antisense strand is any one of Antisense Oligo Nos: 79, 83, 105, 149, 159, 161, 191, 241, 245, 247, 251, 253, 261, 309, 315, 317, 355, 357, 361, 363, 365, 369, 371, 373, 379, 415, 417, 419, 475, 477, 479, 481, 503, 513, 553, 559, 561, 583, 617, 619, 635, 637, 643, 647, 649, 657, 661, 691, 693, 719, 721, 723, 797, 821, 823, 827, 849, 853, 855, 901, 915, 929, 931, 935, 937, 947, 949, 967, 971, 973, 989, 991, 993, 995, 997, 1007, 1021, 1032, 1055, 1057, 1059, 1077, 1089, 1097, 1099, 1111, 1113, 1127, 1215, 1221, 1230, 1307, 1309, 1311, 1319, 1363, 1387, 1395, 1397, 1401, 1405, 1425, 1427, 1449, 1453, 1455, 1507, 1525, 1541, 1547, 1657, 1667, 1675, 1677, 1679, 1723, 1763, 1767, 1769, 1837, 1839, 1843, 1869, 1887, 1889, 1965, 1991, 2031, 2109, 2129, 2231, 2243, 2247, 2255, 2275, 2295, 2331, 2335, 2357, 2361, 2363, 2449, 2503, 2505, 2517, 2519, 2579, 2581, 2593, 2597, 2603, 2655, 2657, 2687, 2763, 2769, 2783, 2845, or 2847, wherein the 5′ nucleotide represented by U can be any nucleotide (e.g., U, A, C, G).
E187. The dsRNA of any one of E110-E135, wherein the antisense strand is any one of Antisense Oligo Nos: 104, 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516, 2518, 2578, 2592, 2596, 2602, 2654, 2782, 2844, or 2846, wherein the 5′ nucleotide represented by U can be any nucleotide (e.g., U, A, C, G).
E188. The dsRNA of any one of E110-E135, wherein the antisense strand is any one of Antisense Oligo Nos: 104, 372, 582, 634, 930, 934, 970, 1054, 1076, 1088, 1098, 1230, 1400, 1506, 1888, 2128, 2230, 2518, 2592, 2654, or 2844, wherein the 5′ nucleotide represented by U can be any nucleotide (e.g., U, A, C, G).
E189. The dsRNA of any one of E110-E135, wherein the antisense strand is any one of Antisense Oligo Nos: 582, 934, 1076, 1088, 1098, 1230, 1400, 1506, 2230, or 2844, wherein the 5′ nucleotide represented by U can be any nucleotide (e.g., U, A, C, G).
E190. The dsRNA of E136, wherein the sense strand is Sense Oligo No: 656.
E191. The dsRNA of E136, wherein the sense strand is Sense Oligo No: 636.
E192. The dsRNA of E136, wherein the sense strand Sense Oligo No: 364.
E193. The dsRNA of E136, wherein the sense strand is Sense Oligo No: 648.
E194. The dsRNA of E136, wherein the sense strand is Sense Oligo No: 1366.
E195. The dsRNA of E136, wherein the sense strand is Sense Oligo No: 550.
E196. The dsRNA of E136, wherein the sense strand Sense Oligo No: 1874.
E197. The dsRNA of E136, wherein the sense strand Sense Oligo No: E1302.
E198. The dsRNA of E136, wherein the sense strand is Sense Oligo No: 420.
E199. The dsRNA of E135, wherein the sense strand Sense Oligo No: 672.
E200. The dsRNA of E136, wherein the sense strand is Sense Oligo No: 832.
E201. The dsRNA of E148, wherein the antisense strand is Antisense Oligo No: 649, wherein the 5′ nucleotide represented by U can be any nucleotide (e.g., U, A, C, G).
E202. The dsRNA of E148, wherein the antisense strand is Antisense Oligo No: 657, wherein the 5′ nucleotide represented by U can be any nucleotide (e.g., U, A, C, G).
E203. The dsRNA of E148, wherein the antisense strand is Antisense Oligo No: 637, wherein the 5′ nucleotide represented by U can be any nucleotide (e.g., U, A, C, G).
E204. The dsRNA of E148, wherein the antisense strand is Antisense Oligo No: 365, wherein the 5′ nucleotide represented by U can be any nucleotide (e.g., U, A, C, G).
E205. The dsRNA of E148, wherein the antisense strand is Antisense Oligo No: E1367, wherein the 5′ nucleotide represented by U can be any nucleotide (e.g., U, A, C, G).
E206. The dsRNA of E148, wherein the antisense strand is Antisense Oligo No: 551, wherein the 5′ nucleotide represented by U can be any nucleotide (e.g., U, A, C, G).
E207. The dsRNA of E148, wherein the antisense strand is Antisense Oligo No: 1875, wherein the 5′ nucleotide represented by U can be any nucleotide (e.g., U, A, C, G).
E208. The dsRNA of E148, wherein the antisense strand is Antisense Oligo No: 1303, wherein the 5′ nucleotide represented by U can be any nucleotide (e.g., U, A, C, G).
E209. The dsRNA of E148, wherein the antisense strand is Antisense Oligo No: 421, wherein the 5′ nucleotide represented by U can be any nucleotide (e.g., U, A, C, G).
E210. The dsRNA of E148, wherein the antisense strand is Antisense Oligo No: 673, wherein the 5′ nucleotide represented by U can be any nucleotide (e.g., U, A, C, G).
E211. The dsRNA of E148, wherein the antisense strand is Antisense Oligo No 833, wherein the 5′ nucleotide represented by U can be any nucleotide (e.g., U, A, C, G).
E212. The dsRNA of any one of E110-E211, wherein the dsRNA exhibits at least 50% mRNA inhibition at a 10 nM dsRNA concentration when determined using a cell assay compared with a control cell.
E213. The dsRNA of any one of E110-E211, wherein the dsRNA exhibits at least 60% mRNA inhibition at a 10 nM dsRNA concentration when determined using a cell assay compared with a control cell.
E214. The dsRNA of any one of E110-E211, wherein the dsRNA exhibits at least 70% mRNA inhibition at a 10 nM dsRNA concentration when determined using a cell assay when compared with a control cell.
E215. The dsRNA of any one of E110-E211, wherein the sense strand is complementary to at least 17 contiguous nucleotides of an MSH3 gene.
E216. The dsRNA of any one of E110-E211, wherein the sense strand is complementary to at least 19 contiguous nucleotides of an MSH3 gene.
E217. The dsRNA of any one of E110-E211, wherein the antisense strand is complementary to 17 contiguous nucleotides of an MSH3 gene.
E218. The dsRNA of any one of E110-E211, wherein the antisense strand is complementary to at least 19 contiguous nucleotides of an MSH3 gene.
E219. The dsRNA of any one of E110-E211, wherein the antisense strand and/or the sense strand comprises a 3′ overhang of at least 1 linked nucleoside; or a 3′ overhang of at least 2 linked nucleosides.
E220. A pharmaceutical composition comprising one or more dsRNAs of any one of E110-E219 and a pharmaceutically acceptable carrier.
E221. A composition comprising one or more dsRNAs of any one of E110-E219 and a lipid nanoparticle, a polyplex nanoparticle, a lipoplex nanoparticle, or a liposome.
E222. A vector encoding at least one strand of the dsRNA of any one of E110-E219.
E223. A cell comprising the vector of E222.
E224. A method of reducing transcription of MSH3 in a cell, the method comprising contacting the cell with the dsRNA of any one of E110-E219, the pharmaceutical composition of E220, the composition of E221, the vector of E222, or the cell of E223 for a time sufficient to obtain degradation of an mRNA transcript of MSH3, thereby reducing expression of MSH3 in the cell.
E225. A method of treating, preventing, or delaying progression of a nucleotide repeat expansion disorder in a subject in need thereof, the method comprising administering to the subject the dsRNA of any one of E110-E219, the pharmaceutical composition of E220, the composition of E221, the vector of E222, or the cell of E223.
E226. A method of reducing the level and/or activity of MSH3 in a cell of a subject identified as having a nucleotide repeat expansion disorder, the method comprising contacting the cell with the dsRNA of any one of E of any one of E110-E219, the pharmaceutical composition of E220, the composition of E221, the vector of E222, or the cell of E223.
E227. A method for reducing expression of MSH3 in a cell comprising contacting the cell with the dsRNA of any one of E of any one of E110-E219, the pharmaceutical composition of E220, the composition of E221, the vector of E222, or the cell of E223 and maintaining the cell for a time sufficient to obtain degradation of an mRNA transcript of MSH3, thereby reducing expression of MSH3 in the cell.

228. A method of decreasing nucleotide repeat expansion in a cell, the method comprising contacting the cell with the dsRNA of any one of E of any one of E110-E219, the pharmaceutical composition of E220, the composition of E221, the vector of E222, or the cell of E223.
E229. The method of any one of E224-E228, wherein the cell is in a subject.
E230. The method of any one of E224-E229, wherein the subject is a human.
E231. The method of any one of E224-E230, wherein the cell is a cell of the central nervous system or a muscle cell
E232. The method of any one of E225-E226 or E229-E231, wherein the subject is identified as having a nucleotide repeat expansion disorder.
E233. The method of any one of E232, wherein the subject is identified as having a trinucleotide repeat expansion disorder.
E234. The method of E233, wherein the trinucleotide repeat expansion disorder is a polyglutamine disease.
E235. The method of E234, wherein the polyglutamine disease is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, and Huntington's disease-like 2.
E236. The method of E233, wherein the trinucleotide repeat expansion disorder is a non-polyglutamine disease.
E237. The method of E236, wherein the non-polyglutamine disease is selected from the group consisting of fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.
E238. A dsRNA of any one of E of any one of E110-E219, the pharmaceutical composition of E220, the composition of E221, the vector of E222, or the cell of E223, for use in prevention or treatment of a nucleotide repeat expansion disorder.
E239. The dsRNA of E238, wherein the nucleotide repeat expansion disorder is a trinucleotide repeat expansion disorder.
E240. The dsRNA, the pharmaceutical composition, the composition, the vector, or the cell of E238 or E239, wherein the nucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.
E241. The dsRNA, the pharmaceutical composition, the composition, the vector, or the cell of E238 or E239, wherein the trinucleotide repeat expansion disorder is Huntington's disease.
E242. The dsRNA, the pharmaceutical composition, the composition, the vector, or the cell of E238 or E239, wherein the trinucleotide repeat expansion disorder is Friedreich's ataxia.
E243. The dsRNA, the pharmaceutical composition, the composition, the vector, or the cell of E238 or E239, wherein the trinucleotide repeat expansion disorder is myotonic dystrophy type 1.
E244. The dsRNA, the pharmaceutical composition, the composition, the vector, or the cell of any of E238 or E239, wherein the dsRNA, pharmaceutical composition, composition, vector, or cell is administered intrathecally.
E245. The dsRNA, the pharmaceutical composition, the composition, the vector, or the cell of any of E238 or E239, wherein the dsRNA, pharmaceutical composition, composition, vector, or cell is administered intraventricularly.
E246. The dsRNA, the pharmaceutical composition, the composition, the vector, or the cell of any of E238 or E239, wherein the dsRNA, pharmaceutical composition, composition, vector, or cell is administered intramuscularly.
E247. The dsRNA, the pharmaceutical composition, the composition, the vector, or the cell of any of E238 or E239, wherein the dsRNA, pharmaceutical composition, composition, vector, or cell is administered intracerebroventricularly.
E248. The dsRNA, the pharmaceutical composition, the composition, the vector, or the cell of any of E238 or E239, wherein the dsRNA, pharmaceutical composition, composition, vector, or cell is administered intraocularly.
E249. A method of treating, preventing, or delaying progression of a disorder in a subject in need thereof wherein the subject is suffering from trinucleotide repeat expansion disorder, comprising administering to said subject the dsRNA of any one of any one of E110-E219, the pharmaceutical composition of E220, the composition of E221, the vector of E222, or the cell of E223.
E250. The method of E249, further comprising administering at least one additional therapeutic agent.
E251. The method of E250, wherein at least one additional therapeutic agent is an antisense oligonucleotide that hybridizes to an mRNA encoding the Huntingtin gene.
E252. A method of preventing or delaying progression of a trinucleotide repeat expansion disorder in a subject, the method comprising administering to the subject the dsRNA of any one of s E110-E219, the pharmaceutical composition of E220, the composition of E221, the vector of 222, or the cell of E223 in an amount effective to delay progression of a nucleotide repeat expansion disorder of the subject.
E253. The method of E252, wherein the nucleotide repeat expansion disorder is a trinucleotide repeat expansion disorder.
E254. The method of E253, wherein the trinucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.
E255. The method of E253 or E254, wherein the trinucleotide repeat expansion disorder is Huntington's disease.
E256. The method of E253 or E254, wherein the trinucleotide repeat expansion disorder is Friedrich's ataxia.
E257. The method of E253 or E254, wherein the trinucleotide repeat expansion disorder is myotonic dystrophy type 1.
E258. The method of E253 or E254, wherein the trinucleotide repeat expansion disorder is a Spinocerebellar ataxia (SCA).
E259. The method of E258, wherein the SCA is Spinocerebellar ataxia type 1 (SCA1).
E260. The method of E258, wherein the SCA is Spinocerebellar ataxia type 10 (SCA10).
E261. The method of E258, wherein the SCA is Spinocerebellar ataxia type 12 (SCA12).
E262. The method of E258, wherein the SCA is Spinocerebellar ataxia type 17 (SCA17).
E263. The method of E258, wherein the SCA is Spinocerebellar ataxia type 2 (SCA2).
E264. The method of E258, wherein the SCA is Spinocerebellar ataxia type 3 (SCA3)/Machado-Joseph Disease.
E265. The method of E258, wherein the SCA is Spinocerebellar ataxia type 45 (SCA45).
E266. The method of E258, wherein the SCA is Spinocerebellar ataxia type 6 (SCA6).
E267. The method of E258, wherein the SCA is Spinocerebellar ataxia type 7 (SCA7).
E268. The method of E258, wherein the SCA is Spinocerebellar ataxia type 8 (SCA8).
E269. The method of any of E249-E268, further comprising administering at least one additional therapeutic agent.
E270. The method of 269, wherein at least one additional therapeutic agent is an antisense oligonucleotide that hybridizes to an mRNA encoding the Huntingtin gene.
E271. The method of any of E249-E270, wherein progression of the trinucleotide repeat expansion disorder is delayed by at least 120 days, for example, at least 6 months, at least 12 months, at least 2 years, at least 3 years, at least 4 years, at least 5 years, at least 10 years or more, when compared with a predicted progression.
E272. A dsRNA of any one of E110-E219, the pharmaceutical composition of E220, the composition of E221, the vector of E222, or the cell of E223, for use in preventing or delaying progression of a nucleotide repeat expansion disorder in a subject
E272. The dsRNA of E272, wherein the nucleotide repeat expansion disorder is a trinucleotide repeat expansion disorder.
E274. The dsRNA, the pharmaceutical composition, the composition, the vector, or the cell for use of E272 or E273, wherein the trinucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.
E275. The dsRNA, the pharmaceutical composition, the composition, the vector, or the cell of E273 or E274, wherein the trinucleotide repeat expansion disorder is Huntington's disease.
E276. The dsRNA, the pharmaceutical composition, the composition, the vector, or the cell of E273 or E274, wherein the trinucleotide repeat expansion disorder is Friedrich's ataxia.
E277. The dsRNA, the pharmaceutical composition, the composition, the vector, or the cell of E273 or E274, wherein the trinucleotide repeat expansion disorder is myotonic dystrophy type 1
E278. The dsRNA, the pharmaceutical composition, the composition, the vector, or the cell of 273 or 274, wherein the trinucleotide repeat expansion disorder is a Spinocerebellar ataxia (SCA).
E279. The dsRNA of E278, wherein the SCA is Spinocerebellar ataxia type 1 (SCA1).
E280. The dsRNA of E278, wherein the SCA is Spinocerebellar ataxia type 10 (SCA10).
E281. The dsRNA of E278, wherein the SCA is Spinocerebellar ataxia type 12 (SCA12).
E282. The dsRNA of E278, wherein the SCA is Spinocerebellar ataxia type 17 (SCA17).
E283. The dsRNA of E278, wherein the SCA is Spinocerebellar ataxia type 2 (SCA2).
E284. The dsRNA of E278, wherein the SCA is Spinocerebellar ataxia type 3 (SCA3)/Machado-Joseph Disease.
E285. The dsRNA of E278, wherein the SCA is Spinocerebellar ataxia type 45 (SCA45).
E286. The dsRNA of E278, wherein the SCA is Spinocerebellar ataxia type 6 (SCA6).
E287. The dsRNA of E278, wherein the SCA is Spinocerebellar ataxia type 7 (SCAT).
E288. The dsRNA of E278, wherein the SCA is Spinocerebellar ataxia type 8 (SCAB).
E289. The dsRNA, the pharmaceutical composition, the composition, the vector, or the cell of any one of E272-E288, wherein progression of the trinucleotide repeat expansion disorder is delayed by at least 120 days, for example, at least 6 months, at least 12 months, at least 2 years, at least 3 years, at least 4 years, at least 5 years, at least 10 years or more, when compared with a predicted progression.

1. A double-stranded ribonucleic acid (dsRNA), wherein the dsRNA comprises a sense strand and an antisense strand, wherein the antisense strand is complementary to at least 15 contiguous nucleobases of an MSH3 gene, and wherein the dsRNA comprises a duplex structure of between 15 and 30 linked nucleosides in length.

2. A dsRNA for reducing expression of MSH3 in a cell, wherein the dsRNA comprises a sense strand and an antisense strand, wherein the antisense strand is complementary to at least 15 contiguous nucleobases of an MSH3 gene, and wherein the dsRNA comprises a duplex structure of between 15 and 30 linked nucleosides in length.

3. The dsRNA of claim 1 or 2 comprising a duplex structure of between 19 and 23 linked nucleosides in length.

4. The dsRNA of any one of claims 1-3, further comprising a loop region joining the sense strand and antisense strand, wherein the loop region is characterized by a lack of base pairing between nucleobases within the loop region.

5. The dsRNA of any one of claims 1-4, wherein the region the sense or antisense strand is complementary to at least 15 contiguous nucleotides of an MSH3 gene corresponding to a sequence of reference mRNA NM_002439.4 at one or more of positions 566-589, 678-701, 875-989, 1019-1088, 1370-1393, 1466-1569, 1721-1849, 1879-2038, 2086-2171, 2783-2806, 2847-2922, 3043-3119, 3241-3314, 3330-3353, or 3701-3792 of the MSH3 gene.

6. The dsRNA of any one of claims 1-4, wherein the region the sense or antisense strand is complementary to at least 15 contiguous nucleotides of an MSH3 gene corresponding to a sequence of reference mRNA NM_002439.4 at one or more of positions 876-989, 1019-1088, 1370-1393, 1889-1938, or 3241-3314 of the MSH3 gene.

7. The dsRNA of any one of claims 1-4, wherein the region the sense or antisense strand is complementary to at least 15 contiguous nucleotides of an MSH3 gene corresponding to a sequence of reference mRNA NM_002439.4 at one or more of positions 876-989, 1019-1088, 1370-1393, 1466-1569, 1756-1849, 1879-2038, 2086-2171, 2783-2806, 2847-2922, 3043-3119, 3241-3314, 3330-3353, or 3701-3792 of the MSH3 gene.

8. The dsRNA of any one of claims 1-4, wherein the region the sense or antisense strand is complementary to at least 15 contiguous nucleotides of an MSH3 gene corresponding to a sequence of reference mRNA NM_002439.4 at one or more of positions 678-701, 875-989, 1019-1088, 1370-1393, 1466-1569, 1721-1849, 1879-2038, 2086-2171, 2783-2806, 2847-2922, 3043-3119, 3241-3314, 3330-3353, or 3701-3792 of the MSH3 gene.

9. The dsRNA of any one of claims 1-4, wherein the region the sense or antisense strand is complementary to at least 15 contiguous nucleotides of an MSH3 gene corresponding to a sequence of reference mRNA NM_002439.4 at position 879-921 of the MSH3 gene.

10. The dsRNA of any one of claims 1-4, wherein the region the sense or antisense strand is complementary to at least 15 contiguous nucleotides of an MSH3 gene corresponding to a sequence of reference mRNA NM_002439.4 at one or more of positions 875-989, 1019-1088, 1370-1393, 1466-1569, 1721-1849, 1879-1970, 2783-2806, 2847-2922, 3043-3119, 3241-3314, 3330-3353, or 3703-3792 of the MSH3 gene.

11. The dsRNA of any one of claims 1-4, wherein the region the sense or antisense strand is complementary to at least 15 contiguous nucleotides of an MSH3 gene corresponding to a sequence of reference mRNA NM_002439.4 at one or more of positions 566-589, 678-701, 875-989, 1019-1088, 1370-1393, 1466-1569, 1721-1849, 1879-2038, 2086-2171, 2783-2806, 2847-2922, 3043-3119, 3241-3314, 3330-3353, or 3701-3792 of the MSH3 gene.

12. The dsRNA of any one of claims 1-4, wherein the antisense strand comprises an antisense nucleobase sequence selected from Table 3, wherein the 5′ nucleotide represented by U can be any nucleotide (e.g., U, A, C, G), and the sense strand comprises a sense nucleobase sequence complementary to the antisense nucleobase sequence.

13. The dsRNA of any one of claims 1-4, wherein the antisense nucleobase sequence consists of an antisense strand in Table 3, wherein the 5′ nucleotide represented by U can be any nucleotide (e.g., U, A, C, G), and the sense nucleobase sequence consists of a sequence complementary to the antisense nucleobase sequence.

14. The dsRNA of any one of claims 1-4, wherein the sense strand comprises a sense nucleobase sequence selected from Table 3, and the antisense strand comprises an antisense nucleobase sequence complementary to the sense nucleobase sequence.

15. The dsRNA of any one of claims 1-4, wherein the sense nucleobase sequence consists of a sense strand in Table 3, and the antisense nucleobase sequence consists of a sequence complementary to the sense nucleobase sequence.

16. The dsRNA of any one of claims 1-4, wherein the sense strand comprises a sense nucleobase sequence selected from Tables 4-10, and the antisense strand comprises an antisense nucleobase sequence complementary to the sense nucleobase sequence.

17. The dsRNA of any one of claims 1-4, wherein the sense nucleobase sequence consists of a sense strand in any one of Tables 4-10, and the antisense nucleobase sequence consists of a sequence complementary to the sense nucleobase sequence.

18. The dsRNA of any one of claims 1-4, wherein the antisense strand comprises an antisense nucleobase sequence selected from a list in Table 11, wherein the 5′ nucleotide represented by U can be any nucleotide (e.g., U, A, C, G), and the sense strand comprises a sense nucleobase sequence complementary to the antisense nucleobase sequence.

19. The dsRNA of any one of claims 1-4, wherein the antisense nucleobase sequence consists of an antisense sense strand in Table 11, wherein the 5′ nucleotide represented by U can be any nucleotide (e.g., U, A, C, G), and the sense nucleobase sequence consists of a sequence complementary to the antisense nucleobase sequence.

20. The dsRNA of any one of claims 1-4, wherein the sense strand comprises a sense nucleobase sequence selected from Table 11, and the antisense strand comprises an antisense nucleobase sequence complementary to the sense nucleobase sequence.

21. The dsRNA of any one of claims 1-4, wherein the sense nucleobase sequence consists of a sense strand in Table 11, and the antisense nucleobase sequence consists of a sequence complementary to the sense nucleobase sequence.

22. The dsRNA of any one of claims 1-21, wherein the dsRNA comprises at least one alternative nucleobase, at least one alternative internucleoside linkage, at least one alternative sugar moiety, or a combination thereof.

23. The dsRNA of claim 22, wherein at least one alternative internucleoside linkage is a phosphorothioate internucleoside linkage.

24. The dsRNA of claim 22, wherein at least one alternative internucleoside linkage is a 2′-alkoxy internucleoside linkage.

25. The dsRNA of claim 22, wherein at least one alternative internucleoside linkage is an alkyl phosphate internucleoside linkage.

26. The dsRNA of claim 22, wherein at least one alternative nucleobase is 5′-methylcytosine, pseudouridine, or 5-methoxyuridine.

27. The dsRNA of claim 22, wherein at least one alternative sugar moiety is 2′-OMe or a bicyclic nucleic acid.

28. The dsRNA of claim 22, wherein the dsRNA comprises at least one 2′-OMe sugar moiety and at least one phosphorothioate internucleoside linkage.

29. The dsRNA of any one of claims 1-28, wherein the dsRNA further comprises a ligand conjugated to the 3′ end of the sense strand through a monovalent or branched bivalent or trivalent linker.

30. The dsRNA of any one of claims 1-29, wherein the sense strand comprises a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

31. The dsRNA of any one of claims 1-29, wherein the sense strand comprises a nucleobase sequence of any one of SEQ ID NOs: 380, 382, 386, 388, 396, 406, 418, 464, 478, 520, 540, 564, 568, 750, 1166, 1168, 1170, 1182, 1192, 1212, 1214, 1216, 1222, 2264, 2290, 2308, or 2318.

32. The dsRNA of any one of claims 1-29, wherein the sense strand comprises a nucleobase sequence of any one of SEQ ID NOs: 380, 382, 386, 388, 396, 406, 418, 464, 478, 520, 540, 564, 568, 750, 822, 830, 844, 868, 870, 874, 904, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

33. The dsRNA of any one of claims 1-29, wherein the sense strand comprises a nucleobase sequence of any one of SEQ ID NOs: 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

34. The dsRNA of any one of claims 1-29, wherein the sense strand comprises a nucleobase sequence of any one of SEQ ID NOs: 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

35. The dsRNA of any one of claims 1-29, wherein the sense strand comprises a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

36. The dsRNA of any one of claims 1-29, wherein the antisense strand comprises a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

37. The dsRNA of any one of claims 1-29, wherein the antisense strand comprises a nucleobase sequence of any one of SEQ ID NOs: 381, 383, 387, 389, 397, 407, 419, 465, 479, 521, 541, 565, 569, 751, 1167, 1169, 1171, 1183, 1193, 1213, 1215, 1217, 1223, 2265, 2291, 2309, or 2319, wherein the 5′ nucleotide represented by U can be any nucleotide (e.g., U, A, C, G).

38. The dsRNA of any one of claims 1-29, wherein the antisense strand comprises a nucleobase sequence of any one of SEQ ID NOs:381, 383, 387, 389, 397, 407, 419, 465, 479, 521, 541, 565, 569, 751, 823, 831, 845, 869, 871, 875, 905, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

39. The dsRNA of any one of claims 1-29, wherein the antisense strand comprises a nucleobase sequence of any one of SEQ ID NOs: 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

40. The dsRNA of any one of claims 1-29, wherein the antisense strand comprises a nucleobase sequence of any one of SEQ ID NOs: 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

41. The dsRNA of any one of claims 1-29, wherein the antisense strand comprises a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

42. The dsRNA of any one of claims 1-29, wherein the sense strand consists of a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

43. The dsRNA of any one of claims 1-29, wherein the sense strand consists of a nucleobase sequence of any one of SEQ ID NOs: 380, 382, 386, 388, 396, 406, 418, 464, 478, 520, 540, 564, 568, 750, 1166, 1168, 1170, 1182, 1192, 1212, 1214, 1216, 1222, 2264, 2290, 2308, or 2318.

44. The dsRNA of any one of claims 1-29, wherein the sense strand consists of a nucleobase sequence of any one of SEQ ID NOs: 380, 382, 386, 388, 396, 406, 418, 464, 478, 520, 540, 564, 568, 750, 822, 830, 844, 868, 870, 874, 904, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

45. The dsRNA of any one of claims 1-29, wherein the sense strand consists of a nucleobase sequence of any one of SEQ ID NOs: 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

46. The dsRNA of any one of claims 1-29, wherein the sense strand consists of a nucleobase sequence of any one of SEQ ID NOs: 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

47. The dsRNA of any one of claims 1-29, wherein the sense strand consists of a nucleobase sequence of any one of SEQ ID NOs: 140, 156, 234, 240, 246, 380, 382, 386, 388, 396, 406, 418, 464, 478, 520, 540, 564, 568, 750, 822, 830, 844, 868, 870, 874, 904, 1042, 1060, 1062, 1064, 1068, 1090, 1096, 1098, 1114, 1116, 1166, 1168, 1170, 1182, 1192, 1212, 1214, 1216, 1222, 1244, 1258, 1292, 1358, 1360, 1374, 1378, 1380, 1400, 1866, 1868, 1870, 1882, 1892, 1926, 1946, 1964, 1970, 2084, 2088, 2090, 2094, 2124, 2130, 2146, 2264, 2290, 2308, 2318, 2324, 2606, 2608, 2610, 2632, 2652, 2678, or 2690.

48. The dsRNA of any one of claims 1-29, wherein the sense strand consists of a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691.

49. The dsRNA of any one of claims 1-29, wherein the antisense strand consists of a nucleobase sequence of any one of SEQ ID NOs: 381, 383, 387, 389, 397, 407, 419, 465, 479, 521, 541, 565, 569, 751, 1167, 1169, 1171, 1183, 1193, 1213, 1215, 1217, 1223, 2265, 2291, 2309, or 2319, wherein the 5′ nucleotide represented by U can be any nucleotide (e.g., U, A, C, G).

50. The dsRNA of any one of claims 1-29, wherein the antisense strand consists of a nucleobase sequence of any one of SEQ ID NOs: 381, 383, 387, 389, 397, 407, 419, 465, 479, 521, 541, 565, 569, 751, 823, 831, 845, 869, 871, 875, 905, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

51. The dsRNA of any one of claims 1-29, wherein the antisense strand consists of a nucleobase sequence of any one of SEQ ID NOs: 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

52. The dsRNA of any one of claims 1-29, wherein the antisense strand consists of a nucleobase sequence of any one of SEQ ID NOs: 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

53. The dsRNA of any one of claims 1-29, wherein the antisense strand consists of a nucleobase sequence of any one of SEQ ID NOs: 141, 157, 235, 241, 247, 381, 383, 387, 389, 397, 407, 419, 465, 479, 521, 541, 565, 569, 751, 823, 831, 845, 869, 871, 875, 905, 1043, 1061, 1063, 1065, 1069, 1091, 1097, 1099, 1115, 1117, 1167, 1169, 1171, 1183, 1193, 1213, 1215, 1217, 1223, 1245, 1259, 1293, 1359, 1361, 1375, 1379, 1381, 1401, 1867, 1869, 1871, 1883, 1893, 1927, 1947, 1965, 1971, 2085, 2089, 2091, 2095, 2125, 2131, 2147, 2265, 2291, 2309, 2319, 2325, 2607, 2609, 2611, 2633, 2653, 2679, or 2691, wherein the 5′ nucleotide represented by U can be any nucleotide (e.g., U, A, C, G).

54. The dsRNA of any one of claims 1-53, wherein the dsRNA exhibits at least 50% mRNA inhibition at a 0.5 nM dsRNA concentration when determined using a cell assay when compared with a control cell.

55. The dsRNA of any one of claims 1-53, wherein the dsRNA exhibits at least 40% mRNA inhibition at a 0.5 nM dsRNA concentration when determined using a cell assay when compared with a control cell.

56. The dsRNA of any one of claims 1-53, wherein the dsRNA exhibits at least 30% mRNA inhibition at a 0.5 nM dsRNA concentration when determined using a cell assay when compared with a control cell.

57. The dsRNA of any one of claims 1-53, wherein the dsRNA exhibits at least 70% mRNA inhibition at a 10 nM dsRNA concentration when determined using a cell assay when compared with a control cell.

58. The dsRNA of any one of claims 1-53, wherein the dsRNA exhibits at least 60% mRNA inhibition at a 10 nM dsRNA concentration when determined using a cell assay when compared with a control cell.

59. The dsRNA of any one of claims 1-53, wherein the dsRNA exhibits at least 50% mRNA inhibition at a 10 nM dsRNA concentration when determined using a cell assay when compared with a control cell.

60. The dsRNA of any one of claims 1-59, wherein the antisense strand is complementary to at least 17 contiguous nucleotides of an MSH3 gene.

61. The dsRNA of any one of claims 1-59, wherein the antisense strand is complementary to at least 19 contiguous nucleotides of an MSH3 gene.

62. The dsRNA of any one of claims 1-59, wherein the antisense strand is complementary to 19 contiguous nucleotides of an MSH3 gene.

63. The dsRNA of any one of claims 1-59, wherein the sense strand is complementary to at least 17 contiguous nucleotides of an MSH3 gene.

64. The dsRNA of any one of claims 1-59, wherein the sense strand is complementary to at least 19 contiguous nucleotides of an MSH3 gene.

65. The dsRNA of any one of claims 1-59, wherein the sense strand is complementary to 19 contiguous nucleotides of an MSH3 gene.

66. The dsRNA of any one of claims 1-65, wherein the antisense strand and/or the sense strand comprises a 3′ overhang of at least 1 linked nucleoside; or a 3′ overhang of at least 2 linked nucleosides.

67. A pharmaceutical composition comprising one or more dsRNAs of any one of claims 1-66 and a pharmaceutically acceptable carrier.

68. A composition comprising one or more dsRNAs of any one of claims 1-66 and a lipid nanoparticle, a polyplex nanoparticle, a lipoplex nanoparticle, or a liposome.

69. A vector encoding at least one strand of the dsRNA of any one of claims 1-66.

70. A cell comprising the vector of claim 69.

71. A method of reducing transcription of MSH3 in a cell, the method comprising contacting the cell with the dsRNA of any one of claims 1-66, the pharmaceutical composition of claim 67, the composition of claim 68, the vector of claim 69, or the cell of claim 70 for a time sufficient to obtain degradation of an mRNA transcript of MSH3, thereby reducing expression of MSH3 in the cell.

72. A method of treating, preventing, or delaying progression of a nucleotide repeat expansion disorder in a subject in need thereof, the method comprising administering to the subject the dsRNA of any one of claims 1-66, the pharmaceutical composition of claim 67, the composition of claim 68, the vector of claim 69, or the cell of claim 70.

73. A method of reducing the level and/or activity of MSH3 in a cell of a subject identified as having a nucleotide repeat expansion disorder, the method comprising contacting the cell with the dsRNA of any one of claims 1-66, the pharmaceutical composition of claim 67, the composition of claim 68, the vector of claim 69, or the cell of claim 70.

74. A method for reducing expression of MSH3 in a cell comprising contacting the cell with the dsRNA of any one of claims 1-66, the pharmaceutical composition of claim 67, the composition of claim 68, the vector of claim 69, or the cell of claim 70 and maintaining the cell for a time sufficient to obtain degradation of an mRNA transcript of MSH3, thereby reducing expression of MSH3 in the cell.

75. A method of decreasing nucleotide repeat expansion in a cell, the method comprising contacting the cell with the dsRNA of any one of claims 1-66, the pharmaceutical composition of claim 67, the composition of claim 68, the vector of claim 69, or the cell of claim 70.

76. The method of claim 74 or 75, wherein the cell is in a subject.

77. The method of any one of claims 72, 73, and 76, wherein the subject is a human.

78. The method of any one of claims 71 and 73-76, wherein the cell is a cell of the central nervous system or a muscle cell.

79. The method of any one of claims 72, 73, and 76-78, wherein the subject is identified as having a nucleotide repeat expansion disorder.

80. The method of any one of claims 72, 73, and 75-79 wherein the nucleotide repeat expansion disorder is a polyglutamine disease.

81. The method of claim 80, wherein the polyglutamine disease is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, and Huntington's disease-like 2.

82. The method of any one of claims 72, 73, and 75-79, wherein the nucleotide repeat expansion disorder is a non-polyglutamine disease.

83. The method of claim 82, wherein the non-polyglutamine disease is selected from the group consisting of fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.

84. A dsRNA of any one of claims 1-66, the pharmaceutical composition of claim 67, the composition of claim 68, the vector of claim 69, or the cell of claim 70 for use in prevention or treatment of a nucleotide repeat expansion disorder.

85. The dsRNA, the pharmaceutical composition, the composition, the vector, or the cell of claim 84, wherein the nucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.

86. The dsRNA, the pharmaceutical composition, the composition, the vector, or the cell of claim 84 or 85, wherein the nucleotide repeat expansion disorder is Huntington's disease.

87. The dsRNA, the pharmaceutical composition, the composition, the vector, or the cell of claim 84 or 85, wherein the nucleotide repeat expansion disorder is Friedreich's ataxia.

88. The dsRNA, the pharmaceutical composition, the composition, the vector, or the cell of claim 84 or 85, wherein the nucleotide repeat expansion disorder is myotonic dystrophy type 1.

89. The dsRNA, the pharmaceutical composition, the composition, the vector, or the cell of any of claims 84-88, wherein the dsRNA, pharmaceutical composition, composition, vector, or cell is administered intrathecally.

90. The dsRNA, the pharmaceutical composition, the composition, the vector, or the cell of any of claims 84-88, wherein the dsRNA, pharmaceutical composition, composition, vector, or cell is administered intraventricularly.

91. The dsRNA, the pharmaceutical composition, the composition, the vector, or the cell of any of claims 84-88, wherein the dsRNA, pharmaceutical composition, composition, vector, or cell is administered intramuscularly.

92. A method of treating, preventing, or delaying progression of a disorder in a subject in need thereof wherein the subject is suffering from nucleotide repeat expansion disorder, comprising administering to said subject the dsRNA of any one of claims 1-66, the pharmaceutical composition of claim 67, the composition of claim 68, the vector of claim 69, or the cell of claim 70.

93. The method of claim 92, further comprising administering at least one additional therapeutic agent.

94. The method of claim 93, wherein at least one additional therapeutic agent is an antisense oligonucleotide that hybridizes to an mRNA encoding the Huntingtin gene.

95. A method of preventing or delaying progression of a nucleotide repeat expansion disorder in a subject, the method comprising administering to the subject the dsRNA of any one of claims 1-66, the pharmaceutical composition of claim 67, the composition of claim 68, the vector of claim 69, or the cell of claim 70 in an amount effective to delay progression of a nucleotide repeat expansion disorder of the subject.

96. The method of claim 95, wherein the nucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.

97. The method of claim 95 or 96, wherein the nucleotide repeat expansion disorder is Huntington's disease.

98. The method of claim 95 or 96, wherein the nucleotide repeat expansion disorder is Friedrich's ataxia.

99. The method of claim 95 or 96, wherein the nucleotide repeat expansion disorder is myotonic dystrophy type 1.

100. The method of any of claim 95 or 96, further comprising administering at least one additional therapeutic agent.

101. The method of claim 100, wherein at least one additional therapeutic agent is an antisense oligonucleotide that hybridizes to an mRNA encoding the Huntingtin gene.

102. The method of any of claims 95-101, wherein progression of the nucleotide repeat expansion disorder is delayed by at least 120 days, for example, at least 6 months, at least 12 months, at least 2 years, at least 3 years, at least 4 years, at least 5 years, at least 10 years or more, when compared with a predicted progression.

103. A dsRNA of any one of claims 1-66, the pharmaceutical composition of claim 67, the composition of claim 68, the vector of claim 69, or the cell of claim 70, for use in preventing or delaying progression of a nucleotide repeat expansion disorder in a subject.

104. The dsRNA, the pharmaceutical composition, the composition, the vector, or the cell for use of claim 103, wherein the nucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.

105. The dsRNA, the pharmaceutical composition, the composition, the vector, or the cell of claim 103 or 104, wherein the nucleotide repeat expansion disorder is Huntington's disease.

106. The dsRNA, the pharmaceutical composition, the composition, the vector, or the cell of claim 103 or 104, wherein the nucleotide repeat expansion disorder is Friedrich's ataxia.

107. The dsRNA, the pharmaceutical composition, the composition, the vector, or the cell of claim 103 or 104, wherein the nucleotide repeat expansion disorder is myotonic dystrophy type 1.

108. The dsRNA, the pharmaceutical composition, the composition, the vector, or the cell of any one of claims 103-107, wherein progression of the nucleotide repeat expansion disorder is delayed by at least 120 days, for example, at least 6 months, at least 12 months, at least 2 years, at least 3 years, at least 4 years, at least 5 years, at least 10 years or more, when compared with a predicted progression.

109. The dsRNA, the pharmaceutical composition, the composition, the vector, or the cell of any one of claims 103-107, wherein progression of the nucleotide repeat expansion disorder is delayed by at least 120 days, for example, at least 6 months, at least 12 months, at least 2 years, at least 3 years, at least 4 years, at least 5 years, at least 10 years, or at least 20 years or more, when compared with a predicted progression.

110. The dsRNA of any one of claims 1-4, wherein the region the sense or antisense strand is complementary to is at least 15 contiguous nucleotides of an MSH3 gene corresponding to a sequence of reference mRNA NM_002439.4 at one or more of positions 430-453, 508-531, 560-599, 609-632, 681-721, 768-797, 823-856, 882-927, 968-1029, 1039-1096, 1106-1175, 1188-1217, 1272-1297, 1419-1474, 1489-1516, 1540-1627, 1633-1815, 1819-1842, 1899-1937, 2027-2066, 2085-2108, 2117-2156, 2163-2187, 2195-2241, 2293-2343, 2347-2374, 2493-2539, 2567-2590, 2619-2649, 2737-2764, 2779-2820, 2871-2894, 2900-2923, 2949-2972, 3049-3096, 3217-3266, 3272-3309, 3351-3383, 3386-3415, 3537-3560, 3581-3619, 3686-3728, 3754-3778, 3782-3805, 3909-3935, 4287-4310, or 4386-4412 of the MSH3 gene.

111. The dsRNA of any one of claims 1-4, wherein the region the sense or antisense strand is complementary to is at least 15 contiguous nucleotides of an MSH3 gene corresponding to a sequence of reference mRNA NM_002439.4 at one or more of positions 508-531, 827-856, 903-926, 1073-1096, 1126-1149, 1583-1609, 1639-1662, 1727-1750, 1755-1795, 1819-1842, 1905-1937, 2130-2153, 2293-2316, 2505-2528, 2625-2648, 2797-2820, 3073-3096, 3217-3240, 3351-3383, 3686-3728, 3754-3777, 4287-4310, or 4386-4412 of the MSH3 gene.

112. The dsRNA of any one of claims 1-4, wherein the region the sense or antisense strand is complementary to is at least 15 contiguous nucleotides of an MSH3 gene corresponding to a sequence of reference mRNA NM_002439.4 at one or more of positions 508-531, 833-856, 1073-1096, 1126-1149, 1583-1609, 1639-1662, 1727-1750, 1755-1795, 1914-1937, 2130-2153, 2293-2316, 2797-2820, 3073-3096, 3217-3240, 3596-3619, 3700-3723, 3754-3777, or 4386-4409 of the MSH3 gene.

113. The dsRNA of any one of claims 1-4, wherein the region the sense or antisense strand is complementary to is at least 15 contiguous nucleotides of an MSH3 gene corresponding to a sequence of reference mRNA NM_002439.4 at one or more of positions 1073-1096, 1586-1609, 1755-1795, 1914-1937, 2130-2153, 2293-2316, 3217-3240, or 4386-4409 of the MSH3 gene

114. The dsRNA of any one of claims 1-4, wherein the region the sense or antisense strand is complementary to is at least 15 contiguous nucleotides of an MSH3 gene corresponding to a sequence of reference mRNA NM_002439.4 at positions 908-925 of the MSH3 gene.

115. The dsRNA of any one of claims 1-4, wherein the region the sense or antisense strand is complementary to is at least 15 contiguous nucleotides of an MSH3 gene corresponding to a sequence of reference mRNA NM_002439.4 at positions 1167-1184 of the MSH3 gene.

116. The dsRNA of any one of claims 1-4, wherein the region the sense or antisense strand is complementary to is at least 15 contiguous nucleotides of an MSH3 gene corresponding to a sequence of reference mRNA NM_002439.4 at positions 1143-1166 of the MSH3 gene.

117. The dsRNA of any one of claims 1-4, wherein the region the sense or antisense strand is complementary to is at least 15 contiguous nucleotides of an MSH3 gene corresponding to a sequence of reference mRNA NM_002439.4 at positions 1150-1173 of the MSH3 gene

118. The dsRNA of any one of claims 1-4, wherein the region the sense or antisense strand is complementary to is at least 15 contiguous nucleotides of an MSH3 gene corresponding to a sequence of reference mRNA NM_002439.4 at positions 2090-2107 of the MSH3 gene

119. The dsRNA of any one of claims 1-4, wherein the region the sense or antisense strand is complementary to is at least 15 contiguous nucleotides of an MSH3 gene corresponding to a sequence of reference mRNA NM_002439.4 at positions 1040-1057 of the MSH3 gene

120. The dsRNA of any one of claims 1-4, wherein the region the sense or antisense strand is complementary to is at least 15 contiguous nucleotides of an MSH3 gene corresponding to a sequence of reference mRNA NM_002439.4 at positions 2018-2035 of the MSH3 gene

121. The dsRNA of any one of claims 1-4, wherein the region the sense or antisense strand is complementary to is at least 15 contiguous nucleotides of an MSH3 gene corresponding to a sequence of reference mRNA NM_002439.4 at positions 1469-1486 of the MSH3 gene

122. The dsRNA of any one of claims 1-4, wherein the region the sense or antisense strand is complementary to is at least 15 contiguous nucleotides of an MSH3 gene corresponding to a sequence of reference mRNA NM_002439.4 at positions 1128-1151 of the MSH3 gene.

123. The dsRNA of any one of claims 1-4, wherein the region the sense or antisense strand is complementary to is at least 15 contiguous nucleotides of an MSH3 gene corresponding to a sequence of reference mRNA NM_002439.4 at positions 828-851 of the MSH3 gene.

124. The dsRNA of any one of claims 1-4, wherein the antisense strand comprises an antisense nucleobase sequence selected from Table 12, wherein the 5′ nucleotide represented by U can be any nucleotide (e.g., U, A, C, G), and the sense strand comprises a sense nucleobase sequence complementary to the antisense nucleobase sequence.

125. The dsRNA of any one of claims 1-4, wherein the antisense nucleobase sequence consists of an antisense strand in Table 12, wherein the 5′ nucleotide represented by U can be any nucleotide (e.g., U, A, C, G), and the sense nucleobase sequence consists of a sequence complementary to the antisense nucleobase sequence.

126. The dsRNA of any one of claims 1-4, wherein the sense strand comprises a sense nucleobase sequence selected from Table 12, and the antisense strand comprises an antisense nucleobase sequence complementary to the sense nucleobase sequence.

127. The dsRNA of any one of claims 1-4, wherein the sense nucleobase sequence consists of a sense strand in Table 12, and the antisense nucleobase sequence consists of a sequence complementary to the sense nucleobase sequence.

128. The dsRNA of any one of claims 109-127, wherein the dsRNA comprises at least one alternative nucleobase, at least one alternative internucleoside linkage, at least one alternative sugar moiety, or a combination thereof, optionally wherein the sense strand is selected from Table 13 and the antisense strand is selected from Table 14.

129. The dsRNA of claim 127, wherein at least one alternative internucleoside linkage is a phosphorothioate internucleoside linkage.

130. The dsRNA of claim 127, wherein at least one alternative internucleoside linkage is a 2′-alkoxy internucleoside linkage.

131. The dsRNA of claim 127, wherein at least one alternative internucleoside linkage is an alkyl phosphate internucleoside linkage.

132. The dsRNA of claim 127, wherein at least one alternative nucleobase is 5′-methylcytosine, pseudouridine, or 5-methoxyuridine.

133. The dsRNA of claim 127, wherein at least one alternative sugar moiety is 2′-OMe or a bicyclic nucleic acid.

134. The dsRNA of claim 127, wherein the dsRNA comprises at least one 2′-OMe sugar moiety and at least one phosphorothioate internucleoside linkage.

135. The dsRNA of any one of claims 110-134, wherein the dsRNA further comprises a ligand conjugated to the 3′ end of the sense strand through a monovalent or branched bivalent or trivalent linker.

136. The dsRNA of any one of claims 110-135, wherein the sense strand comprises a nucleobase sequence of any one of SEQ ID NOs: 78, 82, 104, 148, 158, 160, 190, 240, 244, 246, 250, 252, 260, 308, 314, 316, 354,356, 360, 362, 364, 368, 370, 372, 396, 414, 416, 418, 474, 476, 478, 480, 502, 512, 552 558, 560, 582, 616, 618, 634, 636, 642, 646, 648, 656, 660, 690, 692, 718, 720, 722, 796, 820, 822, 826, 848, 852, 854, 900, 914, 928, 930, 934, 936, 946, 948, 966, 970,972, 988, 990, 992, 994, 996, 1006, 1020, 1032, 1054, 1056, 1058, 1076, 1088, 1096, 1098, 1110, 1112, 1126, 1214, 1220, 1230, 1306, 1308, 1310, 1318, 1326, 1386, 1394, 1396, 1400, 1404, 1424, 1426, 1448, 1452, 1454, 1506, 1524, 1540, 1546, 1656, 1666, 1674, 1676, 1678, 1722, 1762, 1766, 1768, 1836, 1838, 1842, 1868, 1886, 1888, 1964, 1990, 2030, 2108, 2128, 2230, 2242, 2246, 2254, 2274, 2294, 2330, 2334, 2356, 2360, 2362, 2448, 2502, 2504, 2516, 2518, 2578, 2580, 2592, 2596, 2602, 2654, 2656, 2686, 2762, 2768, 2782, 2844, or 2846.

137. The dsRNA of any one of claims 110-135, wherein the sense strand comprises a nucleobase sequence of any one of SEQ ID NOs: 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516, 2518, 2578, 2592, 2596, 2602, 2654, 2782, 2844, or 2846.

138. The dsRNA of any one of claims 110-135, wherein the sense strand comprises a nucleobase sequence of any one of SEQ ID NOs: 104, 372, 582, 634, 930, 934, 970, 1054, 1076, 1088, 1098, 1230, 1400, 1506, 1888, 2128, 2230, 2518, 2592, 2654, or 2844.

139. The dsRNA of any one of claims 110-135, wherein the sense strand comprises a nucleobase sequence of any one of SEQ ID NOs: 582, 934, 1076, 1088, 1098, 1230, 1400, 1506, 2230, or 2844.

140. The dsRNA of any one of claims 110-135, wherein the sense strand consists of a nucleobase sequence of any one of SEQ ID NOs: 78, 82, 104, 148, 158, 160, 190, 240, 244, 246, 250, 252, 260, 308, 314, 316, 354, 356, 360, 362, 364, 368, 370, 372, 396, 414, 416, 418, 474, 476, 478, 480, 502, 512, 552, 558, 560, 582, 616, 618, 634, 636, 642, 646, 648, 656, 660, 690, 692, 718, 720, 722, 796, 820, 822, 826, 848, 852, 854, 900, 914, 928, 930, 934, 936, 946, 948, 966, 970, 972, 988, 990, 992, 994, 996, 1006, 1020, 1032, 1054, 1056, 1058, 1076, 1088, 1096, 1098, 1110, 1112, 1126, 1214, 1220, 1230, 1306, 1308, 1310, 1318, 1326, 1386, 1394, 1396, 1400, 1404, 1424, 1426, 1448, 1452, 1454, 1506, 1524, 1540, 1546, 1656, 1666, 1674, 1676, 1678, 1722, 1762, 1766, 1768, 1836, 1838, 1842, 1868, 1886, 1888, 1964, 1990, 2030, 2108, 2128, 2230, 2242, 2246, 2254, 2274, 2294, 2330, 2334, 2356, 2360, 2362, 2448, 2502, 2504, 2516, 2518, 2578, 2580, 2592, 2596, 2602, 2654, 2656, 2686, 2762, 2768, 2782, 2844, or 2846.

141. The dsRNA of any one of claims 110-135, wherein the sense strand consists of a nucleobase sequence of any one of SEQ ID NOs: 104, 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516 2518, 2578, 2592, 2596, 2602, 2654, 2782, 2844, or 2846.

142. The dsRNA of any one of claims 110-135, wherein the sense strand consists of a nucleobase sequence of any one of SEQ ID NOs: 104, 372, 582, 634, 930, 934, 970, 1054, 1076, 1088, 1098, 1230, 1400, 1506, 1888, 2128, 2230, 2518, 2592, 2654, or 2844.

143. The dsRNA of any one of claims 110-135, wherein the sense strand consists of a nucleobase sequence of any one of SEQ ID NOs: 582, 934, 1076, 1088, 1098, 1230, 1400, 1506, 2230, or 2844

144. The dsRNA of any one of claims 110-135, wherein the sense strand consists of a nucleobase sequence of any one of SEQ ID NOs: 78, 82, 104, 148, 158, 160, 190, 240, 244, 246, 250, 252, 260, 308, 314, 316, 354, 356, 360, 362, 364, 368, 370, 372, 396, 414, 416, 418, 474, 476,478, 480, 502, 512, 552 558, 560, 582, 616, 618, 634, 636, 642, 646, 648, 656, 660, 690, 692, 718, 720, 722, 796, 820, 822, 826, 848, 852, 854, 900, 914, 928, 930, 934,936, 946, 948, 966, 970,972, 988, 990, 992, 994, 996, 1006, 1020, 1032, 1054, 1056, 1058, 1076, 1088, 1096, 1098, 1110, 1112, 1126, 1214, 1220, 1230, 1306, 1308, 1310, 1318, 1326, 1386, 1394, 1396, 1400, 1404, 1424, 1426, 1448, 1452, 1454, 1506, 1524, 1540, 1546, 1656, 1666, 1674, 1676, 1678, 1722, 1762, 1766, 1768, 1836, 1838, 1842, 1868, 1886, 1888, 1964, 1990, 2030, 2108, 2128, 2230, 2242, 2246, 2254, 2274, 2294, 2330, 2334, 2356, 2360, 2362, 2448, 2502, 2504, 2516, 2518, 2578, 2580, 2592, 2596, 2602, 2654, 2656, 2686, 2762, 2768, 2782, 2844, or 2846 and an overhang of 1-4 nucleotides.

145. The dsRNA of any one of claims 110-135, wherein the sense strand consists of a nucleobase sequence of any one of SEQ ID NOs: 104, 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516, 2518, 2578, 2592, 2596, 2602, 2654, 2782, 2844, or 2846 and an overhang of 1-4 nucleotides.

146. The dsRNA of any one of claims 110-135, wherein the sense strand consists of a nucleobase sequence of any one of SEQ ID NOs: 104, 372, 582, 634,930, 934, 970, 1054, 1076, 1088, 1098, 1230, 1400, 1506, 1888, 2128, 2230, 2518, 2592, 2654, or 2844 and an overhang of 1-4 nucleotides.

147. The dsRNA of any one of claims 110-135, wherein the sense strand consists of a nucleobase sequence of any one of SEQ ID NOs: 582, 934, 1076, 1088, 1098, 1230, 1400, 1506, 2230, or 2844 and an overhang of 1-4 nucleotides.

148. The dsRNA of any one of claims 110-135, wherein the antisense strand comprises a nucleobase sequence of any one of SEQ ID NOs: 79, 83, 105, 149, 159, 161, 191, 241, 245, 247, 251, 253, 261, 309, 315, 317, 355, 357, 361, 363, 365, 369, 371, 373, 379, 415, 417, 419, 475, 477, 479, 481, 503, 513, 553, 559, 561, 583, 617, 619, 635, 637, 643, 647, 649, 657, 661, 691, 693, 719, 721, 723, 797, 821, 823, 827, 849, 853, 855, 901, 915, 929, 931, 935, 937, 947, 949, 967, 971, 973, 989, 991, 993, 995, 997, 1007, 1021, 1032, 1055, 1057, 1059, 1077, 1089, 1097, 1099, 1111, 1113, 1127, 1215, 1221, 1230, 1307, 1309, 1311, 1319, 1363, 1387, 1395, 1397, 1401, 1405, 1425, 1427, 1449, 1453, 1455, 1507, 1525, 1541, 1547, 1657, 1667, 1675, 1677, 1679, 1723, 1763, 1767, 1769, 1837, 1839, 1843, 1869, 1887, 1889, 1965, 1991, 2031, 2109, 2129, 2231, 2243, 2247, 2255, 2275, 2295, 2331, 2335, 2357, 2361, 2363, 2449, 2503, 2505, 2517, 2519, 2579, 2581, 2593, 2597, 2603, 2655, 2657, 2687, 2763, 2769, 2783, 2845, or 2847, wherein the 5′ nucleotide represented by U can be any nucleotide (e.g., U, A, C, G).

149. The dsRNA of any one of claims 110-135, wherein the antisense strand comprises a nucleobase sequence of any one of SEQ ID NOs: 104, 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516, 2518, 2578, 2592, 2596, 2602, 2654, 278, 2844, or 2846, wherein the 5′ nucleotide represented by U can be any nucleotide (e.g., U, A, C, G).

150. The dsRNA of any one of claims 110-135, wherein the antisense strand comprises a nucleobase sequence of any one of SEQ ID NOs: 104, 372, 582, 634, 930, 934, 970, 1054, 1076, 1088, 1098, 1230, 1400, 1506, 1888, 2128, 2230, 2518, 2592, 2654, or 2844, wherein the 5′ nucleotide represented by U can be any nucleotide (e.g., U, A, C, G).

151. The dsRNA of any one of claims 110-135, wherein the antisense strand comprises a nucleobase sequence of any one of SEQ ID NOs: 582, 934, 1076, 1088, 1098, 1230, 1400, 1506, 2230, or 2844, wherein the 5′ nucleotide represented by U can be any nucleotide (e.g., U, A, C, G)

152. The dsRNA of any one of claims 110-135, wherein the antisense strand consists of a nucleobase sequence of any one of SEQ ID NOs: 79, 83, 105, 149, 159, 161, 191, 241, 245, 247, 251, 253, 261, 309, 315, 317, 355, 357, 361, 363, 365, 369, 371, 373, 379, 415, 417, 419, 475, 477, 479, 481, 503, 513, 553, 559, 561, 583, 617, 619, 635, 637, 643, 647, 649, 657, 661, 691, 693, 719, 721, 723, 797, 821, 823, 827, 849, 853, 855, 901, 915, 929, 931, 935, 937, 947, 949, 967, 971, 973, 989, 991, 993, 995, 997, 1007, 1021, 1032, 1055, 1057, 1059, 1077, 1089, 1097, 1099, 1111, 1113, 1127, 1215, 1221, 1230, 1307, 1309, 1311, 1319, 1363, 1387, 1395, 1397, 1401, 1405, 1425, 1427, 1449, 1453, 1455, 1507, 1525, 1541, 1547, 1657, 1667, 1675, 1677, 1679, 1723, 1763, 1767, 1769, 1837, 1839, 1843, 1869, 1887, 1889, 1965, 1991, 2031, 2109, 2129, 2231, 2243, 2247, 2255, 2275, 2295, 2331, 2335, 2357, 2361, 2363, 2449, 2503, 2505, 2517, 2519, 2579, 2581, 2593, 2597, 2603, 2655, 2657, 2687, 2763, 2769, 2783, 2845, or 2847, wherein the 5′ nucleotide represented by U can be any nucleotide (e.g., U, A, C, G).

153. The dsRNA of any one of claims 110-135, wherein the antisense strand consists of a nucleobase sequence of any one of SEQ ID NOs: 104, 362, 370, 372, 416, 582, 634, 930, 934, 970, 1054, 1076, 1088, 1096, 1098, 1126, 1220, 1230, 1400, 1506 1666, 1766, 1888, 2128, 2230, 2330, 2334, 2448, 2504, 2516, 2518, 2578, 2592, 2596, 2602, 2654, 2782, 2844, or 2846, wherein the 5′ nucleotide represented by U can be any nucleotide (e.g., U, A, C, G).

154. The dsRNA of any one of claims 110-135, wherein the antisense strand consists of a nucleobase sequence of any one of SEQ ID NOs: 104, 372, 582, 634, 930, 934, 970, 1054, 1076, 1088, 1098, 1230, 1400, 1506, 1888, 2128, 2230, 2518, 2592, 2654, or 2844, wherein the 5′ nucleotide represented by U can be any nucleotide (e.g., U, A, C, G).

155. The dsRNA of any one of claims 110-135, wherein the antisense strand consists of a 1400, 1506, 2230, or 2844, wherein the 5′ nucleotide represented by U can be any nucleotide (e.g., U, A, C, G)

156. The dsRNA of any one of claims 110-135, wherein the antisense strand consists of a nucleobase sequence of any one of SEQ ID NOs: 79, 83, 105, 149, 159, 161, 191, 241, 245, 247, 251, 253, 261, 309, 315, 317, 355, 357, 361, 363, 365, 369, 371, 373, 379, 415, 417, 419, 475, 477, 479, 481, 503, 513, 553, 559, 561, 583, 617, 619, 635, 637, 643, 647, 649, 657, 661, 691, 693, 719, 721, 723, 797, 821, 823, 827, 849, 853, 855, 901, 915, 929, 931, 935, 937, 947, 949, 967, 971, 973, 989, 991, 993, 995, 997, 1007, 1021, 1032, 1055, 1057, 1059, 1077, 1089, 1097, 1099, 1111, 1113, 1127, 1215, 1221, 1230, 1307, 1309, 1311, 1319, 1363, 1387, 1395, 1397, 1401, 1405, 1425, 1427, 1449, 1453, 1455, 1507, 1525, 1541, 1547, 1657, 1667, 1675, 1677, 1679, 1723, 1763, 1767, 1769, 1837, 1839, 1843, 1869, 1887, 1889, 1965, 1991, 2031, 2109, 2129, 2231, 2243, 2247, 2255, 2275, 2295, 2331, 2335, 2357, 2361, 2363, 2449, 2503, 2505, 2517, 2519, 2579, 2581, 2593, 2597, 2603, 2655, 2657, 2687, 2763, 2769, 2783, 2845, or 2847, wherein the 5′ nucleotide represented by U can be any nucleotide (e.g., U, A, C, G), and an overhang of 1-4 nucleotides.

157. The dsRNA of any one of claims 110-135, wherein the antisense strand consists of a nucleobase sequence of any one of SEQ ID NOs: 104, 362, 370, 372, 416, 582, 634, 930, 934, 970, 1054, 1076, 1088, 1096, 1098, 1126, 1220, 1230, 1400, 1506 1666, 1766, 1888, 2128, 2230, 2330, 2334, 2448, 2504, 2516, 2518, 2578, 2592, 2596, 2602, 2654, 2782, 2844, or 2846, wherein the 5′ nucleotide represented by U can be any nucleotide (e.g., U, A, C, G), and an overhang of 1-4 nucleotides.

158. The dsRNA of any one of claims 110-135, wherein the antisense strand consists of a nucleobase sequence of any one of SEQ ID NOs: 104, 372, 582, 634, 930, 934, 970, 1054, 1076, 1088, 1098, 1230, 1400, 1506, 1888, 2128, 2230, 2518, 2592, 2654, or 2844, wherein the 5′ nucleotide represented by U can be any nucleotide (e.g., U, A, C, G), and an overhang of 1-4 nucleotides.

159. The dsRNA of any one of claims 110-135, wherein the antisense strand consists of a 1400, 1506, 2230, or 2844, wherein the 5′ nucleotide represented by U can be any nucleotide (e.g., U, A, C, G), and an overhang of 1-4 nucleotides.

160. The dsRNA of claim 136, wherein the sense strand comprises a nucleobase sequence of SEQ ID NO: 656.

161. The dsRNA of claim 136, wherein the sense strand comprises a nucleobase sequence of SEQ ID NO: 636.

162. The dsRNA of claim 136, wherein the sense strand comprises a nucleobase sequence of SEQ ID NO: 364.

163. The dsRNA of claim 136, wherein the sense strand comprises a nucleobase sequence of SEQ ID NO: 648.

164. The dsRNA of claim 136, wherein the sense strand comprises a nucleobase sequence of SEQ ID NO: 1366.

165. The dsRNA of claim 136, wherein the sense strand comprises a nucleobase sequence of SEQ ID NO: 550.

166. The dsRNA of claim 136, wherein the sense strand comprises a nucleobase sequence of SEQ ID NO: 1874.

167. The dsRNA of claim 136, wherein the sense strand comprises a nucleobase sequence of SEQ ID NO: 1302.

168. The dsRNA of claim 136, wherein the sense strand comprises a nucleobase sequence of SEQ ID NO: 420.

169. The dsRNA of claim 136, wherein the sense strand comprises a nucleobase sequence of SEQ ID NO: 672.

170. The dsRNA of claim 136, wherein the sense strand comprises a nucleobase sequence of SEQ ID NO: 832.

171. The dsRNA of claim 148, wherein the antisense strand comprises a nucleobase sequence of SEQ ID NO: 649, wherein the 5′ nucleotide represented by U can be any nucleotide (e.g., U, A, C, G).

172. The dsRNA of claim 148, wherein the antisense strand comprises a nucleobase sequence of SEQ ID NO: 657, wherein the 5′ nucleotide represented by U can be any nucleotide (e.g., U, A, C, G).

173. The dsRNA of claim 148, wherein the antisense strand comprises a nucleobase sequence of SEQ ID NO: 637, wherein the 5′ nucleotide represented by U can be any nucleotide (e.g., U, A, C, G).

174. The dsRNA of claim 148, wherein the antisense strand comprises a nucleobase sequence of SEQ ID NO: 365, wherein the 5′ nucleotide represented by U can be any nucleotide (e.g., U, A, C, G).

175. The dsRNA of claim 148, wherein the antisense strand comprises a nucleobase sequence of SEQ ID NO: 1367, wherein the 5′ nucleotide represented by U can be any nucleotide (e.g., U, A, C, G).

176. The dsRNA of claim 148, wherein the antisense strand comprises a nucleobase sequence of SEQ ID NO: 551, wherein the 5′ nucleotide represented by U can be any nucleotide (e.g., U, A, C, G).

177. The dsRNA of claim 148, wherein the antisense strand comprises a nucleobase sequence of SEQ ID NO: 1875, wherein the 5′ nucleotide represented by U can be any nucleotide (e.g., U, A, C, G).

178. The dsRNA of claim 148, wherein the antisense strand comprises a nucleobase sequence of SEQ ID NO: 1303, wherein the 5′ nucleotide represented by U can be any nucleotide (e.g., U, A, C, G).

179. The dsRNA of claim 148, wherein the antisense strand comprises a nucleobase sequence of SEQ ID NO: 421, wherein the 5′ nucleotide represented by U can be any nucleotide (e.g., U, A, C, G).

180. The dsRNA of claim 148, wherein the antisense strand comprises a nucleobase sequence of SEQ ID NO: 673, wherein the 5′ nucleotide represented by U can be any nucleotide (e.g., U, A, C, G).

181. The dsRNA of claim 148, wherein the antisense strand comprises a nucleobase sequence of SEQ ID NO: 833, wherein the 5′ nucleotide represented by U can be any nucleotide (e.g., U, A, C, G).

182. The dsRNA of any one of claims 110-135, wherein the sense strand is any one of Sense Oligo Nos: 78, 82, 104, 148, 158, 160, 190, 240, 244, 246, 250, 252, 260, 308, 314, 316, 354, 356, 360, 362, 364, 368, 370, 372, 396, 414, 416, 418, 474, 476, 478, 480, 502, 512, 552 558, 560, 582, 616, 618, 634, 636, 642, 646, 648, 656, 660, 690, 692, 718, 720, 722, 796, 820, 822, 826, 848, 852, 854, 900, 914, 928, 930, 934, 936, 946, 948, 966, 970, 972, 988, 990, 992, 994, 996, 1006, 1020, 1032, 1054, 1056, 1058, 1076, 1088, 1096, 1098, 1110, 1112, 1126, 1214, 1220, 1230, 1306, 1308, 1310, 1318, 1326, 1386, 1394, 1396, 1400, 1404, 1424, 1426, 1448, 1452, 1454, 1506, 1524, 1540, 1546, 1656, 1666, 1674, 1676, 1678, 1722, 1762, 1766, 1768, 1836, 1838, 1842, 1868, 1886, 1888, 1964, 1990, 2030, 2108, 2128, 2230, 2242, 2246, 2254, 2274, 2294, 2330, 2334, 2356, 2360, 2362, 2448, 2502, 2504, 2516, 2518, 2578, 2580, 2592, 2596, 2602, 2654, 2656, 2686, 2762, 2768, 2782, 2844, or 2846.

183. The dsRNA of any one of claims 110-135, wherein the sense strand is any one of Sense Oligo Nos: 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516 2518, 2578, 2592, 2596, 2602, 2654, 2782, 2844, or 2846.

184. The dsRNA of any one of claims 110-135, wherein the sense strand is any one of Sense Oligo Nos: 104, 372, 582, 634, 930, 934, 970, 1054, 1076, 1088, 1098, 1230, 1400, 1506, 1888, 2128, 2230, 2518, 2592, 2654, or 2844.

185. The dsRNA of any one of claims 110-135, wherein the sense strand is any one of Sense Oligo Nos: 582, 934, 1076, 1088, 1098, 1230, 1400, 1506, 2230, or 2844.

186. The dsRNA of any one of claims 110-135, wherein the antisense strand is any one of Antisense Oligo Nos: 79, 83, 105, 149, 159, 161, 191, 241, 245, 247, 251, 253, 261, 309, 315, 317, 355, 357, 361, 363, 365, 369, 371, 373, 379, 415, 417, 419, 475, 477, 479, 481, 503, 513, 553, 559, 561, 583, 617, 619, 635, 637, 643, 647, 649, 657, 661, 691, 693, 719, 721, 723, 797, 821, 823, 827, 849, 853, 855, 901, 915, 929, 931, 935, 937, 947, 949, 967, 971, 973, 989, 991, 993, 995, 997, 1007, 1021, 1032, 1055, 1057, 1059, 1077, 1089, 1097, 1099, 1111, 1113, 1127, 1215, 1221, 1230, 1307, 1309, 1311, 1319, 1363, 1387, 1395, 1397, 1401, 1405, 1425, 1427, 1449, 1453, 1455, 1507, 1525, 1541, 1547, 1657, 1667, 1675, 1677, 1679, 1723, 1763, 1767, 1769, 1837, 1839, 1843, 1869, 1887, 1889, 1965, 1991, 2031, 2109, 2129, 2231, 2243, 2247, 2255, 2275, 2295, 2331, 2335, 2357, 2361, 2363, 2449, 2503, 2505, 2517, 2519, 2579, 2581, 2593, 2597, 2603, 2655, 2657, 2687, 2763, 2769, 2783, 2845, or 2847, wherein the 5′ nucleotide represented by U can be any nucleotide (e.g., U, A, C, G).

187. The dsRNA of any one of claims 110-135, wherein the antisense strand is any one of Antisense Oligo Nos: 104, 362, 370, 372, 416, 582, 634, 930, 934, 970, 1054, 1076, 1088, 1096, 1098, 1126, 1220, 1230, 1400, 1506, 1666, 1766, 1888, 2128, 2230, 2330, 2334, 2448, 2504, 2516, 2518, 2578, 2592, 2596, 2602, 2654, 2782, 2844, or 2846, wherein the 5′ nucleotide represented by U can be any nucleotide (e.g., U, A, C, G).

188. The dsRNA of any one of claims 110-135, wherein the antisense strand is any one of Antisense Oligo Nos: 104, 372, 582, 634, 930, 934, 970, 1054, 1076, 1088, 1098, 1230, 1400, 1506, 1888, 2128, 2230, 2518, 2592, 2654, or 2844, wherein the 5′ nucleotide represented by U can be any nucleotide (e.g., U, A, C, G).

189. The dsRNA of any one of claims 110-135, wherein the antisense strand is any one of Antisense Oligo Nos: 582, 934, 1076, 1088, 1098, 1230, 1400, 1506, 2230, or 2844, wherein the 5′ nucleotide represented by U can be any nucleotide (e.g., U, A, C, G).

190. The dsRNA of claim 136, wherein the sense strand is Sense Oligo No: 656.

191. The dsRNA of claim 136, wherein the sense strand is Sense Oligo No: 636.

192. The dsRNA of claim 136, wherein the sense strand Sense Oligo No: 364.

193. The dsRNA of claim 136, wherein the sense strand is Sense Oligo No: 648.

194. The dsRNA of claim 136, wherein the sense strand is Sense Oligo No: 1366.

195. The dsRNA of claim 136, wherein the sense strand is Sense Oligo No: 550.

196. The dsRNA of claim 136, wherein the sense strand Sense Oligo No: 1874.

197. The dsRNA of claim 136, wherein the sense strand Sense Oligo No: 1302.

198. The dsRNA of claim 136, wherein the sense strand is Sense Oligo No: 420.

199. The dsRNA of claim 135, wherein the sense strand Sense Oligo No: 672.

200. The dsRNA of claim 136, wherein the sense strand is Sense Oligo No: 832.

201. The dsRNA of claim 148, wherein the antisense strand is Antisense Oligo No: 649, wherein the 5′ nucleotide represented by U can be any nucleotide (e.g., U, A, C, G).

202. The dsRNA of claim 148, wherein the antisense strand is Antisense Oligo No: 657, wherein the 5′ nucleotide represented by U can be any nucleotide (e.g., U, A, C, G).

203. The dsRNA of claim 148, wherein the antisense strand is Antisense Oligo No: 637, wherein the 5′ nucleotide represented by U can be any nucleotide (e.g., U, A, C, G).

204. The dsRNA of claim 148, wherein the antisense strand is Antisense Oligo No: 365, wherein the 5′ nucleotide represented by U can be any nucleotide (e.g., U, A, C, G).

205. The dsRNA of claim 148, wherein the antisense strand is Antisense Oligo No: 1367, wherein the 5′ nucleotide represented by U can be any nucleotide (e.g., U, A, C, G).

206. The dsRNA of claim 148, wherein the antisense strand is Antisense Oligo No: 551, wherein the 5′ nucleotide represented by U can be any nucleotide (e.g., U, A, C, G).

207. The dsRNA of claim 148, wherein the antisense strand is Antisense Oligo No: 1875, wherein the 5′ nucleotide represented by U can be any nucleotide (e.g., U, A, C, G).

208. The dsRNA of claim 148, wherein the antisense strand is Antisense Oligo No: 1303, wherein the 5′ nucleotide represented by U can be any nucleotide (e.g., U, A, C, G).

209. The dsRNA of claim 148, wherein the antisense strand is Antisense Oligo No: 421, wherein the 5′ nucleotide represented by U can be any nucleotide (e.g., U, A, C, G).

210. The dsRNA of claim 148, wherein the antisense strand is Antisense Oligo No: 673, wherein the 5′ nucleotide represented by U can be any nucleotide (e.g., U, A, C, G).

211. The dsRNA of claim 148, wherein the antisense strand is Antisense Oligo No, wherein the 5′ nucleotide represented by U can be any nucleotide (e.g., U, A, C, G).

212. The dsRNA of any one of claims 110-210, wherein the dsRNA exhibits at least 50% mRNA inhibition at a 10 nM dsRNA concentration when determined using a cell assay compared with a control cell.

213. The dsRNA of any one of claims 110-210, wherein the dsRNA exhibits at least 60% mRNA inhibition at a 10 nM dsRNA concentration when determined using a cell assay compared with a control cell.

214. The dsRNA of any one of claims 110-210, wherein the dsRNA exhibits at least 70% mRNA inhibition at a 10 nM dsRNA concentration when determined using a cell assay when compared with a control cell.

215. The dsRNA of any one of claims 110-210, wherein the sense strand is complementary to at least 17 contiguous nucleotides of an MSH3 gene.

216. The dsRNA of any one of claims 110-210, wherein the sense strand is complementary to at least 19 contiguous nucleotides of an MSH3 gene.

217. The dsRNA of any one of claims 110-210, wherein the antisense strand is complementary to 17 contiguous nucleotides of an MSH3 gene.

218. The dsRNA of any one of claims 110-210, wherein the antisense strand is complementary to at least 19 contiguous nucleotides of an MSH3 gene.

219. The dsRNA of any one of claims 110-210, wherein the antisense strand and/or the sense strand comprises a 3′ overhang of at least 1 linked nucleoside; or a 3′ overhang of at least 2 linked nucleosides.

220. A pharmaceutical composition comprising one or more dsRNAs of any one of claims 110-219 and a pharmaceutically acceptable carrier.

221. A composition comprising one or more dsRNAs of any one of claims 110-219 and a lipid nanoparticle, a polyplex nanoparticle, a lipoplex nanoparticle, or a liposome.

222. A vector encoding at least one strand of the dsRNA of any one of claims 110-219.

223. A cell comprising the vector of claim 222.

224. A method of reducing transcription of MSH3 in a cell, the method comprising contacting the cell with the dsRNA of any one of claims 110-219, the pharmaceutical composition of claim 220, the composition of claim 221, the vector of claim 222, or the cell of claim 223 for a time sufficient to obtain degradation of an mRNA transcript of MSH3, thereby reducing expression of MSH3 in the cell.

225. A method of treating, preventing, or delaying progression of a nucleotide repeat expansion disorder in a subject in need thereof, the method comprising administering to the subject the dsRNA of any one of claims 110-219, the pharmaceutical composition of claim 220, the composition of claim 221, the vector of claim 222, or the cell of claim 223.

226. A method of reducing the level and/or activity of MSH3 in a cell of a subject identified as having a nucleotide repeat expansion disorder, the method comprising contacting the cell with the dsRNA of any one of claims of any one of claims 110-219, the pharmaceutical composition of claim 220, the composition of claim 221, the vector of claim 222, or the cell of claim 223.

227. A method for reducing expression of MSH3 in a cell comprising contacting the cell with the dsRNA of any one of claims of any one of claims 110-219, the pharmaceutical composition of claim 220, the composition of claim 221, the vector of claim 222, or the cell of claim 223 and maintaining the cell for a time sufficient to obtain degradation of an mRNA transcript of MSH3, thereby reducing expression of MSH3 in the cell.

228. A method of decreasing nucleotide repeat expansion in a cell, the method comprising contacting the cell with the dsRNA of any one of claims of any one of claims 110-219, the pharmaceutical composition of claim 220, the composition of claim 221, the vector of claim 222, or the cell of claim 223.

229. The method of any one of claims 224-228, wherein the cell is in a subject.

230. The method of any one of claims 224-229, wherein the subject is a human.

231. The method of any one of claims 224-230, wherein the cell is a cell of the central nervous system or a muscle cell

232. The method of any one of claim 225-226 or 229-231, wherein the subject is identified as having a nucleotide repeat expansion disorder.

233. The method of any one of claims claim 232, wherein the subject is identified as having a trinucleotide repeat expansion disorder.

234. The method of claim 233, wherein the trinucleotide repeat expansion disorder is a polyglutamine disease.

235. The method of claim 234, wherein the polyglutamine disease is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, and Huntington's disease-like 2.

236. The method of claim 233, wherein the trinucleotide repeat expansion disorder is a non-polyglutamine disease.

237. The method of claim 236, wherein the non-polyglutamine disease is selected from the group consisting of fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.

238. A dsRNA of any one of claims of any one of claims 110-219, the pharmaceutical composition of claim 220, the composition of claim 221, the vector of claim 222, or the cell of claim 223, for use in prevention or treatment of a nucleotide repeat expansion disorder.

239. The dsRNA of claim 238, wherein the nucleotide repeat expansion disorder is a trinucleotide repeat expansion disorder.

240. The dsRNA, the pharmaceutical composition, the composition, the vector, or the cell of claim 238 or 239, wherein the nucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.

241. The dsRNA, the pharmaceutical composition, the composition, the vector, or the cell of claim 238 or 239, wherein the trinucleotide repeat expansion disorder is Huntington's disease.

242. The dsRNA, the pharmaceutical composition, the composition, the vector, or the cell of claim 238 or 239, wherein the trinucleotide repeat expansion disorder is Friedreich's ataxia.

243. The dsRNA, the pharmaceutical composition, the composition, the vector, or the cell of claim 238 or 239, wherein the trinucleotide repeat expansion disorder is myotonic dystrophy type 1.

244. The dsRNA, the pharmaceutical composition, the composition, the vector, or the cell of any of claim 238 or 239, wherein the dsRNA, pharmaceutical composition, composition, vector, or cell is administered intrathecally.

245. The dsRNA, the pharmaceutical composition, the composition, the vector, or the cell of any of claim 238 or 239, wherein the dsRNA, pharmaceutical composition, composition, vector, or cell is administered intraventricularly.

246. The dsRNA, the pharmaceutical composition, the composition, the vector, or the cell of any of claim 238 or 239, wherein the dsRNA, pharmaceutical composition, composition, vector, or cell is administered intramuscularly.

247. The dsRNA, the pharmaceutical composition, the composition, the vector, or the cell of any of claim 238 or 239, wherein the dsRNA, pharmaceutical composition, composition, vector, or cell is administered intracerebroventricularly.

248. The dsRNA, the pharmaceutical composition, the composition, the vector, or the cell of any of claim 238 or 239, wherein the dsRNA, pharmaceutical composition, composition, vector, or cell is administered intraocularly.

249. A method of treating, preventing, or delaying progression of a disorder in a subject in need thereof wherein the subject is suffering from trinucleotide repeat expansion disorder, comprising administering to said subject the dsRNA of any one of any one of claims 110-219, the pharmaceutical composition of claim 220, the composition of claim 221, the vector of claim 222, or the cell of claim 223.

250. The method of claim 249, further comprising administering at least one additional therapeutic agent.

251. The method of claim 250, wherein at least one additional therapeutic agent is an antisense oligonucleotide that hybridizes to an mRNA encoding the Huntingtin gene.

252. A method of preventing or delaying progression of a trinucleotide repeat expansion disorder in a subject, the method comprising administering to the subject the dsRNA of any one of claims 110-219, the pharmaceutical composition of claim 220, the composition of claim 221, the vector of claim 222, or the cell of claim 223 in an amount effective to delay progression of a nucleotide repeat expansion disorder of the subject

253. The method of claim 252, wherein the nucleotide repeat expansion disorder is a trinucleotide repeat expansion disorder.

254. The method of claim 253, wherein the trinucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.

255. The method of claim 253 or 254, wherein the trinucleotide repeat expansion disorder is Huntington's disease.

256. The method of claim 253 or 254, wherein the trinucleotide repeat expansion disorder is Friedrich's ataxia.

257. The method of claim 253 or 254, wherein the trinucleotide repeat expansion disorder is myotonic dystrophy type 1.

258. The method of claim 253 or 254, wherein the trinucleotide repeat expansion disorder is a Spinocerebellar ataxia (SCA).

259. The method of claim 258, wherein the SCA is Spinocerebellar ataxia type 1 (SCA1)

260. The method of claim 258, wherein the SCA is Spinocerebellar ataxia type 10 (SCA10).

261. The method of claim 258, wherein the SCA is Spinocerebellar ataxia type 12 (SCA12).

262. The method of claim 258, wherein the SCA is Spinocerebellar ataxia type 17 (SCA17).

263. The method of claim 258, wherein the SCA is Spinocerebellar ataxia type 2 (SCA2).

264. The method of claim 258, wherein the SCA is Spinocerebellar ataxia type 3 (SCA3)/Machado-Joseph Disease.

265. The method of claim 258, wherein the SCA is Spinocerebellar ataxia type 45 (SCA45).

266. The method of claim 258, wherein the SCA is Spinocerebellar ataxia type 6 (SCA6).

267. The method of claim 258, wherein the SCA is Spinocerebellar ataxia type 7 (SCAT).

268. The method of claim 258, wherein the SCA is Spinocerebellar ataxia type 8 (SCAB).

269. The method of any of claims 249-268, further comprising administering at least one additional therapeutic agent.

270. The method of claim 269, wherein at least one additional therapeutic agent is an antisense oligonucleotide that hybridizes to an mRNA encoding the Huntingtin gene.

271. The method of any of claims 249-270, wherein progression of the trinucleotide repeat expansion disorder is delayed by at least 120 days, for example, at least 6 months, at least 12 months, at least 2 years, at least 3 years, at least 4 years, at least 5 years, at least 10 years or more, when compared with a predicted progression.

272. A dsRNA of any one of claims 110-219, the pharmaceutical composition of claim 220, the composition of claim 221, the vector of claim 222, or the cell of claim 223, for use in preventing or delaying progression of a nucleotide repeat expansion disorder in a subject

273. The dsRNA of claim 272, wherein the nucleotide repeat expansion disorder is a trinucleotide repeat expansion disorder.

274. The dsRNA, the pharmaceutical composition, the composition, the vector, or the cell for use of claim 272 or 273, wherein the trinucleotide repeat expansion disorder is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.

275. The dsRNA, the pharmaceutical composition, the composition, the vector, or the cell of claim 273 or 274, wherein the trinucleotide repeat expansion disorder is Huntington's disease.

276. The dsRNA, the pharmaceutical composition, the composition, the vector, or the cell of claim 273 or 274, wherein the trinucleotide repeat expansion disorder is Friedrich's ataxia.

277. The dsRNA, the pharmaceutical composition, the composition, the vector, or the cell of claim 273 or 274, wherein the trinucleotide repeat expansion disorder is myotonic dystrophy type 1

278. The dsRNA, the pharmaceutical composition, the composition, the vector, or the cell of claim 273 or 274, wherein the trinucleotide repeat expansion disorder is a Spinocerebellar ataxia (SCA).

279. The dsRNA of claim 278, wherein the SCA is Spinocerebellar ataxia type 1 (SCA1).

280. The dsRNA of claim 278, wherein the SCA is Spinocerebellar ataxia type 10 (SCA10).

281. The dsRNA of claim 278, wherein the SCA is Spinocerebellar ataxia type 12 (SCA12).

282. The dsRNA of claim 278, wherein the SCA is Spinocerebellar ataxia type 17 (SCA17).

283. The dsRNA of claim 278, wherein the SCA is Spinocerebellar ataxia type 2 (SCA2).

284. The dsRNA of claim 278, wherein the SCA is Spinocerebellar ataxia type 3 (SCA3)/Machado-Joseph Disease.

285. The dsRNA of claim 278, wherein the SCA is Spinocerebellar ataxia type 45 (SCA45).

286. The dsRNA of claim 278, wherein the SCA is Spinocerebellar ataxia type 6 (SCA6).

287. The dsRNA of claim 278, wherein the SCA is Spinocerebellar ataxia type 7 (SCAT).

288. The dsRNA of claim 278, wherein the SCA is Spinocerebellar ataxia type 8 (SCAB).

289. The dsRNA, the pharmaceutical composition, the composition, the vector, or the cell of any one of claims 272-288, wherein progression of the trinucleotide repeat expansion disorder is delayed by at least 120 days, for example, at least 6 months, at least 12 months, at least 2 years, at least 3 years, at least 4 years, at least 5 years, at least 10 years or more, when compared with a predicted progression.
